SEQUENCE LISTING (197 sequences)

SEQ ID NO: 1 - DNA, 4254 bp, synthetic construct
  source 1..4254: mol_type = other DNA
ctagcaccgcctatctcgtgtgagataggcggagatacgaactttaagaaggagatataccatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgagatctaactaaaaaggccgctctgcggccttttttcttttcactgtaacaacggaaaccggccattgcgccggttttttttggcct

SEQ ID NO: 2 - DNA, 260 bp, synthetic construct
  source 1..260: mol_type = other DNA
ttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagct

SEQ ID NO: 3 - DNA, 1040 bp, synthetic construct
  source 1..1040: mol_type = other DNA
aacaaacagacaatctggtctgtttgtattatggaaaatttttctgtataatagattcaacaaacagacaatctggtctgtttgtattattctagaattaaagaggagaaattaaccatgagtaaaggagaagaacttttcactggagttgtcccaattcttgttgaattagatggtgatgttaatgggcacaaattttctgttagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttt

SEQ ID NO: 4 - DNA, 1009 bp, synthetic construct
  source 1..1009: mol_type = other DNA
tttacagctagctcagtcctaggtattatgctagcctgaagtacgtctgagcgtgatacccgctcactgaagatggcccggtagggccgaaacgtacctctacaaataattttgtttaatcacacatctagaattaaagaggagaaattaagcatggtttcgaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgactgtgacccaggactcctccttgcaggacggcgagttcatctacaaggtgaagctgcgcggcaccaacttcccctccgacggccccgtaatgcagaagaagacgatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataatactcgaacccctagcccgctcttatcagcttaattagctgagcttggactcctgttgatagatccagtaatgacctcagaactccatctggatttgttcagaacgctcggttgccgccgggcgttttttattggtgagaat

SEQ ID NO: 5 - DNA, 612 bp, synthetic construct
  source 1..612: mol_type = other DNA
atgagcccgaaacgtcgtacccaggcagaacgtgcaatggaaacccagggtaaactgattgcagcagcactgggtgttctgcgtgaaaaaggttatgcaggttttcgtattgcagatgttccgggtgcagccggtgttagccgtggtgcacagagccatcattttccgaccaaactggaactgctgctggcaacctttgaatggctgtatgagcagattaccgaacgtagccgtgcacgtctggcaaaactgaaaccggaagatgatgttattcagcagatgctggatgatgcagcagaattttttctggatgatgattttagcatcagcctggatctgattgttgcagcagatcgtgatccggcactgcgtgaaggtattcagcgtaccgttgaacgtaatcgttttgttgttgaagatatgtggctgggtgtgctggtgagccgtggtctgagccgtgatgatgccgaagatattctgtggctgatttttaacagcgttcgtggtctggcagttcgtagcctgtggcagaaagataaagaacgttttgaacgtgtgcgtaatagcaccctggaaattgcacgtgaacgttatgcaaaattcaaacgttga

SEQ ID NO: 6 - DNA, 539 bp, synthetic construct
  source 1..539: mol_type = other DNA
tttacagctagctcagtcctaggtattatgctagcctgaaggagtcaattaatgtgcttttaattctgatgagacggtgacgtcgaaactccctctacaaataattttgtttaacaggttataaggcctgcagcgtatgtcaaacacgataagcgagaagatagtcttaatgcgaaaatcagagtatttgagcagacaacaacttgctgatttaacaggggttccgtatggcacgctgagttactatgaaagtggtcgttcaacacctccaacagatgtcatgatgaacatcctgcagaccccacaattcaccaaatacactttatggttcatgaccaatcagatcgctcctgagtccgggcaaattgcgcccgctctcgcacactttgggcaaaacgaaacaacgtcgccccactccggtcaaaagactggttaataatcattcttagcgtgaccgggaagtcggtcacgctacctcttctgaagaaacagcaaacaatccaaaacgccgcgttcagcggcgttttttctgcttttct

SEQ ID NO: 7 - DNA, 576 bp, synthetic construct
  source 1..576: mol_type = other DNA
tttacagctagctcagtcctaggtattatgctagcctgactgaagtcgtcaagtgctgtgcttgcacttctgatgaggcagtgatgccgaaacgacctctacaaataattttgtttaattatcttaggtacagggcacataaatgtcaatagacgtttcggagaagttgaagctaatccgtgaatctgaaaggttaaaccgtaaagaattcagtgaattaactggtgtagcctacagctcactttcgagctatgagagccggtcaaaaaacgctggagttgaagccataatgaaggtcttacaacatcctagatttactaaatatactttgtggttcatgactgatcaggtagctccagaagccgggcaaattgcgcccgctctcgcacactttgggcaaaacgaaacaacgtcgccccactccggtcaaaagactggttaataccaccgtcaaaaaaaacggcgctttttagcgccgtttttatttttcaaccttccaggcatcaaataaaacgaaaggctcagtcgaaagactgggcctttcgttttatctgttgtttgtcggtgaacgctctc

SEQ ID NO: 8 - DNA, 582 bp, synthetic construct
  source 1..582: mol_type = other DNA
tttacagctagctcagtcctaggtattatgctagcctgagactgtcgccggatgtgtatccgacctgacgatggcccaaaagggccgaaacagtcctctacaaataattttgtttaatgccacttacattagcgtttttgaatgcagacattcgaaaaactgaaagcgattaggaaagcagaaggcttaacacaggcgaaattcagcgaaattagcgggatagctctaggaacagtcaaaaattacgaaagtgggcataaagaccctggtctgagcatcgttatgcgagtcacaaatacgcctttatttaaaaaatatacgctctggttaatgactggtgatacgtcaccacaagctggtcagatcgcgccggctctcgcacacattgggcaaaaaccaacagaatcagaccactccgaaaaacagactggttaattcagccaaaaaacttaagaccgccggtcttgtccactaccttgcagtaatgcggtggacaggatcggcggttttcttttctcttctcaactcggtaccaaagacgaacaataagacgctgaaaagcgtcttttttcgttttggtcc

SEQ ID NO: 9 - DNA, 816 bp, synthetic construct
  source 1..816: mol_type = other DNA
atgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 10 - DNA, 1119 bp, synthetic construct
  source 1..1119: mol_type = other DNA
atgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccctgcacgcaccattccttgcggcggcggtgctcaacggcctcaacctactactgggctgcttcctaatgcaggagtcgcataagggagagcgccgtccgatgcccttgagagccttcaacccagtcagctccttccggtgggcgcggggcatgaccattgtggccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctga

SEQ ID NO: 11 - DNA, 841 bp, synthetic construct
  source 1..841: mol_type = other DNA
ccttgtcgccttgcgtataatatttgcccatggacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggagagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagat

SEQ ID NO: 12 - DNA, 5687 bp, synthetic construct
  source 1..5687: mol_type = other DNA
gcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgatcccccgatgtgtagcagtggcggaccatataggcagatcagaaggcgcggttctcctacatgttcgccggtgaacgcgttgaggaagccgggcagtgcctcggcaaaatccttgcgtgtagacaagacatctgcgtagcagttgtcctcaacaacgatgtcgaaatccaaatcggagtgctcatcgagtcctccgtgaacgtaagagccgccgatcagaagagcgcggaagcgaacatcggaagcgaccgcatcgcggatgcggttcaagaaagttgcatgagcttgtggaagtgtgctgagcataaatgattctcctagctgttctttgggtaagtacgccatcaggacgttgtgagtggcgcgatttttagcggctgaaatcagcccttgagcctgtcggcaagtcgcgtcatgaggtccatgcgctcatgcaggatcgccacgaccaacgcgggttcgcccgcacgcggcaggcaaaaaacgtagtggtgttcgcagcgggccatccgcagcgcgggaaagagttcgctcatgtccttaaacgggccttcgccggcggcaagcctggctatgccctgttccagcttagcgatatagcggcgcacctgcgccgcgccccactcccggcgcgtgtagcggatgatgccgcgtagatcggcttcggcctcagccgtgaggatgtaggccgtcaagcgcgatccccgctgagttcttcatcaagaatttcgccgacgctcttggtggacaccttgccggcaagcccatcgttgatgcggttccccagcatggttttcagttcctgccatgcctgatcggcatcagcgtcaccggggaacagacgttcgagggcgtattgcttaatggtcttgccctgcaaggcggccagggctttcaggctctggtgctgctggtccgtcatgtcgattgtcaggcggctcattggataacctccataaaatacacgtaaccacattagcacatatgtgggcgtgaggctacagcgcgaggcgcattaaggtcgggaaaatgcgctaggcgcatttaaattgcgtattgctgtaatgcgccatgccggctagactaggcccaaatgggtatacccaatttgaccaagggggacgcgatgagggcggccaagcactaccgacaacttctatccatcgacttcaacatcgaggcgctggccttcgtgcctggacccgacggcacacgcggccggcgcatccacgtcctggggcgcgaggtccgcgaccggcccggcctggtcgagtacctttcgccggcgttcggctcgcgggtggcgctggacggctactgcaaggccaatttcgatgcagtgctgcacctggcgtaccccgatcatcagcaatggggccacgcatgaagcgccgaagctacgccatgctgcgcgccgctgccgcgctggccgtcctggtcgttgcctcgccggcatgggccgagctgcgcggcgaggtcgtgcgcatcatcgacggcgacaccatcgacgtgctggtagacaagcagccggtgcgcgtgcgcctggtggacattgacgcgccggaaaagcggcaagccttcggcgaacgtgcgcgccaggcgctggccggcatggtgttccgccggcacgtcctggtcgacgagaaggacaccgaccgttacggccgcacgctgggcaccgtgtgggtcaacatggagctggccagccggccgccgcagccgcgcaacgtcaacgccgcgatggttcaccagggcatggcgtgggcctatcgcttccacggccgcgcggccgaccctgaaatgctgcggctcgaacaggaggcgcgaggcaagcgcgtcggcctctggtccgatccgcacgccgtcgagccgtggaaatggcgacgcgagagcaacaaccggagggacgaaggttgaaggtcgcccgcatctacctgcgcgccagtacggacgagcagaatcttgaacgccaggagagccttgtagcggccacgcgggccgccgggtactacgtcgccggcatctaccgcgagaaggcgtccggcgcacgcgccgaccggcccgagctgctgcgcatgatcgcggacctgcaacctggtgaagtcgtcgttgcggagaagatcgaccgcatcagccgcttgccgttggccgaggccgagcgcctggttgcgtcgatccgggccaaaggggccaagctggccgtgcctggcgtggtggacctgtcggagctggccgccgaggcgaacggagtggcgaaaatcgttctggaatccgtccaggacatgcttttgaagctcgccttgcagatggcccgcgacgactacgaggatcggcgcgagcgtcaacgtcagggtgtccagttggcgaaggccgccggccgctacaccggccgcaaacgtgacgccggcatgcacgaccgcatcatcacgcttcgctccggcggatcgagcattgccaagacggccaagctggtcggatgcagcccgagccaggtcaaacgagtgtgggcggcctggaacgcgcagcagcaaaaataaagccgggcagtgcccggcttttctcaccttttcgcgtcccgcagggccgctgcgagcgccctacctagatcctcgctttccccctcggtgtagtccgcctaggctgctggatacgctgcttaaggtcatgcagcaggagaactaaaggcccgcggatcccaggcaacgtcttcgtactgcggtaccgggttgcggaagcttttggtcacacgcatgagaaagcccccggaagatcaccttccgggggctcattactcgcatccattctcaggctgtctcgtctcgtctcctcgagattagttagttagcccttagtgactctaatacgactcactgagctcttggggcctctaaacgggtcttgaggggttttttgtactagtagcggccgctcgagccaggatacatagattaccacaactccgagcccttccacccctaggcttctcaatcataggcaatacgatcgcatgtccactagtactgcaggtggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaactgcacgacgatttcctcgtgcatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccggaattccccggatcgatccgtcgatcttgatcccctgcgccatcagatccttggcggcaagaaagccatccagtttactttgcagggcttcccaaccttaccagagggcgccccagctggcaattccggttcgcttgctggccataaaaccgcccagtctagctatcgccatgtaagcccactgcaagctaggtcgtttgtgtttgcgcttgcgttttcccttgtccagatagcccagtacgtgacattcatccggggtcagcaccgtttctgcggactggctttctacgtgttccgcttcctttagcagcccttgcgccctgagtgcttgcggcagcgtgaagctgacggatcgatccggggaattaattcacccccgaacacgagcacggcacccgcgaccactatgccaagaatgcccaaggtaaaaattgcccgccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaagggcgcagccggggggatgggaggcccgcgttagccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcatcactctgcccctcaagtgtcaaggatcgcgcccctcatctgtcagtagtcgcgcccctcaagtgtcaataccgcagggcacttatccccaggcttgtccacatcatgtctgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgtggtatccacaacgccggcggccgcggtgtctcgcacacggcttcgacggcgtttctgctagagatctgtttagcttgcctcgtcccc

SEQ ID NO: 13 - DNA, 779 bp, synthetic construct
  source 1..779: mol_type = other DNA
caccacttcaagaactctgtagcaccgcctacatacctcgctctgctaatcctgttaccagccggttgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacttgagagcttagtacgtgaaacatgagagcttagtacgtactatcaacaggttgaactgcccatgttctttcctgcgttatcagagcttatcggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaagtataccttaacatgttctttcctgcgttatcccctgattctgtggataaccgtattaccgcct

SEQ ID NO: 14 - DNA, 1236 bp, synthetic construct
  source 1..1236: mol_type = other DNA
ctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggagg

SEQ ID NO: 15 - DNA, 436 bp, synthetic construct
  source 1..436: mol_type = other DNA
tgtgtataaggggacactgtatctgcgtcccacaatacaacaaatccgtccctttacaacaacaaatccgtcccttcttaacaacaaatccgtcccttaatggcaacaaatccgtccctttttaaactctacaggccacggattacgtggcctgtagacgtcctaaaaggtttaaaagggaaaaggaagaaaagggtggaaacgcaaaaaacgcaccactacgtggccccgttggggccgcatttgtgcccctgaaggggcgggggaggcgtctgggcaatccccgttttaccagtcccctatcgccgcctgagagggcgcaggaagcgagtaatcagggtatcgaggcggattcacccttggcgtccaaccagcggcaccagcggctcgacaacccttaatataacttcgtataatgtatgctatacgaagttat

SEQ ID NO: 16 - DNA, 357 bp, synthetic construct
  source 1..357: mol_type = other DNA
aaaaaaaaccccgccctgtcaggggcggggttttttttttttcttttgggtatagcgtcgtggacagtcattcatctttctgcccctccaaaagcaaaaacccgccgaagcgggtttttacgtaaatcaggtgaaactgaccgataagccgggacgagctgagcagtatgtcgacggtccggcgaagagctactccccttctgcgccgtccgaagttcctatactttctagagaataggaacttcggaccaaaacgaaaaaacaccctttcgggtgtcttttctggaatttggtaccgagcctttggtcgaaaaaaaaagcccgcactgtcaggtgcgggcttttttctgtgtttcc

SEQ ID NO: 17 - DNA, 352 bp, synthetic construct
  source 1..352: mol_type = other DNA
acatttaataaaaaaagggcggtcgcaagatcgcccttttttacgtatgacacagtgaaaaatggcgcccatcggcgccatttttttatggaagttcctattctctagaaagtataggaacttcagacgagaaacgttccgtccgtctgggtcagttgggcaaagttgatgaccgggtcgtccgttgcagacaaaaaaaatggcgcacaatgtgcgccatttttcacttcacaggtactattgttttgaattgaaaagggcgcttcggcgccctttttgcatttgttgacggcatatatttgtatatcgaagcgccctgatgggcgctttttttatttaatcgataaccaga

SEQ ID NO: 18 - DNA, 335 bp, synthetic construct
  source 1..335: mol_type = other DNA
gataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaggctccttttggagccttttttttt

SEQ ID NO: 19 - DNA, 299 bp, synthetic construct
  source 1..299: mol_type = other DNA
tttacagctagctcagtcctaggtattattactagagaaagaggggaaatactagatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaggctccttttggagccttttttttt

SEQ ID NO: 20 - DNA, 299 bp, synthetic construct
  source 1..299: mol_type = other DNA
tttacggctagctcagccctaggtattattactagagaaagaggggaaatactagatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaggctccttttggagccttttttttt

SEQ ID NO: 21 - DNA, 233 bp, synthetic construct
  source 1..233: mol_type = other DNA
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_difference 47: note = a, c, t, g, unknown or other
  misc_difference 48: note = a, c, t, g, unknown or other
  misc_difference 49: note = a, c, t, g, unknown or other
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 57: note = a, c, t, g, unknown or other
  misc_difference 58: note = a, c, t, g, unknown or other
  misc_difference 59: note = a, c, t, g, unknown or other
  misc_difference 60: note = a, c, t, g, unknown or other
  misc_difference 61: note = a, c, t, g, unknown or other
  misc_difference 62: note = a, c, t, g, unknown or other
ttgacagctagctcagtcctaggtataatactagtggagaccnnnnnnnnnnnnnnnnnnnnggtctagttttagagctagaaatagcaagttaaaataaggctagtccgttatcaacttgaaaaagtggcaccgagtcggtgctagagatcaagccttaacgaactaagacccccgcaccgaaaggtccgggggttttttttgaccttaaaaacataaccgaggagcagaca

SEQ ID NO: 22 - DNA, 222 bp, synthetic construct
  source 1..222: mol_type = other DNA
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_difference 47: note = a, c, t, g, unknown or other
  misc_difference 48: note = a, c, t, g, unknown or other
  misc_difference 49: note = a, c, t, g, unknown or other
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 57: note = a, c, t, g, unknown or other
  misc_difference 58: note = a, c, t, g, unknown or other
  misc_difference 59: note = a, c, t, g, unknown or other
  misc_difference 60: note = a, c, t, g, unknown or other
  misc_difference 61: note = a, c, t, g, unknown or other
  misc_difference 62: note = a, c, t, g, unknown or other
ttgacagctagctcagtcctaggtattgtgctagcggagaccnnnnnnnnnnnnnnnnnnnnggtctcagttgtagatctagaaatagaatgttacaattaggctagtccgttatgaacatgaaaatgtgagaaaagaggccgcgaaagcggccttttttcgtttctcggtaccaaagacgaacaataagacgctgaaaagcgtcttttttcgttttggtcc

SEQ ID NO: 23 - DNA, 192 bp, synthetic construct
  source 1..192: mol_type = other DNA
ttgacagctagctcagtcctaggtattgtgctagcgcgttctgaacaaatccagagttttagagctagaaatagcaagttaaaataaggctagtccgttatcaacttgaaaaagtggcaccgagtcggtgcctcggtaccaaattccagaaaagaggcctcccgaaaggggggccttttttcgttttggtcc

SEQ ID NO: 24 - DNA, 212 bp, synthetic construct
  source 1..212: mol_type = other DNA
ctgacagctagctcagtcctaggtataatgctagcgcgttctgaacaaatccagaggtttagagttagaaataacaagttaaactaaggctagtccgttataaacttgaaaaagtcagtcaaaagcctccggtcggaggcttttgactttcctcggtaccaaattccagaaaagaggcctcccgaaaggggggccttttttcgttttggtcc

SEQ ID NO: 25 - DNA, 212 bp, synthetic construct
  source 1..212: mol_type = other DNA
ctgacagctagctcagtcctaggtataatgctagcgcgttctgaacaaatccagagttgtagatctagaaatagaatgttacaattaggctagtccgttatgaacatgaaaatgtgagaaaagaggccgcgaaagcggccttttttcgtttctcggtaccaaattccagaaaagaggcctcccgaaaggggggccttttttcgttttggtcc

SEQ ID NO: 26 - DNA, 1083 bp, synthetic construct
  source 1..1083: mol_type = other DNA
ttctttcaggccgggtaacaccgtgcgtgttgactattttacctctggcggtgataatagctgtcaccggatgtgctttccggtctgatgagtccgtgaggacgaaacagcctctacaaataattttgtttaatctagaattaaagaggagaaattaaccatgagtaaaggagaagaacttttcactggagttgtcccaattcttgttgaattagatggtgatgttaatgggcacaaattttctgttagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttt

SEQ ID NO: 27 - DNA, 779 bp, synthetic construct
  source 1..779: mol_type = other DNA
tgttgacaattaatcatcggctcgtataatgtgtggaattgtgagcgctcacaattagctgtcaaggcgtgtgcttcgccttctgatgagcccgtgagggcgaaacagcctctacaaataattttgtttaagctggtactgctcgagtatcttatgaagactgacacatcaaatcgcttaaagcagatcatggctgagcgcaaccttaaacaagtggatatcctgaatcttagcatcccttttcaaaaaaaattcgggatcaaattgagcaagtccaccctttcgcaatacgtcaactcggtacaatccccagaccagaaccgtatttacctgttagccaagaccttgggggtaagtgaagcatggttgatgggattcgacgttccaatggtggaatcctcgaagattgagaatgattcagagaacattgaggagacgatcaccgtcatgaagaagctggaagagccgcgtcagaaagtggtcttggatacagcaaaaattcagttaaaggagcaggatgaacagaataaggtaaagcagatcgaggactatcgcttatccgacgaatatttggaggagcagatcagcaaggcgtcggcttacggaggggggcaacttaacgataatgataaggaatttttcaaacgtctgctgaaaaacacccttaaggagaaaattgacaaaggcgatctttgagtaatcgttaatccgcaaataacgtaaaaacccgcttcggcgggtttttttatggggggagtttagggaaagagcatttgtca

SEQ ID NO: 28 - DNA, 958 bp, synthetic construct
  source 1..958: mol_type = other DNA
tgttgacaattaatcatcggctcgtataatgtgtggaattgtgagcgctcacaattcgtaagtcataagtctgggctaagcccactgatgagtcgctgaaatgcgacgaaacttatgacctctacaaataattttgtttaagcactacggaattaagcgctcaaatggttcagaatgaaaaagtgcgcaaagaattcgcccagcggctagcgcaagcctgtaaagaagctggtcttgatgaacatggtaggggaatggctatagcccgtgccctttctctttcgtccaaaggcgttagcaaatggtttaatgctgagtctttaccgcgtcaggaaaaaatgaatgcgcttgcgaaatttctaaacgttgatgttgtttggcttcagcacggcacttcgttaaatggagcgaatgatgaagatactctttcatttgttggcaaattaaaaaaagggttagtgcgcgtggttggtgaggcaattcttggtgttgatggtgccatcgagatgaccgaagaacgcgatgggtggctcaaaatttatagcgatgatccagatgcctttggtctgcgtgtgaaaggagacagcatgtggcccagaataaaatcaggagaatatgtactcattgagcctaacaccaaagtattcccgggtgatgaggtgtttgtcagaaccgttgaaggacacaacatgattaaggttcttggctatgacagagatggagaataccaatttacaagcattaaccaggatcacaggcctataacgttgccttatcatcaagtagcaaaggtggagtatgtagctggtattctgaagcaatctcgccatctggatgacatcgaggcaagggagtggctgaaaagttcgtgagatctaactaaaaaggccgctctgcggccttttttcttttcactgtaacaacggaaaccggccattgcgccggttttttttggcct

SEQ ID NO: 29 - DNA, 597 bp, synthetic construct
  source 1..597: mol_type = other DNA
tgttgacaattaatcatcggctcgtataatgtgtggaattgtgagcgctcacaattcgtactgaagtcgtcaagtgctgtgcttgcacttctgatgaggcagtgatgccgaaacgacctctacaaataattttgtttaattatcttaggtacagggcacataaatgtcaatagacgtttcggagaagttgaagctaatccgtgaatctgaaaggttaaaccgtaaagaattcagtgaattaactggtgtagcctacagctcactttcgagctatgagagccggtcaaaaaacgctggagttgaagccataatgaaggtcttacaacatcctagatttactaaatatactttgtggttcatgactgatcaggtagctccagaagccgggcaaattgcgcccgctctcgcacactttgggcaaaacgaaacaacgtcgccccactccggtcaaaagactggttaataccaccgtcaaaaaaaacggcgctttttagcgccgtttttatttttcaaccttccaggcatcaaataaaacgaaaggctcagtcgaaagactgggcctttcgttttatctgttgtttgtcggtgaacgctctc

SEQ ID NO: 30 - DNA, 561 bp, synthetic construct
  source 1..561: mol_type = other DNA
tgttgacaattaatcatcggctcgtataatgtgtggaattgtgagcgctcacaattcgtaaggagtcaattaatgtgcttttaattctgatgagacggtgacgtcgaaactccctctacaaataattttgtttaatttttctccactacgatggtaatatgtcaaacacgataagcgagaagatagtcttaatgcgaaaatcagagtatttgagcagacaacaacttgctgatttaacaggggttccgtatggcacgctgagttactatgaaagtggtcgttcaacacctccaacagatgtcatgatgaacatcctgcagaccccacaattcaccaaatacactttatggttcatgaccaatcagatcgctcctgagtccgggcaaattgcgcccgctctcgcacactttgggcaaaacgaaacaacgtcgccccactccggtcaaaagactggttaataatcattcttagcgtgaccgggaagtcggtcacgctacctcttctgaagaaacagcaaacaatccaaaacgccgcgttcagcggcgttttttctgcttttct

SEQ ID NO: 31 - DNA, 603 bp, synthetic construct
  source 1..603: mol_type = other DNA
tgttgacaattaatcatcggctcgtataatgtgtggaattgtgagcgctcacaattcgtagactgtcgccggatgtgtatccgacctgacgatggcccaaaagggccgaaacagtcctctacaaataattttgtttaatgccacttacattagcgtttttgaatgcagacattcgaaaaactgaaagcgattaggaaagcagaaggcttaacacaggcgaaattcagcgaaattagcgggatagctctaggaacagtcaaaaattacgaaagtgggcataaagaccctggtctgagcatcgttatgcgagtcacaaatacgcctttatttaaaaaatatacgctctggttaatgactggtgatacgtcaccacaagctggtcagatcgcgccggctctcgcacacattgggcaaaaaccaacagaatcagaccactccgaaaaacagactggttaattcagccaaaaaacttaagaccgccggtcttgtccactaccttgcagtaatgcggtggacaggatcggcggttttcttttctcttctcaactcggtaccaaagacgaacaataagacgctgaaaagcgtcttttttcgttttggtcc

SEQ ID NO: 32 - DNA, 819 bp, synthetic construct
  source 1..819: mol_type = other DNA
ttgacagctagctcagtcctaggtataatgctagcaatggaatattcatagtaaataccaaacggagattcttatgagcccgaaacgtcgtacccaggcagaacgtgcaatggaaacccagggtaaactgattgcagcagcactgggtgttctgcgtgaaaaaggttatgcaggttttcgtattgcagatgttccgggtgcagccggtgttagccgtggtgcacagagccatcattttccgaccaaactggaactgctgctggcaacctttgaatggctgtatgagcagattaccgaacgtagccgtgcacgtctggcaaaactgaaaccggaagatgatgttattcagcagatgctggatgatgcagcagaattttttctggatgatgattttagcatcggcctggatctgattgttgcagcagatcgtgatccggcactgcgtgaaggtattcagcgtaccgttgaacgtaatcgttttgttgttgaagatatgtggctgggtgtgctggtgagccgtggtctgagccgtgatgatgccgaagatattctgtggctgatttttaacagcgttcgtggtctggtagttcgtagcctgtggcagaaagataaagaacgttttgaacgtgtgcgtaatagcaccctggaaattgcacgtgaacgttatgcaaaattcaaacgttgataccaccgtcaaaaaaaacggcgctttttagcgccgtttttatttttcaaccttccaggcatcaaataaaacgaaaggctcagtcgaaagactgggcctttcgttttatctgttgtttgtcggtgaacgctctc

SEQ ID NO: 33 - DNA, 808 bp, synthetic construct
  source 1..808: mol_type = other DNA
ttgacagctagctcagtcctaggtataatgctagcctatggactatgtttgaaagggagaaatactagatggcacgtaccccgagccgtagcagcattggtagcctgcgtagtccgcatacccataaagcaattctgaccagcaccattgaaatcctgaaagaatgtggttatagcggtctgagcattgaaagcgtggcacgtcgcgccggtgcaggcaaaccgaccatttatcgttggtggaccaacaaagcagcactgattgccgaagtgtatgaaaatgaaatcgaacaggtacgtaaatttccggatttgggtagctttaaagccgatctggattttctgctgcataatctgtggaaagtttggcgtgaaaccatttgtggtgaagcatttcgttgtgttattgcagaagcacagttggaccctgtaaccctgacccaactgaaagatcagtttatggaacgtcgtcgtgagataccgaaaaaactggttgaagatgccattagcaatggtgaactgccgaaagatatcaatcgtgaactgctgctggatatgatttttggtttttgttggtatcgcctgctgaccgaacagttgaccgttgaacaggatattgaagaatttaccttcctgctgattaatggtgtttgtccgggtacacagtgttgataataccaccgtcaaaaaaaacggcgctttttagcgccgtttttatttttcaaccttccaggcatcaaataaaacgaaaggctcagtcgaaagactgggcctttcgttttatctgttgtttgtcggtgaacgctctc

SEQ ID NO: 34 - DNA, 43 bp, synthetic construct
  source 1..43: mol_type = other DNA
taacaccgtgcgtgttgactattttacctctggcggtgataat

SEQ ID NO: 35 - DNA, 65 bp, synthetic construct
  source 1..65: mol_type = other DNA
agttcatgaaacgtgaacttgacagctagctcagtcctaggtataaagttcatgaaacgtgaact

SEQ ID NO: 36 - DNA, 68 bp, synthetic construct
  source 1..68: mol_type = other DNA
attgtcagaaatggcacttgcaaaaactttcagttcaaccataatacgtactgaaagtacgaaaaagg

SEQ ID NO: 37 - DNA, 70 bp, synthetic construct
  source 1..70: mol_type = other DNA
aactactacgcaaaattcaacttctccgaaacgtctattgacataattactccgattgcgtaatttcttg

SEQ ID NO: 38 - DNA, 89 bp, synthetic construct
  source 1..89: mol_type = other DNA
atgattggcgactcggtttagatctcattgacatggtgtttagatctcaagtactgttagtttagatgtacaattttgttagtttagat

SEQ ID NO: 39 - DNA, 77 bp, synthetic construct
  source 1..77: mol_type = other DNA
tcctataaggacacgcatttgacagtaacctattggtgacttatattcccgtcaaaaggttgtgtattggtgacctt

SEQ ID NO: 40 - DNA, 90 bp, synthetic construct
  source 1..90: mol_type = other DNA
aacaaacagacaatctggtctgtttgtattatggaaaatttttctgtataatagattcaacaaacagacaatctggtctgtttgtattat

SEQ ID NO: 41 - DNA, 49 bp, synthetic construct
  source 1..49: mol_type = other DNA
ttgacatgatacgaaacgtaccgtatcgttaaggttactagagtctaga

SEQ ID NO: 42 - DNA, 834 bp, synthetic construct
  source 1..834: mol_type = other DNA
gtggcctcacccccaatggtttacaaaagcaatgccctggtcgaggccgcgtatcgcctcagtgttcaggaacagcggatcgttctggcctgtattagccaggtgaagaggagcgagcctgtcaccgatgaagtgatgtattcagtgacggcggaggacatagcgacgatggcgggtgtccctatcgaatcttcctacaaccagctcaaagaagcggccctgcgcctgaaacggcgggaagtccggttaacccaagagcccaatggcaaggggaaaagaccgagtgtgatgattaccggctgggtgcaaacaatcatctaccgggagggtgagggccgtgtagaactcaggttcaccaaagacatgctgccgtacctgacggaactcaccaaacagttcaccaaatacgccttggctgacgtggccaagatggacagcacccacgcgatcaggctttacgagctgctcatgcaatgggacagcatcggccagcgcgaaatagaaattgaccagctgcgaaagtggtttcaactggaaggccggtatccctcgatcaaggacttcaagttgcgagtgcttgatccagccgtgacgcagatcaacgagcacagcccgctacaggtggagtgggcgcagcgaaagaccgggcgcaaggtcacacatctgttgttcagttttggaccgaagaagcccgccaaggcggtgggtaaggccccagcgaagcgcaaggccgggaagatttcagatgctgagatcgcgaaacaggctcgccctggtgagacatgggaagcggcccgcgctcgactaacccagatgccgctggatctggcctag

SEQ ID NO: 43 - DNA, 4231 bp, synthetic construct
  source 1..4231: mol_type = other DNA
ctgcatttacgttgacaccatgcccgtagggttgcggcccttcgggtgactcggcgctgtgcgctatggcgatctggaagtgtcgggacgaggggcgtcacggcggtaaattcgacgtgcctgcggcgcgtcgaacaaggggcgtgcccggtgtcagttcgtggcatttttcgaggcgcgacgccatttccaaggctcctgagcattcgggtctgaccaaaggccgagccgttggcggcgggcctctttttcatactcgtacatctgcgcgtcggttggtttgccgtaataacggtaagcccaggccatgccttcttgaaccatgatcgcattgatgttggtgagttgtgtttggccgccggggtattgcaacggcgcgtaaacgaccccaagagtgcggccataccgatcaacctctttttcggtcacttgaacctcttggcgaaaggtcaagtcggcgagccgttggcgagcacgggagccgaaggcttggccgctttccggtgcgtcaatatcggccaatctcacgcggatggtctgacggttcaccaaaacgtcgatagtgtcaccgtcaaggattcggacgacttcaccccggaagtcggcccaagcgggcacactgacgattaggacgacagcggccgcgaccgcgcgaagggcggcaagggcgcttttcattgtttgcctcctgttttcaagacggctgtgagattggcgacctgctctttgagggcttccacctgaccttgcagactggcggcgcgctcgatggcctccttggcctgtttgcgggcctcgatagcctcgttatcgcgttgggtgagcttttccatgcagcggtttagttcttcgccgctgcggcgtttcacttcggcaagctggtcggccagcttgtcgcgctcgcgttccataggttcgagctgattcactcgttcgcggagctggtcgttttcgcgggtgaaggtgtcggctagttcgattgcttcggcaagctgctggctgatggccgctttgtcggcctcgatctgtttccgatcttcgtcaaaccgggcgttggcgtgcgccagggcgatagcccatagcgcattgccaagctcggcaagatgctcgttgactgcaaccggcaatgggtctgatgagggcagggtggcggtcttgcggtttttccattcagccattgcatcggaaatggttgtgaagctaccgcttccgagtttcttgcgcacggcggccaaagtgggccggatgccttcggcgtccagttcgtcggctgctcgccaaatgtcttgtttagtgattgccattcttgcgggcctctgtactgtagtatgttgtatgatactacatactacaacaatttaacagagccatcttggaatctggtgtctctgcgcctataattctggaacagctactttccgaacgactcctgcgttgatcggaaatccagaagcccgagaggttgccgcctttcgggctttttctttttcaaaaaaaaaaatttataaaacgatctgttgcggccgccgggttgtgggcaaaggcgctcgacggtgggcaaccgcttgcggttgtccacgggcggagccggtgcgcgtagcgcattgtccacaagccaagggcgaccaataattgatatatatattcataattgaaaagctaattgaacatactacttgctgtaactacttgccggagcgaggggtgtttgcaagctgttgatctgaaagggctattagcgttctcacgtgcctttttgattagcgatttcacgtgaccttattagcgatttcacgtactccgattagcgatttcacgtaccctgattagcgatttcacgtggatagtttttggagcgggccggaaagccccgtgaatcaaggctttgcggggcattagcggtttcacgtggataactaccctctatccacaggcttccggggataaaaaagcccgctcgacggcgggctgttggatgggaaggcttgaccaagccaagcgtagcgttggcctggtcaagtcggaggggggccgatgcgagcgcccttgccgggtgcgcgggtgacatgcaggcgtgtggatttgatgcgcaggcattcgccgtcatcttcgatgcagtcgcttgcctcgggatagacaatcaacacttcgcgtaggcgctttttgaagttgtatttgaagctggcgagtgctgcccgctctgcccgctctcgggccttatcgtccagttcgggcgagttgcgtgcgcggctgccataggatgagccgaattgcgcttgcagggcgacccaagggatttgcacgaaggggcggcccttggcccgcaacaggaacacgcgataggtcagccacgtgtaaatgtccatcgcaagcggagactgccgcaaggcatgcaggtagtcgattcggataggaaccggtgagcgggtgacttcctcgaagaaatcgcctgtgagggtgagggtgctatcccatagcgcccgatcttctggccgcttgggattccagaatagaaaagcgcgcttggcaatgacgacgttctcaatgccgaagtcattgccttgctcgccggcaagcgaaatcatggatgaaaacaggcgttgcgcctgattgcgaagggtggccgtgtaacggccatcggtgtgcattccgagcctttgtagaaattccgattgcgaccggccaaggttcaacacggggtctttcgttcgcacggcctcggtgcatatccaagcaagcaaggtgcgcggcatagaaccgtagggcaggccgatgctcggcttgcccatgatcgacaaggtgacgatgccattggtgcgctcaaagtagctggtcttggggtcggtgtggggcatggtcgcttgcacaaggcaacgggccatgtagccgactaagccagcttcgcgggcatcctccatttcgagcgcgaggctcgtcttgatgatctcgttgatacgatggccgggggctttgttgttcttaggcatgttgttccctccccggcatggtgatggttggtctagtgtttgtgggtttgatgttccggcgtttgatgaacaggcgcaaggtgtgagggctgacgcctaacaactcggctgcgcgactttgcggcaagccaaggttcacgtatgcctgtacttcatcaatacggctgtccagcttcaaggcgctcgatttgctgcccttgggtcgcccgagcgtcttgccgcgctctctggcgacttgtagcgcctcggtggtacgtgcctgaatgaaatgccgctcgatctgtgcagccaagccaagcacggttgccatgatgtcgctttgtaggctgccgtccatgatgatcttctgtttggtcacatggacgattaggccgcgctcgctcgccgctttgagaatttccaaggcggcgagggcggaaccggcaatgcgcgtaatctccggcgtcagtagcacgtcgccacgctcggccttttcgatgattgctccgagcttgcgcttgcgccagtcctttgctctgctggcaatttcttcctcgatctgtagcggcgcgaagcctttggcgttcgcgtattcgagcaaaccgtatttttggttttccgggtcttggccgtcacgcgaaacccggagataggcatagtattttggcatttgcagggaaaacgtcagattcggttaaacatgcctcattctagcgcagattaaataggaattaaataccctgttgcggtatagataaaacgttggtttgcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaacctaggagtgcggttggaacgttggcccagccagatactcccgatcacgagcaggacgccga

SEQ ID NO: 44 - DNA, 708 bp, synthetic construct
  source 1..708: mol_type = other DNA
gtcagcaaaggcgaggaagacaatatggcttcgctcccggctacccacgaactccacatctttgggagcatcaatggcgtggactttgacatggtggggcaagggaccggtaaccccaacgacggctacgaggaactcaatttgaagtccacgaaaggcgatctccaatttagcccctggattttggtgccacacatcggctacgggtttcatcaatacttgccatatcctgatggtatgagcccttttcaagcggctatggtggatggttccggttatcaggtccaccgcaccatgcaatttgaagatggtgcgtccttgacggtgaattatcggtatacgtatgaaggtagccacatcaaaggcgaggcgcaagtgaaggggacgggttttcccgcagatggcccagtgatgaccaatagcctgacggccgctgattggtgtcggtccaaaaaaacctatcctaatgacaagacgatcatctcgacctttaagtggtcgtatacgaccggtaacgggaagcgctatcgctcgacggcccgtaccacctatacgtttgccaaacctatggcggccaattacctgaaaaaccagcctatgtacgtcttccgtaaaaccgaactgaaacactcgaaaaccgagttgaacttcaaggaatggcagaaagccttcacggacgtgatgggcatggacgagttgtacaaataa

SEQ ID NO: 45 - DNA, 1146 bp, synthetic construct
  source 1..1146: mol_type = other DNA
atgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgc

SEQ ID NO: 46 - protein, 382 aa, synthetic construct
MNRTFDRKAYRQELIDAGFSAEDAETIASRTVMRAPRETFQSVGSMVQQATAKIERDSVQLAPPALPAPSAAVERSRRLEQEAAGLAKSMTIDTRGTMTTKKRKTAGEDLAKQVSEAKQAALLKHTKQQIKEMQLSLFDIAPWPDTMRAMPNDTARSALFTTRNKKIPREALQNKVIFHVNKDVKITYTGVELRADDDELVWQQVLEYAKRTPIGEPITFTFYELCQDLGWSINGRYYTKAEECLSRLQATAMGFTSDRVGHLESVSLLHRFRVLDRGKKTSRCQVLIDEEIVVLFAGDHYTKFIWEKYRKLSPTARRMFDYFSSHREPYPLKLETFRLMCGSDSTRVKKWREQVGEACEELRGSGLVEHAWVNDDLVHCKR

SEQ ID NO: 47 - DNA, 1630 bp, synthetic construct
  source 1..1630: mol_type = other DNA
gcccggcgacatcttttgttacccgccaaacaaaacccaaaaacaacccatacccaacccaataaaacaccaaaacaagacaaataatcattgattgatggttgaaatggggtaaacttgacaaacaaacccacttaaaacccaaaacatacccaaacacacaccaaaaaaacaccataaggagttttataaatgttggtattcattgatgacggttcaacaaacatcaaactacagtggcaggaaagcgacggaacaattaaacagcacattagcccgaacagcttcaaacgcgagtgggcagtctcttttggtgataaaaaggtctttaactacacactgaacggcgaacagtattcatttgatccaatcagcccggatgctgtagtcacaaccaatatcgcatggcaatacagcgacgttaatgtcgttgcagtgcatcacgccttactgaccagtggtctgccggtaagcgaagtggatattgtttgcacacttcctctgacagagtattacgacagaaataaccaacccaatacggaaaatattgagcgtaagaaagcaaacttccggaaaaaaattacattaaatggcggggatacattcacaataaaagatgtaaaagtcatgcctgaatctataccggcaggttatgaagttctacaagaactggatgagttagattctttattaattatagatctcgggggcaccacattagatatttctcaggtaatggggaaattatcggggatcagtaaaatatacggagactcatctcttggtgtctctctggttacatctgcagtaaaagatgccctttctcttgcgagaacaaaaggaagtagctatcttgctgacgatataatcattcacagaaaagataataactatctgaagcaacgaattaatgatgagaacaaaatatcaatagtcaccgaagcaatgaatgaagcacttcgtaaacttgagcaacgtgtattaaatacgctcaatgaattttctggttatactcatgttatggttataggcggtggcgcagaattaatatgcgatgcagtaaaaaaacacacacagattcgtgatgaacgttttttcaaaaccaataactctcaatatgatttagttaacggtatgtatctcataggtaattaatgatggacaagcgcagaaccattgccttcaaactaaatccagatgtaaatcaaacagataaaattgtttgtgatacactggacagtatcccgcaaggggaacgaagccgccttaaccgggccgcactgacggcaggtctggccttatacagacaagatccccggacccctttccttttatgtgagctgctgacgaaagaaaccacattttcagatatcgtgaatatattgagatcgctatttccaaaagagatggccgattttaattcttcaatagtcactcaatcctcttcacaacaagagcaaaaaagtgatgaagagaccaaaaaaaatgcgatgaagctaataaattaattcaattattattgagttccctttatccactatcaggctggataaagggaactcaatcaagttattttcttaccagtcattacataatcgttattatgaaataatcgtttgcactgtctctg

SEQ ID NO: 48 - DNA, 888 bp, synthetic construct
  source 1..888: mol_type = other DNA
agatcccgttcatcaagatgaaaatagcgcgccagttgcacgtcattaggttatgcagcataacaaccgtaattctgtttttggtcagaggttagaaagtcgacaggcatatcaggctccctacctgacagttattcattaacaattttgcaaccgtccgaaatattataaattatcgcacacataaaaacagtgctgttaatgtgtctattaaatcgattttttgttataacagacactgcttgtccgatatttgatttaggatacatttttatgcgactttttggttacgcacgggtatcaaccagccagcaatctctcgatattcaggttcgggcactcaaagacgcaggcgtgaaagcaaatcgcatctttactgacaaggcatcgggcagttcaagcgatcggaaagggctggacttgctgaggatgaaggtggaggaaggtgacgtcatcttggtgaagaaacttgaccgccttgggcgcgatactgctgacatgatccagttaataaaagagtttgacgcccaaggtgtatccattcggtttattgatgacggaatcagtaccgatggggagatgggtaaaatggttgtcactattctatctgcggtggcccaggcagaacgacagagaatactagagcgtaccaatgaaggtcgccaagaggcaatggcaaaaggagttgtttttggtagaaaaagaaaaatagatagagatgcagtattaaatatgtggcaacaggggttaggtgcctcacatatatcaaaaacaatgaatattgctcgttcaacagtatataaagtaataaatgaaagcaactaacataaaaggttgaatatggaattgaatattacttcaaagtctaatccgtttggtgatacgaca

SEQ ID NO: 49 - DNA, 918 bp, synthetic construct
  source 1..918: mol_type = other DNA
atgagactcaaggtcatgatggacgtgaacaaaaaaacgaaaattcgccaccgaaacgagctaaatcacaccctggctcaacttcctttgcccgcaaagcgagtgatgtatatggcgcttgctcccattgatagcaaggaacctcttgaacgagggcgagttttcaaaattagggctgaagaccttgcagcgctcgccaaaatcaccccatcgcttgcttatcgacaattaaaagagggtggtaagttacttggtgccagcaaaatttcgctaagaggggatgatatcattgcttcagctaaagagcttaacctgctctttactgctaaagactcccctgaagagttagatcttaacattattgagtggatagcttattcaaatgatgaaggatacttgtctttaaaattcaccagaaccatagaaccatatatctctagccttattgggaaaaaaaataaattcacaacgcaattgttaacggcaagcttacgcttaagtagccagtattcatcttctctttatcaacttatcaggaagcattactctaattttaagaagaaaaattattttattatttccgttgatgagttaaaggaagagttaatagcttatacttttgataaagatggaagtattgagtacaaataccctgactttcctatttttaaaagggatgtattaaataaagccattgctgaaattaaaaagaaaacagaaatatcgtttgttggctttactgttcatgaaaaagaaggaagaaaaattagtaagctgaagttcgaatttgtcgttgatgaagatgaattttctggcgataaagatgatgaagctttttttatgaatttatctgaagctaatgcagcttttctcaaggtatttgatgaaaccgtacctcccaaaaaagctaaggggtga

SEQ ID NO: 50 - DNA, 20 bp, synthetic construct
  source 1..20: mol_type = other DNA
gtctccccaatgcatactat

SEQ ID NO: 51 - DNA, 20 bp, synthetic construct
  source 1..20: mol_type = other DNA
gctgcttcctagactactat

SEQ ID NO: 52 - DNA, 20 bp, synthetic construct
  source 1..20: mol_type = other DNA
atcctagtcctaggaattcc

SEQ ID NO: 53 - DNA, 20 bp, synthetic construct
  source 1..20: mol_type = other DNA
cggaagatcccgccggcgaa

SEQ ID NO: 54 - DNA, 20 bp, synthetic construct
  source 1..20: mol_type = other DNA
ggcgattaatcttgtcgatc

SEQ ID NO: 55 - DNA, 20 bp, synthetic construct
  source 1..20: mol_type = other DNA
gtgccccgcgtataatatgt

SEQ ID NO: 56 - DNA, 20 bp, synthetic construct
  source 1..20: mol_type = other DNA
ggctggagcttactcactaa

SEQ ID NO: 57 - DNA, 20 bp, synthetic construct
  source 1..20: mol_type = other DNA
acatattatacgcggggcac

SEQ ID NO: 58 - DNA, 20 bp, synthetic construct
  source 1..20: mol_type = other DNA
gtctgggtactatataggag

SEQ ID NO: 59 - DNA, 20 bp, synthetic construct
  source 1..20: mol_type = other DNA
ctcctatatagtacccagac

SEQ ID NO: 60 - DNA, 20 bp, synthetic construct
  source 1..20: mol_type = other DNA
ggcctacgggtaggtatatc

SEQ ID NO: 61 - DNA, 20 bp, synthetic construct
  source 1..20: mol_type = other DNA
ttaagagtccacccaatgac

SEQ ID NO: 62 - DNA, 20 bp, synthetic construct
  source 1..20: mol_type = other DNA
gctaggcgagtcgttaattc

SEQ ID NO: 63 - DNA, 20 bp, synthetic construct
  source 1..20: mol_type = other DNA
ctcttagggacactactctc

SEQ ID NO: 64 - DNA, 20 bp, synthetic construct
  source 1..20: mol_type = other DNA
aatggaacttaccaatgacg

SEQ ID NO: 65 - DNA, 20 bp, synthetic construct
  source 1..20: mol_type = other DNA
cttcaggaatgctcgccttg

SEQ ID NO: 66 - DNA, 20 bp, synthetic construct
  source 1..20: mol_type = other DNA
cgagctgagcagtatgtcga

SEQ ID NO: 67 - DNA, 20 bp, synthetic construct
  source 1..20: mol_type = other DNA
ggaactttggcagcagtgcg

SEQ ID NO: 68 - DNA, 20 bp, synthetic construct
  source 1..20: mol_type = other DNA
ccaagcccccctcgtcctgg

SEQ ID NO: 69 - DNA, 20 bp, synthetic construct
  source 1..20: mol_type = other DNA
gagcctgcgcaaacagtacc

SEQ ID NO: 70 - DNA, 20 bp, synthetic construct
  source 1..20: mol_type = other DNA
ggaactttggcagcagtgcg

SEQ ID NO: 71 - DNA, 20 bp, synthetic construct
  source 1..20: mol_type = other DNA
tcgccctgggcattggtcag

SEQ ID NO: 72 - DNA, 20 bp, synthetic construct
  source 1..20: mol_type = other DNA
gacgaccttgatcaccgcgt

SEQ ID NO: 73 - DNA, 20 bp, synthetic construct
  source 1..20: mol_type = other DNA
gaactccacgccatcgacgt

SEQ ID NO: 74 - DNA, 20 bp, synthetic construct
  source 1..20: mol_type = other DNA
atcacactttgatggatgct

SEQ ID NO: 75 - DNA, 20 bp, synthetic construct
  source 1..20: mol_type = other DNA
gacctggatgatttccttgg

SEQ ID NO: 76 - DNA, 20 bp, synthetic construct
  source 1..20: mol_type = other DNA
gtgacacgtcactcaataac

SEQ ID NO: 77 - DNA, 20 bp, synthetic construct
  source 1..20: mol_type = other DNA
gtgcgtactaggtaaatcga

SEQ ID NO: 78 - DNA, 20 bp, synthetic construct
  source 1..20: mol_type = other DNA
gatggtaacgctcaacccat

SEQ ID NO: 79 - DNA, 20 bp, synthetic construct
  source 1..20: mol_type = other DNA
gccgacctgatcgtgcgccc

SEQ ID NO: 80 - DNA, 20 bp, synthetic construct
  source 1..20: mol_type = other DNA
cttctgcgccgcctgaatcg

SEQ ID NO: 81 - DNA, 20 bp, synthetic construct
  source 1..20: mol_type = other DNA
caatgtggcctacatcctga

SEQ ID NO: 82 - DNA, 20 bp, synthetic construct
  source 1..20: mol_type = other DNA
gaacctaacagctacttggt

SEQ ID NO: 83 - DNA, 20 bp, synthetic construct
  source 1..20: mol_type = other DNA
gatgcatcatagctagcaag

SEQ ID NO: 84 - DNA, 20 bp, synthetic construct
  source 1..20: mol_type = other DNA
tcatcacggaacgttttacc

SEQ ID NO: 85 - DNA, 20 bp, synthetic construct
  source 1..20: mol_type = other DNA
gacgtggtggaagccaccga

SEQ ID NO: 86 - DNA, 20 bp, synthetic construct
  source 1..20: mol_type = other DNA
ggaagtgatggccaaacggc

SEQ ID NO: 87 - DNA, 20 bp, synthetic construct
  source 1..20: mol_type = other DNA
ggacctgcccaacgattacc

SEQ ID NO: 88 - DNA, 20 bp, synthetic construct
  source 1..20: mol_type = other DNA
gcccgagccggcccccgcag

SEQ ID NO: 89 - DNA, 20 bp, synthetic construct
  source 1..20: mol_type = other DNA
accacctgcagcagcacgtc

SEQ ID NO: 90 - DNA, 20 bp, synthetic construct
  source 1..20: mol_type = other DNA
gggagtgctataattctgag

SEQ ID NO: 91 - DNA, 20 bp, synthetic construct
  source 1..20: mol_type = other DNA
gcgcaagttaatactagagc

SEQ ID NO: 92 - DNA, 20 bp, synthetic construct
  source 1..20: mol_type = other DNA
aggtagcttaaccttcggga

SEQ ID NO: 93 - DNA, 20 bp, synthetic construct
  source 1..20: mol_type = other DNA
gcgtcatcgtgttccaggaa

SEQ ID NO: 94 - DNA, 20 bp, synthetic construct
  source 1..20: mol_type = other DNA
tgactgtccacgacagaacc

SEQ ID NO: 95 - DNA, 20 bp, synthetic construct
  source 1..20: mol_type = other DNA
gatgtggatcatcttggtgc

SEQ ID NO: 96 - DNA, 20 bp, synthetic construct
  source 1..20: mol_type = other DNA
gacctgtccgggtatctgta

SEQ ID NO: 97 - DNA, 20 bp, synthetic construct
  source 1..20: mol_type = other DNA
gatccccgtcacttttatag

SEQ ID NO: 98 - DNA, 20 bp, synthetic construct
  source 1..20: mol_type = other DNA
accgacctcacccttatcag

SEQ ID NO: 99 - DNA, 20 bp, synthetic construct
  source 1..20: mol_type = other DNA
gtttttggtaccgcgatccc

SEQ ID NO: 100 - DNA, 20 bp, synthetic construct
  source 1..20: mol_type = other DNA
cttacaggacctaatatggg

SEQ ID NO: 101 - DNA, 20 bp, synthetic construct
  source 1..20: mol_type = other DNA
gatcttgcaggatttgctgt

SEQ ID NO: 102 - DNA, 20 bp, synthetic construct
  source 1..20: mol_type = other DNA
ggccatgcttatgaagctaa

SEQ ID NO: 103 - DNA, 20 bp, synthetic construct
  source 1..20: mol_type = other DNA
cgttaactgaataccctcgg

SEQ ID NO: 104 - DNA, 20 bp, synthetic construct
  source 1..20: mol_type = other DNA
gttctggcatgggaaagttt

SEQ ID NO: 105 - DNA, 20 bp, synthetic construct
  source 1..20: mol_type = other DNA
agcgttaactgaataccctc

SEQ ID NO: 106 - DNA, 20 bp, synthetic construct
  source 1..20: mol_type = other DNA
gttcttggttggggagtagg

SEQ ID NO: 107 - DNA, 20 bp, synthetic construct
  source 1..20: mol_type = other DNA
gagcgttaactgaataccct

SEQ ID NO: 108 - DNA, 20 bp, synthetic construct
  source 1..20: mol_type = other DNA
gctccttcttggtatcatcc

SEQ ID NO: 109 - DNA, 20 bp, synthetic construct
  source 1..20: mol_type = other DNA
gcgttaactgaataccctcg

SEQ ID NO: 110 - DNA, 20 bp, synthetic construct
  source 1..20: mol_type = other DNA
ggaaggtgtagagactggac

SEQ ID NO: 111 - DNA, 17 bp, synthetic construct
  source 1..17: mol_type = other DNA
cgttaactgaataccct

SEQ ID NO: 112 - DNA, 20 bp, synthetic construct
  source 1..20: mol_type = other DNA
gagcgttaactgaataccct

SEQ ID NO: 113 - DNA, 20 bp, synthetic construct
  source 1..20: mol_type = other DNA
gcgttaactgaataccctcg

SEQ ID NO: 114 - DNA, 17 bp, synthetic construct
  source 1..17: mol_type = other DNA
cttacaggacctaatat

SEQ ID NO: 115 - DNA, 20 bp, synthetic construct
  source 1..20: mol_type = other DNA
gttctggcatgggaaagttt

SEQ ID NO: 116 - DNA, 20 bp, synthetic construct
  source 1..20: mol_type = other DNA
accttcgccaacttcgtcga

SEQ ID NO: 117 - DNA, 20 bp, synthetic construct
  source 1..20: mol_type = other DNA
caagacccacctgatgttcg

SEQ ID NO: 118 - DNA, 20 bp, synthetic construct
  source 1..20: mol_type = other DNA
tgaaggacctgctgtccaag

SEQ ID NO: 119 - DNA, 20 bp, synthetic construct
  source 1..20: mol_type = other DNA
tacgggttcggtcctccaat

SEQ ID NO: 120 - DNA, 20 bp, synthetic construct
  source 1..20: mol_type = other DNA
tacggggctatcacccactt

SEQ ID NO: 121 - DNA, 20 bp, synthetic construct
  source 1..20: mol_type = other DNA
acaagcgagcttaagccgtt

SEQ ID NO: 122 - DNA, 20 bp, synthetic construct
  source 1..20: mol_type = other DNA
tgatatgctgcgataagctg

SEQ ID NO: 123 - DNA, 20 bp, synthetic construct
  source 1..20: mol_type = other DNA
gaatgcctacaaacagtcgg

SEQ ID NO: 124 - DNA, 20 bp, synthetic construct
  source 1..20: mol_type = other DNA
tgtaccgggacaccgcacat

SEQ ID NO: 125 - DNA, 20 bp, synthetic construct
  source 1..20: mol_type = other DNA
tctcgtttagcacttggacg

SEQ ID NO: 126 - DNA, 20 bp, synthetic construct
  source 1..20: mol_type = other DNA
attacctcgcacacgtgcta

SEQ ID NO: 127 - DNA, 20 bp, synthetic construct
  source 1..20: mol_type = other DNA
atgtaccgggggttacaaga

SEQ ID NO: 128 - DNA, 20 bp, synthetic construct
  source 1..20: mol_type = other DNA
gtagtccgtcttgtaacccc

SEQ ID NO: 129 - DNA, 20 bp, synthetic construct
  source 1..20: mol_type = other DNA
cgtatcgtgatgctcctcct

SEQ ID NO: 130 - DNA, 20 bp, synthetic construct
  source 1..20: mol_type = other DNA
aaatctggcaacttaccgta

SEQ ID NO: 131 - DNA, 20 bp, synthetic construct
  source 1..20: mol_type = other DNA
cccttctatgttatgcgttg

SEQ ID NO: 132 - DNA, 20 bp, synthetic construct
  source 1..20: mol_type = other DNA
aatgctgataacgaccaacc

SEQ ID NO: 133 - DNA, 20 bp, synthetic construct
  source 1..20: mol_type = other DNA
cgttctcatcgttctactga

SEQ ID NO: 134 - DNA, 20 bp, synthetic construct
  source 1..20: mol_type = other DNA
ctttgcgccaagagatcgcg

SEQ ID NO: 135 - DNA, 20 bp, synthetic construct
  source 1..20: mol_type = other DNA
tacagtaacgatacgattac

SEQ ID NO: 136 - DNA, 60 bp, synthetic construct
  source 1..60: mol_type = other DNA
caaaaaaaaaggctccaaaaggagcctcaacgtttgaattttgcataacgttcacgtgca

SEQ ID NO: 137 - DNA, 55 bp, synthetic construct
  source 1..55: mol_type = other DNA
agccctctttttgataaattttctcgaggtgtacctgcaattcgcgcgcgaaggc

SEQ ID NO: 138 - DNA, 57 bp, synthetic construct
  source 1..57: mol_type = other DNA
gagtcagctaggaggtgactgacaaataaaacgaaaggctcagtcgaaagactgggc

SEQ ID NO: 139 - DNA, 59 bp, synthetic construct
  source 1..59: mol_type = other DNA
agctagctgtcaaagatctttagaattccagaattcaaaaggccatccgtcaggatggc

SEQ ID NO: 140 - DNA, 59 bp, synthetic construct
  source 1..59: mol_type = other DNA
tggaattctaaagatctttgacagctagctcagtcctaggtataatactagtggagacc

SEQ ID NO: 141 - DNA, 59 bp, synthetic construct; identical to SEQ ID NO: 140
  source 1..59: mol_type = other DNA

SEQ ID NO: 142 - DNA, 90 bp, synthetic construct
  source 1..90: mol_type = other DNA
tgtctgctcctcggttatgtttttaaggtcaaaaaaaacccccggacctttcggtgcgggggtcttagttcgttaaggcttgatctctag

SEQ ID NO: 143 - DNA, 47 bp, synthetic construct
  source 1..47: mol_type = other DNA
agagcttcaatttaattatatcagttattcacgtggagcttatcggc

SEQ ID NO: 144 - DNA, 42 bp, synthetic construct
  source 1..42: mol_type = other DNA
acctcgagaaaatttatcaaaaagagggctaaacaaacagac

SEQ ID NO: 145 - DNA, 60 bp, synthetic construct
  source 1..60: mol_type = other DNA
atctccgcctatctcataatacaaacagaccagattgtctgtttgttgaatctattatac

SEQ ID NO: 146 - DNA, 31 bp, synthetic construct
  source 1..31: mol_type = other DNA
ctagagatctgtttagcttgcctcgtccccg

SEQ ID NO: 147 - DNA, 31 bp, synthetic construct; identical to SEQ ID NO: 146
  source 1..31: mol_type = other DNA

SEQ ID NO: 148 - DNA, 41 bp, synthetic construct
  source 1..41: mol_type = other DNA
tgtctgctcctcggttatgtttttaaggtcaaaaaaaaccc

SEQ ID NO: 149 - DNA, 41 bp, synthetic construct; identical to SEQ ID NO: 148
  source 1..41: mol_type = other DNA

SEQ ID NO: 150 - DNA, 60 bp, synthetic construct
  source 1..60: mol_type = other DNA
gaccttaaaaacataaccgaggagcagacagcgcaacgcaattaatgtgagttagctcac

SEQ ID NO: 151 - DNA, 54 bp, synthetic construct
  source 1..54: mol_type = other DNA
ggggacgaggcaagctaaacagatctctagcagaaacgccgtcgaagccgtgtg

SEQ ID NO: 152 - DNA, 60 bp, synthetic construct
  source 1..60: mol_type = other DNA
tgcgaggctggccgataagctccacgtgaataactgatataattaaattgaagctctggg

SEQ ID NO: 153 - DNA, 56 bp, synthetic construct
  source 1..56: mol_type = other DNA
aaggtctcgaagggctcggcccttccggctggctggtttattgctgataaatctgg

SEQ ID NO: 154 - DNA, 33 bp, synthetic construct
  source 1..33: mol_type = other DNA
ggtctcgtgatttcctgggtgtcgtcgtcaagc

SEQ ID NO: 155 - DNA, 60 bp, synthetic construct
  source 1..60: mol_type = other DNA
agccggaagggccgagcccttcgagaccttgtgtctcaaaatctctgatgtacattgcac

SEQ ID NO: 156 - DNA, 60 bp, synthetic construct
  source 1..60: mol_type = other DNA
tgacgacgacacccaggaaatcacgagacctaattggtaacgaatcagacaattgacggc

SEQ ID NO: 157 - DNA, 90 bp, synthetic construct
  source 1..90: mol_type = other DNA
ggctacggtctcgatcacgcgcggcacggtcaagctgctgcgcgagttcctggacgagaagggccgcgatcccaacaccgtcgatgcctt

SEQ ID NO: 158 - DNA, 69 bp, synthetic construct
  source 1..69: mol_type = other DNA
ggctacggtctcagagcgtcccgtcggtcgccgtcgccgcctgtggcgttgagggtggttctggctgcg

SEQ ID NO: 159 - DNA, 60 bp, synthetic construct
  source 1..60: mol_type = other DNA
accgtgccgcgcgtgatcgagaccgtagccttgtgtctcaaaatctctgatgtacattgc

SEQ ID NO: 160 - DNA, 60 bp, synthetic construct
  source 1..60: mol_type = other DNA
cgaccgacgggacgctctgagaccgtagcctaattggtaacgaatcagacaattgacggc

SEQ ID NO: 161 - DNA, 50 bp, synthetic construct
  source 1..50: mol_type = other DNA
ggctacggtctcagctcgcgccgtcagcgccggcggaaccgtcgcactcc

SEQ ID NO: 162 - DNA, 42 bp, synthetic construct
  source 1..42: mol_type = other DNA
ggctacggtctctggtgcttcttgccgtccatgatggggtgc

SEQ ID NO: 163 - DNA, 60 bp, synthetic construct
  source 1..60: mol_type = other DNA
gcgctgacggcgcgagctgagaccgtagccttgtgtctcaaaatctctgatgtacattgc

SEQ ID NO: 164 - DNA, 60 bp, synthetic construct
  source 1..60: mol_type = other DNA
ggacggcaagaagcaccagagaccgtagcctaattggtaacgaatcagacaattgacggc

SEQ ID NO: 165 - DNA, 31 bp, synthetic construct
  source 1..31: mol_type = other DNA
ggctacggtctctcaccgcatcgagaccgag

SEQ ID NO: 166 - DNA, 35 bp, synthetic construct
  source 1..35: mol_type = other DNA
ggctacggtctcaactgctgaacatccccatgccg

SEQ ID NO: 167 - DNA, 60 bp, synthetic construct
  source 1..60: mol_type = other DNA
tccgtctcgatgcggtgagagaccgtagccttgtgtctcaaaatctctgatgtacattgc

SEQ ID NO: 168 - DNA, 60 bp, synthetic construct
  source 1..60: mol_type = other DNA
tggggatgttcagcagttgagaccgtagcctaattggtaacgaatcagacaattgacggc

SEQ ID NO: 169 - DNA, 31 bp, synthetic construct
  source 1..31: mol_type = other DNA
ggctacggtctcacagtgcgcagccggacag

SEQ ID NO: 170 - DNA, 42 bp, synthetic construct
  source 1..42: mol_type = other DNA
ggctacggtctcgtctacgaggtagacaagctcgggcagacg

SEQ ID NO: 171 - DNA, 60 bp, synthetic construct
  source 1..60: mol_type = other DNA
cttgtctacctcgtagacgagaccgtagcctaattggtaacgaatcagacaattgacggc

SEQ ID NO: 172 - DNA, 60 bp, synthetic construct; identical to SEQ ID NO: 171
  source 1..60: mol_type = other DNA

SEQ ID NO: 173 - DNA, 35 bp, synthetic construct
  source 1..35: mol_type = other DNA
ggctacggtctcgtagacatagggcacgaacttcg

SEQ ID NO: 174 - DNA, 55 bp, synthetic construct
  source 1..55: mol_type = other DNA
ggtctcgtgtagttgcgtcgcggtgcatggagccgggccacctcgacctgaatgg

SEQ ID NO: 175 - DNA, 60 bp, synthetic construct
  source 1..60: mol_type = other DNA
ttcgtgccctatgtctacgagaccgtagccttgtgtctcaaaatctctgatgtacattgc

SEQ ID NO: 176 - DNA, 60 bp, synthetic construct
  source 1..60: mol_type = other DNA
ccatgcaccgcgacgcaactacacgagacctaattggtaacgaatcagacaattgacggc

SEQ ID NO: 177 - DNA, 51 bp, synthetic construct
  source 1..51: mol_type = other DNA
cgcggccgcttctagagggtctcgtagacatagggcacgaacttcgactgg

SEQ ID NO: 178 - DNA, 50 bp, synthetic construct
  source 1..50: mol_type = other DNA
gctactagtaggtctcgtttgttgatctgttgatggcctggaacaccggg

SEQ ID NO: 179 - DNA, 43 bp, synthetic construct
  source 1..43: mol_type = other DNA
gcttctagagggtctcgcaaagggcttcggggccacgtacctc

SEQ ID NO: 180 - DNA, 44 bp, synthetic construct
  source 1..44: mol_type = other DNA
cgctactagtaggtctcgtgtagttgcgtcgcggtgcatggagc

SEQ ID NO: 181 - DNA, 18 bp, synthetic construct
  source 1..18: mol_type = other DNA
tcatggctctgccctcgg

SEQ ID NO: 182 - DNA, 20 bp, synthetic construct
  source 1..20: mol_type = other DNA
tgggttgaaggtgaagccgg

SEQ ID NO: 183 - DNA, 82 bp, synthetic construct
  source 1..82: mol_type = other DNA
cctggcggcgttgtgacaatttaccgaacaactccgcggccgggaagccgtaaacttggtctgacagttaccttagaaaaac

SEQ ID NO: 184 - DNA, 72 bp, synthetic construct
  source 1..72: mol_type = other DNA
gaagccatcacaaacggcatgatgaacctgaatcgccagcggcatcagcacaattccacacaacatacgagc

SEQ ID NO: 185 - DNA, 89 bp, synthetic construct
  source 1..89: mol_type = other DNA
tcggcgtcctgctcgtgatcgggagtatctggctgggccaacgttccaaccgcactcctattaattaaacacgcgtttgtccttttcgg

SEQ ID NO: 186 - DNA, 78 bp, synthetic construct
  source 1..78: mol_type = other DNA
gcaccccaggctttacactttatgcttccggctcgtatgttgtgtggaattgttaattaacggccggcccttttcatc

SEQ ID NO: 187 - DNA, 90 bp, synthetic construct
  source 1..90: mol_type = other DNA
gatgttgtctacatggctctgctgtagtgagtgggttgcgctccggcagcggtcctgataaacttggtctgacagttaccttagaaaaac

SEQ ID NO: 188 - DNA, 85 bp, synthetic construct
  source 1..85: mol_type = other DNA
ttaaaaattttaaattataattatttttatagcacgtgatgaaaagggccggccgttaattaacaattccacacaacatacgagc

SEQ ID NO: 189 - DNA, 20 bp, synthetic construct
  source 1..20: mol_type = other DNA
gcggctacagccgatagtct

SEQ ID NO: 190 - DNA, 29 bp, synthetic construct
  source 1..29: mol_type = other DNA
gggaaactggagaaattgtctgggataaa

SEQ ID NO: 191 - DNA, 24 bp, synthetic construct
  source 1..24: mol_type = other DNA
ggcaagtccagtcacaatgtcagg

SEQ ID NO: 192 - DNA, 18 bp, synthetic construct
  source 1..18: mol_type = other DNA
gatggcgagccgttggag

SEQ ID NO: 193 - DNA, 28 bp, synthetic construct
  source 1..28: mol_type = other DNA
cgcctgtctgtacttctgttttcttgac

SEQ ID NO: 194 - DNA, 20 bp, synthetic construct
  source 1..20: mol_type = other DNA
gtttgctgggcaaccagctg

SEQ ID NO: 195 - DNA, 34 bp, synthetic construct
  source 1..34: mol_type = other DNA
  misc_feature 1: note = ABY fluorescein
  modified_base 34: mod_base = OTHER; note = QSY quencher modified nucleotide
acttacgggttgctgcgcaacccaagtgctaccg

SEQ ID NO: 196 - DNA, 41 bp, synthetic construct
  source 1..41: mol_type = other DNA
  modified_base 1: mod_base = OTHER; note = 6-Carboxyfluorescein modified nucleotide
  modified_base 41: mod_base = OTHER; note = minor groove binder quencher modified nucleotide
gcgagattttgccacagtgcgcaaagtattgtccatgcccc

SEQ ID NO: 197 - DNA, 32 bp, synthetic construct
  source 1..32: mol_type = other DNA
  modified_base 1: mod_base = OTHER; note = 6-Carboxyfluorescein modified nucleotide
  modified_base 32: mod_base = OTHER; note = minor groove binder quencher modified nucleotide
aaatggctcgacccggtactgcgctggccata

(19) United States
(12) Patent Application Publication
(10) Pub. No.: US 2025/0340894 A1
(43) Pub. Date: Nov. 6, 2025

(54) PLANT MICROBES AND USES THEREOF

(71) Applicant: Robigo, Inc., Cambridge, MA (US)

(72) Inventors: Jai Phiroze PADMAKUMAR, Cambridge, MA (US); Andrea Kimi WALLACE, Sommerville, MA (US); Louis John PAPA, III, Worcester, MA (US); Yannick BRUNET, Cambridge, MA (US); Mary THOMPSON, Cambridge, MA (US)

(21) Appl. No.: 19/264,004

(22) Filed: Jul. 9, 2025

Related U.S. Application Data
(63) Continuation of application No. PCT/US24/10882, filed on Jan. 9, 2024.
(60) Provisional application No. 63/438,072, filed on Jan. 10, 2023.
(60) Provisional application No. 63/541,448, filed on Sep. 29, 2023.

(51) Int. Cl. C12N 15/78; C07K 14/245; C12N 15/82

(52) U.S. Cl. CPC C12N 15/78; C07K 14/245; C12N 15/8202

(57) ABSTRACT

Provided herein are compositions and methods for use in challenging pathogenic bacteria on plants. Optional features include modification of a donor bacteria to include exogenous nucleic acids encoding for conjugation machinery and gene modification components, such as guide sequence for use in CRISPR. The compositions and methods provided herein can be used for delivery to a wide variety of crops and for targeting one or more pathogens.

### CROSS-REFERENCE

This application claims the benefit of PCT application PCT/US2024/010882 filed Jan. 9, 2024, which claims the benefit of U.S. Provisional Application No. 63/438,072 filed Jan. 10, 2023, and U.S. Provisional Application No. 63/541,448 filed Sep. 29, 2023, all of which are incorporated by reference herein in their entirety.

### SEQUENCE LISTING

The instant application contains a Sequence Listing which has been submitted electronically in ST.26 xml format and is hereby incorporated by reference in its entirety. Said xml copy, created on Jul. 8, 2025, is named 213238-701301_SL.xml and is 234,426 bytes in size.

### BACKGROUND

Facing increasing disease pressure and a changing climate, growers spend $80 billion on nearly six billion pounds of pesticides each year. Pests and disease cause 20-40% yield losses annually. The cost of bacterial diseases alone causes losses of over $1 billion. Current treatments include broad spectrum bactericidal treatments, such as antibiotics and heavy metals. These treatments disrupt the phyto- and rhizo-microbiomes of plants and contaminate the environment. In addition, current treatment methods target general populations of bacteria, to the detriment of the broader plant microbiome. Long term use of these treatments can lead to treatment-resistant bacteria. Thus, there is a need for solutions to challenge microorganisms that compromise plants growth and yield.

### BRIEF SUMMARY

Provided herein are modified bacteria, wherein the modified bacteria comprise: a first expression vector comprising a first exogenous nucleic acid, wherein the first exogenous nucleic acid comprises: a sequence encoding for an endonuclease; and a sequence encoding for at least one guide nucleic acid, wherein the at least one guide nucleic acid targets a sequence in a genome of one or more species of plant-associated bacteria; and a second expression vector comprising a second exogenous nucleic acid, wherein the second exogenous nucleic acid encodes for a bacterial conjugation machinery, wherein the first expression vector and the second expression vector each independently comprises a low copy origin of replication.

Provided herein are modified bacteria, wherein the modified bacteria comprise: an expression vector comprising a first exogenous nucleic acid, wherein the first exogenous nucleic acid comprises: a sequence encoding for an endonuclease; a sequence encoding for at least one 1 guide nucleic acid, wherein the at least one guide nucleic acid targets a sequence in a genome of one or more species of plant-associated bacteria; and a second exogenous nucleic acid, wherein the second exogenous nucleic acid encodes for a bacterial conjugation machinery, wherein the second exogenous nucleic acid is incorporated in a genome of the modified bacteria.

Provided herein are compositions, wherein the compositions comprise: the modified bacteria described herein; and a plant.

Provided herein are compositions, wherein the composition comprises: a first expression vector comprising a first exogenous nucleic acid, wherein the first exogenous nucleic acid comprises: a sequence encoding for an endonuclease; a sequence encoding for at least one guide nucleic acid, wherein the at least one guide nucleic acid targets a sequence in a genome of one or more species of plant-associated bacteria; a second expression vector comprising a second exogenous nucleic acid, wherein the second exogenous nucleic acid encodes for a bacterial conjugation machinery, wherein the first expression vector and the second expression vector each independently comprises a low copy origin of replication.

Provided herein are methods of horizontal gene transfer (HGT), wherein the method of HGT comprises: introducing a modified bacteria to an ecosystem of a plant, wherein the modified bacteria comprises: a first expression vector comprising a first exogenous nucleic acid, wherein the first exogenous nucleic acid comprises: a sequence encoding for an endonuclease; and a sequence encoding for at least one guide nucleic acid, wherein the at least one guide nucleic acid targets a sequence in a genome of one or more species of plant-associated bacteria; and a second expression vector comprising a second exogenous nucleic acid, wherein the second exogenous nucleic acid encodes for a bacterial conjugation machinery, wherein the first expression vector and the second expression vector each independently comprises a low copy origin of replication.

Provided herein are bacterial formulations, wherein the bacterial formulation comprises: the modified bacteria described herein, and an adjuvant.

Provided herein are methods for improving a condition on a plant, the methods comprising applying to a plant a bacterial formulation described herein, wherein application of the bacterial formulation improves the condition on the plant.

Provided herein are methods for improving the growth of a plant, the methods comprising applying to a plant a modified bacteria described herein, wherein application of the modified bacteria improves the growth of the plant.

Provide herein are methods of manufacture, the methods comprise: generating the modified bacteria described herein; growing the modified bacteria; and formulating the modified bacteria with an adjuvant.

Provided herein are modified bacteria, wherein the modified bacteria comprise: a modified Pseudomonas putida comprising: a modified pTAmob plasmid, wherein the modified pTAmob plasmid is modified to comprise a low copy origin of replication; an RK2 plasmid comprising a cassette, wherein the cassette comprises: a promoter, a gRNA sequence, a gRNA scaffold, and a terminator, and a domain encoding for a Cas9 endonuclease.

Provided herein are modified bacteria comprising a modified pTAmob plasmid, wherein the modified pTAmob plasmid comprises a broad host low copy origin of replication.

Provided herein are methods of improving a condition on a plant, the methods comprise applying to a plant the modified bacteria described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a workflow starting with development of a modified donor bacteria, including target identification, donor bacteria selection, and vector design, and continuing to illustrate use of the bacteria in an application, wherein the genetic material from the vector is transferred to a pathogen, resulting in cell death from nuclease expression in the pathogen.

FIGS. 2A-2B illustrate two-plasmid systems for targeting plant-associated bacteria. FIG. 2A illustrates a two-plasmid system, comprising a nucleic acid encoding a conjugation machinery on a first plasmid and nucleic acids encoding a Cas endonuclease and a gRNA on a second plasmid; following transfer of the second plasmid to a pathogenic bacteria, the expressed Cas endonuclease is directed to a genomic binding site by the encoded gRNA, the genomic DNA is cleaved at the target site, and the cell dies. FIG. 2B illustrates a single plasmid system, where both conjugation machinery and genetic payload are on a single plasmid; additional expression modulation features are encoded on the donor genome, including at toxin-antitoxin system, a repressor/promoter feature, or a suicide vector gene; upon transfer to a pathogenic bacteria, the expressed Cas endonuclease is directed to a genomic binding site by the encoded gRNA, the genomic DNA is cleaved at the target site, and the cell dies.

FIG. 3 shows the cell viability of programmable Cas-gRNA constructs. Plasmids pR.036 (polA), pR.037 (ftsZ1), pR.038 (ftsZ2), pR.039 (dnaA), pR.040 (gyrA), and pR.041 (non-target gRNA) were transformed into competent wild-type Xanthomonas campestris (WT). Transformants were enumerated on selective media and cell viability was normalized to transformants that received the non-target gRNA plasmid (n=3).

FIGS. 4A-4D shows graphs of cell transformants (a proxy for cell viability) when X. perforans (GEV) is electroporated with plasmids containing variants of Cas9-gRNA constructs (FIGS. 4A-4B) and compared to the same plasmids electroporated into P. putida (FIGS. 4C-4D). ΔCas9 (pR.220) is pR.029 with cas9 gene knocked out. ΔsgRNA (pR.221) is pR.029 with the gRNA cassette knocked out. pR.029 contains a randomized 28 nucleotide spacer in the location of the gRNA site. pR.036 and pR.211 are two Cas9-gRNA plasmids with two gRNA designed to target the X. perforans genome. pR.242 and pR.243 are two Cas9-gRNA plasmids with two randomized 20 nucleotide gRNA designed to not target the X. perforans nor P. putida genomes.

FIGS. 5A-5B illustrate the schematic design of the horizontal gene transfer (FIG. 5A) and calculated transfer efficiency (FIG. 5B) of the TAmob plasmid.

FIGS. 6A-6B are graphs illustrating the killing of pathogenic bacteria X. campestris after the conjugation with modified P. putida. FIG. 6A is a photo of X. campestris cultures untreated (“No Treatment”) or treated with modified P. putida (“With Treatment”). FIG. 6B is a bar graph showing >99% killing efficacy of modified P. putida against pathogens X. campestris, X. perforans, X. euvesicatoria, and Erwinia amylovora using two different gRNA to target each pathogen.

FIGS. 7A-7B show graphs of killing efficacy (FIG. 7A) and conjugation efficiencies (FIG. 7B) at different ratios of engineered P. putida (“Donor cells”) to X. campestris (“Pathogen”).

FIG. 7A is a bar graph illustrating increasing killing efficacy reaching 100% killing at a 25:1 treatment ratio. FIG. 7B illustrates the transfer efficiency of different ratios of modified bacteria:pathogens, averaging 2.5×10⁴ across tested ratios.

FIGS. 8A-8B are images showing impact modified of P. putida on non-target organisms. FIG. 8A is an image showing fluorescence of X. perforans (GEV) treated with wildtype P. putida (top) and X. perforans treated with P. putida engineered with the two-plasmid system (bottom). FIG. 8B is an image showing fluorescence of X. perforans treated with P. alloputida engineered with (1) the two-plasmid system (top, P.048), (2) genome-integrated TAmob and a CRISPR plasmid carrying a random gRNA (middle, P.046), and (3) genome-integrated TAmob and a CRISPR plasmid carrying gRNA21 (bottom).

FIG. 9A is a photo of plated cultures of bacterial strains mixed with P. putida co-transformed with pTAmob and pR.036 and with (+ind.) and without (−ind.) Cas9 expression.

FIGS. 9B and 9C are bargraphs showing the killing efficacy of targeted pathogenic bacteria and non-target bacteria treated with modified P. putida: FIG. 9B is a bargraph showing complete killing of X. campestris treated with P. putida cotransformed with pTAmob and pR.036 and minimal to no killing of non-target bacteria. FIG. 9C is a bargraph showing complete killing of E. amylovora treated with P. putida cotransformed with pTAmob and pR.118 and minimal to no killing of non-target bacteria.

FIGS. 10A-10B are images showing fluorescence of X. campestris (Xcc) (FIG. 10A) X. perforans (GEV) (FIG. 10B) untreated, treated with wildtype P. putida (“WT P. putida”), treated with P. putida engineered with a TAmob-only plasmid (“TAmob”), and treated with P. putida engineered with a two-plasmid system containing TAmob and a ΔCas9 plasmid (pR.220) (“TAmob+ΔCas9 killer”).

FIGS. 11A-11B show a schematic of tomato plant needle inoculation and node sampling locations (FIG. 11A) and graph of bacterial colonization after two weeks (FIG. 11B). 10 μL of wild-type P. putida (1×10⁸ CFU/mL) was needle inoculated into the xylem and plants were allowed to grow normally for 2 weeks. Destructive plant samples were taken after 14 days at the point of inoculation (POI) and one and two nodes above the POI. Plant samples were weighed and macerated and P. putida cells were enumerated on LB media (n=3).

FIG. 12A shows images of detached cabbage leaves 1 and 7 days after syringe infiltration with phosphate buffered saline (No Disease), X. campestris (Untreated), X. campestris and P. putida co-transformed with pTAmob and pR.036 (Treated), and X. campestris and wild-type P. putida (Wildtype P. putida) (n=5).

FIG. 12B shows images of detached cabbage leaves 7 days after syringe infiltration with phosphate buffered saline (“No Disease”) and X. campestris (“Untreated”), X. campestris and P. putida co-transformed with pTAmob and pR.036 (“Treatment”) under moderate disease pressure (1×10³ CFU/mL X. campestris) and high disease pressure (1×10⁵ CFU/mL X. campestris).

FIGS. 13A-13B illustrate results in detached cabbage leaves after treatment of modified P. putida. FIG. 13A is a graph showing black rot (X. campestris) disease severity in detached cabbage leaves over time. FIG. 13B shows the disease rating scale for black rot in cabbage. Detached cabbage leaves were syringe infiltrated with X. campestris (Untreated), X. campestris and wild-type P. putida (Wildtype P. putida), and X. campestris and P. putida co-transformed with pTAmob and pR.036 (Treated). Disease progression was visually monitored over 7 days (n=5).

FIGS. 14A-14C are graphs showing killing of pathogenic bacteria on cabbage leaves: FIG. 14A is a log-scale graph showing the amount of pathogen cells/gram plant tissue collected over 7 days post inoculation. FIG. 14B is a bar graph showing killing efficiency of engineered P. putida against X. campestris as percent of cells killed at 1 and 21 days compared to an untreated control. FIG. 14C is a bar chart showing transfer efficiency calculated as transfer events per bacteria after one day of incubation.

FIGS. 15A-15B illustrate the disease control on detached crab apple leaves after the treatment. FIG. 15A shows images of detached crab apple leaves 0 and 10 days after syringe infiltration with phosphate buffered saline (Buffer), E. amylovora, E. amylovora and P. putida co-transformed with pTAmob and pR.118 (Treated), and E. amylovora and wild-type P. putida (P. putida) (n=3). Two injection sites were performed on each leaf. FIG. 15B shows disease progression of fire blight (E. amylovora) in detached crab apple leaves over time. Detached apple leaves were syringe infiltrated at two sites on each leaf with phosphate buffered saline (PBS), E. amylovora, E. amylovora and wild-type P. putida (P. putida WT), E. amylovora and P. putida co-transformed with pTAmob and pR.118 (Treatment 1), E. amylovora and P. putida co-transformed with pTAmob and pR.119 (Treatment 2), and Pantoea agglomerans (a known biocontrol against Erwinia). Disease progression was visually monitored over 7 days (n=5).

FIG. 16A illustrates the application of bacterial cultures to tomato leaves using a hand pump sprayer. FIG. 16B is a bargraph illustrating bacterial colonization in tomato leaf tissue 7 days after inoculation with different bacterial formulations, indicating increased colonization in bacteria formulated with 0.10% Tween 20 compared to formulations comprising 0.1% Triton X-100 or 10 mM MgCl₂. Tween and Triton formulations provided for colonization at a level higher than the theoretical threshold for effective killing. An overnight culture of wild-type P. putida was used to prepare 2 mL of each formulation at 1×10⁸ CFU/mL concentration. Formulated P. putida was applied to tomato leaves using a hand pump sprayer, after which plants were allowed to grow normally for 7 days. Destructive leaf samples were taken after 7 days. Leaf samples were weighed and macerated and P. putida cells were enumerated on LB media (n=3). Results from the best three formulations are shown.

FIG. 17 is a graph showing the number of cells per gram plant tissue in destructive leaf samples taken after 24 hours, after 7 days, and in new growth leaves on day 7 that had not been treated at the start of the experiment. Leaf samples were weighed and macerated and cells were enumerated on LB media (n=3).

FIGS. 18A-18B are photos and bar plots demonstrating biocontrol of X. perforans in tomato in growth chamber. FIG. 18A shows the photos of untreated tomato (left) and treated tomato (right). FIG. 18B shows the level of killing efficacy of pathogen (dark bars) and the height of the plant (light bars) for untreated tomatoes (“Untreated Control”), tomatoes treated with the engineered P. alloputida P.048 (“Engineered microbe”), and tomatoes treated with the wild-type P. alloputida (“Wild-type microbe”).

FIGS. 19A-19B shows the results of controlling diseases in tomato plants with P. putida. FIG. 19A is a plot showing the disease severity score over days post inoculation of buffer (negative control), Actigard®, Kocide®, engineered P. putida, and P. putida wild-type, respectively. FIG. 19B is a graph showing pathogen titer per treatment with buffer (negative control), Actigard®, Kocide®, engineered P. putida, and P. putida wild-type, respectively.

FIGS. 20A-20C illustrate the disease control on Jolene tomato plants under greenhouse conditions after treatment with modified donor bacteria compared to Actigard®. FIG. 20A shows images of tomato plant leaves 13 days after spray inoculation with X. perforans (Untreated), X. perforans and P. putida co-transformed with TAmob and pR.093, and X. perforans and 0.05 g/L Actigard® (10 plants per treatment). X. perforans was applied at 10\hat{ }6 CFU/mL and modified donor bacteria were applied at 10\hat{ }7 and 10\hat{ }8 CFU/mL. FIGS. 20B and 20C are bar graphs of average disease severity as measured by the number of lesions per square centimeter from two independent greenhouse experiments.

FIGS. 21A-21B show graphs of plasmid retention in engineered P. putida (FIG. 21A) and colonization of P. putida in tomato plants (FIG. 21B). P. putida co-transformed with pTAmob and pR.093 (Engineered P. putida) and wild-type P. putida (P. putida WT) were spray inoculated into tomato plants and plants were allowed to grow normally under lab conditions for 14 days. Destructive leaf samples were taken daily for 7 days, and then again at 14, 21, and 28 days to measure bacterial colonization and plasmid maintenance. Leaf tissue samples were weighed, macerated, and P. putida cells were enumerated on LB and selective media. Plasmid maintenance in the Engineered P. putida strain was calculated as the percentage of cells that retained both plasmids (pTAmob and pR.093) divided by the total number of cells enumerated on LB media (n=5).

FIGS. 22A-22B show a graph of wild-type P. putida persistence in outdoor tomato and cabbage plants (FIG. 22A) and images of the outdoor tomato plants after spray inoculation (FIG. 22B). Wild-type P. putida was spray inoculated onto outdoor tomato and cabbage plants and plants were allowed to grow normally under summer environmental conditions (subject to sun, rain, wind, etc.). Destructive leaf samples were harvested at 1, 7, 14, and 21 days. Leaf tissue samples were weighed, macerated, and P. putida cells were enumerated on LB media (n=10 leaves per time point for tomatoes, and n=5 leaves per time point for cabbage).

FIG. 23 is a plot illustrating persistence of P. putida in a tomato field trial. P. putida co-transformed with TAmob and pR.093 with (+) and without (−) X. perforans challenge were spray inoculated onto tomato plants at the Gulf Coast Research Education Center at the University of Florida in Balm, FL. Destructive leaf samples were harvested at 1, 3, 7, 14, and 21 days. Leaf tissue samples were weighed, macerated, and P. putida cells were enumerated on LB median=10 leaves per time point for tomatoes.

FIGS. 24A-24C illustrate the disease control on cabbage plants after treatment with modified donor bacteria compared to other bactericidal treatments. FIG. 24A shows images of whole cabbage plants 1 and 7 days, side and top views, after spray inoculation with X. campestris (Untreated), X. campestris and P. putida co-transformed with pTAmob and pR.036 (Treated), X. campestris and Actigard®, X. campestris and Kocide® (Copper), and wild-type P. putida (WT Microbe) (n=5). Arrows indicate early formation of necrotic lesions indicative of black rot disease.

FIG. 24B is a bar graph of the percent killing efficiency in whole cabbage plants of the different tested groups, showing greater than 90% reduction in bacterial count in plants treated with modified bacteria as described herein. FIG. 24C is a bar graph showing the percent of biomass and plant height following treatment, as compared to a control group, showing greater than 15% increase in both biomass and plant height following treatment with modified bacteria as described herein.

FIG. 25A shows images of detached cabbage leaves 5 days after spray inoculation with X. campestris. Leaf images were analyzed using ImageJ to quantify total leaf area and necrotic leaf area. The percentage of necrotic leaf area is calculated as the necrotic leaf area divided by the total leaf area.

FIG. 25B is a plot showing the percentage of necrotic leaf area (measurement of disease symptoms) on cabbage plants under growth chamber conditions after spray inoculation with 1×PBS with 0.2% Tween (Buffer, No Pathogen), P. putida co-transformed with TAmob and pR.036 (Microbe, No pathogen), X. campestris (Buffer), X. campestris and GE P. putida (Microbe) (average of 3 leaves per treatment). X. campestris was applied at 10\hat{ }8 CFU/mL and P. putida was applied at 10\hat{ }9 CFU/mL. When plants are exposed to X. campestris, the treatment reduces disease symptoms.

FIG. 25C is a plot showing the total leaf area of cabbage plants under growth chamber conditions after spray inoculation with 1×PBS with 0.2% Tween (Buffer, No Pathogen), P. putida co-transformed with TAmob and pR.036 (Microbe, No pathogen), X. campestris (Buffer), X. campestris and GE P. putida (Microbe) (average of 3 leaves per treatment). X. campestris was applied at 10\hat{ }8 CFU/mL and P. putida was applied at 10\hat{ }9 CFU/mL. Treatment with microbes increases plant leaf size compared to Buffer and Buffer with X. campestris.

FIG. 26 is a graph of the percentage of necrotic leaf area (measurement of disease symptoms) on cabbage plants under growth chamber conditions after spray inoculation with X. campestris (Buffer), X. campestris and P. putida co-transformed with TAmob and pR.036 (GE P. putida) (10 plants per treatment). X. campestris was applied at 10\hat{ }8 CFU/mL and P. putida was applied at 10\hat{ }7, 10\hat{ }8, and 10\hat{ }9 CFU/mL.

FIG. 27 is a graph of the percentage of necrotic leaf area (measurement of disease symptoms) on cabbage plants under growth chamber conditions after spray inoculation with X. campestris (Buffer), X. campestris and P. putida co-transformed with TAmob and pR.036 (Plasmid) and X. campestris and P. putida with genome integrate TAmob and transformed with pR.036 (Integrated) (10 plants per treatment). X. campestris was applied at 10\hat{ }8 CFU/mL and P. putida was applied at 10\hat{ }9 CFU/mL.

FIGS. 28A-28D illustrate the disease control of turnips after treatment. FIG. 28A shows images of turnips illustrating the disease severity rating scale ranging from 0 to 5. FIG. 28B is a line graph of disease severity in turnips treated with Xanthomonas, 10\hat{ }3:1 Wild type P. putida, 10\hat{ }2:1 modified bacteria, 10\hat{ }3:1 modified bacteria, and PBS control out to 11 days post inoculation. FIG. 28C is a line graph of disease severity in turnips treated with Kocide, Xcc, Actigard, 10\hat{ }3:1 modified bacteria, and PBS control measured out to 11 days post inoculation.

FIG. 28D shows a bar chart of turnip above and below ground biomass in 5 plants per treatment group, 14 days after treatment with PBS control, Xcc (untreated), Actigard, Copper, 100:1 modified bacteria, 1000:1 modified bacteria, 100:1 Wild type P. putida, and 1000:1 Wild type P. putida.

FIGS. 29A-29B are photo and graph showing the biocontrol of E. amylovora in apple trees. FIG. 29A is a photo of two apple trees treated with GE P. alloputida (strain P.064). FIG. 29B is a graph showing the fire blight disease progression curve for apple trees under different treatments, including a genetically engineered Pantoea agglomerans co-transformed with pTAmob and pR.119. The Y-axis corresponds to the value of disease rating, whereas the X-axis indicates days post inoculation.

FIGS. 30A-30B illustrate the motility testing in vitro and in planta. FIG. 30A shows a schematic for soft agar motility experiments to isolate faster moving bacteria. FIG. 30B shows a schematic illustrating sampling at the site of injection as well as 1 cm increments along the stem after inoculation for measuring the bacterial movement in the xylem.

FIG. 31A is a plot showing the soft agar motility assay result performed on seven gram-negative bacteria. The Y axis corresponds to the average growth rate (mm/h), whereas the X axis indicates different bacteria.

FIG. 31B is a plot showing the movement of bacterial strains in citrus vasculature after needle injection at the base of the trunk. The Y axis corresponds to the concentration of bacterial cells in log form, whereas the X axis indicates the internodes the bacterial strains moved above the point of inoculation.

FIG. 31C is a bar plot showing percentage of vessels occluded by tylose in citrus trees inoculated with different strains. The Y-axis corresponds to the percentage of vessels occluded, whereas the X axis indicates different strains, including P. vagans, M. oryzae, and P. alloputida.

FIG. 32 is a graph showing conjugation efficiency in different conjugative systems. 20 natural conjugative plasmids isolated from different bacteria were tested for conjugation efficiency and the top five performing plasmids, TAmob, RP4, Rsa, R702, and pIP113, are shown.

FIGS. 33A-33C illustrate directed evolution on modified bacteria to improve conjugation efficiency. FIG. 33A illustrates the mutagenesis cycle leveraged to improve conjugation efficiency. FIG. 33B illustrates the workflow to induce selective pressure evolution.

FIG. 33C shows a graph of modified bacterial populations that retain a conjugative plasmid (RP4, Rsa, R702, pIP113).

FIG. 34A is a gel image showing truncation of TAmob-pBBR1 plasmid (shown in white boxes) after transformation in P. alloputida.

FIG. 34B is a schematic showing the modification of TAmob plasmid, replacing pBBR1 origin of replication and gentamicin resistance marker with an RSA origin and tetracycline resistance marker.

FIG. 34C is a schematic of the pTAmob-RSA plasmid with conjugation machinery and tetracycline resistance marker.

FIG. 34D shows predicted KPN1 digest fragments for pTAmob-RSA and pR.119 (Cas9 plasmid) in a hypothetical gel image in the left panel and a gel image showing actual sample digest fragments of P. alloputida transformed with pTAmob-RSA and Cas9 plasmids (pR.036, pR.093, pR.119) in the right panel.

FIGS. 35A-35B are pCI plasmid maps. FIG. 35A is a schematic of the cumate-inducible Cas9-gRNA plasmid (pR.029). FIG. 35B is a schematic of the cI transcriptional repressor cassette, expressed cI protein, and the cI protein-repressible Cas9-gRNA plasmid (pR.141)

FIGS. 36A-36B are plots illustrating the validation of the cI repressor/activator system. FIG. 36A provides plots showing the time dependent UV absorbance of strains engineered with cI repressor integrated onto the genome and transformed with the pCI-GFP plasmid. The Y-axis corresponds to the UV absorbance (proxy for cell viability), whereas the X axis indicates the time. FIG. 36B provides bar plots showing the level of GFP fluorescence of the strains in the absence (unrepressed) and presence (repressed) of cI repressor in early-log phase (left), mid-log phase (middle), and stationary phase (right).

FIGS. 37A-37B are plots showing the qPCR assays for the pTAmob, Cas9, and P. alloputida with primer+TaqMan pairs probes. FIG. 37A is a plot showing the quantification cycle (Cq) values for primer+prob pairs against the pTAmob plasmid, Cas9 plasmid, and genomic P. alloputida with a limit of detection of approximately 10² CFU/g soil (3 technical replicates) and primer efficiencies of 93%, 115%, and 97%, respectively. Y axis corresponds to Cq values, whereas X axis indicates the concentration of bacterial cells in complex soil (CFU/g soil). FIG. 37B is a plot showing the quantification cycle (Cq) values (Y axis) against the DNA concentrations in log form (X axis) representing the result of non-specific amplification for pTAmob primers+probe in complex soil without GE P. alloputida.

### DETAILED DESCRIPTION OF THE INVENTION

Provided herein are compositions, methods, and uses thereof for a modified bacteria comprising an expression vector encoding an endonuclease and a nucleic acid complementary to a domain on the genome of a targeting bacteria. Compositions described herein provide several advantages over current treatments of bacteria, including species-specific targeting and killing bacteria without disruption of the broader microbiome environment. Briefly, further described herein are (1) recipient bacteria, (2) modified donor bacteria, (3) methods of horizontal gene transfer for treatment or prevention of a targeting bacteria, (4) combinations with hosts; (5) payloads for delivery to targeting bacteria, (6) methods of use and formulation, (7) production methods, and (8) applications for compositions and methods as described herein.

Also provided herein are compositions, methods, and uses thereof for a modified bacteria comprising an expression vector encoding an endonuclease and a nucleic acid complementary to a domain on the genome of a plant-associated bacteria. Compositions described herein provide several advantages over current agricultural treatments of pathogenic bacteria, including species-specific targeting and killing pathogenic bacteria without disruption of the broader microbiome environment. Briefly, further described herein are (1) bacteria for targeting, (2) modified donor bacteria, (3) methods of horizontal gene transfer for treatment or prevention of a plant-associated bacteria, (4) combinations with plant hosts; (5) payloads for delivery to pathogenic bacteria, (6) methods of use and formulation, (7) production methods, and (8) applications for compositions and methods as described herein.

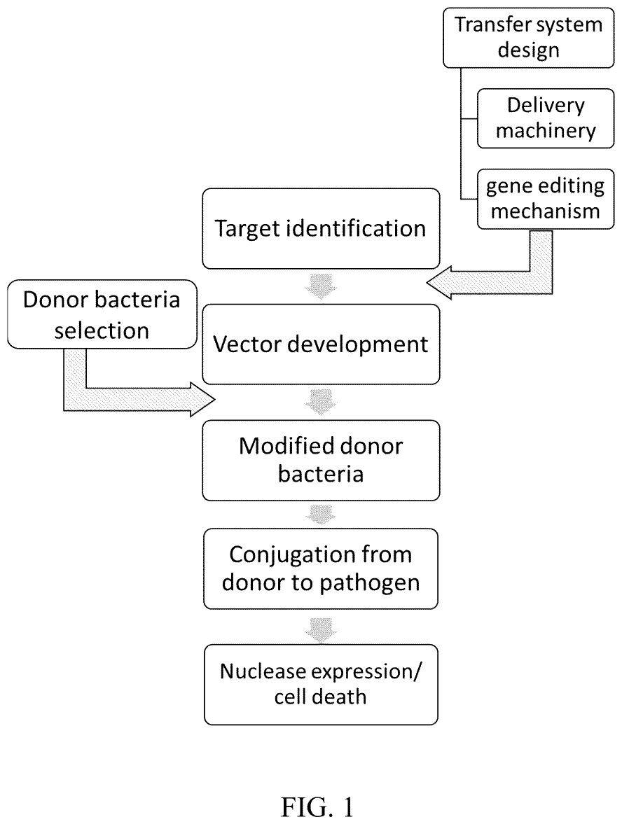

A general workflow describing development of a modified bacteria and treatment of a pathogen is shown in FIG. 1. Briefly, a target pathogen is identified for treatment. Identification includes determination of relevant sites for cleavage on the pathogen genome. Concurrently, a transfer system is designed to encode a delivery machinery and a gene editing mechanism. Concurrently, a donor microbe is selected. The donor microbe is modified to include the engineered vector(s). The donor microbe is introduced to the plant environment and the vector is conjugated into the pathogen via gene transfer. Upon activation in the target pathogen, the target genome is cleaved, preventing further replication and causing cell death.

### Definitions

The terminology used herein is for the purpose of describing particular cases only and is not intended to be limiting. As used herein, the singular forms “a”, “an” and “the” can include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

Unless specifically stated, as used herein, the term “about” in reference to a number or range of numbers is understood to mean the stated number and numbers+/−10% thereof, or 10% below the lower listed limit and 10% above the higher listed limit for the values listed for a range.

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The term “effective amount” or “therapeutically effective amount” refers to an amount that is sufficient to achieve or at least partially achieve the desired effect.

Provided herein are compositions which include bacteria having a percent identity based on 16S rRNA bacterial genetic sequence, a hypervariable region of the 16S rRNA, or whole genome comparison to a reference strain. Typically, comparison of the 16S rRNA bacterial genetic sequence allows a strain to be identified as within the same species as another strain by comparing sequences with known bacterial DNA sequences using NCBI BLAST search. The level of identity in relation to a nucleotide sequence may be determined for at least 20 contiguous nucleotides, for at least 30 contiguous nucleotides, for at least at least 40 contiguous nucleotides, for at least 50 contiguous nucleotides, for at least 60 contiguous nucleotides, or for at least 100 contiguous nucleotides. A level of identity in relation to a nucleotide sequence can be determined for the entire sequence searched. Percent identity can be at least 70%, 80%, 85%, 90%, 91%, 92%, 93%, 94%, 95%, 96%, 97%, 98%, 99%, or 100% to a reference bacterial 16S rRNA sequence, 16S rRNA V4 region sequence, or whole genome sequence. Percent identity can be at least 70%, 80%, 85%, 90%, 91%, 92%, 93%, 94%, 95%, 96%, 97%, 98%, 99%, or 100% to a reference bacteria 16S rRNA: V1 region, V2 region, V3 region, V5 region, V6 region, V7 region, V8 region or V9 region sequence.

Reference to a population of bacteria or a purified population refers to a plurality of bacteria. A purified bacteria can be enriched from a source sample. A population of bacteria can comprise about: 10%, 20%, 30%, 40%, 50%, 60%, 70% or more of a single strain of bacteria.

As used herein, a substance is “pure” or “substantially pure” if it is substantially free of other components. The terms “purify,” “purifying” and “purified”, when applied to a bacterium, can refer to a bacterium that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, or by passage through culture, and a purified bacterium or bacterial population may contain other materials up to at least about 10%, about 20%, about 30%, about 40%, about 50%, about 60%, about 70%, about 80%, about 90%, or above about 90% and still be considered “isolated.” Purified bacteria and bacterial populations can be more than at least about 80%, about 85%, about 90%, about 91%, about 92%, about 93%, about 94%, about 95%, about 96%, about 97%, about 98%, about 99%, or more than at least about 99% pure by weight (w/w). In the instance of microbial compositions provided herein, the one or more bacterial types, species, or strains present in the composition can be independently purified from one or more other bacteria produced and/or present in the material or environment containing the bacterial type. Microbial compositions and the bacterial components thereof are generally purified from residual habitat products.

As used herein, “plant-associated bacteria” refers to any bacteria found in the phytosphere or soil in the region of a plant. Plant-associated bacteria, as used herein, include bacteria found in any interior or exterior region of the plant. In some embodiments, plant-associated bacteria are found in the phyllosphere, the rhizosphere, the root system, the shoot system, the flowers, the leaves, the fruit, the stem, or the roots. Plant-associated bacteria, as used herein, include bacteria found in any layer of soil that will affect the growth or productivity of a plant. In some embodiments, a plant-associated bacteria is found in the humus layer, the topsoil layer, the eluviation layer, or the subsoil layer.

An isolated bacterium may have been (1) separated from at least some of the components with which it was associated when initially obtained (whether in nature or in an experimental setting), and/or (2) produced, prepared, purified, and/or manufactured by the hand of man, e.g. using artificial culture conditions such as (but not limited to) culturing on a plate and/or in a fermentor. Isolated bacteria can include those bacteria that are cultured, even if such cultures are not monocultures. Isolated bacteria can be separated from at least about 10%, about 20%, about 30%, about 40%, about 50%, about 60%, about 70%, about 80%, about 90%, or more of the other components with which they were initially associated. Isolated bacteria can be more than about 80%, about 85%, about 90%, about 91%, about 92%, about 93%, about 94%, about 95%, about 96%, about 97%, about 98%, about 99%, or more than about 99% pure. A bacterial population of a biological sample provided herein can comprise one or more bacteria, which may then be isolated from such sample. Isolated bacteria may be provided in a form that is not naturally occurring.

### I. Recipient Bacteria

Recipient bacteria are plant-associated bacteria comprising a sequence complementary to a gRNA sequence in the genome modification mechanism. In some embodiments, the plant-associated bacteria are phytobacteria. In some embodiments, the plant-associated bacteria are soil bacteria. In some embodiments, the bacteria are pathogenic bacteria. In some embodiments, the plant-associated bacteria negatively affect a feature of a plant. In some embodiments, the plant-associated bacteria positively affect a feature of a plant. In some embodiments, the plant-associated bacteria have one or more deleterious effects on a plant. The deleterious effects on plants include, but are not limited to, suppression of delivery of water and nutrients through the xylem of the host plant, removal of water and nutrients from plants, and secretion of toxins. In some embodiments, the suppression, removal, or secretion effect described herein is associated with wilt. In some embodiments, the suppression, removal, or secretion effect described herein is associated with the overgrowth of plants. In some embodiments, the suppression, removal, or secretion effect described herein is associated with reduced yield of vegetables, leafy greens, or fruits. In some embodiments, the suppression, removal, or secretion effect described herein is associated with reduced size of fruits or vegetables.

In some embodiments, the bacteria being targeted as described herein are pathogens. Pathogenic bacteria cause significant losses in crops around the world. In some cases, the pathogenic bacteria are pathogenic outright when colonized on a plant. Pathogenic bacteria are categorized in a range of genotypic genera. Selected genera with non-limiting pathogenic species examples are listed in Table 1. Targets for challenge can include strains and variants of a named species. In some embodiments, provided herein are compositions for use in targeting genus bacteria, optionally exemplary species, listed in Table 1.

TABLE 1
Exemplary Pathogenic Bacteria

| Genus | Exemplary species |
|---|---|
| Acidovorax spp. | Acidovorax citrulli, Acidovorax avenae |
| Acinetobacter spp. | Acinetobacter lactucae, Acinetobacter |
| | oryzae |
| Agrobacterium spp. | Agrobacterium tumefaciens, Agrobacterium |
| | vitis |
| Bacillus spp. | Bacillus subtilis, Bacillus coagulans |
| Burkholderia spp. | Burkholderia cenocepacia |
| Citrobacter spp. | Citrobacter amalonaticus |
| Clavibacter spp. | Clavibacter michiganensis |
| Dickeya spp. | Dickeya dadantii, Dickeya solani |
| Enterobacter spp. | Enterobacter cowanii, Pluralibacter |
| | pyrinus |
| Erwinia spp. | Erwinia amylovora, Erwinia caratovora |
| Guignardia spp. | Guignardia bidwellii |
| Liberibacter spp. | Liberibacter americanus, Liberibacter |
| | africanus, and Liberibacter asiaticus |
| Lysinibacillus spp. | Lysinibacillus tabacifolii |
| Microdochium spp. | Microdochium oryzae |
| Pantoea spp. | Pantoea stewartii |
| Pectobacterium spp. | Pectobacterium carotovorum |
| Phytoplasma spp. | Phytoplasma australasiaticum, |
| | Phytoplasma costaricanum |
| Pseudomonas spp. | P. syringae, P. savastanoi, and P. |
| | rubrilineans |
| Ralstonia spp. | Ralstonia solanacearum |
| Rathayibacter spp. | Rathayibacter toxicus |
| Serratia spp. | Serratia marcescens, Serratia |
| | proteamaculans |
| Spiroplasma spp. | Spiroplasma citri, Spiroplasma kunkelii |
| Stenotrophomonas spp. | Stenotrophomonas pavanii |
| Streptomyces spp. | Streptomyces scabiei, Streptomyces |
| | acidiscabies, Streptomyces |
| | europaeiscabiei |
| Xanthomonas spp. | X. campestris, X. euvesicatoria, X. |
| | perforans, X. citri, X. oryzae, X. |
| | axonopodis, and X. albileneans |
| Xylella spp. | Xylella fastidiosa |

In some embodiments, a pathogenic bacterial population for treatment as described herein comprises one species of bacteria. In some embodiments, a pathogenic bacterial population comprises multiple species within a genotypic family. In some embodiments, a pathogenic bacterial population comprises one or species from more than one genotypic family. Provided herein are compositions comprising modified bacteria for targeting one or more specific species or strain of bacteria. In some embodiments, the one or more target species is a single species. In some embodiments, the one or more target species are within a single genotypic family. In some embodiments, the one or more target species are within more than one genotypic family. In some embodiments, a species of pathogenic bacteria can be found in multiple host plants. In some embodiments, a pathogenic bacteria causes disease in a single species of plant. In some embodiments, a pathogenic bacteria causes disease in multiple species of plants. In some embodiments, a pathogenic bacteria causes disease in one or more host plants, and does not cause disease in other host plants.

Bacterial species of the genus Xanthomonas cause disease in up to 400 plant species. In tomato crops, Xanthomonas species (spp.) are a major component of the microbiome, representing 10-40% of the bacterial communities in the fruits, leaves, and flowers of tomatoes in the United States. Upon transmission, Xanthomonas spp. grow epiphytically on the leaf surface and then enter into the plant through stomata, hydathodes, or wounds to colonize in the mesophyll parenchyma or to spread systemically through the vascular system. The bacteria grow to high abundance in the plant tissue, resulting in necrosis of leaves, fruits, and ultimately defoliation. A selection of Xanthomonas species for targeting using compositions and methods described herein and associated host/diseases are shown in Table 2.

TABLE 2
Selected Xanthomonas species and associated Plant Diseases

| Xanthomonas | | | | | |
|---|---|---|---|---|---|
| spp. | Pathovar | Acronym | Host | Disease | Taxonomy ID |
| X. albilineans | | | Sugarcane | Leaf scald | NCBI: txid29447 |
| X. alfalfae | | | Alfalfa | Bacterial | NCBI: txid366650 |
| | | | | leaf spot | |
| X. arboricola | X. arboricola pv. | Xap | Prunus | Bacterial | NCBI: txid69929 |
| | pruni | | | spot | |
| | X. arboricola pv. | Xcp | Pomegranate | Leaf blight | NCBI: txid487838 |
| | punicae | | (Punica granatum) | | |
| | X. arboricola pv. | Xaj | Persian (English) | Walnut blight | NCBI: txid195709 |
| | juglandis | | walnut (Juglans | | |
| | | | regia) | | |
| X. axonopodis | X. axonopodis pv. | Xam | Cassava | Bacterial | NCBI: txid43353 |
| | manihotis | | | blight | |
| X. campestris | X. campestris pv. | Xca | Horseradish | Bacterial | NCBI: txid329463 |
| | armoraciae | | | leaf spot | |
| | X. campestris pv. | Xcc | Brassicaceae | Black rot | NCBI: txid340 |
| | campestris | | | | |
| | X. campestris pv. | Xcl | Perennial grass | Bacterial | NCBI: txid487875 |
| | leersiae | | | streak | |
| | X. campestris pv. | Xvm | Banana | Enset wilt | NCBI: txid454958 |
| | musacearum | | | | |
| | X. campestris pv. | Xcr | Brassica | Bacterial | NCBI: txid359385 |
| | raphani | | oleracea | leaf spot | |
| | X. campestris pv. | Xcv | Lettuce | Bacterial | NCBI: txid83224 |
| | vitians | | | leaf spot | |
| X. cannabis | | | Cannabis (Cannabis | Bacterial | NCBI: txid1885674 |
| | | | sativa L.) | leaf spot | |
| X. citri | X. citri pv. citri | Xcci | Citrus | Citrus canker | NCBI: txid611301 |
| | X. citri pv. fuscans | Xcf | Bean (Phaseolus | Bacterial | NCBI: txid366649 |
| | | | vulgaris L.) | blight | |
| | X. citri pv. glycines | Xcg | Soybean (Glycine | Bacterial | NCBI: txid473421 |
| | | | max) | pustule | |
| | X. citri pv. | Xcm | Cotton (Gossypium | Bacterial | NCBI: txid86040 |
| | malvacearum | | spec.) | blight | |
| | X. citri pv. | Xmi | Mango (Mangifera | Bacterial | NCBI: txid454594 |
| | mangiferaeindicae | | indica) | black spot | |
| | X. citri pv. punicae | Xcp | Pomegranate | Leaf blight | NCBI: txid487838 |
| | | | (Punica granatum) | | |
| X. cucurbitae | | | Cucurbits | Bacterial spot | NCBI: txid56453 |
| X. cynarae | | | Artichoke (Cynara | Bacterial | NCBI: txid10214 |
| | | | scolymus L.) | bract spot | |
| X. euvesicatoria | X. campetris pv. | Xav | Pepper and tomato | Bacterial | NCBI: txid456327 |
| | vesicatoria | | | leaf spot | |
| X. floridensis | | | Watercress | — | NCBI: txid1843580 |
| X. fragariae | | | Strawberry | Bacterial angular | NCBI: txid48664 |
| | | | | leaf spot | |
| X. gardneri | | | Pepper and tomato | Bacterial spot | NCBI: txid90270 |
| X. maliensis | | | Rice | — | NCBI: txid1321368 |
| X. nasturtii | | | Watercress | — | NCBI: txid1843581 |
| X. oryzae | X. oryzae pv. oryzae | Xoo | Rice | Bacterial blight | NCBI: txid64187 |
| | X. oryzae pv. | Xoc | Rice | Bacterial streak | NCBI: txid129394 |
| | oryzicola | | | — | |
| X. perforans | | | Tomato | Bacterial spot | NCBI: txid442694 |
| X. phaseoli | X. phaseoli pv. | Xcp | Bean (Phaseolus | Bacterial blight | NCBI: txid1985254 |
| | phaseoli | | vulgaris L) | | |
| X. prunicola | | | nectarine (Prunus | — | NCBI: txid2053930 |
| | | | persica var. | | |
| | | | nectarina) trees | | |
| X. | | | | | |
| pseudoalbilineans | | | | | |
| X. sacchari | | | Sugarcane | Chlorotic | NCBI: txid56458 |
| | | | | streak disease | |
| X. translucens | X. translucens pv. | Xtt | Wheat | Black chaff | NCBI: txid134875 |
| | translucens | | | | |
| | X. translucens pv. | Xtu | Wheat | Black chaff | NCBI: txid487909 |
| | undulosa | | | | |
| X. vasicola | X. vasicola pv. | Xvv | Sugarcane | Gumming | NCBI: txid325776 |
| | vasculorum | | | disease | |

Note:
Xanthomonas arboricola pv. punicae is currently listed as X. citri pv. punicae in NCBI taxonomy.

Targeting recipient bacteria with compositions described herein improve a condition in a plant. In some embodiments, targeting a recipient bacteria reduces or removes a deleterious effect on a plant. In some embodiments, targeting a recipient bacteria improves a condition on a plant. In some embodiments, the condition is availability of a nutrient to the plant, availability of water to the plant, or level of toxins.

### II. Donor Plant-Associated Bacteria

Provided herein are donor bacteria for use in suppression of a targeting bacteria. Provided herein are donor bacteria which are modified to comprise exogenous nucleic acids. In some embodiments, the exogenous nucleic acid comprises DNA. In some embodiments, the exogenous nucleic acid comprises RNA. In some instances, the suppression is preventative. In some embodiments, the suppression prevents an increase in the abundance across many plants. In some embodiments, the suppression prevents an increase in an individual plant. In some embodiments, the suppression restricts spread within a population of plants of the same species. In some embodiments, the suppression prevents spread from one species to a different species. In some embodiments, the suppression is a challenge to one or more existing pathogens on a plant. In some embodiments, the suppression is to an established colonization within the plant tissue. In some embodiments, the suppression is to an established colonization on the surface of the plant, within the plant, within or on the roots of the plant, or in the soil surrounding the plant. In some embodiments, the suppression is to an established colonization in soil. In some embodiments, the suppression is to an established colonization in roots. In some embodiments, the suppression comprises a reduction in growth of the plant-associated bacteria. In some embodiments, the suppression comprises a reduction in total abundance of the target plant-associated bacteria across many plants. In some embodiments, the suppression comprises a reduction in abundance of the plant-associated bacteria in an individual plant. In some embodiments, the suppression comprises a reduction in bacterial concentration as measured by colony forming units (CFU) per gram of plant tissue. In some embodiments, the suppression promotes the growth of one or more beneficial microbes. Beneficial microbes include, but are not limited to bacteria or fungi. In some embodiments, beneficial microbes include saprophytes. In some embodiments, beneficial microbes include mycorrhizae. In some embodiments, beneficial microbes include Rhizobia spp., Bacillus spp., Pseudomonas spp., Enterobacter cloacae, Endophytic diazotrophic, Arthrobacter spp., Burkholderia spp., or Citrobacter freundii.

Methods for suppression of plant-associated bacteria using compositions described herein, provide for an improvement in a condition or production of a plant. In some embodiments, suppression of plant-associated bacteria impedes or disrupts plant disease progression. Common symptoms of plant disease caused by pathogenic bacteria in plants includes, but not limited to, black rot, blight, necrotic lesions, defoliation, stunted growth, and citrus greening. In some embodiments, suppression of plant-associated bacteria causes increased leaf integrity. In some embodiments, suppression of plant-associated bacteria causes increased height. In some embodiments, suppression of plant-associated bacteria causes increased growth rate. In some embodiments, suppression of plant-associated bacteria causes increased yield of a product of the plant. In some embodiments, suppression of plant-associated bacteria causes increased biomass. In some embodiments, suppression of plant-associated bacteria causes increased crop quantity. In some embodiments, suppression of plant-associated bacteria causes increased vegetable quantity. In some embodiments, suppression of plant-associated bacteria causes increased fruit quantity. In some embodiments, suppression of plant-associated bacteria causes increased fruit mass. In some embodiments, suppression of plant-associated bacteria causes improved plant quality. In some embodiments, suppression of plant-associated bacteria causes improved fruit quality. In some embodiments, suppression of plant-associated bacteria causes improved vegetable quality. In some embodiments, suppression of plant-associated bacteria causes increased longevity. In some embodiments, suppression of plant-associated bacteria causes increased tolerance to environmental stress. In some embodiments, suppression of plant-associated bacteria causes increased salinity tolerance. In some embodiments, suppression of plant-associated bacteria causes increased drought tolerance.

Environmental bacteria found in the plant microbiome are used herein for modification as a donor vehicle for compositions for treatment of plant-associated bacteria. In some embodiments, a donor bacteria found in a plant phytomicrobiome or rhizomicrobiome is selected for modification according to methods described herein. In some embodiments, the donor bacteria provides a beneficial mechanism of action to plant growth. In some embodiments, the donor bacteria does not affect the growth or production of the plant. In some embodiments, the donor bacteria is not native to the plant microbiome. Selected genera and exemplary species of bacteria considered for modification according to methods described herein are listed in Table 3. Any named species is understood to additionally include any variant or strain thereof.

TABLE 3
Exemplary bacteria for modification.

| Genus | Exemplary species |
|---|---|
| Acidovorax spp. | |
| Acinetobacter spp. | |
| Actinobacteria spp. | |
| Agrobacterium spp. | |
| Alcaligenes spp. | |
| Aneurinibacillus spp. | Aneurinibacillus aneurinilyticus |
| Azospirillum spp. | Azospirillum brasilense |
| Azotobacter spp. | |
| Bacillus spp. | Bacillus amyloliquefaciens, Bacillus |
| | cereus, Bacillus cereus TCR17, Bacillus |
| | megaterium, Bacillus paranthracis, |
| | Bacillus pumilus, Bacillus subtilis, |
| | Bacillus thuringiensi |
| Bradyrhizobium spp. | |
| Burkholderia spp. | Burkholderia ambifaria |
| Buttiauxella spp. | Buttiauxella agrestis |
| Chromobacter spp. | |
| Chryseobacterium spp. | |
| Citrobacter spp. | |
| Clavibacter spp. | |
| Curtobacterium spp. | Curtobacterium albidum |
| Dickeya spp. | |
| Enterobacter spp. | |
| Erwinia spp. | |
| Escherichia spp. | Escherichia coli |
| Firmicutes spp. | |
| Flavobacterium spp. | |
| Frankia spp. | |
| Gluconacetobacter spp. | Gluconacetobacter diazotrophicus |
| Herbaspirillum spp. | Herbaspirillum seropedicae |
| Klebsiella spp. | |
| Kocuria spp. | |
| Kosakonia spp. | |
| Lysinibacillus spp. | |
| Methylobacterium spp. | Methylobacterium radiotolerans, |
| | Methylobacterium oryzae, |
| | Methylobacterium nodulans, |
| | Methylobacterium mesophilicum, |
| | Methylobacterium phylosphaerae, |
| | Methylobacterium oxalidis, |
| | Methylobacterium thiyocyanatum |
| Methylorubrum spp. | Methylorubrum aminovorans, |
| | Methylorubrum extorquens, Methylorubrum |
| | podarium, Methylorubrum populi, |
| | Methylorubrum pseudosasae, Methylorubrum |
| | rhodesianum, Methylorubrum rhodinum, |
| | Methylorubrum salsuginis |
| Microbacterium sp. | |
| Myroides spp. | Myroides odoratimimus |
| Pantoea spp. | Pantoea alli, Pantoea agglomerans |
| Paraburkholderia spp. | Paraburkholderia phytofirmans |
| Pectobacterium spp. | |
| Phytoplasma spp. | |
| Piriformospora spp. | Piriformospora indica |
| Providencia spp. | Providencia rettgeri TCR21 |
| Proteobacteria spp. | |
| Pseudomonas spp. | Pseudomonas putida, Pseudomonas |
| | alloputida, Pseudomonas aeruginosa, |
| | Pseudomonas fluorescens, Pseudomonas |
| | koreensis, Pseudomonas reactans |
| Ralstonia spp. | Ralstonia pickettii |
| Rathayibacter spp. | |
| Rhizobium spp. | |
| Rhizoglomus spp. | Rhizoglomus irregulare |
| Serratia spp. | |
| Sphingobium spp. | |
| Sphinomonas spp. | |
| Spiroplasma spp. | |
| Stenotrophomonas spp. | |
| Streptomyces spp. | |
| Tatumella spp. | |
| Vibrio spp. | |
| Xanthomonas spp. | |
| Xylella spp. | |

It is noted that phylogenomic analysis of P. putida strain KT2440 described herein resulted in reclassification to a new species, Pseudomonas alloputida. For the purposes of this disclosure, terms are considered interchangeable. In some embodiments, a modified donor bacteria comprises modifications as shown in Table 4.

TABLE 4
Modified donor bacterial strains

| Identifier | Strain | Features |
|---|---|---|
| E.022 | E. coli TAmob, GFP | pR.003, pR.002 |
| E.027 | E. coli | TetR |
| MG1655 | Escherichia coli MG1655 | Landing pad, TetR |
| | Pantoea agglomerans | |
| Pf-5 TetR | Pseudomonas protegens | TetR |
| P.020 | P. putida TAmob, gRNA_6 | pR.003, pR.037 |
| P.034 | P. putida TAmob, GFP | pR.003, pR.002 |
| P.036 | P. putida TAmob | pR.003 |
| P.044 | P. putida TAmob, gRNA_5 | pR.003, pR.036 |
| P.046 | P. putida TAmob, gRNA_empty | pR.003, pR.029 |
| P.047 | P. putida | GmR |
| P.048 | P. putida TAmob, gRNA_21 | pR.003, pR.093 |
| | P. putida TAmob, | pR.036, pR.093 |
| P.049 | P. putida TAmob, gRNA_22 | pR.003, pR.094 |
| P.063 | P. putida TAmob, gRNA_23 | pR.003, pR.118 |
| P.064 | P. putida TAmob, gRNA_24 | pR.003, pR. 119 |
| P.105 | P. putida Tamob.v2, gRNA24 | pR.119, pR160 |
| Test 172 | P. putida TAmob.v2, gRNA5 | |
| Test 173 | P. putida TAmob.v2, gRNA21 | |
| | P. putida TAmob.v2, gRNA47 | |
| Test 236 | P. putida with TAmob | |
| | integrated on genome | |
| | P. putida intTAmob, gRNA5 | |
| | P. putida intTAmob, gRNA21 | |
| | P. putida intTAmob, gRNA47 | |
| | P. putida TAmob, ΔCas9 | |
| | P. agglomerans TAmob, gRNA24 | |
| | Pseudomonas syringae pv. | |
| | glycinea M92 | |
| Xcc TetR | Xanthomonas campestris, | TetR |
| | J23101-mNeonGreen-GentR | |
| | Xanthomonas campestris | |
| E3 TetR | Xanthomonas euvesicatoria | TetR |
| GEV TetR | Xanthomonas perforans | TetR |
| | Xylella fastidiosa | |

### III. Horizontal Gene Transfer

Horizontal Gene Transfer (HGT) is the transfer of genetic material between organisms, rather than from parent to offspring. HGT is an important evolutionary process, for example driving development of antibiotic resistance. Mechanisms of HGT include conjugation, transformation, transduction, and gene transfer agents. In embodiments described herein, an expression vector encoding guide and nuclease components are transferred between bacterial species.

### Bacterial Conjugation

In bacterial conjugation, a donor cell produces a pilus. The pilus attaches to a recipient cell, connecting the two cells by a bridge. Plasmid DNA from the donor is transferred to the recipient cell. Expression vectors described herein comprise a domain encoding for a conjugation machinery and a gene or combination of genes for targeted bacterial genome modulation, including killing of bacteria or metabolic manipulation of bacteria and augmentation of beneficial bacteria. Essential components for a conjugation machinery include the relaxosome and the type 4 secretion system. The relaxosome, composed of the relaxase and other DNA transfer replication (dtr) genes, nicks the origin of transfer (oriT) and where the ssDNA will be unwound and transferred from the donor to the recipient. The type 4 secretion system is encoded by the mating pair formation (mpf) genes and is essential for bringing the donor and recipient cells into close contact, usually through a pilus, and forming a pore through which the plasmid DNA is transferred. In some embodiments, the plasmid comprises CRISPR genome editing genes.

In some embodiments, the conjugation machinery is incorporated in the bacterial genome. In some embodiments, the conjugation machinery comprises components essential for creating genome modifications. In some embodiments, the conjugation machinery comprises Tn7. In some embodiments, the conjugation machinery comprises site-specific recombination sequences (“landing pads”) for site-specific integration of recombinant constructs.

In some embodiments described herein, an expression vector comprises a trans, or two-plasmid system. In some embodiments of a two-plasmid system, the conjugation machinery is expressed separately from the transferred gene editing domains. In some embodiments of a two-plasmid system, a Cas endonuclease and a gRNA are encoded on a first plasmid. In some embodiments, the conjugation machinery is encoded on a second plasmid. Following transfer of the first plasmid to a plant-associated bacterial cell, the expressed Cas endonuclease is directed to a genomic binding site by the encoded gRNA, the genomic DNA is cleaved at the target site, and consequently the plant-associated bacterial cell dies. In some embodiments, an expression vector comprises a cis, or single plasmid system. In some embodiments of a single plasmid system, the conjugation machinery is expressed on the same plasmid as the delivered gene editing domains.

### Vectors

Vectors can be used for horizontal gene transfer. In some embodiments, vectors used for horizontal gene transfer are plasmids. Plasmids are unlike virus vectors, as they do not encode a protein coat. In some embodiments, a plasmid encodes for a conjugation machinery. In some embodiments, a plasmid encodes for a conjugative pilus to conjoin two bacteria. In some embodiments, a modified donor bacterial genome encodes for a conjugation machinery. In some embodiments, a plasmid described herein encodes for a bacterial genome modulation mechanism. In some embodiments, a plasmid described herein encodes for a guide RNA and an endonuclease. In some embodiments, a plasmid described herein encodes for both a conjugation machinery and a bacterial genome modulation mechanism. In some embodiments, a plasmid described herein is a modified TAmob plasmid. In some embodiments, a conjugation machinery and a bacterial genome modulation mechanism are encoded on separate plasmids. In some embodiments, a conjugation machinery is encoded on the modified donor bacteria genome. In further embodiments, the modified donor bacteria encoding the conjugation machinery comprises a plasmid encoding the bacterial genome modulation mechanism.

In some embodiments, the modified donor bacteria and plasmid system comprise domains encoding for a toxin-antitoxin (TA) system. In some embodiments, a toxin is encoded by the bacterial genome. The antitoxin is encoded by the plasmid system. Survival of the modified donor bacteria is dependent on maintaining the engineered plasmid system, in order to counteract the toxin produced by the donor bacterial genome. In some embodiments, the TA system is a type I TA system. In some embodiments, the TA system is a type II TA system. In some embodiments, the TA system is a type III TA system. In some embodiments, the TA system is a type IV TA system. In some embodiments, the TA system is a type V TA system. In some embodiments, the modified donor bacteria comprises the ccdB toxin gene, to produce ccdB toxin. In further embodiments, a plasmid comprises the ccdA antitoxin gene, to produce ccdA antitoxin. In a plasmid system comprising a single plasmid encoding both conjugation and genome modulation machinery, the plasmid further encodes for an antitoxin. In a plasmid system comprising separate plasmids independently encoding the conjugation machinery and genome modulation mechanism, an antitoxin is encoded on the plasmid encoding genome modulation mechanism. In a modified donor bacteria comprising a genome encoding the conjugation machinery and a plasmid encoding the genome modulation machinery, an antitoxin is encoded on the plasmid encoding genome modulation mechanism. Non-limiting examples of TA systems for use in compositions and methods described herein are shown in Table 5.

TABLE 5
TA toxins and antitoxins.

| | Antitoxin/molecular | | | Cellular |
|---|---|---|---|---|
| Toxin | species | Type | Toxin activity | process |
| Hok | Sok/RNA | I | Integrates into the inner cell membrane | ATP synthesis |
| TisB | IstR-1/RNA | I | Integrates into the inner cell membrane | ATP synthesis |
| SymE | SymR/RNA | I | mRNA cleavage | Translation |
| CcdB | CcdA/Protein | II | Inhibition of DNA gyrase | Replication |
| ParE | ParD/Protein | II | Inhibition of DNA gyrase | Replication |
| MazF | MazE/Protein | II | Ribosome-independent mRNA cleavage and | Translation |
| | | | cleavage of 16S rRNA | |
| MazF- | MazE-mt6/Protein | II | Ribosome-independent mRNA cleavage and | Translation |
| mt6 | | | cleavage of 23S rRNA | |
| Kid | Kis/Protein | II | Ribosome-independent mRNA cleavage | Translation |
| HicA | HicB/Protein | II | Ribosome-independent mRNA cleavage | Translation |
| RelE | RelB/Protein | II | Cleavage of ribosome-bound mRNA | Translation |
| VapC | VapB/Protein | II | Cleavage of tRNAfMet | Translation |
| Doc | Phd/Protein | II | Binds to the 30S ribosomal subunit | Translation |
| RatA | RatB/Protein | II | Binds to the 50S ribosomal subunit | Translation |
| HipA | HipB/Protein | II | Phosphorylation of EF-Tu | Translation |
| zeta | epsilon/Protein | II | Phosphorylation of UDP-N-acetylglucosamine | Peptidoglycan |
| | | | | synthesis |
| ToxN | ToxI/RNA | III | RNA cleavage | Translation |
| YeeV | YeeU/Protein | IV | Inhibition of FtsZ and MreB polymerization | Cytoskeleton |
| CptA 57 | CptB/Protein | IV | Inhibition of FtsZ and MreB polymerization | Cytoskeleton |
| GhoT | GhoS/Protein | V | Integrates into the inner cell membrane | ATP synthesis |

In some embodiments, the plasmid system described herein comprises a segregation system. In some embodiments, the segregation system is ParCMR segregation system (SEQ ID NO: 47). In some embodiments, the plasmid system described herein comprises a gamma-delta resolvase cassette (SEQ ID NO: 48). In some embodiments, the plasmid system described herein comprises plasmid R1 derived ParCMR segregation system and the gamma-delta multimer resolvase cassette from the E. coli F plasmid Tn1000 transposon.

A single plasmid system comprises the conjugation machinery on the transferred plasmid. In some embodiments, it is desirable to limit replication of the plasmid system in recipient plant-associated microbes. In some embodiments, limiting replication of the plasmid system in recipient plant-associated microbes reduces metabolic burden on the recipient plant-associated microbes. Benefits of limiting replication of the plasmid system include, but are not limited to, making the cells more competitive and reducing deleterious mutations within the conjugation machinery. In some embodiments, a modified donor bacteria and plasmid system are designed to provide a gene essential for plasmid replication in the donor bacterial genome. In some embodiments, the essential gene is a pirA gene. A pirA gene encodes a second receptor for ferrienterobactin (SEQ ID NO: 49). In some embodiments, a pirA gene comprises a sequence comprising at least 80%, 85%, 90%, 95%, 97%, 98%, or 99% sequence identity to SEQ ID NO: 49. In some embodiments, a pirA gene comprises a sequence of SEQ ID NO: 49. In some embodiments, the single plasmid system described herein further comprises a partitioning locus. In some embodiments, the partitioning locus is parABCDE operon.

Plasmids or fragments thereof for use in compositions and methods described herein optionally include additional features. In some embodiments, a plasmid described herein is a mobilizable plasmid, a reporter plasmid, or a conjugative plasmid. In some embodiments, the conjugative plasmid is a pTAmob plasmid, an RP4 plasmid, an Rsa plasmid, an R702 plasmid, or an pIP113 plasmid. In some embodiments, the conjugative plasmid is a pTAmob plasmid. A pTAmob plasmid is a mobilization helper plasmid. In some embodiments, a pTAmob plasmid comprises one of more of the following elements: a resistance gene, pBBR1 replication protein gene (rep), ori, pBBR1 replication origin (ori), replication initiation protein gene from the RK2 replicon (trfA), regions containing the tra genes necessary for conjugative transfer of oriT containing plasmids (Tra1 and Tra2), stabilization region encoding the gene products ParA, B, C, D and E (parABCDE), and central control operon of RK2 (Ctl). In some embodiments, a pTAmob plasmid is modified to comprise a pRSA origin of replication, a tetracycline resistance gene, and Tra1 and Tra2 regions. In some embodiments, a pTAmob plasmid is modified to comprise other genes as described herein.

In some embodiments, one or more additional modifications are incorporated into plasmids, such as an origin of transfer (oriT) sequence, transcription regulator genes, reporter genes, or antibiotic resistance genes. In some embodiments, the reporter gene is a fluorescent reporter gene, a luminescent reporter gene, or another type of reporter gene. In some embodiments, the fluorescent reporter gene encodes for Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP), or far-red fluorescent protein. In some embodiments, the luminescent reporter gene encodes for luciferase. In some embodiments, the reporter gene encodes beta-galactosidase or chloramphenicol acetyltransferase (CAT).

In some embodiments, a vector described herein comprises a nucleic acid sequence encoding an antibiotic resistance gene or antibiotic resistance marker. In some embodiments, the antibiotic resistance gene is a tetracycline resistance gene, a kanamycin resistance gene, a gentamicin resistance gene, a rifampicin resistance gene, or a carbenicillin resistance gene.

In some embodiments, a vector described herein comprises short flippase recognition target (FRT) sites. In some embodiments, the resistance genes described herein are between a pair of FRT sites such that the resistance genes can be removed by a flippase.

The origin of replication, or replication origin, is a particular sequence at which replication is initiated. In some embodiments, the origin of replication is species-specific. In some embodiments, the origin of replication is functional across a broad range of species or hosts. In some embodiments, the plasmid comprises a broad host origin of replication. A broad host origin of replication is functional in a number of hosts in addition to the species from which it was isolated. In some embodiments, the broad host origin of replication comprises RK2, RSAOri, pRO1600, pBC1, pEP2, pWVO1, pLF1311, pAP1, pBBR1, pWKS1, pLS1, pUB6060, pJD4, RSF1010, R1162, R300B, pIJ101, pSN22, pAMP1, pIP501, ZM6100(Sa), pUC1, RA3, pMOL98, RP4, RP1, R68, pB10, or any derivative thereof.

The origin of replication determines the vector copy number. An origin directing a low copy number typically reduces metabolic burden on the host. In some embodiments, vectors described herein are larger than 50 kb. In some embodiments, vectors described herein encode 40 proteins or more. In such embodiments, a vector directing for a low copy number provides for higher competitive advantage of the host cell compared to wild type cells. In some embodiments, a vector directing for a low copy number increase stability of exogenous DNA in the host cell, reducing deleterious mutations from arising therein. In embodiments employing a high copy origin of replication, about 150-200 copies/cell are generated. In embodiments employing a low copy origin of replication, about 25-50 copies/cell are generated. In some embodiments, a low copy origin of replication generates about 1 to about 50, about 1 to about 40, about 1 to about 30, about 1 to about 20, about 1 to about 10, about 1-5, about 10 to about 20, about 20 to about 30, about 30 to about 40, or about 40 to about 50 copies/cell. In some embodiments, a plasmid comprises a low copy origin of replication. In some embodiments, the low copy origin of replication comprises, RK2, RSAOri, pRO1600, pBR322, pACYC, pSC101, or any functional variant thereof. In some embodiments, a plasmid described herein comprises a low copy broad-host origin of replication. In some embodiments, the low copy broad-host origin of replication is RSAOri, RK2, or pRO1600 origin of replication. Sequences of modifications included in plasmids as described herein are listed in Table 6. Plasmids used in embodiments described herein are provided in Table 7 noting relevant features incorporated as noted.

In some embodiments, a modified donor bacteria and plasmid system described herein comprises additional regulatory features. Such features can include one or more repressors expressed on the donor genome, paired with a promoter on a plasmid. In a plasmid system comprising a single plasmid encoding both conjugation and genome modulation machinery, a repressor acts on a promoter on the plasmid. In some embodiments, in a plasmid system comprising separate plasmids encoding each of the conjugation and genome modulation machinery, a repressor acts on a promoter on one or both plasmids. In some embodiments, in a plasmid system comprising separate plasmids encoding each of the conjugation and genome modulation machinery, one or more repressors act independently on a promoter on the gene modulation machinery plasmid and on a promoter on the conjugation machinery plasmid. In some embodiments, in a plasmid system comprising separate plasmids encoding each of the conjugation machinery and genome modulation machinery, the plasmid encoding the genome modulation machinery does not replicate autonomously. In some embodiments, the plasmid encoding the genome modulation machinery comprises a deletion or mutation of plasmid replication initiator protein (TrfA). In some embodiments, the plasmid encoding the conjugation machinery comprises a plasmid replication initiator protein (TrfA). In some embodiments, the TrfA comprises a sequence comprising at least 80%, 85%, 90%, 95%, 97%, 98%, or 99% sequence identity to SEQ ID NO: 46. In some embodiments, the TrfA comprises a sequence of SEQ ID NO: 46. In some embodiments, the plasmid encoding the genome modulation machinery only replicates replying on TrfA encoded by the plasmid encoding the conjugation machinery. In some embodiments, in a modified bacteria comprising conjugation machinery integrated into the genome of the modified bacteria and a plasmid encoding a genome modulation mechanism, a repressor acts on a promoter on the plasmid encoding the conjugation machinery. Expression of the repressor protein in the modified donor bacteria prevents expression of the promoter on the plasmid. In some embodiments, the promoter on the plasmid controls expression of a nuclease. Following transfer to a plant-associated bacteria, the promoter is no longer under the effect of the expressed repressor protein and the promoter is available to drive expression of the associated protein or RNA. In some embodiments, the repressor or promoter used in the compositions and methods described herein are encoded by a nucleic acid comprising at least 80%, 85%, 90%, 95%, 97%, 98%, or 99% sequence identity to one of SEQ ID NOs: 26-41. In some embodiments, the repressor/promoter pair is cI/cIPro. In some embodiments, the nucleic acid encoding the repressor protein cI comprises at least 80%, 85%, 90%, 95%, 97%, 98%, or 99% sequence identity to SEQ ID NO: 26, and the nucleic acid encoding the promoter cIPro comprises at least 80%, 85%, 90%, 95%, 97%, 98%, or 99% sequence identity to SEQ ID NO: 34. In some embodiments, the repressor/promoter pair is TP901/TP901Pro. In some embodiments, the nucleic acid encoding repressor protein TP901 comprises at least 80%, 85%, 90%, 95%, 97%, 98%, or 99% sequence identity to SEQ ID NO: 27, and the promoter comprises TP901Pro, as represented by SEQ ID NO: 35. In some embodiments, the repressor/promoter pair is 933W/933WPro. In some embodiments, the nucleic acid encoding the repressor protein 933W comprises at least 80%, 85%, 90%, 95%, 97%, 98%, or 99% sequence identity to SEQ ID NO: 28, and the nucleic acid encoding the promoter 933WPro comprises at least 80%, 85%, 90%, 95%, 97%, 98%, or 99% sequence identity to SEQ ID NO: 36. In some embodiments, the repressor/promoter pair is P2hd/P2hdPro. In some embodiments, the nucleic acid encoding the repressor protein P2hd comprises at least 80%, 85%, 90%, 95%, 97%, 98%, or 99% sequence identity to SEQ ID NO: 29, and the nucleic acid encoding the promoter P2hdPro comprises at least 80%, 85%, 90%, 95%, 97%, 98%, or 99% sequence identity to SEQ ID NO: 37. In some embodiments, the repressor/promoter pair is P2/P2Pro. In some embodiments, the nucleic acid encoding the repressor protein P2 comprises at least 80%, 85%, 90%, 95%, 97%, 98%, or 99% sequence identity to SEQ ID NO: 30, and the nucleic acid encoding the promoter P2Pro comprises at least 80%, 85%, 90%, 95%, 97%, 98%, or 99% sequence identity to SEQ ID NO: 38. In some embodiments, the repressor/promoter pair is Wphi/WphiPro. In some embodiments, the nucleic acid encoding the repressor protein Wphi comprises at least 80%, 85%, 90%, 95%, 97%, 98%, or 99% sequence identity to SEQ ID NO: 31 and the nucleic acid encoding the promoter WphiPro comprises at least 80%, 85%, 90%, 95%, 97%, 98%, or 99% sequence identity to SEQ ID NO: 39. In some embodiments, the repressor/promoter pair is CymR/pCymRC. The CymR transcriptional repressor protein is constitutively expressed and blocks transcription of Cas9. In some embodiments, Cas9 expression is titrated by supplementing cells with cuminic acid. In additional embodiments, a low level of transcriptional leakiness is sufficient for functional Cas9 expression. In some embodiments, the nucleic acid encoding the repressor protein CymR comprises at least 80%, 85%, 90%, 95%, 97%, 98%, or 99% sequence identity to SEQ ID NO: 32, and the nucleic acid encoding the promoter pCymRC comprises at least 80%, 85%, 90%, 95%, 97%, 98%, or 99% sequence identity to SEQ ID NO: 40. In some embodiments, the repressor/promoter pair is PhlF/PhlFPro. In some embodiments, the nucleic acid encoding the repressor protein PhlF comprises at least 80%, 85%, 90%, 95%, 97%, 98%, or 99% sequence identity to SEQ ID NO: 33, and the nucleic acid encoding the promoter PhlFPro comprises at least 80%, 85%, 90%, 95%, 97%, 98%, or 99% sequence identity to SEQ ID NO: 41. Sequences are shown in Table 6.

TABLE 6
Sequences of plasmid modifications.

| SEQ | | | |
|---|---|---|---|
| ID | | | |
| NO: | Identifier | Description | Sequence |
| 1 | Cas9 | pCymRC- | CTAGCACCGCCTATCTCGTGTGAGATAGGCGGAGATACGAACTTT |
| | cassette | RBS-Cas9- | AAGAAGGAGATATACCATGGATAAGAAATACTCAATAGGCTTAGA |
| | | DT36 | TATCGGCACAAATAGCGTCGGATGGGCGGTGATCACTGATGAATA |
| | | | TAAGGTTCCGTCTAAAAAGTTCAAGGTTCTGGGAAATACAGACCG |
| | | | CCACAGTATCAAAAAAAATCTTATAGGGGCTCTTTTATTTGACAG |
| | | | TGGAGAGACAGCGGAAGCGACTCGTCTCAAACGGACAGCTCGTAG |
| | | | AAGGTATACACGTCGGAAGAATCGTATTTGTTATCTACAGGAGAT |
| | | | TTTTTCAAATGAGATGGCGAAAGTAGATGATAGTTTCTTTCATCG |
| | | | ACTTGAAGAGTCTTTTTTGGTGGAAGAAGACAAGAAGCATGAACG |
| | | | TCATCCTATTTTTGGAAATATAGTAGATGAAGTTGCTTATCATGA |
| | | | GAAATATCCAACTATCTATCATCTGCGAAAAAAATTGGTAGATTC |
| | | | TACTGATAAAGCGGATTTGCGCTTAATCTATTTGGCCTTAGCGCA |
| | | | TATGATTAAGTTTCGTGGTCATTTTTTGATTGAGGGAGATTTAAA |
| | | | TCCTGATAATAGTGATGTGGACAAACTATTTATCCAGTTGGTACA |
| | | | AACCTACAATCAATTATTTGAAGAAAACCCTATTAACGCAAGTGG |
| | | | AGTAGATGCTAAAGCGATTCTTTCTGCACGATTGAGTAAATCAAG |
| | | | ACGATTAGAAAATCTCATTGCTCAGCTCCCCGGTGAGAAGAAAAA |
| | | | TGGCTTATTTGGGAATCTCATTGCTTTGTCATTGGGTTTGACCCC |
| | | | TAATTTTAAATCAAATTTTGATTTGGCAGAAGATGCTAAATTACA |
| | | | GCTTTCAAAAGATACTTACGATGATGATTTAGATAATTTATTGGC |
| | | | GCAAATTGGAGATCAATATGCTGATTTGTTTTTGGCAGCTAAGAA |
| | | | TTTATCAGATGCTATTTTACTTTCAGATATCCTAAGAGTAAATAC |
| | | | TGAAATAACTAAGGCTCCCCTATCAGCTTCAATGATTAAACGCTA |
| | | | CGATGAACATCATCAAGACTTGACTCTTTTAAAAGCTTTAGTTCG |
| | | | ACAACAACTTCCAGAAAAGTATAAAGAAATCTTTTTTGATCAATC |
| | | | AAAAAACGGATATGCAGGTTATATTGATGGGGGAGCTAGCCAAGA |
| | | | AGAATTTTATAAATTTATCAAACCAATTTTAGAAAAAATGGATGG |
| | | | TACTGAGGAATTATTGGTGAAACTAAATCGTGAAGATTTGCTGCG |
| | | | CAAGCAACGGACCTTTGACAACGGCTCTATTCCCCATCAAATTCA |
| | | | CTTGGGTGAGCTGCATGCTATTTTGAGAAGACAAGAAGACTTTTA |
| | | | TCCATTTTTAAAAGACAATCGTGAGAAGATTGAAAAAATCTTGAC |
| | | | TTTTCGAATTCCTTATTATGTTGGTCCATTGGCGCGTGGCAATAG |
| | | | TCGTTTTGCATGGATGACTCGGAAGTCTGAAGAAACAATTACCCC |
| | | | ATGGAATTTTGAAGAAGTTGTCGATAAAGGTGCTTCAGCTCAATC |
| | | | ATTTATTGAACGCATGACAAACTTTGATAAAAATCTTCCAAATGA |
| | | | AAAAGTACTACCAAAACATAGTTTGCTTTATGAGTATTTTACGGT |
| | | | TTATAACGAATTGACAAAGGTCAAATATGTTACTGAAGGAATGCG |
| | | | AAAACCAGCATTTCTTTCAGGTGAACAGAAGAAAGCCATTGTTGA |
| | | | TTTACTCTTCAAAACAAATCGAAAAGTAACCGTTAAGCAATTAAA |
| | | | AGAAGATTATTTCAAAAAAATAGAATGTTTTGATAGTGTTGAAAT |
| | | | TTCAGGAGTTGAAGATAGATTTAATGCTTCATTAGGTACCTACCA |
| | | | TGATTTGCTAAAAATTATTAAAGATAAAGATTTTTTGGATAATGA |
| | | | AGAAAATGAAGATATCTTAGAGGATATTGTTTTAACATTGACCTT |
| | | | ATTTGAAGATAGGGAGATGATTGAGGAAAGACTTAAAACATATGC |
| | | | TCACCTCTTTGATGATAAGGTGATGAAACAGCTTAAACGTCGCCG |
| | | | TTATACTGGTTGGGGACGTTTGTCTCGAAAATTGATTAATGGTAT |
| | | | TAGGGATAAGCAATCTGGCAAAACAATATTAGATTTTTTGAAATC |
| | | | AGATGGTTTTGCCAATCGCAATTTTATGCAGCTGATCCATGATGA |
| | | | TAGTTTGACATTTAAAGAAGACATTCAAAAAGCACAAGTGTCTGG |
| | | | ACAAGGCGATAGTTTACATGAACATATTGCAAATTTAGCTGGTAG |
| | | | CCCTGCTATTAAAAAAGGTATTTTACAGACTGTAAAAGTTGTTGA |
| | | | TGAATTGGTCAAAGTAATGGGGCGGCATAAGCCAGAAAATATCGT |
| | | | TATTGAAATGGCACGTGAAAATCAGACAACTCAAAAGGGCCAGAA |
| | | | AAATTCGCGAGAGCGTATGAAACGAATCGAAGAAGGTATCAAAGA |
| | | | ATTAGGAAGTCAGATTCTTAAAGAGCATCCTGTTGAAAATACTCA |
| | | | ATTGCAAAATGAAAAGCTCTATCTCTATTATCTCCAAAATGGAAG |
| | | | AGACATGTATGTGGACCAAGAATTAGATATTAATCGTTTAAGTGA |
| | | | TTATGATGTCGATCACATTGTTCCACAAAGTTTCCTTAAAGACGA |
| | | | TTCAATAGACAATAAGGTCTTAACGCGTTCTGATAAAAATCGTGG |
| | | | TAAATCGGATAACGTTCCAAGTGAAGAAGTAGTCAAAAAGATGAA |
| | | | AAACTATTGGAGACAACTTCTAAACGCCAAGTTAATCACTCAACG |
| | | | TAAGTTTGATAATTTAACGAAAGCTGAACGTGGAGGTTTGAGTGA |
| | | | ACTTGATAAAGCTGGTTTTATCAAACGCCAATTGGTTGAAACTCG |
| | | | CCAAATCACTAAGCATGTGGCACAAATTTTGGATAGTCGCATGAA |
| | | | TACTAAATACGATGAAAATGATAAACTTATTCGAGAGGTTAAAGT |
| | | | GATTACCTTAAAATCTAAATTAGTTTCTGACTTCCGAAAAGATTT |
| | | | CCAATTCTATAAAGTACGTGAGATTAACAATTACCATCATGCCCA |
| | | | TGATGCGTATCTAAATGCCGTCGTTGGAACTGCTTTGATTAAGAA |
| | | | ATATCCAAAACTTGAATCGGAGTTTGTCTATGGTGATTATAAAGT |
| | | | TTATGATGTTCGTAAAATGATTGCTAAGTCTGAGCAAGAAATAGG |
| | | | CAAAGCAACCGCAAAATATTTCTTTTACTCTAATATCATGAACTT |
| | | | CTTCAAAACAGAAATTACACTTGCAAATGGAGAGATTCGCAAACG |
| | | | CCCTCTAATCGAAACTAATGGGGAAACTGGAGAAATTGTCTGGGA |
| | | | TAAAGGGCGAGATTTTGCCACAGTGCGCAAAGTATTGTCCATGCC |
| | | | CCAAGTCAATATTGTCAAGAAAACAGAAGTACAGACAGGCGGATT |
| | | | CTCCAAGGAGTCAATTTTACCAAAAAGAAATTCGGACAAGCTTAT |
| | | | TGCTCGTAAAAAAGACTGGGATCCAAAAAAATATGGTGGTTTTGA |
| | | | TAGTCCAACGGTAGCTTATTCAGTCCTAGTGGTTGCTAAGGTGGA |
| | | | AAAAGGGAAATCGAAGAAGTTAAAATCCGTTAAAGAGTTACTAGG |
| | | | GATCACAATTATGGAAAGAAGTTCCTTTGAAAAAAATCCGATTGA |
| | | | CTTTTTAGAAGCTAAAGGATATAAGGAAGTTAAAAAAGACTTAAT |
| | | | CATTAAACTACCTAAATATAGTCTTTTTGAGTTAGAAAACGGTCG |
| | | | TAAACGGATGCTGGCTAGTGCCGGAGAATTACAAAAAGGAAATGA |
| | | | GCTGGCTCTGCCAAGCAAATATGTGAATTTTTTATATTTAGCTAG |
| | | | TCATTATGAAAAGTTGAAGGGTAGTCCAGAAGATAACGAACAAAA |
| | | | ACAATTGTTTGTGGAGCAGCATAAGCATTATTTAGATGAGATTAT |
| | | | TGAGCAAATCAGTGAATTTTCTAAGCGTGTTATTTTAGCAGATGC |
| | | | CAATTTAGATAAAGTTCTTAGTGCATATAACAAACATAGAGACAA |
| | | | ACCAATACGTGAACAAGCAGAAAATATTATTCATTTATTTACGTT |
| | | | GACGAATCTTGGAGCTCCCGCTGCTTTTAAATATTTTGATACAAC |
| | | | AATTGATCGTAAACGATATACGTCTACAAAAGAAGTTTTAGATGC |
| | | | CACTCTTATCCATCAATCCATCACTGGTCTTTATGAAACACGCAT |
| | | | TGATTTGAGTCAGCTAGGAGGTGACTGAGATCTAACTAAAAAGGC |
| | | | CGCTCTGCGGCCTTTTTTCTTTTCACTGTAACAACGGAAACCGGC |
| | | | CATTGCGCCGGTTTTTTTTGGCCT |
| 2 | OriT | Origin of | TTAAGGTATACTTTCCGCTGCATAACCCTGCTTCGGGGTCATTAT |
| | | transfer | AGCGATTTTTTCGGTATATCCATCCTTTTTCGCACGATATACAGG |
| | | (TAmob) | ATTTTGCCAAAGGGTTCGTGTAGACTTTCCTTGGTGTATCCAACG |
| | | | GCGTCAGCCGGGCAGGATAGGTGAAGTAGGCCCACCCGCGAGCGG |
| | | | GTGTTCCTTCTTCACTGTCCCTTATTCGCACCTGGCGGTGCTCAA |
| | | | CGGGAATCCTGCTCTGCGAGGCTGGCCGATAAGCT |
| 3 | GFP | Reporter, | AACAAACAGACAATCTGGTCTGTTTGTATTATGGAAAATTTTTCT |
| | | pCymRC- | GTATAATAGATTCAACAAACAGACAATCTGGTCTGTTTGTATTAT |
| | | RBS-GFP- | TCTAGAATTAAAGAGGAGAAATTAACCATGAGTAAAGGAGAAGAA |
| | | rrnBT1T2 | CTTTTCACTGGAGTTGTCCCAATTCTTGTTGAATTAGATGGTGAT |
| | | terminator | GTTAATGGGCACAAATTTTCTGTTAGTGGAGAGGGTGAAGGTGAT |
| | | | GCAACATACGGAAAACTTACCCTTAAATTTATTTGCACTACTGGA |
| | | | AAACTACCTGTTCCATGGCCAACACTTGTCACTACTTTCGGTTAT |
| | | | GGTGTTCAATGCTTTGCGAGATACCCAGATCATATGAAACAGCAT |
| | | | GACTTTTTCAAGAGTGCCATGCCCGAAGGTTATGTACAGGAAAGA |
| | | | ACTATATTTTTCAAAGATGACGGGAACTACAAGACACGTGCTGAA |
| | | | GTCAAGTTTGAAGGTGATACCCTTGTTAATAGAATCGAGTTAAAA |
| | | | GGTATTGATTTTAAAGAAGATGGAAACATTCTTGGACACAAATTG |
| | | | GAATACAACTATAACTCACACAATGTATACATCATGGCAGACAAA |
| | | | CAAAAGAATGGAATCAAAGTTAACTTCAAAATTAGACACAACATT |
| | | | GAAGATGGAAGCGTTCAACTAGCAGACCATTATCAACAAAATACT |
| | | | CCAATTGGCGATGGCCCTGTCCTTTTACCAGACAACCATTACCTG |
| | | | TCCACACAATCTGCCCTTTCGAAAGATCCCAACGAAAAGAGAGAC |
| | | | CACATGGTCCTTCTTGAGTTTGTAACAGCTGCTGGGATTACACAT |
| | | | GGCATGGATGAACTATACAAATAGCAAATAAAACGAAAGGCTCAG |
| | | | TCGAAAGACTGGGCCTTTCGTTTTATCTGTTGTTTGTCGGTGAAC |
| | | | GCTCTCCTGAGTAGGACAAATCCGCCGGGAGCGGATTTGAACGTT |
| | | | GCGAAGCAACGGCCCGGAGGGTGGCGGGCAGGACGCCCGCCATAA |
| | | | ACTGCCAGGCATCAAATTAAGCAGAAGGCCATCCTGACGGATGGC |
| | | | CTTTT |
| 4 | mCherry | Reporter, | TTTACAGCTAGCTCAGTCCTAGGTATTATGCTAGCCTGAAGTACG |
| | | J23101- | TCTGAGCGTGATACCCGCTCACTGAAGATGGCCCGGTAGGGCCGA |
| | | LtsvJ-RBS- | AACGTACCTCTACAAATAATTTTGTTTAATCACACATCTAGAATT |
| | | mCherry- | AAAGAGGAGAAATTAAGCATGGTTTCGAAGGGCGAGGAGGATAAC |
| | | lambda t0 | ATGGCCATCATCAAGGAGTTCATGCGCTTCAAGGTGCACATGGAG |
| | | terminator | GGCTCCGTGAACGGCCACGAGTTCGAGATCGAGGGCGAGGGCGAG |
| | | | GGCCGCCCCTACGAGGGCACCCAGACCGCCAAGCTGAAGGTGACC |
| | | | AAGGGTGGCCCCCTGCCCTTCGCCTGGGACATCCTGTCCCCTCAG |
| | | | TTCATGTACGGCTCCAAGGCCTACGTGAAGCACCCCGCCGACATC |
| | | | CCCGACTACTTGAAGCTGTCCTTCCCCGAGGGCTTCAAGTGGGAG |
| | | | CGCGTGATGAACTTCGAGGACGGCGGCGTGGTGACTGTGACCCAG |
| | | | GACTCCTCCTTGCAGGACGGCGAGTTCATCTACAAGGTGAAGCTG |
| | | | CGCGGCACCAACTTCCCCTCCGACGGCCCCGTAATGCAGAAGAAG |
| | | | ACGATGGGCTGGGAGGCCTCCTCCGAGCGGATGTACCCCGAGGAC |
| | | | GGCGCCCTGAAGGGCGAGATCAAGCAGAGGCTGAAGCTGAAGGAC |
| | | | GGCGGCCACTACGACGCTGAGGTCAAGACCACCTACAAGGCCAAG |
| | | | AAGCCCGTGCAGCTGCCCGGCGCCTACAACGTCAACATCAAGTTG |
| | | | GACATCACCTCCCACAACGAGGACTACACCATCGTGGAACAGTAC |
| | | | GAACGCGCCGAGGGCCGCCACTCCACCGGCGGCATGGACGAGCTG |
| | | | TACAAGTAATAATACTCGAACCCCTAGCCCGCTCTTATCAGCTTA |
| | | | ATTAGCTGAGCTTGGACTCCTGTTGATAGATCCAGTAATGACCTC |
| | | | AGAACTCCATCTGGATTTGTTCAGAACGCTCGGTTGCCGCCGGGC |
| | | | GTTTTTTATTGGTGAGAAT |
| 5 | CymR | Repressor, | ATGAGCCCGAAACGTCGTACCCAGGCAGAACGTGCAATGGAAACC |
| | cassette | LacIQ- | CAGGGTAAACTGATTGCAGCAGCACTGGGTGTTCTGCGTGAAAAA |
| | | CymR-F10 | GGTTATGCAGGTTTTCGTATTGCAGATGTTCCGGGTGCAGCCGGT |
| | | terminator | GTTAGCCGTGGTGCACAGAGCCATCATTTTCCGACCAAACTGGAA |
| | | | CTGCTGCTGGCAACCTTTGAATGGCTGTATGAGCAGATTACCGAA |
| | | | CGTAGCCGTGCACGTCTGGCAAAACTGAAACCGGAAGATGATGTT |
| | | | ATTCAGCAGATGCTGGATGATGCAGCAGAATTTTTTCTGGATGAT |
| | | | GATTTTAGCATCAGCCTGGATCTGATTGTTGCAGCAGATCGTGAT |
| | | | CCGGCACTGCGTGAAGGTATTCAGCGTACCGTTGAACGTAATCGT |
| | | | TTTGTTGTTGAAGATATGTGGCTGGGTGTGCTGGTGAGCCGTGGT |
| | | | CTGAGCCGTGATGATGCCGAAGATATTCTGTGGCTGATTTTTAAC |
| | | | AGCGTTCGTGGTCTGGCAGTTCGTAGCCTGTGGCAGAAAGATAAA |
| | | | GAACGTTTTGAACGTGTGCGTAATAGCACCCTGGAAATTGCACGT |
| | | | GAACGTTATGCAAAATTCAAACGTTGA |
| 6 | P2C | Repressor, | TTTACAGCTAGCTCAGTCCTAGGTATTATGCTAGCCTGAAGGAGT |
| | | P2C (with | CAATTAATGTGCTTTTAATTCTGATGAGACGGTGACGTCGAAACT |
| | | J23101, | CCCTCTACAAATAATTTTGTTTAACAGGTTATAAGGCCTGCAGCG |
| | | RiboJ64, | TATGTCAAACACGATAAGCGAGAAGATAGTCTTAATGCGAAAATC |
| | | RBS, P2C, | AGAGTATTTGAGCAGACAACAACTTGCTGATTTAACAGGGGTTCC |
| | | DT86 | GTATGGCACGCTGAGTTACTATGAAAGTGGTCGTTCAACACCTCC |
| | | terminator) | AACAGATGTCATGATGAACATCCTGCAGACCCCACAATTCACCAA |
| | | | ATACACTTTATGGTTCATGACCAATCAGATCGCTCCTGAGTCCGG |
| | | | GCAAATTGCGCCCGCTCTCGCACACTTTGGGCAAAACGAAACAAC |
| | | | GTCGCCCCACTCCGGTCAAAAGACTGGTTAATAATCATTCTTAGC |
| | | | GTGACCGGGAAGTCGGTCACGCTACCTCTTCTGAAGAAACAGCAA |
| | | | ACAATCCAAAACGCCGCGTTCAGCGGCGTTTTTTCTGCTTTTCT |
| 7 | Ph2d | Repressor, | TTTACAGCTAGCTCAGTCCTAGGTATTATGCTAGCCTGACTGAAG |
| | | Ph2d | TCGTCAAGTGCTGTGCTTGCACTTCTGATGAGGCAGTGATGCCGA |
| | | (J23101, | AACGACCTCTACAAATAATTTTGTTTAATTATCTTAGGTACAGGG |
| | | RiboJ60, | CACATAAATGTCAATAGACGTTTCGGAGAAGTTGAAGCTAATCCG |
| | | RBS, Ph2d, | TGAATCTGAAAGGTTAAACCGTAAAGAATTCAGTGAATTAACTGG |
| | | DT56 | TGTAGCCTACAGCTCACTTTCGAGCTATGAGAGCCGGTCAAAAAA |
| | | terminator) | CGCTGGAGTTGAAGCCATAATGAAGGTCTTACAACATCCTAGATT |
| | | | TACTAAATATACTTTGTGGTTCATGACTGATCAGGTAGCTCCAGA |
| | | | AGCCGGGCAAATTGCGCCCGCTCTCGCACACTTTGGGCAAAACGA |
| | | | AACAACGTCGCCCCACTCCGGTCAAAAGACTGGTTAATACCACCG |
| | | | TCAAAAAAAACGGCGCTTTTTAGCGCCGTTTTTATTTTTCAACCT |
| | | | TCCAGGCATCAAATAAAACGAAAGGCTCAGTCGAAAGACTGGGCC |
| | | | TTTCGTTTTATCTGTTGTTTGTCGGTGAACGCTCTC |
| 8 | WphiC | Repressor, | TTTACAGCTAGCTCAGTCCTAGGTATTATGCTAGCCTGAGACTGT |
| | | WphiC | CGCCGGATGTGTATCCGACCTGACGATGGCCCAAAAGGGCCGAAA |
| | | (J23101, | CAGTCCTCTACAAATAATTTTGTTTAATGCCACTTACATTAGCGT |
| | | SarJ, RBS, | TTTTGAATGCAGACATTCGAAAAACTGAAAGCGATTAGGAAAGCA |
| | | WphiC, | GAAGGCTTAACACAGGCGAAATTCAGCGAAATTAGCGGGATAGCT |
| | | DT19 | CTAGGAACAGTCAAAAATTACGAAAGTGGGCATAAAGACCCTGGT |
| | | terminator) | CTGAGCATCGTTATGCGAGTCACAAATACGCCTTTATTTAAAAAA |
| | | | TATACGCTCTGGTTAATGACTGGTGATACGTCACCACAAGCTGGT |
| | | | CAGATCGCGCCGGCTCTCGCACACATTGGGCAAAAACCAACAGAA |
| | | | TCAGACCACTCCGAAAAACAGACTGGTTAATTCAGCCAAAAAACT |
| | | | TAAGACCGCCGGTCTTGTCCACTACCTTGCAGTAATGCGGTGGAC |
| | | | AGGATCGGCGGTTTTCTTTTCTCTTCTCAACTCGGTACCAAAGAC |
| | | | GAACAATAAGACGCTGAAAAGCGTCTTTTTTCGTTTTGGTCC |
| 9 | KanR | Resistance | ATGAGCCATATTCAACGGGAAACGTCTTGCTCCAGGCCGCGATTA |
| | | Marker, | AATTCCAACATGGATGCTGATTTATATGGGTATAAATGGGCTCGC |
| | | Kanamycin | GATAATGTCGGGCAATCAGGTGCGACAATCTATCGATTGTATGGG |
| | | phospho- | AAGCCCGATGCGCCAGAGTTGTTTCTGAAACATGGCAAAGGTAGC |
| | | transferase | GTTGCCAATGATGTTACAGATGAGATGGTCAGACTAAACTGGCTG |
| | | | ACGGAATTTATGCCTCTTCCGACCATCAAGCATTTTATCCGTACT |
| | | | CCTGATGATGCATGGTTACTCACCACTGCGATCCCCGGGAAAACA |
| | | | GCATTCCAGGTATTAGAAGAATATCCTGATTCAGGTGAAAATATT |
| | | | GTTGATGCGCTGGCAGTGTTCCTGCGCCGGTTGCATTCGATTCCT |
| | | | GTTTGTAATTGTCCTTTTAACAGCGATCGCGTATTTCGTCTCGCT |
| | | | CAGGCGCAATCACGAATGAATAACGGTTTGGTTGATGCGAGTGAT |
| | | | TTTGATGACGAGCGTAATGGCTGGCCTGTTGAACAAGTCTGGAAA |
| | | | GAAATGCATAAGCTTTTGCCATTCTCACCGGATTCAGTCGTCACT |
| | | | CATGGTGATTTCTCACTTGATAACCTTATTTTTGACGAGGGGAAA |
| | | | TTAATAGGTTGTATTGATGTTGGACGAGTCGGAATCGCAGACCGA |
| | | | TACCAGGATCTTGCCATCCTATGGAACTGCCTCGGTGAGTTTTCT |
| | | | CCTTCATTACAGAAACGGCTTTTTCAAAAATATGGTATTGATAAT |
| | | | CCTGATATGAATAAATTGCAGTTTCATTTGATGCTCGATGAGTTT |
| | | | TTCTAA |
| 10 | TetR | Resistance | ATGCCGGTACTGCCGGGCCTCTTGCGGGATATCGTCCATTCCGAC |
| | | Marker, | AGCATCGCCAGTCACTATGGCGTGCTGCTAGCGCTATATGCGTTG |
| | | Tetra- | ATGCAATTTCTATGCGCACCCGTTCTCGGAGCACTGTCCGACCGC |
| | | cycline | TTTGGCCGCCGCCCAGTCCTGCTCGCTTCGCTACTTGGAGCCACT |
| | | | ATCGACTACGCGATCATGGCGACCACACCCGTCCTGTGGATTCTC |
| | | | TACGCCGGACGCATCGTGGCCGGCATCACCGGCGCCACAGGTGCG |
| | | | GTTGCTGGCGCCTATATCGCCGACATCACCGATGGGGAAGATCGG |
| | | | GCTCGCCACTTCGGGCTCATGAGCGCTTGTTTCGGCGTGGGTATG |
| | | | GTGGCAGGCCCCGTGGCCGGGGGACTGTTGGGCGCCATCTCCCTG |
| | | | CACGCACCATTCCTTGCGGCGGCGGTGCTCAACGGCCTCAACCTA |
| | | | CTACTGGGCTGCTTCCTAATGCAGGAGTCGCATAAGGGAGAGCGC |
| | | | CGTCCGATGCCCTTGAGAGCCTTCAACCCAGTCAGCTCCTTCCGG |
| | | | TGGGCGCGGGGCATGACCATTGTGGCCGCACTTATGACTGTCTTC |
| | | | TTTATCATGCAACTCGTAGGACAGGTGCCGGCAGCGCTCTGGGTC |
| | | | ATTTTCGGCGAGGACCGCTTTCGCTGGAGCGCGACGATGATCGGC |
| | | | CTGTCGCTTGCGGTATTCGGAATCTTGCACGCCCTCGCTCAAGCC |
| | | | TTCGTCACTGGTCCCGCCACCAAACGTTTCGGCGAGAAGCAGGCC |
| | | | ATTATCGCCGGCATGGCGGCCGACGCGCTGGGCTACGTCTTGCTG |
| | | | GCGTTCGCGACGCGAGGCTGGATGGCCTTCCCCATTATGATTCTT |
| | | | CTCGCTTCCGGCGGCATCGGGATGCCCGCGTTGCAGGCCATGCTG |
| | | | TCCAGGCAGGTAGATGACGACCATCAGGGACAGCTTCAAGGATCG |
| | | | CTCGCGGCTCTTACCAGCCTAACTTCGATCACTGGACCGCTGATC |
| | | | GTCACGGCGATTTATGCCGCCTCGGCGAGCACATGGAACGGGTTG |
| | | | GCATGGATTGTAGGCGCCGCCCTATACCTTGTCTGCCTCCCCGCG |
| | | | TTGCGTCGCGGTGCATGGAGCCGGGCCACCTCGACCTGA |
| 11 | GmR | Resistance | CCTTGTCGCCTTGCGTATAATATTTGCCCATGGACGCACACCGTG |
| | | Marker, | GAAACGGATGAAGGCACGAACCCAGTTGACATAAGCCTGTTCGGT |
| | | Gentamicin | TCGTAAACTGTAATGCAAGTAGCGTATGCGCTCACGCAACTGGTC |
| | | | CAGAACCTTGACCGAACGCAGCGGTGGTAACGGCGCAGTGGCGGT |
| | | | TTTCATGGCTTGTTATGACTGTTTTTTTGTACAGTCTATGCCTCG |
| | | | GGCATCCAAGCAGCAAGCGCGTTACGCCGTGGGTCGATGTTTGAT |
| | | | GTTATGGAGCAGCAACGATGTTACGCAGCAGCAACGATGTTACGC |
| | | | AGCAGGGCAGTCGCCCTAAAACAAAGTTAGGTGGCTCAAGTATGG |
| | | | GCATCATTCGCACATGTAGGCTCGGCCCTGACCAAGTCAAATCCA |
| | | | TGCGGGCTGCTCTTGATCTTTTCGGTCGTGAGTTCGGAGACGTAG |
| | | | CCACCTACTCCCAACATCAGCCGGACTCCGATTACCTCGGGAACT |
| | | | TGCTCCGTAGTAAGACATTCATCGCGCTTGCTGCCTTCGACCAAG |
| | | | AAGCGGTTGTTGGCGCTCTCGCGGCTTACGTTCTGCCCAAGTTTG |
| | | | AGCAGCCGCGTAGTGAGATCTATATCTATGATCTCGCAGTCTCCG |
| | | | GAGAGCACCGGAGGCAGGGCATTGCCACCGCGCTCATCAATCTCC |
| | | | TCAAGCATGAGGCCAACGCGCTTGGTGCTTATGTGATCTACGTGC |
| | | | AAGCAGATTACGGTGACGATCCCGCAGTGGCTCTCTATACAAAGT |
| | | | TGGGCATACGGGAAGAAGTGATGCACTTTGATATCGACCCAAGTA |
| | | | CCGCCACCTAACAATTCGTTCAAGCCGAGAT |
| 12 | RK2-par | RK2 vector | GCGCAACGCAATTAATGTGAGTTAGCTCACTCATTAGGCACCCCA |
| | locus | with OriV | GGCTTTACACTTTATGCTTCCGGCTCGTATGTTGTGTGGAATTGT |
| | | origin of | GAGCGGATAACAATTTCACACAGGAAACAGCTATGACCATGATTA |
| | | replication | CGCCAAGCTGATCCCCCGATGTGTAGCAGTGGCGGACCATATAGG |
| | | and | CAGATCAGAAGGCGCGGTTCTCCTACATGTTCGCCGGTGAACGCG |
| | | partitioning | TTGAGGAAGCCGGGCAGTGCCTCGGCAAAATCCTTGCGTGTAGAC |
| | | locus | AAGACATCTGCGTAGCAGTTGTCCTCAACAACGATGTCGAAATCC |
| | | (parABCDE) | AAATCGGAGTGCTCATCGAGTCCTCCGTGAACGTAAGAGCCGCCG |
| | | | ATCAGAAGAGCGCGGAAGCGAACATCGGAAGCGACCGCATCGCGG |
| | | | ATGCGGTTCAAGAAAGTTGCATGAGCTTGTGGAAGTGTGCTGAGC |
| | | | ATAAATGATTCTCCTAGCTGTTCTTTGGGTAAGTACGCCATCAGG |
| | | | ACGTTGTGAGTGGCGCGATTTTTAGCGGCTGAAATCAGCCCTTGA |
| | | | GCCTGTCGGCAAGTCGCGTCATGAGGTCCATGCGCTCATGCAGGA |
| | | | TCGCCACGACCAACGCGGGTTCGCCCGCACGCGGCAGGCAAAAAA |
| | | | CGTAGTGGTGTTCGCAGCGGGCCATCCGCAGCGCGGGAAAGAGTT |
| | | | CGCTCATGTCCTTAAACGGGCCTTCGCCGGCGGCAAGCCTGGCTA |
| | | | TGCCCTGTTCCAGCTTAGCGATATAGCGGCGCACCTGCGCCGCGC |
| | | | CCCACTCCCGGCGCGTGTAGCGGATGATGCCGCGTAGATCGGCTT |
| | | | CGGCCTCAGCCGTGAGGATGTAGGCCGTCAAGCGCGATCCCCGCT |
| | | | GAGTTCTTCATCAAGAATTTCGCCGACGCTCTTGGTGGACACCTT |
| | | | GCCGGCAAGCCCATCGTTGATGCGGTTCCCCAGCATGGTTTTCAG |
| | | | TTCCTGCCATGCCTGATCGGCATCAGCGTCACCGGGGAACAGACG |
| | | | TTCGAGGGCGTATTGCTTAATGGTCTTGCCCTGCAAGGCGGCCAG |
| | | | GGCTTTCAGGCTCTGGTGCTGCTGGTCCGTCATGTCGATTGTCAG |
| | | | GCGGCTCATTGGATAACCTCCATAAAATACACGTAACCACATTAG |
| | | | CACATATGTGGGCGTGAGGCTACAGCGCGAGGCGCATTAAGGTCG |
| | | | GGAAAATGCGCTAGGCGCATTTAAATTGCGTATTGCTGTAATGCG |
| | | | CCATGCCGGCTAGACTAGGCCCAAATGGGTATACCCAATTTGACC |
| | | | AAGGGGGACGCGATGAGGGCGGCCAAGCACTACCGACAACTTCTA |
| | | | TCCATCGACTTCAACATCGAGGCGCTGGCCTTCGTGCCTGGACCC |
| | | | GACGGCACACGCGGCCGGCGCATCCACGTCCTGGGGCGCGAGGTC |
| | | | CGCGACCGGCCCGGCCTGGTCGAGTACCTTTCGCCGGCGTTCGGC |
| | | | TCGCGGGTGGCGCTGGACGGCTACTGCAAGGCCAATTTCGATGCA |
| | | | GTGCTGCACCTGGCGTACCCCGATCATCAGCAATGGGGCCACGCA |
| | | | TGAAGCGCCGAAGCTACGCCATGCTGCGCGCCGCTGCCGCGCTGG |
| | | | CCGTCCTGGTCGTTGCCTCGCCGGCATGGGCCGAGCTGCGCGGCG |
| | | | AGGTCGTGCGCATCATCGACGGCGACACCATCGACGTGCTGGTAG |
| | | | ACAAGCAGCCGGTGCGCGTGCGCCTGGTGGACATTGACGCGCCGG |
| | | | AAAAGCGGCAAGCCTTCGGCGAACGTGCGCGCCAGGCGCTGGCCG |
| | | | GCATGGTGTTCCGCCGGCACGTCCTGGTCGACGAGAAGGACACCG |
| | | | ACCGTTACGGCCGCACGCTGGGCACCGTGTGGGTCAACATGGAGC |
| | | | TGGCCAGCCGGCCGCCGCAGCCGCGCAACGTCAACGCCGCGATGG |
| | | | TTCACCAGGGCATGGCGTGGGCCTATCGCTTCCACGGCCGCGCGG |
| | | | CCGACCCTGAAATGCTGCGGCTCGAACAGGAGGCGCGAGGCAAGC |
| | | | GCGTCGGCCTCTGGTCCGATCCGCACGCCGTCGAGCCGTGGAAAT |
| | | | GGCGACGCGAGAGCAACAACCGGAGGGACGAAGGTTGAAGGTCGC |
| | | | CCGCATCTACCTGCGCGCCAGTACGGACGAGCAGAATCTTGAACG |
| | | | CCAGGAGAGCCTTGTAGCGGCCACGCGGGCCGCCGGGTACTACGT |
| | | | CGCCGGCATCTACCGCGAGAAGGCGTCCGGCGCACGCGCCGACCG |
| | | | GCCCGAGCTGCTGCGCATGATCGCGGACCTGCAACCTGGTGAAGT |
| | | | CGTCGTTGCGGAGAAGATCGACCGCATCAGCCGCTTGCCGTTGGC |
| | | | CGAGGCCGAGCGCCTGGTTGCGTCGATCCGGGCCAAAGGGGCCAA |
| | | | GCTGGCCGTGCCTGGCGTGGTGGACCTGTCGGAGCTGGCCGCCGA |
| | | | GGCGAACGGAGTGGCGAAAATCGTTCTGGAATCCGTCCAGGACAT |
| | | | GCTTTTGAAGCTCGCCTTGCAGATGGCCCGCGACGACTACGAGGA |
| | | | TCGGCGCGAGCGTCAACGTCAGGGTGTCCAGTTGGCGAAGGCCGC |
| | | | CGGCCGCTACACCGGCCGCAAACGTGACGCCGGCATGCACGACCG |
| | | | CATCATCACGCTTCGCTCCGGCGGATCGAGCATTGCCAAGACGGC |
| | | | CAAGCTGGTCGGATGCAGCCCGAGCCAGGTCAAACGAGTGTGGGC |
| | | | GGCCTGGAACGCGCAGCAGCAAAAATAAAGCCGGGCAGTGCCCGG |
| | | | CTTTTCTCACCTTTTCGCGTCCCGCAGGGCCGCTGCGAGCGCCCT |
| | | | ACCTAGATCCTCGCTTTCCCCCTCGGTGTAGTCCGCCTAGGCTGC |
| | | | TGGATACGCTGCTTAAGGTCATGCAGCAGGAGAACTAAAGGCCCG |
| | | | CGGATCCCAGGCAACGTCTTCGTACTGCGGTACCGGGTTGCGGAA |
| | | | GCTTTTGGTCACACGCATGAGAAAGCCCCCGGAAGATCACCTTCC |
| | | | GGGGGCTCATTACTCGCATCCATTCTCAGGCTGTCTCGTCTCGTC |
| | | | TCCTCGAGATTAGTTAGTTAGCCCTTAGTGACTCTAATACGACTC |
| | | | ACTGAGCTCTTGGGGCCTCTAAACGGGTCTTGAGGGGTTTTTTGT |
| | | | ACTAGTAGCGGCCGCTCGAGCCAGGATACATAGATTACCACAACT |
| | | | CCGAGCCCTTCCACCCCTAGGCTTCTCAATCATAGGCAATACGAT |
| | | | CGCATGTCCACTAGTACTGCAGGTGGCTGCTGAACCCCCAGCCGG |
| | | | AACTGACCCCACAAGGCCCTAGCGTTTGCAATGCACCAGGTCATC |
| | | | ATTGACCCAGGCGTGTTCCACCAGGCCGCTGCCTCGCAACTCTTC |
| | | | GCAGGCTTCGCCGACCTGCTCGCGCCACTTCTTCACGCGGGTGGA |
| | | | ATCCGATCCGCACATGAGGCGGAAGGTTTCCAGCTTGAGCGGGTA |
| | | | CGGCTCCCGGTGCGAGCTGAAATAGTCGAACATCCGTCGGGCCGT |
| | | | CGGCGACAGCTTGCGGTACTTCTCCCATATGAATTTCGTGTAGTG |
| | | | GTCGCCAGCAAACTGCACGACGATTTCCTCGTGCATCAGGACCTG |
| | | | GCAACGGGACGTTTTCTTGCCACGGTCCAGGACGCGGAAGCGGTG |
| | | | CAGCAGCGACACCGATTCCAGGTGCCCAACGCGGTCGGACGTGAA |
| | | | GCCCATCGCCGTCGCCTGTAGGCGCGACAGGCATTCCTCGGCCTT |
| | | | CGTGTAATACCGGCCATTGATCGACCAGCCCAGGTCCTGGCAAAG |
| | | | CTCGTAGAACGTGAAGGTGATCGGCTCGCCGATAGGGGTGCGCTT |
| | | | CGCGTACTCCAACACCTGCTGCCACACCAGTTCGTCATCGTCGGC |
| | | | CCGCAGCTCGACGCCGGTGTAGGTGATCTTCACGTCCTTGTTGAC |
| | | | GTGGAAAATGACCTTGTTTTGCAGCGCCTCGCGCGGGATTTTCTT |
| | | | GTTGCGCGTGGTGAACAGGGCAGAGCGGGCCGTGTCGTTTGGCAT |
| | | | CGCTCGCATCGTGTCCGGCCACGGCGCAATATCGAACAAGGAAAG |
| | | | CTGCATTTCCTTGATCTGCTGCTTCGTGTGTTTCAGCAACGCGGC |
| | | | CTGCTTGGCCTCGCTGACCTGTTTTGCCAGGTCCTCGCCGGCGGT |
| | | | TTTTCGCTTCTTGGTCGTCATAGTTCCTCGCGTGTCGATGGTCAT |
| | | | CGACTTCGCCAAACCTGCCGCCTCCTGTTCGAGACGACGCGAACG |
| | | | CTCCACGGCGGCCGATGGCGCGGGCAGGGCAGGGGGAGCCAGTTG |
| | | | CACGCTGTCGCGCTCGATCTTGGCCGTAGCTTGCTGGACCATCGA |
| | | | GCCGACGGACTGGAAGGTTTCGCGGGGCGCACGCATGACGGTGCG |
| | | | GCTTGCGATGGTTTCGGCATCCTCGGCGGAAAACCCCGCGTCGAT |
| | | | CAGTTCTTGCCTGTATGCCTTCCGGTCAAACGTCCGATTCATTCA |
| | | | CCCTCCTTGCGGGATTGCCCCGGAATTCCCCGGATCGATCCGTCG |
| | | | ATCTTGATCCCCTGCGCCATCAGATCCTTGGCGGCAAGAAAGCCA |
| | | | TCCAGTTTACTTTGCAGGGCTTCCCAACCTTACCAGAGGGCGCCC |
| | | | CAGCTGGCAATTCCGGTTCGCTTGCTGGCCATAAAACCGCCCAGT |
| | | | CTAGCTATCGCCATGTAAGCCCACTGCAAGCTAGGTCGTTTGTGT |
| | | | TTGCGCTTGCGTTTTCCCTTGTCCAGATAGCCCAGTACGTGACAT |
| | | | TCATCCGGGGTCAGCACCGTTTCTGCGGACTGGCTTTCTACGTGT |
| | | | TCCGCTTCCTTTAGCAGCCCTTGCGCCCTGAGTGCTTGCGGCAGC |
| | | | GTGAAGCTGACGGATCGATCCGGGGAATTAATTCACCCCCGAACA |
| | | | CGAGCACGGCACCCGCGACCACTATGCCAAGAATGCCCAAGGTAA |
| | | | AAATTGCCCGCCCCGCCATGAAGTCCGTGAATGCCCCGACGGCCG |
| | | | AAGTGAAGGGCAGGCCGCCACCCAGGCCGCCGCCCTCACTGCCCG |
| | | | GCACCTGGTCGCTGAATGTCGATGCCAGCACCTGCGGCACGTCAA |
| | | | TGCTTCCGGGCGTCGCGCTCGGGCTGATCGCCCATCCCGTTACTG |
| | | | CCCCGATCCCGGCAATGGCAAGGACTGCCAGCGCTGCCATTTTTG |
| | | | GGGTGAGGCCGTTCGCGGCCGAAGGGCGCAGCCGGGGGGATGGGA |
| | | | GGCCCGCGTTAGCCGGGAGGGTTCGAGAAGGGGGGGCACCCCCCT |
| | | | TCGGCGTGCGCGGTCACGCGCCAGGGCGCAGCCCTGGTTAAAAAC |
| | | | AAGGTTTATAAATATTGGTTTAAAAGCAGGTTAAAAGACAGGTTA |
| | | | GCGGTGGCCGAAAAACGGGCGGAAACCCTTGCAAATGCTGGATTT |
| | | | TCTGCCTGTGGACAGCCCCTCAAATGTCAATAGGTGCGCCCCTCA |
| | | | TCTGTCATCACTCTGCCCCTCAAGTGTCAAGGATCGCGCCCCTCA |
| | | | TCTGTCAGTAGTCGCGCCCCTCAAGTGTCAATACCGCAGGGCACT |
| | | | TATCCCCAGGCTTGTCCACATCATGTCTGGGAAACTCGCGTAAAA |
| | | | TCAGGCGTTTTCGCCGATTTGCGAGGCTGGCCAGCTCCACGTCGC |
| | | | CGGCCGAAATCGAGCCTGCCCCTCATCTGTCAACGCCGCGCCGGG |
| | | | TGAGTCGGCCCCTCAAGTGTCAACGTCCGCCCCTCATCTGTCAGT |
| | | | GAGGGCCAAGTTTTCCGCGTGGTATCCACAACGCCGGCGGCCGCG |
| | | | GTGTCTCGCACACGGCTTCGACGGCGTTTCTGCTAGAGATCTGTT |
| | | | TAGCTTGCCTCGTCCCC |
| 13 | R6K | R6K | CACCACTTCAAGAACTCTGTAGCACCGCCTACATACCTCGCTCTG |
| | | suicide | CTAATCCTGTTACCAGCCGGTTGTCAGCCGTTAAGTGTTCCTGTG |
| | | vector | TCACTCAAAATTGCTTTGAGAGGCTCTAAGGGCTTCTCAGTGCGT |
| | | | TACATCCCTGGCTTGTTGTCCACAACCGTTAAACCTTAAAAGCTT |
| | | | TAAAAGCCTTATATATTCTTTTTTTTCTTATAAAACTTAAAACCT |
| | | | TAGAGGCTATTTAAGTTGCTGATTTATATTAATTTTATTGTTCAA |
| | | | ACATGAGAGCTTAGTACGTGAAACATGAGAGCTTAGTACGTTAGC |
| | | | CATGAGAGCTTAGTACGTTAGCCATGAGGGTTTAGTTCGTTAAAC |
| | | | ATGAGAGCTTAGTACGTTAAACTTGAGAGCTTAGTACGTGAAACA |
| | | | TGAGAGCTTAGTACGTACTATCAACAGGTTGAACTGCCCATGTTC |
| | | | TTTCCTGCGTTATCAGAGCTTATCGGCCAGCCTCGCAGAGCAGGA |
| | | | TTCCCGTTGAGCACCGCCAGGTGCGAATAAGGGACAGTGAAGAAG |
| | | | GAACACCCGCTCGCGGGTGGGCCTACTTCACCTATCCTGCCCGGC |
| | | | TGACGCCGTTGGATACACCAAGGAAAGTCTACACGAACCCTTTGG |
| | | | CAAAATCCTGTATATCGTGCGAAAAAGGATGGATATACCGAAAAA |
| | | | ATCGCTATAATGACCCCGAAGCAGGGTTATGCAGCGGAAAGTATA |
| | | | CCTTAACATGTTCTTTCCTGCGTTATCCCCTGATTCTGTGGATAA |
| | | | CCGTATTACCGCCT |
| 14 | pBBR1 | Broad host, | CTACCGGCGCGGCAGCGTGACCCGTGTCGGCGGCTCCAACGGCTC |
| | | origin of | GCCATCGTCCAGAAAACACGGCTCATCGGGCATCGGCAGGCGCTG |
| | | replication | CTGCCCGCGCCGTTCCCATTCCTCCGTTTCGGTCAAGGCTGGCAG |
| | | | GTCTGGTTCCATGCCCGGAATGCCGGGCTGGCTGGGCGGCTCCTC |
| | | | GCCGGGGCCGGTCGGTAGTTGCTGCTCGCCCGGATACAGGGTCGG |
| | | | GATGCGGCGCAGGTCGCCATGCCCCAACAGCGATTCGTCCTGGTC |
| | | | GTCGTGATCAACCACCACGGCGGCACTGAACACCGACAGGCGCAA |
| | | | CTGGTCGCGGGGCTGGCCCCACGCCACGCGGTCATTGACCACGTA |
| | | | GGCCGACACGGTGCCGGGGCCGTTGAGCTTCACGACGGAGATCCA |
| | | | GCGCTCGGCCACCAAGTCCTTGACTGCGTATTGGACCGTCCGCAA |
| | | | AGAACGTCCGATGAGCTTGGAAAGTGTCTTCTGGCTGACCACCAC |
| | | | GGCGTTCTGGTGGCCCATCTGCGCCACGAGGTGATGCAGCAGCAT |
| | | | TGCCGCCGTGGGTTTCCTCGCAATAAGCCCGGCCCACGCCTCATG |
| | | | CGCTTTGCGTTCCGTTTGCACCCAGTGACCGGGCTTGTTCTTGGC |
| | | | TTGAATGCCGATTTCTCTGGACTGCGTGGCCATGCTTATCTCCAT |
| | | | GCGGTAGGGGTGCCGCACGGTTGCGGCACCATGCGCAATCAGCTG |
| | | | CAACTTTTCGGCAGCGCGACAACAATTATGCGTTGCGTAAAAGTG |
| | | | GCAGTCAATTACAGATTTTCTTTAACCTACGCAATGAGCTATTGC |
| | | | GGGGGGTGCCGCAATGAGCTGTTGCGTACCCCCCTTTTTTAAGTT |
| | | | GTTGATTTTTAAGTCTTTCGCATTTCGCCCTATATCTAGTTCTTT |
| | | | GGTGCCCAAAGAAGGGCACCCCTGCGGGGTTCCCCCACGCCTTCG |
| | | | GCGCGGCTCCCCCTCCGGCAAAAAGTGGCCCCTCCGGGGCTTGTT |
| | | | GATCGACTGCGCGGCCTTCGGCCTTGCCCAAGGTGGCGCTGCCCC |
| | | | CTTGGAACCCCCGCACTCGCCGCCGTGAGGCTCGGGGGGCAGGCG |
| | | | GGCGGGCTTCGCCCTTCGACTGCCCCCACTCGCATAGGCTTGGGT |
| | | | CGTTCCAGGCGCGTCAAGGCCAAGCCGCTGCGCGGTCGCTGCGCG |
| | | | AGCCTTGACCCGCCTTCCACTTGGTGTCCAACCGGCAAGCGAAGC |
| | | | GCGCAGGCCGCAGGCCGGAGG |
| 15 | pRO1600 | Broad host, | TGTGTATAAGGGGACACTGTATCTGCGTCCCACAATACAACAAAT |
| | | low copy | CCGTCCCTTTACAACAACAAATCCGTCCCTTCTTAACAACAAATC |
| | | origin of | CGTCCCTTAATGGCAACAAATCCGTCCCTTTTTAAACTCTACAGG |
| | | replication | CCACGGATTACGTGGCCTGTAGACGTCCTAAAAGGTTTAAAAGGG |
| | | | AAAAGGAAGAAAAGGGTGGAAACGCAAAAAACGCACCACTACGTG |
| | | | GCCCCGTTGGGGCCGCATTTGTGCCCCTGAAGGGGCGGGGGAGGC |
| | | | GTCTGGGCAATCCCCGTTTTACCAGTCCCCTATCGCCGCCTGAGA |
| | | | GGGCGCAGGAAGCGAGTAATCAGGGTATCGAGGCGGATTCACCCT |
| | | | TGGCGTCCAACCAGCGGCACCAGCGGCTCGACAACCCTTAATATA |
| | | | ACTTCGTATAATGTATGCTATACGAAGTTAT |
| 16 | attB2 | Landing | AAAAAAAACCCCGCCCTGTCAGGGGCGGGGTTTTTTTTTTTTCTT |
| | | Pad #1 - | TTGGGTATAGCGTCGTGGACAGTCATTCATCTTTCTGCCCCTCCA |
| | | DT3-attB2- | AAAGCAAAAACCCGCCGAAGCGGGTTTTTACGTAAATCAGGTGAA |
| | | DT54 | ACTGACCGATAAGCCGGGACGAGCTGAGCAGTATGTCGACGGTCC |
| | | | GGCGAAGAGCTACTCCCCTTCTGCGCCGTCCGAAGTTCCTATACT |
| | | | TTCTAGAGAATAGGAACTTCGGACCAAAACGAAAAAACACCCTTT |
| | | | CGGGTGTCTTTTCTGGAATTTGGTACCGAGCCTTTGGTCGAAAAA |
| | | | AAAAGCCCGCACTGTCAGGTGCGGGCTTTTTTCTGTGTTTCC |
| 17 | attB7 | Landing | ACATTTAATAAAAAAAGGGCGGTCGCAAGATCGCCCTTTTTTACG |
| | | Pad #2 - | TATGACACAGTGAAAAATGGCGCCCATCGGCGCCATTTTTTTATG |
| | | DT60- | GAAGTTCCTATTCTCTAGAAAGTATAGGAACTTCAGACGAGAAAC |
| | | attB7- | GTTCCGTCCGTCTGGGTCAGTTGGGCAAAGTTGATGACCGGGTCG |
| | | DT104 | TCCGTTGCAGACAAAAAAAATGGCGCACAATGTGCGCCATTTTTC |
| | | | ACTTCACAGGTACTATTGTTTTGAATTGAAAAGGGCGCTTCGGCG |
| | | | CCCTTTTTGCATTTGTTGACGGCATATATTTGTATATCGAAGCGC |
| | | | CCTGATGGGCGCTTTTTTTATTTAATCGATAACCAGA |
| 18 | ccdA WT | Antitoxin, | GATAACAGTATGCGTATTTGCGCGCTGATTTTTGCGGTATAAGAA |
| | | pWT-ccdA | TATATACTGATATGTATACCCGAAGTATGTCAAAAAGAGGTGTGC |
| | | | TATGAAGCAGCGTATTACAGTGACAGTTGACAGCGACAGCTATCA |
| | | | GTTGCTCAAGGCATATGATGTCAATATCTCCGGTCTGGTAAGCAC |
| | | | AACCATGCAGAATGAAGCCCGTCGTCTGCGTGCCGAACGCTGGAA |
| | | | AGCGGAAAATCAGGAAGGGATGGCTGAGGTCGCCCGGTTTATTGA |
| | | | AATGAACGGCTCTTTTGCTGACGAGAACAGGGACTGGTGAGGCTC |
| | | | CTTTTGGAGCCTTTTTTTTT |
| 19 | ccdA | Antitoxin, | TTTACAGCTAGCTCAGTCCTAGGTATTATTACTAGAGAAAGAGGG |
| | J23101 | J23101- | GAAATACTAGATGAAGCAGCGTATTACAGTGACAGTTGACAGCGA |
| | | B0064- | CAGCTATCAGTTGCTCAAGGCATATGATGTCAATATCTCCGGTCT |
| | | ccdA | GGTAAGCACAACCATGCAGAATGAAGCCCGTCGTCTGCGTGCCGA |
| | | | ACGCTGGAAAGCGGAAAATCAGGAAGGGATGGCTGAGGTCGCCCG |
| | | | GTTTATTGAAATGAACGGCTCTTTTGCTGACGAGAACAGGGACTG |
| | | | GTGAGGCTCCTTTTGGAGCCTTTTTTTTT |
| 20 | ccdA | Antitoxin, | TTTACGGCTAGCTCAGCCCTAGGTATTATTACTAGAGAAAGAGGG |
| | J23107 | J23107- | GAAATACTAGATGAAGCAGCGTATTACAGTGACAGTTGACAGCGA |
| | | B0064- | CAGCTATCAGTTGCTCAAGGCATATGATGTCAATATCTCCGGTCT |
| | | ccdA | GGTAAGCACAACCATGCAGAATGAAGCCCGTCGTCTGCGTGCCGA |
| | | | ACGCTGGAAAGCGGAAAATCAGGAAGGGATGGCTGAGGTCGCCCG |
| | | | GTTTATTGAAATGAACGGCTCTTTTGCTGACGAGAACAGGGACTG |
| | | | GTGAGGCTCCTTTTGGAGCCTTTTTTTTT |
| 21 | gRNA | J23119- | TTGACAGCTAGCTCAGTCCTAGGTATAATACTAGTGGAGACCNNN |
| | template 1 | BsaI- | NNNNNNNNNNNNNNNNNGGTCTAGTTTTAGAGCTAGAAATAGCAA |
| | | gRNA | GTTAAAATAAGGCTAGTCCGTTATCAACTTGAAAAAGTGGCACCG |
| | | random | AGTCGGTGCTAGAGATCAAGCCTTAACGAACTAAGACCCCCGCAC |
| | | spacer 1- | CGAAAGGTCCGGGGGTTTTTTTTGACCTTAAAAACATAACCGAGG |
| | | BsaI- | AGCAGACA |
| | | gRNA | |
| | | scaffold-ilv | |
| | | GEDA | |
| | | terminator | |
| 22 | gRNA | J23104- | TTGACAGCTAGCTCAGTCCTAGGTATTGTGCTAGCGGAGACCNNN |
| | template 2 | BsaI- | NNNNNNNNNNNNNNNNNGGTCTCAGTTGTAGATCTAGAAATAGAA |
| | | gRNA | TGTTACAATTAGGCTAGTCCGTTATGAACATGAAAATGTGAGAAA |
| | | random | AGAGGCCGCGAAAGCGGCCTTTTTTCGTTTCTCGGTACCAAAGAC |
| | | spacer 2- | GAACAATAAGACGCTGAAAAGCGTCTTTTTTCGTTTTGGTCC |
| | | BsaI- | |
| | | gRNA | |
| | | scaffold- | |
| | | L3S2P55 | |
| 23 | gRNA | J23104- | TTGACAGCTAGCTCAGTCCTAGGTATTGTGCTAGCGCGTTCTGAA |
| | template 3 | gRNA3- | CAAATCCAGAGTTTTAGAGCTAGAAATAGCAAGTTAAAATAAGGC |
| | | gRNA | TAGTCCGTTATCAACTTGAAAAAGTGGCACCGAGTCGGTGCCTCG |
| | | scaffold- | GTACCAAATTCCAGAAAAGAGGCCTCCCGAAAGGGGGGCCTTTTT |
| | | DT21 (not | TCGTTTTGGTCC |
| | | template, | |
| | | gRNA3) | |
| 24 | gRNA | J23108- | CTGACAGCTAGCTCAGTCCTAGGTATAATGCTAGCGCGTTCTGAA |
| | template 3 | gRNA3- | CAAATCCAGAGGTTTAGAGTTAGAAATAACAAGTTAAACTAAGGC |
| | | gRNA | TAGTCCGTTATAAACTTGAAAAAGTCAGTCAAAAGCCTCCGGTCG |
| | | scaffold13- | GAGGCTTTTGACTTTCCTCGGTACCAAATTCCAGAAAAGAGGCCT |
| | | DT21 (not | CCCGAAAGGGGGGCCTTTTTTCGTTTTGGTCC |
| | | template, | |
| | | gRNA3) | |
| 25 | gRNA | J23108- | CTGACAGCTAGCTCAGTCCTAGGTATAATGCTAGCGCGTTCTGAA |
| | template 3 | gRNA3- | CAAATCCAGAGTTGTAGATCTAGAAATAGAATGTTACAATTAGGC |
| | | gRNA | TAGTCCGTTATGAACATGAAAATGTGAGAAAAGAGGCCGCGAAAG |
| | | scaffold17- | CGGCCTTTTTTCGTTTCTCGGTACCAAATTCCAGAAAAGAGGCCT |
| | | DT21 (not | CCCGAAAGGGGGGCCTTTTTTCGTTTTGGTCC |
| | | template, | |
| | | gRNA3) | |
| 26 | cI | Repressor; | TTCTTTCAGGCCGGGTAACACCGTGCGTGTTGACTATTTTACCTC |
| | | Pr-RiboJ- | TGGCGGTGATAATAGCTGTCACCGGATGTGCTTTCCGGTCTGATG |
| | | B0034-cI- | AGTCCGTGAGGACGAAACAGCCTCTACAAATAATTTTGTTTAATC |
| | | rrnB T1-T2 | TAGAATTAAAGAGGAGAAATTAACCATGAGTAAAGGAGAAGAACT |
| | | | TTTCACTGGAGTTGTCCCAATTCTTGTTGAATTAGATGGTGATGT |
| | | | TAATGGGCACAAATTTTCTGTTAGTGGAGAGGGTGAAGGTGATGC |
| | | | AACATACGGAAAACTTACCCTTAAATTTATTTGCACTACTGGAAA |
| | | | ACTACCTGTTCCATGGCCAACACTTGTCACTACTTTCGGTTATGG |
| | | | TGTTCAATGCTTTGCGAGATACCCAGATCATATGAAACAGCATGA |
| | | | CTTTTTCAAGAGTGCCATGCCCGAAGGTTATGTACAGGAAAGAAC |
| | | | TATATTTTTCAAAGATGACGGGAACTACAAGACACGTGCTGAAGT |
| | | | CAAGTTTGAAGGTGATACCCTTGTTAATAGAATCGAGTTAAAAGG |
| | | | TATTGATTTTAAAGAAGATGGAAACATTCTTGGACACAAATTGGA |
| | | | ATACAACTATAACTCACACAATGTATACATCATGGCAGACAAACA |
| | | | AAAGAATGGAATCAAAGTTAACTTCAAAATTAGACACAACATTGA |
| | | | AGATGGAAGCGTTCAACTAGCAGACCATTATCAACAAAATACTCC |
| | | | AATTGGCGATGGCCCTGTCCTTTTACCAGACAACCATTACCTGTC |
| | | | CACACAATCTGCCCTTTCGAAAGATCCCAACGAAAAGAGAGACCA |
| | | | CATGGTCCTTCTTGAGTTTGTAACAGCTGCTGGGATTACACATGG |
| | | | CATGGATGAACTATACAAATAGCAAATAAAACGAAAGGCTCAGTC |
| | | | GAAAGACTGGGCCTTTCGTTTTATCTGTTGTTTGTCGGTGAACGC |
| | | | TCTCCTGAGTAGGACAAATCCGCCGGGAGCGGATTTGAACGTTGC |
| | | | GAAGCAACGGCCCGGAGGGTGGCGGGCAGGACGCCCGCCATAAAC |
| | | | TGCCAGGCATCAAATTAAGCAGAAGGCCATCCTGACGGATGGCCT |
| | | | TTT |
| 27 | TP901 | Repressor; | TGTTGACAATTAATCATCGGCTCGTATAATGTGTGGAATTGTGAG |
| | | pTac- | CGCTCACAATTAGCTGTCAAGGCGTGTGCTTCGCCTTCTGATGAG |
| | | RiboJ69- | CCCGTGAGGGCGAAACAGCCTCTACAAATAATTTTGTTTAAGCTG |
| | | R2-TP901- | GTACTGCTCGAGTATCTTATGAAGACTGACACATCAAATCGCTTA |
| | | rpoC | AAGCAGATCATGGCTGAGCGCAACCTTAAACAAGTGGATATCCTG |
| | | | AATCTTAGCATCCCTTTTCAAAAAAAATTCGGGATCAAATTGAGC |
| | | | AAGTCCACCCTTTCGCAATACGTCAACTCGGTACAATCCCCAGAC |
| | | | CAGAACCGTATTTACCTGTTAGCCAAGACCTTGGGGGTAAGTGAA |
| | | | GCATGGTTGATGGGATTCGACGTTCCAATGGTGGAATCCTCGAAG |
| | | | ATTGAGAATGATTCAGAGAACATTGAGGAGACGATCACCGTCATG |
| | | | AAGAAGCTGGAAGAGCCGCGTCAGAAAGTGGTCTTGGATACAGCA |
| | | | AAAATTCAGTTAAAGGAGCAGGATGAACAGAATAAGGTAAAGCAG |
| | | | ATCGAGGACTATCGCTTATCCGACGAATATTTGGAGGAGCAGATC |
| | | | AGCAAGGCGTCGGCTTACGGAGGGGGGCAACTTAACGATAATGAT |
| | | | AAGGAATTTTTCAAACGTCTGCTGAAAAACACCCTTAAGGAGAAA |
| | | | ATTGACAAAGGCGATCTTTGAGTAATCGTTAATCCGCAAATAACG |
| | | | TAAAAACCCGCTTCGGCGGGTTTTTTTATGGGGGGAGTTTAGGGA |
| | | | AAGAGCATTTGTCA |
| 28 | 933W | Repressor; | TGTTGACAATTAATCATCGGCTCGTATAATGTGTGGAATTGTGAG |
| | | pTac-PlmJ- | CGCTCACAATTCGTAAGTCATAAGTCTGGGCTAAGCCCACTGATG |
| | | R1-933W- | AGTCGCTGAAATGCGACGAAACTTATGACCTCTACAAATAATTTT |
| | | DT36 | GTTTAAGCACTACGGAATTAAGCGCTCAAATGGTTCAGAATGAAA |
| | | | AAGTGCGCAAAGAATTCGCCCAGCGGCTAGCGCAAGCCTGTAAAG |
| | | | AAGCTGGTCTTGATGAACATGGTAGGGGAATGGCTATAGCCCGTG |
| | | | CCCTTTCTCTTTCGTCCAAAGGCGTTAGCAAATGGTTTAATGCTG |
| | | | AGTCTTTACCGCGTCAGGAAAAAATGAATGCGCTTGCGAAATTTC |
| | | | TAAACGTTGATGTTGTTTGGCTTCAGCACGGCACTTCGTTAAATG |
| | | | GAGCGAATGATGAAGATACTCTTTCATTTGTTGGCAAATTAAAAA |
| | | | AAGGGTTAGTGCGCGTGGTTGGTGAGGCAATTCTTGGTGTTGATG |
| | | | GTGCCATCGAGATGACCGAAGAACGCGATGGGTGGCTCAAAATTT |
| | | | ATAGCGATGATCCAGATGCCTTTGGTCTGCGTGTGAAAGGAGACA |
| | | | GCATGTGGCCCAGAATAAAATCAGGAGAATATGTACTCATTGAGC |
| | | | CTAACACCAAAGTATTCCCGGGTGATGAGGTGTTTGTCAGAACCG |
| | | | TTGAAGGACACAACATGATTAAGGTTCTTGGCTATGACAGAGATG |
| | | | GAGAATACCAATTTACAAGCATTAACCAGGATCACAGGCCTATAA |
| | | | CGTTGCCTTATCATCAAGTAGCAAAGGTGGAGTATGTAGCTGGTA |
| | | | TTCTGAAGCAATCTCGCCATCTGGATGACATCGAGGCAAGGGAGT |
| | | | GGCTGAAAAGTTCGTGAGATCTAACTAAAAAGGCCGCTCTGCGGC |
| | | | CTTTTTTCTTTTCACTGTAACAACGGAAACCGGCCATTGCGCCGG |
| | | | TTTTTTTTGGCCT |
| 29 | P2hd | Repressor; | TGTTGACAATTAATCATCGGCTCGTATAATGTGTGGAATTGTGAG |
| | | pTac- | CGCTCACAATTCGTACTGAAGTCGTCAAGTGCTGTGCTTGCACTT |
| | | RiboJ60- | CTGATGAGGCAGTGATGCCGAAACGACCTCTACAAATAATTTTGT |
| | | R1-P2hd- | TTAATTATCTTAGGTACAGGGCACATAAATGTCAATAGACGTTTC |
| | | DT56 | GGAGAAGTTGAAGCTAATCCGTGAATCTGAAAGGTTAAACCGTAA |
| | | | AGAATTCAGTGAATTAACTGGTGTAGCCTACAGCTCACTTTCGAG |
| | | | CTATGAGAGCCGGTCAAAAAACGCTGGAGTTGAAGCCATAATGAA |
| | | | GGTCTTACAACATCCTAGATTTACTAAATATACTTTGTGGTTCAT |
| | | | GACTGATCAGGTAGCTCCAGAAGCCGGGCAAATTGCGCCCGCTCT |
| | | | CGCACACTTTGGGCAAAACGAAACAACGTCGCCCCACTCCGGTCA |
| | | | AAAGACTGGTTAATACCACCGTCAAAAAAAACGGCGCTTTTTAGC |
| | | | GCCGTTTTTATTTTTCAACCTTCCAGGCATCAAATAAAACGAAAG |
| | | | GCTCAGTCGAAAGACTGGGCCTTTCGTTTTATCTGTTGTTTGTCG |
| | | | GTGAACGCTCTC |
| 30 | P2 | Repressor; | TGTTGACAATTAATCATCGGCTCGTATAATGTGTGGAATTGTGAG |
| | | pTac- | CGCTCACAATTCGTAAGGAGTCAATTAATGTGCTTTTAATTCTGA |
| | | RiboJ64- | TGAGACGGTGACGTCGAAACTCCCTCTACAAATAATTTTGTTTAA |
| | | R3-P2- | TTTTTCTCCACTACGATGGTAATATGTCAAACACGATAAGCGAGA |
| | | DT86 | AGATAGTCTTAATGCGAAAATCAGAGTATTTGAGCAGACAACAAC |
| | | | TTGCTGATTTAACAGGGGTTCCGTATGGCACGCTGAGTTACTATG |
| | | | AAAGTGGTCGTTCAACACCTCCAACAGATGTCATGATGAACATCC |
| | | | TGCAGACCCCACAATTCACCAAATACACTTTATGGTTCATGACCA |
| | | | ATCAGATCGCTCCTGAGTCCGGGCAAATTGCGCCCGCTCTCGCAC |
| | | | ACTTTGGGCAAAACGAAACAACGTCGCCCCACTCCGGTCAAAAGA |
| | | | CTGGTTAATAATCATTCTTAGCGTGACCGGGAAGTCGGTCACGCT |
| | | | ACCTCTTCTGAAGAAACAGCAAACAATCCAAAACGCCGCGTTCAG |
| | | | CGGCGTTTTTTCTGCTTTTCT |
| 31 | Wphi | Repressor; | TGTTGACAATTAATCATCGGCTCGTATAATGTGTGGAATTGTGAG |
| | | pTac-SarJ- | CGCTCACAATTCGTAGACTGTCGCCGGATGTGTATCCGACCTGAC |
| | | R1-Wphi- | GATGGCCCAAAAGGGCCGAAACAGTCCTCTACAAATAATTTTGTT |
| | | DT19 | TAATGCCACTTACATTAGCGTTTTTGAATGCAGACATTCGAAAAA |
| | | | CTGAAAGCGATTAGGAAAGCAGAAGGCTTAACACAGGCGAAATTC |
| | | | AGCGAAATTAGCGGGATAGCTCTAGGAACAGTCAAAAATTACGAA |
| | | | AGTGGGCATAAAGACCCTGGTCTGAGCATCGTTATGCGAGTCACA |
| | | | AATACGCCTTTATTTAAAAAATATACGCTCTGGTTAATGACTGGT |
| | | | GATACGTCACCACAAGCTGGTCAGATCGCGCCGGCTCTCGCACAC |
| | | | ATTGGGCAAAAACCAACAGAATCAGACCACTCCGAAAAACAGACT |
| | | | GGTTAATTCAGCCAAAAAACTTAAGACCGCCGGTCTTGTCCACTA |
| | | | CCTTGCAGTAATGCGGTGGACAGGATCGGCGGTTTTCTTTTCTCT |
| | | | TCTCAACTCGGTACCAAAGACGAACAATAAGACGCTGAAAAGCGT |
| | | | CTTTTTTCGTTTTGGTCC |
| 32 | CymR | Repressor; | TTGACAGCTAGCTCAGTCCTAGGTATAATGCTAGCAATGGAATAT |
| | | J23119- | TCATAGTAAATACCAAACGGAGATTCTTATGAGCCCGAAACGTCG |
| | | cym2- | TACCCAGGCAGAACGTGCAATGGAAACCCAGGGTAAACTGATTGC |
| | | CymR- | AGCAGCACTGGGTGTTCTGCGTGAAAAAGGTTATGCAGGTTTTCG |
| | | AM-DT56 | TATTGCAGATGTTCCGGGTGCAGCCGGTGTTAGCCGTGGTGCACA |
| | | | GAGCCATCATTTTCCGACCAAACTGGAACTGCTGCTGGCAACCTT |
| | | | TGAATGGCTGTATGAGCAGATTACCGAACGTAGCCGTGCACGTCT |
| | | | GGCAAAACTGAAACCGGAAGATGATGTTATTCAGCAGATGCTGGA |
| | | | TGATGCAGCAGAATTTTTTCTGGATGATGATTTTAGCATCGGCCT |
| | | | GGATCTGATTGTTGCAGCAGATCGTGATCCGGCACTGCGTGAAGG |
| | | | TATTCAGCGTACCGTTGAACGTAATCGTTTTGTTGTTGAAGATAT |
| | | | GTGGCTGGGTGTGCTGGTGAGCCGTGGTCTGAGCCGTGATGATGC |
| | | | CGAAGATATTCTGTGGCTGATTTTTAACAGCGTTCGTGGTCTGGT |
| | | | AGTTCGTAGCCTGTGGCAGAAAGATAAAGAACGTTTTGAACGTGT |
| | | | GCGTAATAGCACCCTGGAAATTGCACGTGAACGTTATGCAAAATT |
| | | | CAAACGTTGATACCACCGTCAAAAAAAACGGCGCTTTTTAGCGCC |
| | | | GTTTTTATTTTTCAACCTTCCAGGCATCAAATAAAACGAAAGGCT |
| | | | CAGTCGAAAGACTGGGCCTTTCGTTTTATCTGTTGTTTGTCGGTG |
| | | | AACGCTCTC |
| 33 | Ph1F | Repressor; | TTGACAGCTAGCTCAGTCCTAGGTATAATGCTAGCCTATGGACTA |
| | | J23119- | TGTTTGAAAGGGAGAAATACTAGATGGCACGTACCCCGAGCCGTA |
| | | ph13-ph1F- | GCAGCATTGGTAGCCTGCGTAGTCCGCATACCCATAAAGCAATTC |
| | | AM-DT56 | TGACCAGCACCATTGAAATCCTGAAAGAATGTGGTTATAGCGGTC |
| | | | TGAGCATTGAAAGCGTGGCACGTCGCGCCGGTGCAGGCAAACCGA |
| | | | CCATTTATCGTTGGTGGACCAACAAAGCAGCACTGATTGCCGAAG |
| | | | TGTATGAAAATGAAATCGAACAGGTACGTAAATTTCCGGATTTGG |
| | | | GTAGCTTTAAAGCCGATCTGGATTTTCTGCTGCATAATCTGTGGA |
| | | | AAGTTTGGCGTGAAACCATTTGTGGTGAAGCATTTCGTTGTGTTA |
| | | | TTGCAGAAGCACAGTTGGACCCTGTAACCCTGACCCAACTGAAAG |
| | | | ATCAGTTTATGGAACGTCGTCGTGAGATACCGAAAAAACTGGTTG |
| | | | AAGATGCCATTAGCAATGGTGAACTGCCGAAAGATATCAATCGTG |
| | | | AACTGCTGCTGGATATGATTTTTGGTTTTTGTTGGTATCGCCTGC |
| | | | TGACCGAACAGTTGACCGTTGAACAGGATATTGAAGAATTTACCT |
| | | | TCCTGCTGATTAATGGTGTTTGTCCGGGTACACAGTGTTGATAAT |
| | | | ACCACCGTCAAAAAAAACGGCGCTTTTTAGCGCCGTTTTTATTTT |
| | | | TCAACCTTCCAGGCATCAAATAAAACGAAAGGCTCAGTCGAAAGA |
| | | | CTGGGCCTTTCGTTTTATCTGTTGTTTGTCGGTGAACGCTCTC |
| 34 | cIPro | promoter | TAACACCGTGCGTGTTGACTATTTTACCTCTGGCGGTGATAAT |
| 35 | TP901Pro | promoter | AGTTCATGAAACGTGAACTTGACAGCTAGCTCAGTCCTAGGTATA |
| | | | AAGTTCATGAAACGTGAACT |
| 36 | 993Wpro | promoter | ATTGTCAGAAATGGCACTTGCAAAAACTTTCAGTTCAACCATAAT |
| | | | ACGTACTGAAAGTACGAAAAAGG |
| 37 | P2hdPro | promoter | AACTACTACGCAAAATTCAACTTCTCCGAAACGTCTATTGACATA |
| | | | ATTACTCCGATTGCGTAATTTCTTG |
| 38 | P2Pro | promoter | ATGATTGGCGACTCGGTTTAGATCTCATTGACATGGTGTTTAGAT |
| | | | CTCAAGTACTGTTAGTTTAGATGTACAATTTTGTTAGTTTAGAT |
| 39 | wPhiPro | promoter | TCCTATAAGGACACGCATTTGACAGTAACCTATTGGTGACTTATA |
| | | | TTCCCGTCAAAAGGTTGTGTATTGGTGACCTT |
| 40 | pCymRC | promoter | AACAAACAGACAATCTGGTCTGTTTGTATTATGGAAAATTTTTCT |
| | | | GTATAATAGATTCAACAAACAGACAATCTGGTCTGTTTGTATTAT |
| 41 | Ph1FPro | promoter | TTGACATGATACGAAACGTACCGTATCGTTAAGGTTACTAGAGTC |
| | | | TAGA |
| 42 | pRO1600 | Broad host, | GTGGCCTCACCCCCAATGGTTTACAAAAGCAATGCCCTGGTCGAG |
| | | low copy | GCCGCGTATCGCCTCAGTGTTCAGGAACAGCGGATCGTTCTGGCC |
| | | origin of | TGTATTAGCCAGGTGAAGAGGAGCGAGCCTGTCACCGATGAAGTG |
| | | replication | ATGTATTCAGTGACGGCGGAGGACATAGCGACGATGGCGGGTGTC |
| | | | CCTATCGAATCTTCCTACAACCAGCTCAAAGAAGCGGCCCTGCGC |
| | | | CTGAAACGGCGGGAAGTCCGGTTAACCCAAGAGCCCAATGGCAAG |
| | | | GGGAAAAGACCGAGTGTGATGATTACCGGCTGGGTGCAAACAATC |
| | | | ATCTACCGGGAGGGTGAGGGCCGTGTAGAACTCAGGTTCACCAAA |
| | | | GACATGCTGCCGTACCTGACGGAACTCACCAAACAGTTCACCAAA |
| | | | TACGCCTTGGCTGACGTGGCCAAGATGGACAGCACCCACGCGATC |
| | | | AGGCTTTACGAGCTGCTCATGCAATGGGACAGCATCGGCCAGCGC |
| | | | GAAATAGAAATTGACCAGCTGCGAAAGTGGTTTCAACTGGAAGGC |
| | | | CGGTATCCCTCGATCAAGGACTTCAAGTTGCGAGTGCTTGATCCA |
| | | | GCCGTGACGCAGATCAACGAGCACAGCCCGCTACAGGTGGAGTGG |
| | | | GCGCAGCGAAAGACCGGGCGCAAGGTCACACATCTGTTGTTCAGT |
| | | | TTTGGACCGAAGAAGCCCGCCAAGGCGGTGGGTAAGGCCCCAGCG |
| | | | AAGCGCAAGGCCGGGAAGATTTCAGATGCTGAGATCGCGAAACAG |
| | | | GCTCGCCCTGGTGAGACATGGGAAGCGGCCCGCGCTCGACTAACC |
| | | | CAGATGCCGCTGGATCTGGCCTAG |
| 43 | pRSAori | Broad host, | CTGCATTTACGTTGACACCATGCCCGTAGGGTTGCGGCCCTTCGG |
| | | low copy | GTGACTCGGCGCTGTGCGCTATGGCGATCTGGAAGTGTCGGGACG |
| | | origin of | AGGGGCGTCACGGCGGTAAATTCGACGTGCCTGCGGCGCGTCGAA |
| | | replication | CAAGGGGCGTGCCCGGTGTCAGTTCGTGGCATTTTTCGAGGCGCG |
| | | | ACGCCATTTCCAAGGCTCCTGAGCATTCGGGTCTGACCAAAGGCC |
| | | | GAGCCGTTGGCGGCGGGCCTCTTTTTCATACTCGTACATCTGCGC |
| | | | GTCGGTTGGTTTGCCGTAATAACGGTAAGCCCAGGCCATGCCTTC |
| | | | TTGAACCATGATCGCATTGATGTTGGTGAGTTGTGTTTGGCCGCC |
| | | | GGGGTATTGCAACGGCGCGTAAACGACCCCAAGAGTGCGGCCATA |
| | | | CCGATCAACCTCTTTTTCGGTCACTTGAACCTCTTGGCGAAAGGT |
| | | | CAAGTCGGCGAGCCGTTGGCGAGCACGGGAGCCGAAGGCTTGGCC |
| | | | GCTTTCCGGTGCGTCAATATCGGCCAATCTCACGCGGATGGTCTG |
| | | | ACGGTTCACCAAAACGTCGATAGTGTCACCGTCAAGGATTCGGAC |
| | | | GACTTCACCCCGGAAGTCGGCCCAAGCGGGCACACTGACGATTAG |
| | | | GACGACAGCGGCCGCGACCGCGCGAAGGGCGGCAAGGGCGCTTTT |
| | | | CATTGTTTGCCTCCTGTTTTCAAGACGGCTGTGAGATTGGCGACC |
| | | | TGCTCTTTGAGGGCTTCCACCTGACCTTGCAGACTGGCGGCGCGC |
| | | | TCGATGGCCTCCTTGGCCTGTTTGCGGGCCTCGATAGCCTCGTTA |
| | | | TCGCGTTGGGTGAGCTTTTCCATGCAGCGGTTTAGTTCTTCGCCG |
| | | | CTGCGGCGTTTCACTTCGGCAAGCTGGTCGGCCAGCTTGTCGCGC |
| | | | TCGCGTTCCATAGGTTCGAGCTGATTCACTCGTTCGCGGAGCTGG |
| | | | TCGTTTTCGCGGGTGAAGGTGTCGGCTAGTTCGATTGCTTCGGCA |
| | | | AGCTGCTGGCTGATGGCCGCTTTGTCGGCCTOGATCTGTTTCCGA |
| | | | TCTTCGTCAAACCGGGCGTTGGCGTGCGCCAGGGCGATAGCCCAT |
| | | | AGCGCATTGCCAAGCTCGGCAAGATGCTCGTTGACTGCAACCGGC |
| | | | AATGGGTCTGATGAGGGCAGGGTGGCGGTCTTGCGGTTTTTCCAT |
| | | | TCAGCCATTGCATCGGAAATGGTTGTGAAGCTACCGCTTCCGAGT |
| | | | TTCTTGCGCACGGCGGCCAAAGTGGGCCGGATGCCTTCGGCGTCC |
| | | | AGTTCGTCGGCTGCTCGCCAAATGTCTTGTTTAGTGATTGCCATT |
| | | | CTTGCGGGCCTCTGTACTGTAGTATGTTGTATGATACTACATACT |
| | | | ACAACAATTTAACAGAGCCATCTTGGAATCTGGTGTCTCTGCGCC |
| | | | TATAATTCTGGAACAGCTACTTTCCGAACGACTCCTGCGTTGATC |
| | | | GGAAATCCAGAAGCCCGAGAGGTTGCCGCCTTTCGGGCTTTTTCT |
| | | | TTTTCAAAAAAAAAAATTTATAAAACGATCTGTTGCGGCCGCCGG |
| | | | GTTGTGGGCAAAGGCGCTCGACGGTGGGCAACCGCTTGCGGTTGT |
| | | | CCACGGGCGGAGCCGGTGCGCGTAGCGCATTGTCCACAAGCCAAG |
| | | | GGCGACCAATAATTGATATATATATTCATAATTGAAAAGCTAATT |
| | | | GAACATACTACTTGCTGTAACTACTTGCCGGAGCGAGGGGTGTTT |
| | | | GCAAGCTGTTGATCTGAAAGGGCTATTAGCGTTCTCACGTGCCTT |
| | | | TTTGATTAGCGATTTCACGTGACCTTATTAGCGATTTCACGTACT |
| | | | CCGATTAGCGATTTCACGTACCCTGATTAGCGATTTCACGTGGAT |
| | | | AGTTTTTGGAGOGGGCCGGAAAGCCCCGTGAATCAAGGCTTTGCG |
| | | | GGGCATTAGCGGTTTCACGTGGATAACTACCCTCTATCCACAGGC |
| | | | TTCCGGGGATAAAAAAGCCCGCTCGACGGCGGGCTGTTGGATGGG |
| | | | AAGGCTTGACCAAGCCAAGCGTAGCGTTGGCCTGGTCAAGTOGGA |
| | | | GGGGGGCCGATGCGAGCGCCCTTGCCGGGTGCGCGGGTGACATGC |
| | | | AGGCGTGTGGATTTGATGCGCAGGCATTCGCCGTCATCTTCGATG |
| | | | CAGTCGCTTGCCTCGGGATAGACAATCAACACTTCGCGTAGGCGC |
| | | | TTTTTGAAGTTGTATTTGAAGCTGGCGAGTGCTGCCCGCTCTGCC |
| | | | CGCTCTCGGGCCTTATCGTCCAGTTCGGGCGAGTTGCGTGCGCGG |
| | | | CTGCCATAGGATGAGCCGAATTGCGCTTGCAGGGCGACCCAAGGG |
| | | | ATTTGCACGAAGGGGCGGCCCTTGGCCCGCAACAGGAACACGCGA |
| | | | TAGGTCAGCCACGTGTAAATGTCCATCGCAAGCGGAGACTGCCGC |
| | | | AAGGCATGCAGGTAGTCGATTCGGATAGGAACCGGTGAGCGGGTG |
| | | | ACTTCCTCGAAGAAATCGCCTGTGAGGGTGAGGGTGCTATCCCAT |
| | | | AGCGCCCGATCTTCTGGCCGCTTGGGATTCCAGAATAGAAAAGCG |
| | | | CGCTTGGCAATGACGACGTTCTCAATGCCGAAGTCATTGCCTTGC |
| | | | TCGCCGGCAAGCGAAATCATGGATGAAAACAGGCGTTGCGCCTGA |
| | | | TTGCGAAGGGTGGCCGTGTAACGGCCATCGGTGTGCATTCCGAGC |
| | | | CTTTGTAGAAATTCCGATTGCGACCGGCCAAGGTTCAACACGGGG |
| | | | TCTTTCGTTCGCACGGCCTCGGTGCATATCCAAGCAAGCAAGGTG |
| | | | CGCGGCATAGAACCGTAGGGCAGGCCGATGCTCGGCTTGCCCATG |
| | | | ATCGACAAGGTGACGATGCCATTGGTGCGCTCAAAGTAGCTGGTC |
| | | | TTGGGGTCGGTGTGGGGCATGGTCGCTTGCACAAGGCAACGGGCC |
| | | | ATGTAGCCGACTAAGCCAGCTTCGCGGGCATCCTCCATTTCGAGC |
| | | | GCGAGGCTCGTCTTGATGATCTCGTTGATACGATGGCCGGGGGCT |
| | | | TTGTTGTTCTTAGGCATGTTGTTCCCTCCCCGGCATGGTGATGGT |
| | | | TGGTCTAGTGTTTGTGGGTTTGATGTTCCGGCGTTTGATGAACAG |
| | | | GCGCAAGGTGTGAGGGCTGACGCCTAACAACTCGGCTGCGCGACT |
| | | | TTGCGGCAAGCCAAGGTTCACGTATGCCTGTACTTCATCAATACG |
| | | | GCTGTCCAGCTTCAAGGCGCTCGATTTGCTGCCCTTGGGTCGCCC |
| | | | GAGCGTCTTGCCGCGCTCTCTGGCGACTTGTAGCGCCTCGGTGGT |
| | | | ACGTGCCTGAATGAAATGCCGCTCGATCTGTGCAGCCAAGCCAAG |
| | | | CACGGTTGCCATGATGTCGCTTTGTAGGCTGCCGTCCATGATGAT |
| | | | CTTCTGTTTGGTCACATGGACGATTAGGCCGCGCTCGCTCGCCGC |
| | | | TTTGAGAATTTCCAAGGCGGCGAGGGCGGAACCGGCAATGCGCGT |
| | | | AATCTCCGGCGTCAGTAGCACGTCGCCACGCTCGGCCTTTTCGAT |
| | | | GATTGCTCCGAGCTTGCGCTTGCGCCAGTCCTTTGCTCTGCTGGC |
| | | | AATTTCTTCCTCGATCTGTAGCGGCGCGAAGCCTTTGGCGTTCGC |
| | | | GTATTCGAGCAAACCGTATTTTTGGTTTTCCGGGTCTTGGCCGTC |
| | | | ACGCGAAACCCGGAGATAGGCATAGTATTTTGGCATTTGCAGGGA |
| | | | AAACGTCAGATTCGGTTAAACATGCCTCATTCTAGCGCAGATTAA |
| | | | ATAGGAATTAAATACCCTGTTGCGGTATAGATAAAACGTTGGTTT |
| | | | GCACTTGGGTTGCGCAGCAACCCGTAAGTGCGCTGTTCCAGACTA |
| | | | TCGGCTGTAGCCGCCTCGCCGCCCTATACCTTGTCTGCCTCCCCG |
| | | | CGTTGCGTCGCGGTGCATGGAGCCGGGCCACCTCGACCTGAATGG |
| | | | AAGCCGGCGGCACCTCGCTAACGGATTCACCGTTTTTATCAGGCT |
| | | | CTGGGAGGCAGAATAAATGATCATATCGTCAATTATTACCTCCAC |
| | | | GGGGAGAGCCTGAGCAAACTGGCCTCAGGCATTTGAGAAGCACAC |
| | | | GGTCACACTGCTTCCGGTAGTCAATAAACCGGTAAACCAGCAATA |
| | | | GACATAAGCGGCTATTTAACGACCCTGCCCTGAACCGACGACCGG |
| | | | GTCGAATTTGCTTTCGAATTTCTGCCATTCATCCGCTTATTATCA |
| | | | CTTATTCAGGCGTAGCACCAGGCGTTTAACCTAGGAGTGCGGTTG |
| | | | GAACGTTGGCCCAGCCAGATACTCCCGATCACGAGCAGGACGCCG |
| | | | A |
| 44 | mNG | fluorescent | GTCAGCAAAGGCGAGGAAGACAATATGGCTTCGCTCCCGGCTACC |
| | | reporter | CACGAACTCCACATCTTTGGGAGCATCAATGGCGTGGACTTTGAC |
| | | | ATGGTGGGGCAAGGGACCGGTAACCCCAACGACGGCTACGAGGAA |
| | | | CTCAATTTGAAGTCCACGAAAGGCGATCTCCAATTTAGCCCCTGG |
| | | | ATTTTGGTGCCACACATCGGCTACGGGTTTCATCAATACTTGCCA |
| | | | TATCCTGATGGTATGAGCCCTTTTCAAGCGGCTATGGTGGATGGT |
| | | | TCCGGTTATCAGGTCCACCGCACCATGCAATTTGAAGATGGTGCG |
| | | | TCCTTGACGGTGAATTATCGGTATACGTATGAAGGTAGCCACATC |
| | | | AAAGGCGAGGCGCAAGTGAAGGGGACGGGTTTTCCCGCAGATGGC |
| | | | CCAGTGATGACCAATAGCCTGACGGCCGCTGATTGGTGTCGGTCC |
| | | | AAAAAAACCTATCCTAATGACAAGACGATCATCTCGACCTTTAAG |
| | | | TGGTCGTATACGACCGGTAACGGGAAGCGCTATCGCTCGACGGCC |
| | | | CGTACCACCTATACGTTTGCCAAACCTATGGCGGCCAATTACCTG |
| | | | AAAAACCAGCCTATGTACGTCTTCCGTAAAACCGAACTGAAACAC |
| | | | TCGAAAACCGAGTTGAACTTCAAGGAATGGCAGAAAGCCTTCACG |
| | | | GACGTGATGGGCATGGACGAGTTGTACAAATAA |
| 45 | trfA | Plasmid | ATGAATCGGACGTTTGACCGGAAGGCATACAGGCAAGAACTGATC |
| | | replication | GACGCGGGGTTTTCCGCCGAGGATGCCGAAACCATCGCAAGCCGC |
| | | initiator | ACCGTCATGCGTGCGCCCCGCGAAACCTTCCAGTCCGTCGGCTCG |
| | | protein | ATGGTCCAGCAAGCTACGGCCAAGATOGAGCGCGACAGCGTGCAA |
| | | | CTGGCTCCCCCTGCCCTGCCCGCGCCATOGGCCGCCGTGGAGCGT |
| | | | TCGCGTCGTCTCGAACAGGAGGCGGCAGGTTTGGCGAAGTCGATG |
| | | | ACCATCGACACGCGAGGAACTATGACGACCAAGAAGCGAAAAACC |
| | | | GCCGGCGAGGACCTGGCAAAACAGGTCAGCGAGGCCAAGCAGGCC |
| | | | GCGTTGCTGAAACACACGAAGCAGCAGATCAAGGAAATGCAGCTT |
| | | | TCCTTGTTCGATATTGCGCCGTGGCCGGACACGATGCGAGCGATG |
| | | | CCAAACGACACGGCCCGCTCTGOCCTGTTCACCACGCGCAACAAG |
| | | | AAAATCCCGCGCGAGGCGCTGCAAAACAAGGTCATTTTCCACGTC |
| | | | AACAAGGACGTGAAGATCACCTACACCGGCGTCGAGCTGCGGGCC |
| | | | GACGATGACGAACTGGTGTGGCAGCAGGTGTTGGAGTACGCGAAG |
| | | | CGCACCCCTATCGGCGAGCCGATCACCTTCACGTTCTACGAGCTT |
| | | | TGCCAGGACCTGGGCTGGTCGATCAATGGCCGGTATTACACGAAG |
| | | | GCCGAGGAATGCCTGTCGCGCCTACAGGCGACGGCGATGGGCTTC |
| | | | ACGTCCGACCGCGTTGGGCACCTGGAATCGGTGTCGCTGCTGCAC |
| | | | CGCTTCCGCGTCCTGGACCGTGGCAAGAAAACGTCCCGTTGCCAG |
| | | | GTCCTGATCGACGAGGAAATCGTCGTGCTGTTTGCTGGCGACCAC |
| | | | TACACGAAATTCATATGGGAGAAGTACCGCAAGCTGTCGCCGACG |
| | | | GCCCGACGGATGTTCGACTATTTCAGCTCGCACCGGGAGCCGTAC |
| | | | CCCGTCAAGCTGGAAACCTTCCGCCTCATGTGCGGATCGGATTCC |
| | | | ACCCGCGTGAAGAAGTGGCGCGAGCAGGTCGGCGAAGCCTGCGAA |
| | | | GAGTTGCGAGGCAGCGGCCTGGTGGAACACGCCTGGGTCAATGAT |
| | | | GACCTGGTGCATTGCAAACGC |
| 46 | trfA | Plasmid | MNRTFDRKAYRQELIDAGFSAEDAETIASRTVMRAPRETFQSVGS |
| | | replication | MVQQATAKIERDSVQLAPPALPAPSAAVERSRRLEQEAAGLAKSM |
| | | initiator | TIDTRGTMTTKKRKTAGEDLAKQVSEAKQAALLKHTKQQIKEMQL |
| | | protein - | SLFDIAPWPDTMRAMPNDTARSALFTTRNKKIPREALQNKVIFHV |
| | | amino acid | NKDVKITYTGVELRADDDELVWQQVLEYAKRTPIGEPITFTFYEL |
| | | sequence | CQDLGWSINGRYYTKAEECLSRLQATAMGFTSDRVGHLESVSLLH |
| | | | RFRVLDRGKKTSRCQVLIDEEIVVLFAGDHYTKFIWEKYRKLSPT |
| | | | ARRMFDYFSSHREPYPLKLETFRLMCGSDSTRVKKWREQVGEACE |
| | | | ELRGSGLVEHAWVNDDLVHCKR |
| 47 | R1- | segregation | GCCCGGCGACATCTTTTGTTACCCGCCAAACAAAACCCAAAAACA |
| | ParCMR | locus | ACCCATACCCAACCCAATAAAACACCAAAACAAGACAAATAATCA |
| | | | TTGATTGATGGTTGAAATGGGGTAAACTTGACAAACAAACCCACT |
| | | | TAAAACCCAAAACATACCCAAACACACACCAAAAAAACACCATAA |
| | | | GGAGTTTTATAAATGTTGGTATTCATTGATGACGGTTCAACAAAC |
| | | | ATCAAACTACAGTGGCAGGAAAGCGACGGAACAATTAAACAGCAC |
| | | | ATTAGCCCGAACAGCTTCAAACGCGAGTGGGCAGTCTCTTTTGGT |
| | | | GATAAAAAGGTCTTTAACTACACACTGAACGGCGAACAGTATTCA |
| | | | TTTGATCCAATCAGCCCGGATGCTGTAGTCACAACCAATATCGCA |
| | | | TGGCAATACAGCGACGTTAATGTCGTTGCAGTGCATCACGCCTTA |
| | | | CTGACCAGTGGTCTGCCGGTAAGCGAAGTGGATATTGTTTGCACA |
| | | | CTTCCTCTGACAGAGTATTACGACAGAAATAACCAACCCAATACG |
| | | | AATGAAATTGAGCGTAAGAAAGCAAACTTCCGGAAAAAAATTACA |
| | | | TTAAATGGCGGGGATACATTCACAATAAAAGATGTAAAAGTCATG |
| | | | CCTGAATCTATACCGGCAGGTTATGAAGTTCTACAAGAACTGGAT |
| | | | GAGTTAGATTCTTTATTAATTATAGATCTCGGGGGCACCACATTA |
| | | | GATATTTCTCAGGTAATGGGGAAATTATCGGGGATCAGTAAAATA |
| | | | TACGGAGACTCATCTCTTGGTGTCTCTCTGGTTACATCTGCAGTA |
| | | | AAAGATGCCCTTTCTCTTGCGAGAACAAAAGGAAGTAGCTATCTT |
| | | | GCTGACGATATAATCATTCACAGAAAAGATAATAACTATCTGAAG |
| | | | CAACGAATTAATGATGAGAACAAAATATCAATAGTCACCGAAGCA |
| | | | ATGAATGAAGCACTTCGTAAACTTGAGCAACGTGTATTAAATACG |
| | | | CTCAATGAATTTTCTGGTTATACTCATGTTATGGTTATAGGCGGT |
| | | | GGCGCAGAATTAATATGCGATGCAGTAAAAAAACACACACAGATT |
| | | | CGTGATGAACGTTTTTTCAAAACCAATAACTCTCAATATGATTTA |
| | | | GTTAACGGTATGTATCTCATAGGTAATTAATGATGGACAAGCGCA |
| | | | GAACCATTGCCTTCAAACTAAATCCAGATGTAAATCAAACAGATA |
| | | | AAATTGTTTGTGATACACTGGACAGTATCCCGCAAGGGGAACGAA |
| | | | GCCGCCTTAACCGGGCCGCACTGACGGCAGGTCTGGCCTTATACA |
| | | | GACAAGATCCCCGGACCCCTTTCCTTTTATGTGAGCTGCTGACGA |
| | | | AAGAAACCACATTTTCAGATATCGTGAATATATTGAGATCGCTAT |
| | | | TTCCAAAAGAGATGGCCGATTTTAATTCTTCAATAGTCACTCAAT |
| | | | CCTCTTCACAACAAGAGCAAAAAAGTGATGAAGAGACCAAAAAAA |
| | | | ATGCGATGAAGCTAATAAATTAATTCAATTATTATTGAGTTCCCT |
| | | | TTATCCACTATCAGGCTGGATAAAGGGAACTCAATCAAGTTATTT |
| | | | TCTTACCAGTCATTACATAATCGTTATTATGAAATAATCGTTTGC |
| | | | ACTGTCTCTG |
| 48 | γδ | resolvase | AGATCCCGTTCATCAAGATGAAAATAGCGCGCCAGTTGCACGTCA |
| | | | TTAGGTTATGCAGCATAACAACCGTAATTCTGTTTTTGGTCAGAG |
| | | | GTTAGAAAGTCGACAGGCATATCAGGCTCCCTACCTGACAGTTAT |
| | | | TCATTAACAATTTTGCAACCGTCCGAAATATTATAAATTATCGCA |
| | | | CACATAAAAACAGTGCTGTTAATGTGTCTATTAAATCGATTTTTT |
| | | | GTTATAACAGACACTGCTTGTCCGATATTTGATTTAGGATACATT |
| | | | TTTATGCGACTTTTTGGTTACGCACGGGTATCAACCAGCCAGCAA |
| | | | TCTCTCGATATTCAGGTTCGGGCACTCAAAGACGCAGGCGTGAAA |
| | | | GCAAATCGCATCTTTACTGACAAGGCATCGGGCAGTTCAAGCGAT |
| | | | CGGAAAGGGCTGGACTTGCTGAGGATGAAGGTGGAGGAAGGTGAC |
| | | | GTCATCTTGGTGAAGAAACTTGACCGCCTTGGGCGCGATACTGCT |
| | | | GACATGATCCAGTTAATAAAAGAGTTTGACGCCCAAGGTGTATCC |
| | | | ATTCGGTTTATTGATGACGGAATCAGTACCGATGGGGAGATGGGT |
| | | | AAAATGGTTGTCACTATTCTATCTGCGGTGGCCCAGGCAGAACGA |
| | | | CAGAGAATACTAGAGCGTACCAATGAAGGTCGCCAAGAGGCAATG |
| | | | GCAAAAGGAGTTGTTTTTGGTAGAAAAAGAAAAATAGATAGAGAT |
| | | | GCAGTATTAAATATGTGGCAACAGGGGTTAGGTGCCTCACATATA |
| | | | TCAAAAACAATGAATATTGCTCGTTCAACAGTATATAAAGTAATA |
| | | | AATGAAAGCAACTAACATAAAAGGTTGAATATGGAATTGAATATT |
| | | | ACTTCAAAGTCTAATCCGTTTGGTGATACGACA |
| 49 | pirA | Ferrientero | ATGAGACTCAAGGTCATGATGGACGTGAACAAAAAAACGAAAATT |
| | | bactin | CGCCACCGAAACGAGCTAAATCACACCCTGGCTCAACTTCCTTTG |
| | | receptor | CCCGCAAAGCGAGTGATGTATATGGCGCTTGCTCCCATTGATAGC |
| | | PirA | AAGGAACCTCTTGAACGAGGGCGAGTTTTCAAAATTAGGGCTGAA |
| | | | GACCTTGCAGCGCTCGCCAAAATCACCCCATCGCTTGCTTATCGA |
| | | | CAATTAAAAGAGGGTGGTAAGTTACTTGGTGCCAGCAAAATTTCG |
| | | | CTAAGAGGGGATGATATCATTGCTTCAGCTAAAGAGCTTAACCTG |
| | | | CTCTTTACTGCTAAAGACTCCCCTGAAGAGTTAGATCTTAACATT |
| | | | ATTGAGTGGATAGCTTATTCAAATGATGAAGGATACTTGTCTTTA |
| | | | AAATTCACCAGAACCATAGAACCATATATCTCTAGCCTTATTGGG |
| | | | AAAAAAAATAAATTCACAACGCAATTGTTAACGGCAAGCTTACGC |
| | | | TTAAGTAGCCAGTATTCATCTTCTCTTTATCAACTTATCAGGAAG |
| | | | CATTACTCTAATTTTAAGAAGAAAAATTATTTTATTATTTCCGTT |
| | | | GATGAGTTAAAGGAAGAGTTAATAGCTTATACTTTTGATAAAGAT |
| | | | GGAAGTATTGAGTACAAATACCCTGACTTTCCTATTTTTAAAAGG |
| | | | GATGTATTAAATAAAGCCATTGCTGAAATTAAAAAGAAAACAGAA |
| | | | ATATCGTTTGTTGGCTTTACTGTTCATGAAAAAGAAGGAAGAAAA |
| | | | ATTAGTAAGCTGAAGTTCGAATTTGTCGTTGATGAAGATGAATTT |
| | | | TCTGGCGATAAAGATGATGAAGCTTTTTTTATGAATTTATCTGAA |
| | | | GCTAATGCAGCTTTTCTCAAGGTATTTGATGAAACCGTACCTCCC |
| | | | AAAAAAGCTAAGGGGTGA |

TABLE 7
Plasmids with modifications.

| Identifier | Name | Relevant Features |
|---|---|---|
| pR.002 | GFP reporter plasmid | pCym-GFP-oriT-kanR |
| pR.003 | pTAmob | Tamob conjugative plasmid, GmR |
| pR.007 | TetR-Tn7 | J23101-mCherry-TetR-Tn7, R6K origin |
| | GentR-mNG-Tn7 | pR.029 CRISPR-gRNA_template J23101-mN |
| pR.029 | CRISPR-gRNA_template | pCym-cas9-gRNA random template-RK2-par_locus-oriT-kanR |
| pR.036 | CRISPR-gRNA_5 | pCym-cas9-gRNA5 (polA_Xcc)_RK2_par_locus |
| pR.037 | CRISPR-gRNA_6 | pCym-cas9-gRNA6 (ftsZ_Xcc)_RK2_par_locus |
| pR.051 | GmR-Tn7 | J23101-mCherry-GmR-Tn7, R6K origin |
| pR.056 | Kunoichi P1 | Cloning plasmid for Kunoichi assembly, Km, Gm |
| pR.057 | Kunoichi P2 | Subfragment 1 of pTAmob, Km |
| pR.058 | Kunoichi P3 | Subfragment 2 of pTAmob, Km |
| pR.059 | Kunoichi P4 | Subfragment 3 of pTAmob, Km |
| pR.060 | Kunoichi P5 | Subfragment 4 of pTAmob, Km |
| pR.061 | Kunoichi P6 | Subfragment 5 of pTAmob, Km |
| pR.077 | g420 repressor | Tn7 with Ph2d repressor system, GmR |
| pR.078 | g422 repressor | Tn7 with WphiC repressor system, GmR |
| pR.079 | g421 repressor | Tn7 with P2 C repressor system, GmR |
| pR.093 | CRISPR-gRNA_21 | pCym-cas9-gRNA21 (dnaA_GEV)_RK2_par_locus |
| pR.094 | CRISPR-gRNA_22 | pCym-cas9-gRNA22 (gyrA_GEV)_RK2_par_locus |
| pR.112 | 2 gRNA plasmid | cas9-gRNA template 1 + gRNA template 2_RK2_par_locus |
| pR.116 | CRISPR-gRNA_15 | pCym-cas9-gRNA15 (Euvesicatoria_1)_RK2_par_locus |
| pR.117 | CRISPR-gRNA_17 | pCym-cas9-gRNA17 (Euvesicatoria_2)_RK2_par_locus |
| pR.118 | CRISPR-gRNA_23/Ea1 | pCym-cas9-gRNA21 (Erwinia_1)_RK2_par_locus |
| pR.119 | CRISPR-gRNA_24/Ea2 | pCym-cas9-gRNA21 (Erwinia_2)_RK2_par_locus |
| pR.120 | RP4 | IncP conjugative plasmid, Tc, Amp, Km |
| pR.121 | Rsa | IncW conjugative plasmid, Sm, Km, Cm, Sul |
| pR.122 | R702 | IncP conjugative plasmid, Sm, Km |
| pR.123 | pIP113 | IncN conjugative plasmid, Tc, Km |
| pR.124 | Kunoichi 1A − Cas9 split | Cloning plasmid with Cas9-gRNA insert, partial Cas9 |
| pR.125 | Kunoichi 1B − Cas9 split | Cloning plasmid with Cas9-gRNA insert, partial Cas9 |
| pR.127 | Kunoichi P7 | Subfragment 6 of pTAmob, Km |
| pR.128 | Kunoichi P8 | Subfragment 7 of pTAmob, Km |
| pR.141 | CRISPR-gRNA_template | pCI-cas9-gRNA random template-RK2_par_locus-oriT-kanR |
| pR.142 | CRISPR-gRNA | pCI-cas9-gRNA (po1A_Xcc)_RK2_par_locus |
| pR.149 | TAmob-oriT | TAmob-Rsa origin of replication, TetR |
| pR.150 | TAmob-CRISPR-oriT | TAmob-pCym-cas9-gRNA21-oriT |
| pR.151 | RP4-CRISPR | RP4-pCymR-cas9-gRNA5-oriT |
| pR.160 | Tamob.V2 | TAmob-RsaOri |
| | F′ lac | Family IncFI; Pasteur Institute No. CRBIP19.25 |
| | pIP162 | Family IncFI; Pasteur Institute No. CIP pIP162 |
| | R1 | Family IncFII; Pasteur Institute No. CRBIP19.58 |
| | R1drd19 | Family IncFII; Pasteur Institute No. CRBIP19.59 |
| | pIP24 | Family IncFII; Pasteur Institute No. CRBIP19.29 |
| | R64 | Family IncIl; Pasteur Institute No. CRBIP19.30 |
| | R391 | Family IncJ; Pasteur Institute No. CRBIP19.39 |
| | PIP175 | Family IncI2; Pasteur Institute No. CRBIP19.75 |
| | RIP71A | Family Inc9 (IncFII?); Pasteur Institute No. CRBIP19.47 |
| | TP116 | Family IncH2; Pasteur Institute No. CIP TP116 |
| | pIP1100 | Family IncX; Pasteur Institute No. CIP pIP1100/HB101 |
| | R6K | Family IncX; Pasteur Institute No. CRBIP19.49 |
| | pIP72 | Family Inc10; Pasteur Institute No. CRBIP19.42 |
| | pIP55 | Family IncA/C; Pasteur Institute No. CIP pIP55 |
| | pIP69 | Family Inc7/L/M; Pasteur Institute No. CIP pIP69 |
| | pIP113 | Family IncN; Pasteur Institute No. CIP pIP113 |
| | RN3 | Family IncN; Pasteur Institute No. CRBIP19.54 |
| pR.198 | intTAmob | intTAmob is derived from TAmob and engineered to integrate |
| | | irreversibly into the chromosome of the chassis |
| pR.211 | CRISPR-gRNA47 | pCym-cas9-gRNA47 (X. perforans)_RK2_par_locus |
| pR.220 | ΔCas9-gRNA template | pCym-gRNA random template-RK2-par_locus-oriT-kanR |
| pR.221 | CRISPR-ΔgRNA | pCym-cas9-RK2-par locus-oriT-kanR |
| pR.240 | CRISPR-gRNA template v2 | pCym-cas9-gRNA random template-RK2-par locus-oriT-kanR |
| pR.241 | CRISPR-gRNA template v3 | pCym-cas9-gRNA random template-RK2-par locus-oriT-kanR ΔtrfA |
| pR.242 | CRISPR-gRNA74 | pCym-cas9-gRNA74 (random)_RK2_par_locus |
| pR.243 | CRISPR-gRNA75 | pCym-cas9-gRNA75 (random)_RK2_par_locus |

### Transfer Efficiency

An important feature in the success of the donor bacteria and plasmid transfer system is the efficiency of transfer of plasmid DNA between organisms. One method for improving the transfer efficiency is through directed evolution. In some embodiments, the modified donor bacteria comprising an engineered plasmid as described herein undergoes mutagenesis, yielding a plasmid having improved conjugation properties. Mutagenesis can be induced by a number of means. In some embodiments, mutagenesis is induced by a mutagen. In some embodiments, the mutagen is MP1, MP2, MP3, MP4, MP5, or MP6. Improved plasmids are selected by screening for increased transfer efficiency into a second strain of bacteria. Multiple rounds of mutagenesis and transfer selection are performed until the desired transfer efficiency is achieved.

In some embodiments, the transfer efficiency of any method of HGT described herein is about 10\hat{ }-5, about 10\hat{ }-4, about 10\hat{ }-3, about 10\hat{ }-2, about 10\hat{ }-1, or about 1. In some embodiments, the HGT transfer efficiency is from about 10\hat{ }-5 to about 1. In some embodiments, the HGT transfer efficiency is from about 10\hat{ }-4 to about 1. In some embodiments, the HGT transfer efficiency is from about 10\hat{ }-3 to about 1. In some embodiments, the HGT transfer efficiency is from about 10\hat{ }-5 to about 1.

### Transduction

Transduction provides for transfer of DNA into a cell by a virus or viral vector. Transduction does not require contact between cells. In some embodiments, exogenous DNA is introduced into a bacterium via a bacteriophage. In some embodiments of compositions and methods described herein, an expression vector as described herein is transferred from a modified donor bacteria to a plant-associated bacteria by transduction.

### Transformation

Transformation is the transfer of exogenous nucleic acid directly into a bacteria from its surroundings. For nucleic acid to cross the cell membrane, the recipient bacterium must be competent, in response to environmental or induced conditions. In some embodiments, bacterial cells are made competent by chemical treatment, exposing cells to divalent cations and heat shock. In some embodiments, bacterial cells are made competent by electroporation, shocking cells with an electric field, creating temporary holes in the cell membrane. In some embodiments, nuclei acid is introduced into the cells by micro-injection. In some embodiments, bacterial cells are naturally competent.

### Gene Transfer Agents

Gene transfer agents (GTA) are virus-like particles containing DNA. After release from a donor cell, particles are available to attach to recipient cells and inject the DNA into the recipient cell's cytoplasm. In some embodiments, the GTA is RcGTA. In some embodiments, the GTA is DsGTA, from Dinoroseobacter shibae. In some embodiments, the GTA is BaGTA, from Bartonella species. In some embodiments, the GTA is VSH-1, from Brachyspira hyodysenteriae. In some embodiments, the GTA is Dd1, from Desulfovibrion desulfuricans. In some embodiments, the GTA is VTA, from Methanococcus voltae.

IV. Combinations with Plant Hosts

A modified bacteria as described herein colonizes or persists in hosts such as (but are not limited to) soil and plants. In some embodiments, a modified bacteria as described herein colonizes or persists on a plant. In some embodiments, a modified bacteria as described herein colonizes or persists on roots. In some embodiments, a modified bacteria as described herein colonizes lateral root emergence sites or root cortex. In some embodiments, a modified bacteria as described herein colonizes or persists outer cell layers of plants. In some embodiments, a modified bacteria as described herein colonizes or persists phloem. In some embodiments, a modified bacteria as described herein colonizes or persists xylem. In some embodiments, the plant is of agricultural value. Treatment of a bacteria having the deleterious effects as described herein with compositions and methods as described herein can comprise treatment of a plant of agricultural value with a modified donor bacteria as described herein. In some embodiments, plants treated using the compositions and methods described herein can prevent the plants from being affected by the deleterious effects as described herein. In some embodiments, the plant is further colonized with a bacteria having the deleterious effects as described herein. In some embodiments, the plant is treated prior to an established colonization of a bacteria having the deleterious effects as described herein. Plants treated using compositions and methods as described herein can produce crops for industrial or food use. Plants for treatment using compositions and methods described herein can produce corn and other feed grains, cotton, fruit, tree nuts, rice, soybeans and oil crops, sugar and sweeteners, vegetables, pulses, and wheat. Corn and feed grains can comprise corn, grain sorghum, barley, oats, rye, millet, or hay. In some embodiments, fruit comprises, without limitation, apples, apricots, avocados, bananas, blackberries, blueberries, boysenberries, cantaloupe, cherries, cranberries, dates, figs, grapefruit, grapes, guavas, kiwifruit, lemons, limes, loganberries, mandarins, mangoes, nectarines, olives, oranges, papayas, peaches, pears, pineapples, plums and prunes, raspberries, strawberries, tangerines, tomatoes, or watermelon. In some embodiments, tree nuts comprise, without limitation, almonds, hazelnuts, macadamia nuts, pecans, pistachios, or walnuts. In some embodiments, soybeans and oil crops comprise, without limitation, soybeans, canola, and corn. In some embodiments, sugar and sweeteners comprise, without limitation, sugarcane and sugarbeets. In some embodiments, vegetables comprise, without limitation, lettuce, onions, sweet potatoes, cassava, carrots, pumpkins, cabbage, celery, sweet corn, broccoli, bell peppers, cauliflower, squash, spinach, collard greens, garlic, cucumbers, snap beans, kale, radishes, mustard greens, eggplant, brussel sprouts, turnip greens turnips, artichokes, endive, escarole, asparagus, okra, rhubarb, lima beans, mushrooms, or potatoes. In some embodiments, pulses comprise kidney beans, lima beans, navy beans, black beans, pinto beans, small red beans, cranberry beans, great northern beans, pink beans, mung beans, black eyed beans, dry peas, lentils, or chickpeas. In some embodiments, a plant treated with a modified donor bacteria as described herein is of the genus Brassica, Solanum, Malus, Citrus, Vitis, Saccharum, Zea, Oryza, or Triticum. In some embodiments, a plant treated with a modified donor bacteria as described herein is of the genus Brassica oleracea, Brassica rapa, Solanum lycopersicum, Solanum tuberosum, Malus domestica, Citrus reticulata, Vitis vinifera, Saccharum officinarum, Zea mays, Oryza sativa, Triticum aestivum, Glycine max, or any combination, strain, or variant thereof.

In some embodiments, a modified donor bacteria comprises a species, strain or variant of a species found on a healthy plant. In some embodiments, the species is a commensal bacterial, wherein the bacteria imparts a benefit to the plant. In some embodiments, the species is not native to the plant microbiome. In some embodiments, a bacteria from a community as noted in column 1 of Table 8 is modified for use in compositions and methods described herein as a modified donor bacteria, for treatment of a plant-associated bacteria on the corresponding plant system as shown in column 2 of Table 8.

In some embodiments, a modified donor bacteria described herein exhibits strong mobility and persistence, as determined by its growth rate in soft agar or its movement in host vasculature. In some embodiments, the average speed of a modified donor bacteria is higher than 0.04 mm/h. In some embodiments, the average speed of a modified donor bacteria is higher than 0.05 mm/h. In some embodiments, after inoculating the host, a modified donor bacteria described herein moved more than 8 internodes above the point of inoculation within 7 days. In some embodiments, after inoculating the host, a modified donor bacteria described herein moved more than 9 internodes above the point of inoculation within 7 days.

TABLE 8
Representative bacterial communities on plant systems

| Bacterial Community | Plant System |
|---|---|
| Diaphorina citri, Phyllosticta citricarpa, Xanthomonas citri, | Citrus |
| and Candidatus Liberibacter asiaticus, Chryseobacterium | |
| spp. | |
| Actinobacteria and Alphaproteobacteria | Spear grass |
| Aprospirales, Actinomycetales, Rhizobiales, | Wild blueberry |
| and Xanthomonadales | |
| 35 bacteria in the rhizosphere and 1 bacteria in the endosphere | Populus deltoides |
| Sphingomonas, Rhizobium, Pseudomonas, and Variovorax | Lettuce (Lactuca sativa) |
| Proteobacteria and Actinobacteria | Maize |
| Enterobacteriaceae and Moraxellaceae families | Lettuce plants (Lactuca sativa) |
| Bacterial colonization of leaves | Rocket salad (Diplotaxis tenuifolia) |
| (Proteobacteria, Actinobacteria, Firmicutes, | and lettuce (Lactuca sativa) |
| and Bacteroidetes) | |
| Proteobacteria and Firmicutes | Spinach |
| Caulobacter sp., Flavobacterium | Arabidopsis thaliana |
| Nitrososphaerales, Methanobacteriales, E2 | Willows (Salix purpurea “Fish Creek”) |
| group, Methanosarcinales, and Methanomicrobiales | |
| Bacterial communities (Firmicutes, Bacteroidetes, Thermi, | Bean, soybean, and canola |
| and Chloroflexi) | |
| Bacterial communities | Arabidopsis thaliana |
| (Acinetobacter, Variovorax, Pseudomonas, | |
| Sphingobacteriaceae, Rhodococcus, Ochrobactrum, | |
| and Chryseobacterium) | |
| Pseudomonas sp., Enterobacter sp. | Oryza sativa |
| Pseudomonas koreensis, Ralstonia pickettii, Bacillus cereus | Brassica oleracea |
| Bacillus paranthracis and Bacillus megaterium | Solanum nigrum |
| Bacillus cereus, Bacillus thuringiensis, Buttiauxella agrestis | Musa paradisiaca |
| Azospirillum brasilense, Gluconacetobacter diazotrophicus, | Allium cepa |
| Herbaspirillum seropedicae, and Burkholderia ambifaria | |
| Pseudomonas sp., Enterobacter sp. | Lycopersicon esculentum |
| Bacillus, Brevibacillus, Agrobacterium, and Paenibacillus | Vicia faba |
| Bacillus subtilis | Zingiber officinale |
| Bacillus pumilus, Curtobacterium albidum | Oryza sativa |
| Actinomycetes, Streptomyces | Triticum durum |
| Pseudomonas reactans, Pantoea alli, Rhizoglomus irregulare | Zea mays |
| Bacillus cereus TCR17, Providencia rettgeri TCR21 and | Sorghum bicolor |
| Myroides odoratimimus | |
| Aneurinibacillus aneurinilyticus, Paenibacillus sp. | Phaseolus vulgaris |
| Bacillus subtilis, B. amyloliquefaciens, Pseudomonas | Clavibacter michiganensis |
| fluorescens, and P. aeruginosa | |
| Asprgillus fumigatus, Fusarium proliferatum | Oxalis orniculate. |
| Bacillus spp. | Capsicum annum |
| Pseudomonas sp., Sphingomonous sp., Xanthomonas sp. | Solanum lycopersicum |

### V. Gene Modification Payload

HGT systems described herein deliver a mechanism for altering a feature of a plant-associated bacteria. In some embodiments, the HGT system delivers a payload for altering the pathogenic bacterial genome. In some embodiments, the HGT system delivers a nuclease to induce a fatal cleavage of the pathogenic bacterial genome. In some embodiments, the HGT system delivers CRISPR components to selectively cleave DNA of the pathogenic bacteria. In some embodiments, the CRISPR system comprises a guide nucleic acid and a nuclease. In some embodiments, the nuclease is an artificial nuclease. In some embodiments, the nuclease is a dual nuclease.

### Guide Nucleic Acid

Guide nucleic acids function as a guide for RNA- or DNA-targeting enzymes. In some embodiments, guide nucleic acids form complexes with enzymes to affect specific cleavages, deletions, insertions, or other alterations. As described herein, a “guide nucleic acid”, “guide RNA” or “gRNA”, comprises a sequence of about 20 nucleotides homologous to a target RNA or DNA sequence. In some embodiments, the guide nucleic acid has about 80%, about 85%, about 90%, about 95%, or about 100% sequence identity to a target sequence.

A “scaffold” or “gRNA scaffold”, as described herein, describes a region of a nucleic acid sequence that complexes with an endonuclease to affect a target binding and cleavage.

A “cassette”, as used herein, describes a nucleic acid sequence comprising a promoter sequence, an expression region, and a terminator sequence. In some embodiments, a “template cassette” comprises enzyme cleavage sites flanking the expression region. In some embodiments, a specific cassette is generated from the template cassette by enzymatic cleavage at the flanking cleavage sites and insertion of a specific nucleic acid sequence. As described herein, a “gRNA template cassette” describes a sequence comprising a promoter, a random spacer sequence flanked by enzyme cleavage sites, a gRNA scaffold, and a terminator sequence. Representative gRNA template cassettes are described by SEQ ID NOs: 21 and 22. In some embodiments, a gRNA cassette is generated by enzymatic cleavage and insertion of a gRNA sequence. In some embodiments, a gRNA comprising a sequence having about 80%, about 85%, about 90%, or about 95% sequence identity to any one of SEQ ID NOs: 50-135 is inserted in the gRNA cassette template. In some embodiments, a gRNA comprising a sequence as in SEQ ID NOs: 50-135 is inserted in the gRNA cassette template.

The guide nucleic acid sequences described herein are identified by selecting guide nucleic acid sequences according to criteria comprising: instance of a PAM sequence, % GC content of the proposed guide nucleic acid sequence, specificity of the proposed guide nucleic acid to a target domain in a pathogenic bacterial genome, off-target binding within the pathogenic bacterial genome, binding affinity of the proposed guide nucleic acid to a domain in a pathogenic bacterial genome, melting temperature of the proposed guide nucleic acid sequence, and incidence of guanine in the first nucleotide position of the proposed guide nucleic acid. In some embodiments, the proposed guide nucleic acid sequence is assessed against a single pathogenic bacteria genome. In some embodiments, the proposed guide nucleic acid sequence is assessed against more than one pathogenic bacteria genome. In some embodiments, the proposed guide nucleic acid sequence is assessed against about 2, about 3, about 4, about 5, about 6, about 7, about 8, about 9, or about 10 pathogenic bacteria genome.

In some embodiments, the pathogenic bacterial genome is scanned for instances of PAM sequences. In some embodiments, instances of PAM sequences include, but are not limited to, 5′-NGG-3′, 5′-NAG-3′, and 5′-TTTN-3′. Associated nucleic acid sequences are then filtered for percent GC content. In some embodiments described herein, the % GC content of a guide nucleic acid is from about 30% to about 70%. In some embodiments, the GC content of the gRNA is about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%. In further embodiments, proposed guide nucleic acid is assessed for alignment with non-target DNA sequences in one or more bacteria. In some embodiments, a proposed guide nucleic acid sequence is assessed for non-target alignment in one bacterial genome. In some embodiments, a proposed guide nucleic acid sequence is assessed for non-target alignment, or “mismatch” in two bacterial genome, in three bacterial genomes, in four bacterial genomes, in five bacterial genomes, in six bacterial genomes, in seven bacterial genomes, in eight bacterial genomes, in nine bacterial genomes, in ten bacterial genomes. In some embodiments, guide nucleic acid sequences that have 2 or more instances of mismatch are eliminated from consideration. In some embodiments, gRNA sequences that have 3 or more, 4 or more, 5 or more, 6 or more, 7 or more, or 8 or more instances of mismatch are eliminated from consideration. In some embodiments, proposed guide nucleic acid sequences are further analyzed for melting temperature. In some embodiments, proposed gRNA sequences are further analyzed for the presence of guanine in the first nucleotide position. In some embodiments, a single guide nucleic acid is selected to target one type of bacteria. In some embodiments, a single guide nucleic acid is selected to target more than one type of bacteria. In some embodiments, one guide nucleic acid is selected to target 2, 3, 4, 5, 6, 7, 8, 9, or 10 types of bacteria. In some embodiments, multiple guide nucleic acids are selected to target multiple sites within one genome of one type of bacteria. In some embodiments, multiple guide nucleic acids are selected to target sites within multiple species of bacteria. In some embodiments, a guide nucleic acid selected targets at least one essential gene of a bacteria. Essential genes are a class of genes the product of which are necessary for organisms to grow and reproduce. In some embodiments, a guide nucleic acid selected does not target any essential gene of a bacteria. In some embodiments, a guide nucleic acid selected targets genes selected from the group consisting of dnaA, polA, gyrA, ftsZ, rpoB, rnpB, mutS, acnA, ygcE, ydjX, accD, and pheT. In some embodiments, a guide nucleic acid targets genes encoding a dnaA protein, DNA polymerase, DNA gyrase, ftsZ protein, RNA polymerases, triose-phosphate isomerase, Flp family type IVb pilin, FMN-binding protein MioC, galactose/glucose ABC transporter substrate-binding protein MglB, EscR/YscR/HrcR family type III secretion system export apparatus protein, EscR/YscR/HrcR family type III secretion system export apparatus protein, FGGY-family carbohydrate kinase, TVP38/TMEM64 family protein, acetyl-CoA carboxylase, carboxyltransferase, carboxyltransferase subunit beta, YdiU family protein, glycosyltransferase, rRNA-16S ribosomal RNA, a membrane protein, DNA mismatch repair protein MutS, phosphoribosylformylglycinamidine cyclo-ligase, cysteinyl-tRNA synthetase, rRNA-23S ribosomal RNA, phosphoserine transaminase, aconitase A, phenylalanyl-tRNA synthetase, or a phenylalanyl-tRNA synthetase beta subunit. In some embodiments, a guide nucleic acid selected targets a transfer RNA (tRNA). In some embodiments, a guide nucleic acid selected targets a hypothetical protein in a bacteria. In some embodiments, a guide nucleic acid selected targets a non-coding region in a bacteria.

In some embodiments, a guide nucleic acid described herein comprises a sequence that is greater than 90% identical to the sequence in the genome of the host bacteria and less than 90% identical to an unmodified genome of the modified donor bacteria. In some embodiments, a guide nucleic acid described herein comprises a sequence that is greater than 90%, greater than 91%, greater than 92%, greater than 93%, greater than 94%, greater than 95%, greater than 96%, greater than 97%, greater than 98%, or greater than 99% identical to the sequence in the genome of one or more species of plant-associated bacteria. In some embodiments, a guide nucleic acid described herein is a guide RNA (gRNA).

In some embodiments, guide nucleic acids used in the compositions and methods described herein are listed in Table 9 below. In some embodiments, at least one guide nucleic acid used in the compositions and methods described herein comprises at least 80%, 85%, 90%, 95%, 97%, 98%, or 99% sequence identity to SEQ ID NOS: 50-135. In some embodiments, at least one guide nucleic acid used in the compositions and methods described herein is one of SEQ ID NOS: 50-135.

TABLE 9
gRNA sequences

| SEQ ID | | | | Target |
|---|---|---|---|---|
| NO: | Identifier | Target Organism | Sequence | Gene/Protein |
| 50 | | Candidatus Liberibacter | GTCTCCCCAATGCATACTAT | Triose- |
| | | | | phosphate |
| | | | | isomerase |
| 51 | | Candidatus Liberibacter | GCTGCTTCCTAGACTACTAT | Flp family type |
| | | | | IVb pilin |
| 52 | | Candidatus Liberibacter | ATCCTAGTCCTAGGAATTCC | |
| 53 | gRNA_23 | E. amylovora | CGGAAGATCCCGCCGGCGAA | FMN-binding |
| | | | | protein MioC |
| 54 | gRNA_24 | E. amylovora | GGCGATTAATCTTGTCGATC | Galactose/ |
| | | | | glucose ABC |
| | | | | transporter |
| | | | | substrate- |
| | | | | binding protein |
| | | | | MgIB |
| 55 | gRNA_26 | E. amylovora | GTGCCCCGCGTATAATATGT | Non-coding |
| | | | | region |
| 56 | gRNA_27 | E. amylovora | GGCTGGAGCTTACTCACTAA | EscR/YscR/HrcR |
| | | | | family type |
| | | | | III secretion |
| | | | | system export |
| | | | | apparatus |
| | | | | protein |
| 57 | | E. amylovora | ACATATTATACGCGGGGCAC | Non-coding |
| | | | | region |
| 58 | gRNA_25 | E. coli | GTCTGGGTACTATATAGGAG | Non-coding |
| | | | | region |
| 59 | | E. coli | CTCCTATATAGTACCCAGAC | Non-coding |
| | | | | region |
| 60 | | E. coli | GGCCTACGGGTAGGTATATC | |
| 61 | | E. coli | TTAAGAGTCCACCCAATGAC | Non-coding |
| | | | | region |
| 62 | | E. coli | GCTAGGCGAGTCGTTAATTC | ygcE; FGGY- |
| | | | | family |
| | | | | carbohydrate |
| | | | | kinase |
| 63 | | E. coli | CTCTTAGGGACACTACTCTC | ydjX; |
| | | | | TVP38/TMEM64 |
| | | | | family protein |
| 64 | gRNA_10 | E. coli DH10b | AATGGAACTTACCAATGACG | ftsZ |
| 65 | gRNA_11 | E. coli DH10b | CTTCAGGAATGCTCGCCTTG | accD; acetyl-CoA |
| | | | | carboxylase, |
| | | | | carboxyl- |
| | | | | transferase |
| | | | | subunit beta |
| 66 | gRNA_12 | E. coli MG1655 landing | CGAGCTGAGCAGTATGTCGA | Engineered |
| | | pad | | genome- |
| | | | | integrated site |
| 67 | gRNA_1 | P. protegens | GGAACTTTGGCAGCAGTGCG | dnaA |
| 68 | gRNA_2 | P. protegens | CCAAGCCCCCCTCGTCCTGG | polA |
| 69 | gRNA_3 | P. protegens | GAGCCTGCGCAAACAGTACC | polA |
| 70 | gRNA_4 | P. protegens | GGAACTTTGGCAGCAGTGCG | dnaA |
| 71 | gRNA_5 | X. campestris | TCGCCCTGGGCATTGGTCAG | Non-coding |
| | | | | region |
| 72 | gRNA_6 | X. campestris | GACGACCTTGATCACCGCGT | FtsZ |
| 73 | gRNA_7 | X. campestris | GAACTCCACGCCATCGACGT | FtsZ |
| 74 | gRNA_8 | X. campestris | ATCACACTTTGATGGATGCT | dnaA |
| 75 | gRNA_9 | X. campestris | GACCTGGATGATTTCCTTGG | Non-coding |
| | | | | region |
| 76 | gRNA_28 | X. campestris | GTGACACGTCACTCAATAAC | |
| 77 | gRNA_29 | X. campestris | GTGCGTACTAGGTAAATCGA | Non-coding |
| | | | | region |
| 78 | gRNA_13 | X. euvesicatoria | GATGGTAACGCTCAACCCAT | polA |
| 79 | gRNA_15 | X. euvesicatoria | GCCGACCTGATCGTGCGCCC | ftsZ |
| 80 | gRNA_16 | X. euvesicatoria | CTTCTGCGCCGCCTGAATCG | dnaA |
| 81 | gRNA_17 | X. euvesicatoria | CAATGTGGCCTACATCCTGA | gyrA |
| 82 | gRNA_30 | X. euvesicatoria | GAACCTAACAGCTACTTGGT | YdiU family |
| | | | | protein |
| 83 | gRNA_31 | X. euvesicatoria | GATGCATCATAGCTAGCAAG | Non-coding |
| | | | | region |
| 84 | gRNA_14 | X. euvesicatoria, X. | TCATCACGGAACGTTTTACC | DNA |
| | | perforans | | polymerase I |
| 85 | gRNA_18 | X. euvesicatoria, X. | GACGTGGTGGAAGCCACCGA | gyrA |
| | | perforans | | |
| 86 | gRNA_19 | X. perforans | GGAAGTGATGGCCAAACGGC | polA |
| 87 | gRNA_20 | X. perforans | GGACCTGCCCAACGATTACC | ftsZ |
| 88 | gRNA_21 | X. perforans | GCCCGAGCCGGCCCCCGCAG | dnaA |
| 89 | gRNA_22 | X. perforans | ACCACCTGCAGCAGCACGTC | gyrA |
| 90 | gRNA_32 | X. perforans | GGGAGTGCTATAATTCTGAG | Glycosyl- |
| | | | | transferase |
| 91 | gRNA_33 | X. perforans | GCGCAAGTTAATACTAGAGC | |
| 92 | gRNA_34 | X. campestris, X. | AGGTAGCTTAACCTTCGGGA | rRNA-16S |
| | | perforans, E. coli, E. | | ribosomal RNA |
| | | euvesicatoria, E. | | |
| | | amylovora | | |
| 93 | gRNA_35 | E. coli, X. campestris, E. | GCGTCATCGTGTTCCAGGAA | rpoB |
| | | amylovora | | |
| 94 | gRNA_36 | X. campestris, X. | TGACTGTCCACGACAGAACC | rnpB |
| | | perforans, E. coli, X. | | |
| | | euvesicatoria, E. | | |
| | | amylovora | | |
| 95 | gRNA_37 | X. campestris, X. | GATGTGGATCATCTTGGTGC | sufB |
| | | perforans, E. coli, X. | | |
| | | euvesicatoria | | |
| 96 | gRNA_38 | X. campestris, X. | GACCTGTCCGGGTATCTGTA | membrane |
| | | perforans, X. | | protein |
| | | euvesicatoria | | |
| 97 | gRNA_39 | X. campestris, X. | GATCCCCGTCACTTTTATAG | Non-coding |
| | | perforans, X. | | region |
| | | euvesicatoria | | |
| 98 | gRNA_40 | X. campestris, X. | ACCGACCTCACCCTTATCAG | tRNA-Ile |
| | | perforans, E. coli, X. | | |
| | | euvesicatoria, E. | | |
| | | amylovora, C. | | |
| | | liberibacter spp. | | |
| 99 | 76627 | Candidatus Liberibacter | GTTTTTGGTACCGCGATCCC | tRNA-Gln |
| 100 | 278208 | Candidatus Liberibacter | CTTACAGGACCTAATATGGG | mutS; DNA |
| | | | | mismatch repair |
| | | | | protein MutS |
| 101 | 118222 | Candidatus Liberibacter | GATCTTGCAGGATTTGCTGT | CysS; |
| | | | | Phosphoribosyl- |
| | | | | formylglycinami |
| | | | | dine cyclo- |
| | | | | ligase |
| 102 | 118302 | Candidatus Liberibacter | GGCCATGCTTATGAAGCTAA | Cysteiny1-tRNA |
| | | | | synthetase |
| 103 | 8725 | Candidatus Liberibacter | CGTTAACTGAATACCCTCGG | rRNA-23S |
| | | | | ribosomal RNA |
| 104 | 310038 | Candidatus Liberibacter | GTTCTGGCATGGGAAAGTTT | Phosphoserine |
| | | | | transaminase |
| 105 | 8723 | Candidatus Liberibacter | AGCGTTAACTGAATACCCTC | rRNA-23S |
| | | | | ribosomal RNA |
| 106 | 122783 | Candidatus Liberibacter | GTTCTTGGTTGGGGAGTAGG | acnA; Aconitase |
| | | | | A |
| 107 | 8722 | Candidatus Liberibacter | GAGCGTTAACTGAATACCCT | rRNA-23S |
| | | | | ribosomal RNA |
| 108 | 97966 | Candidatus Liberibacter | GCTCCTTCTTGGTATCATCC | pheT; |
| | | | | Phenylalanyl- |
| | | | | tRNA |
| | | | | synthetase beta |
| | | | | subunit |
| 109 | 8724 | Candidatus Liberibacter | GCGTTAACTGAATACCCTCG | rRNA-23S |
| | | | | ribosomal RNA |
| 110 | 118170 | Candidatus Liberibacter | GGAAGGTGTAGAGACTGGAC | Non-coding |
| | | | | region |
| 111 | gRNA_41 | Candidatus Liberibacter | CGTTAACTGAATACCCT | rRNA-23S |
| | | | | ribosomal RNA |
| 112 | gRNA_42 | Candidatus Liberibacter | GAGCGTTAACTGAATACCCT | rRNA-23S |
| | | | | ribosomal RNA |
| 113 | gRNA_43 | Candidatus Liberibacter | GCGTTAACTGAATACCCTCG | rRNA-23S |
| | | | | ribosomal RNA |
| 114 | gRNA_44 | Candidatus Liberibacter | CTTACAGGACCTAATAT | mutS |
| 115 | gRNA_45 | Candidatus Liberibacter | GTTCTGGCATGGGAAAGTTT | phat |
| 116 | gRNA_46 | X. perforans | ACCTTCGCCAACTTCGTCGA | dnaA |
| 117 | gRNA_47 | X. perforans | CAAGACCCACCTGATGTTCG | dnaA |
| 118 | gRNA_48 | X. perforans | TGAAGGACCTGCTGTCCAAG | dnaA |
| 119 | gRNA_49 | Candidatus Liberibacter | TACGGGTTCGGTCCTCCAAT | rRNA-23S |
| | | | | ribosomal RNA |
| 120 | gRNA_50 | Candidatus Liberibacter | TACGGGGCTATCACCCACTT | rRNA-23S |
| | | | | ribosomal RNA |
| 121 | gRNA_51 | Candidatus Liberibacter | ACAAGCGAGCTTAAGCCGTT | rRNA-23S |
| | | | | ribosomal RNA |
| 122 | gRNA_52 | Candidatus Liberibacter | TGATATGCTGCGATAAGCTG | rRNA-23S |
| | | | | ribosomal RNA |
| 123 | gRNA_53 | Candidatus Liberibacter | GAATGCCTACAAACAGTCGG | rRNA-23S |
| | | | | ribosomal RNA |
| 124 | gRNA_54 | Candidatus Liberibacter | TGTACCGGGACACCGCACAT | mutS |
| 125 | gRNA_55 | Candidatus Liberibacter | TCTCGTTTAGCACTTGGACG | mutS |
| 126 | gRNA_56 | Candidatus Liberibacter | ATTACCTCGCACACGTGCTA | mutS |
| 127 | gRNA_57 | Candidatus Liberibacter | ATGTACCGGGGGTTACAAGA | mutS |
| 128 | gRNA_58 | Candidatus Liberibacter | GTAGTCCGTCTTGTAACCCC | mutS |
| 129 | gRNA_59 | Candidatus Liberibacter | CGTATCGTGATGCTCCTCCT | phat |
| 130 | gRNA_60 | Candidatus Liberibacter | AAATCTGGCAACTTACCGTA | phat |
| 131 | gRNA_61 | Candidatus Liberibacter | CCCTTCTATGTTATGCGTTG | phat |
| 132 | gRNA_62 | Candidatus Liberibacter | AATGCTGATAACGACCAACC | phat |
| 133 | gRNA_63 | Candidatus Liberibacter | CGTTCTCATCGTTCTACTGA | phat |
| 134 | gRNA_74 | non targeting gRNA | CTTTGCGCCAAGAGATCGCG | — |
| 135 | gRNA_75 | non targeting gRNA | TACAGTAACGATACGATTAC | — |

In some embodiments, a CRISPR system provided in compositions described herein comprises a nuclease to cleave the pathogenic bacteria DNA. In some embodiments, the nuclease used in the HGT is a Cas enzyme. In some embodiments, the Cas enzyme is a Type I, Type II, Type III, Type IV, Type V, or Type VI Cas enzyme. In some embodiments, the Cas enzyme used in the HGT is a Cas3 endonuclease. In some embodiments, the Cas enzyme used in the HGT is a Cas9 endonuclease. In some embodiments, the Cas9 endonuclease is from S. thermophilus, S. pyogenes, or S. aureus. In some embodiments, the Cas enzyme used in the HGT is a Cas12a (Cpf1) endonuclease. In some embodiments, the Cas12a (Cpf1) endonuclease is Lachnospiraceae bacterium ND2006 Cas12a (LbCas12a). In some embodiments, the Cas enzyme used in the HGT is a Cas13 endonuclease. In some embodiments, the Cas13 endonuclease is Leptotrichia wadei Cas13a. In some embodiments, the Cas13 endonuclease is Ruminococcus flavefaciens XPD3002 Cas13d In some embodiments, the nuclease used in the HGT is a dual nuclease comprising a Cas enzyme. In some embodiments, the nuclease used in the HGT is TevSpCas9 dual nuclease.

In some embodiments, the HGT system delivers a synthetic nuclease. In some embodiments, the HGT system delivers a TAL/TALEN endonuclease. In some embodiments, the HGT system delivers a zinc finger nuclease (ZFN). In some embodiments, the HGT system delivers an argonaute protein from Pyrococcus furiosus (PfAgo). TAL/TALEN system as a component of the payload for selectively cleaving the DNA of a pathogenic bacteria described herein. A TALEN includes the TALE domain that binds specific sequences and a catalytic domain of an endonuclease that introduces DNA double strand breaks. The DNA binding domain of each TALE is capable of targeting large recognition sites with high specific (15-30 nucleotides). The catalytic domain is fused to the TALE DNA-binding domain. Therefore, in some embodiments, the TAL/TALEN system comprises a transcriptional activator like effector (TALE) fused to an endonuclease. In some embodiments, the endonuclease is the FokI endonuclease.

In some embodiments, the HGT system delivers a zinc finger nuclease (ZFN) as a component of the payload for selectively cleaving the DNA of the pathogenic bacteria described herein. ZFN are artificial restriction enzymes generated by fusing a zinc finger DNA-binding domain to a DNA-cleavage domain. The DNA-binding domain contains three to six individual zinc finger repeats and each can recognize from 9 to 18 base pairs. The DNA-cleavage domain comprises the type II restriction endonuclease FokI, which dimerizes to cleave DNA. In some embodiments, the ZFNs comprises linker sequences between the zinc finger domain and the cleavage domain.

In some embodiments, the payload comprises a gene encoding a toxin, wherein the toxin selectively kills the pathogenic bacteria. In some embodiments, the payload comprises a gene encoding a plant growth regulator. In some embodiments, the payload comprises a gene encoding plant growth promoters. Plant growth promoters promote cell division, cell enlargement, flowering, fruiting and seed formation. In some embodiments, the plant growth promoter is auxin, gibberellin, or cytokinin. In some embodiments, the auxin described herein is indole-3-acetic acid, 4-chloroindole-3-acetic acid, phenylacetic acid, indole-3-butyric acid, or indole-3-propionic acid.

In some embodiments, the payload further comprises a gene encoding a plant immune stimulator, which trigger plant defenses before or upon plant pathogen attack. In some embodiments, the payload further comprises a gene or gene cluster regulating nitrogen fixation. In some embodiments, the gene regulating nitrogen fixation is a gene responsible for nodule formation. In some embodiments, the gene responsible for nodule formation is a Nod gene. In some embodiments, the gene regulating nitrogen fixation is a gene responsible for nitrogen uptake. In some embodiments, the gene responsible for nitrogen uptake is a Hup gene. In some embodiments, the gene regulating nitrogen fixation is a gene responsible for nitrogen fixation. In some embodiments, the gene responsible for nitrogen fixation is a Nif gene.

### VI. Methods of Use

Compositions described herein are applied, in some embodiments, to a plant, to a soil, to a seed. Methods of application to a plant include a foliar spray, a root drench, a needle inoculation, a syringe infiltration, a mist, a fog, a spray, or an injection. Methods of application to a soil include a root drench or a soil amendment. Methods of application to a seed include a seed coating. In some embodiments, the application is pre-emergent to the plant growth. In some embodiments, the application is post-emergent to the plant growth.

### Dose

Compositions described herein are applied at a dose effective to suppress or reduce growth of a pathogenic bacteria. In some embodiments, modified donor bacteria are applied in a ratio of CFU donor bacteria vs CFU pathogenic bacteria. In some embodiments, the application comprises 1000-fold more modified donor bacteria than pathogenic bacteria on the plant. In some embodiments, the application comprises 100-fold more modified donor bacteria than pathogenic bacteria on the plant. In some embodiments, the application comprises 50-fold more donor bacteria than pathogenic bacteria on the plant. In some embodiments, the application comprises 40-fold more donor bacteria than pathogenic bacteria on the plant. In some embodiments, the application comprises 30-fold more donor bacteria than pathogenic bacteria on the plant. In some embodiments, the application comprises 20-fold more donor bacteria than pathogenic bacteria on the plant. In some embodiments, the application comprises 10-fold more donor bacteria than pathogenic bacteria on the plant. In some embodiments, the application comprises 5-fold more donor bacteria than pathogenic bacteria on the plant. In some embodiments, the application comprises 1-fold more donor bacteria than pathogenic bacteria on the plant. In some embodiments, the application comprises 0.5-fold more donor bacteria than pathogenic bacteria on the plant. In some embodiments, the application comprises 0.1-fold more donor bacteria than pathogenic bacteria on the plant.

In some embodiments, compositions of modified donor bacteria described herein are applied in an amount of total CFU per mL liquid. In some embodiments, modified donor bacteria are applied at 10\hat{ }2 CFU/mL liquid. In some embodiments, modified donor bacteria are applied at 10′CFU/mL liquid. In some embodiments, modified donor bacteria are applied at 10∝CFU/mL liquid. In some embodiments, modified donor bacteria are applied at 10\hat{ }5 CFU/mL liquid. In some embodiments, modified donor bacteria are applied at 10\hat{ }6 CFU/mL liquid. In some embodiments, modified donor bacteria are applied at 10\hat{ }7 CFU/mL liquid. In some embodiments, modified donor bacteria are applied at 10\hat{ }8 CFU/mL liquid. In some embodiments, modified donor bacteria are applied at 10\hat{ }9 CFU/mL liquid. In some embodiments, modified donor bacteria are applied at 10\hat{ }10 CFU/mL liquid.

In some embodiments, compositions of modified donor bacteria described herein are applied at a ratio of CFU modified bacteria/mL liquid to CFU plant-associated bacteria/mL liquid. In some embodiments, the ratio is from about 10,000:1 to about 1:1. In some embodiments, the ratio is about 10,000:1, about 5000:1, 1000:1, about 500:1, about 100:1, about 50:1, about 25:1, 20:1, about 19:1, about 18:1, about 17:1, about 16:1, about 15:1, about 14:1, about 13:1, about 12:1, about 11:1, about 10:1 about 9:1, about 8:1, about 7:1, about 6:1, about 5:1, about 4:1, about 3:1, about 2:1, or about 1:1. In some embodiments, the ratio is from about 1:10 to about 1:10,000. In some embodiments, the ratio is about 1:10, about 1:50, about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, or about 1:10,000.

In some embodiments, compositions of modified donor bacteria described herein are applied at a ratio of CFU modified bacteria/g plant biomass to CFU plant-associated bacteria/g plant biomass. In some embodiments, the ratio is from about 10,000:1 to about 1:1. In some embodiments, the ratio is about 10,000:1, about 5000:1, about 1000:1, about 500:1, about 100:1, about 50:1, about 25:1, 20:1, about 19:1, about 18:1, about 17:1, about 16:1, about 15:1, about 14:1, about 13:1, about 12:1, about 11:1, about 10:1 about 9:1, about 8:1, about 7:1, about 6:1, about 5:1, about 4:1, about 3:1, about 2:1, or about 1:1. In some embodiments, the ratio is from about 1:10 to about 1:10,000. In some embodiments, the ratio is about 1:10, about 1:50, about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, or about 1:10,000.

In some embodiments, compositions of modified donor bacteria described herein are applied at a ratio of CFU modified bacteria/mL to CFU plant-associated bacteria/g plant biomass. In some embodiments, the ratio is from about 10,000:1 to about 1:1. In some embodiments, the ratio is about 10,000:1, about 5000:1, about 1000:1, about 500:1, about 100:1, about 50:1, about 25:1, 20:1, about 19:1, about 18:1, about 17:1, about 16:1, about 15:1, about 14:1, about 13:1, about 12:1, about 11:1, about 10:1 about 9:1, about 8:1, about 7:1, about 6:1, about 5:1, about 4:1, about 3:1, about 2:1, or about 1:1. In some embodiments, the ration is from about 1:10 to about 1:10,000. In some embodiments, the ratio is about 1:10, about 1:50, about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, or about 1:10,000

### Formulation

Formulations of compositions described herein are provided in forms for improved delivery, accuracy of delivery, or reduced off-target delivery. In some embodiments, the formulations of compositions described herein are provided in forms for improved microbial stabilization. In some embodiments, the formulations of compositions described herein are provided in forms for improved cell viability. In some embodiments, the formulations of compositions described herein are provided in forms for improved wettability. In some embodiments, the formulations of compositions described herein are provided in forms for improved colonization. In some embodiments, a composition described herein can be provided as an emulsion, a colloid, a dust, a granule, a pellet, a powder, a spray, a mist, a gel, a paste, a fog, or a solution.

Compositions described herein provide for maintenance of the modified donor bacteria in the plant environment. In some embodiments, the donor bacteria are maintained for a week, two weeks, three weeks, four weeks, five weeks, or six weeks after inoculation. Modifications to donor bacteria and compositions described herein provide for increased duration of colonization compared to wild type bacteria. In some embodiments, a composition comprises a formulation for increased duration of colonization.

In some embodiments, compositions described herein further comprise an adjuvant. An adjuvant is a substance added to enhance the performance and/or physical properties of the composition. An adjuvant can comprise a surfactant, oil, compatibility agent, buffering agent, conditioning agent, defoaming agent, deposition agent, drift control agent, or thickener. In some embodiments, the composition comprises magnesium chloride. In some embodiments, the composition comprises from about 1 mM to about 1M magnesium chloride. In some embodiments, the composition comprises about 1 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, or about 1M magnesium chloride. In some embodiments, the concentration of magnesium chloride is in a solution before drying the composition. In some embodiments, the composition comprises Tween 20. In some embodiments, the composition comprises from about 0.01% to about 10% Tween 20 by volume. In some embodiments, the composition comprises about 0.01%, 0.05%, 0.06%, 0.07%, 0.08%, 0.09%, 0.1%, 0.2%, 0.3%, 0.4%, 0.5%, 1%, 5%, 10% Tween 20 by volume. In some embodiments, the composition comprises Triton X-100. In some embodiments, the composition comprises from about 0.01% to about 10% Triton X-100 by volume. In some embodiments, the composition comprises about 0.01%, 0.05%, 0.06%, 0.07%, 0.08%, 0.09%, 0.1%, 0.2%, 0.3%, 0.4%, 0.5%, 1%, 5%, 10% Triton X-100 by volume. In some embodiments, the composition comprises Silwet L77. In some embodiments, the composition comprises from about 0.002% to about 2% Silwet L77 by volume. In some embodiments, the composition comprises about 0.002%, 0.005%, 0.006%, 0.007%, 0.008%, 0.009%, 0.01%, 0.02%, 0.03%, 0.04%, 0.05%, 0.1%, 0.5%, 1%, 2% Silwet L77.

### VII. Production

Modified donor bacteria as described herein can be produced in liquid culture or solid phase culture. In some embodiments, a liquid culture is grown in a fermentor. A fermentor is also called a bioreactor. A fermentor can be an airlift fermentor, a continuous stirred tank fermentor, a packed bed fermentor, a fluidized bed fermentor, a continuous flow fermentor, or a bubble column fermentor. In some embodiments, the modified donor bacteria culture is further processed. In some embodiments, the modified donor bacteria is lyophilized, spray dried, concentrated, frozen, thickened, or gelled.

### VIII. Additional Uses

Compositions and methods described herein can be used to improve production or growth of a plant. In some embodiments, compositions or methods described herein treat an established disease in a plant. In some embodiments, compositions or methods described herein prevent a disease on a plant. In some embodiments, compositions or methods described herein kill a pathogenic plant-associated bacteria. In some embodiments, compositions or methods described herein, prevent growth of a plant-associated bacteria. In some embodiments, compositions or methods described herein provide for increased killing or growth prevention of a plant-associated bacteria than other treatment modalities. In some embodiments, the other treatment modalities comprise Actigard®, copper, Kocide® 3000, antibiotics, a plant immune activator, or natural microbes. In some embodiments, compositions or methods described herein are used in conjunction with other treatment modalities. In some embodiments, use of compositions and methods described herein provides for reduced use of other treatments, such as pesticides, insecticides, herbicides, fungicides, bactericides, or any other treatment. In some embodiments, compositions and methods described herein can be used as a biostimulant, providing for increased growth or production of a plant. In some embodiments, compositions and methods described herein provide factors for increased tolerance to environmental stress in a plant. In some embodiments, compositions and methods described herein provides factors for increased drought tolerance in plants. In some embodiments, compositions and methods described herein provides factors for increased heat tolerance in plants. In some embodiments, compositions and methods described herein provides factors for increased cold tolerance in plants. In some embodiments, compositions and methods described herein provides factors for increased flood tolerance in plants. In some embodiments, compositions and methods described herein provides factors for increased salinity tolerance in plants. In some embodiments, a composition provides for root signal recognition, stomatal closure, signal transport, osmotic adjustment, increased root growth, increased absorption area, or reduced transpiration area.

### EXEMPLARY EMBODIMENTS

Provided herein are modified bacteria, wherein the modified bacteria comprises: a first expression vector comprising a first exogenous nucleic acid, wherein the first exogenous nucleic acid comprises: a sequence encoding for an endonuclease; and a sequence encoding for at least one guide nucleic acid, wherein the at least one guide nucleic acid targets a sequence in a genome of one or more species of plant-associated bacteria; and a second expression vector comprising a second exogenous nucleic acid, wherein the second exogenous nucleic acid encodes for a bacterial conjugation machinery, wherein the first expression vector and the second expression vector each independently comprises a low copy origin of replication. Further provided herein are modified bacteria, wherein the at least one guide nucleic acid comprises at least 90% sequence identity to the sequence in the genome of one or more species of plant-associated bacteria. Further provided herein are modified bacteria, wherein the at least one guide nucleic acid comprises less than 90% sequence identity to a sequence in a bacterial species other than that of the one or more species of plant-associated bacteria. Further provided herein are modified bacteria, wherein the at least one guide nucleic acid comprises 100% sequence identity to the sequence in the genome of one or more species of plant-associated bacteria. Further provided herein are modified bacteria, wherein the sequence in the genome of one or more species of plant-associated bacteria is in a region of an essential gene. Further provided herein are modified bacteria, wherein the essential gene comprises dnaA, gyrA, polA, or ftsZ. Further provided herein are modified bacteria, wherein the first expression vector does not replicate autonomously. Further provided herein are modified bacteria, wherein the first expression vector comprises an oriV origin of replication. Further provided herein are modified bacteria, wherein the first expression vector further comprises deletion or mutation of a sequence encoding for A plasmid replication initiator protein (TrfA). Further provided herein are modified bacteria, wherein the second expression vector comprises a sequence encoding for A plasmid replication initiator protein (TrfA). Further provided herein are modified bacteria, wherein the TrfA comprises a sequence comprising at least 80%, 85%, 90%, 95%, 97%, 98%, or 99% sequence identity to SEQ ID NO: 46. Further provided herein are modified bacteria, wherein the TrfA comprises a sequence of SEQ ID NO: 46. Further provided herein are modified bacteria, wherein the sequence in the genome of one or more species of plant-associated bacteria is in a region of a non-essential gene. Further provided herein are modified bacteria, wherein the sequence in the genome of one or more species of plant-associated bacteria is in a non-coding region of the genome. Further provided herein are modified bacteria, wherein the plant-associated bacteria is a soil bacteria. Further provided herein are modified bacteria, wherein the plant-associated bacteria is a plant bacteria. Further provided herein are modified bacteria, wherein the plant-associated bacteria is a plant pathogenic bacteria. Further provided herein are modified bacteria, wherein the second expression vector is a conjugative plasmid. Further provided herein are modified bacteria, wherein the conjugative plasmid is a TAmob plasmid, an RP4 plasmid, an Rsa plasmid, an R702 plasmid, or an IP113 plasmid. Further provided herein are modified bacteria, wherein the conjugative plasmid is the TAmob plasmid. Further provided herein are modified bacteria, further comprising a genome modification comprising a domain essential to the replication of at least one of the first or second expression vectors. Further provided herein are modified bacteria, wherein the domain comprises a pirA gene. Further provided herein are modified bacteria, wherein the pirA gene comprises a sequence comprising at least 80%, 85%, 90%, 95%, 97%, 98%, or 99% sequence identity to SEQ ID NO: 49. Further provided herein are modified bacteria, wherein the pirA gene comprises a sequence of SEQ ID NO: 49. The modified bacteria of any one of claims 1-31, wherein each low copy origin of replication independently comprises RK2, RSAOri, pRO1600, pBR322, pACYC, pSC101, or any functional variant thereof.

Provided herein are modified bacteria, wherein the modified bacteria comprises: an expression vector comprising a first exogenous nucleic acid, wherein the first exogenous nucleic acid comprises: a sequence encoding for an endonuclease; a sequence encoding for at least one guide nucleic acid, wherein the at least one guide nucleic acid targets a sequence in a genome of one or more species of plant-associated bacteria; and a second exogenous nucleic acid, wherein the second exogenous nucleic acid encodes for a bacterial conjugation machinery, wherein the second exogenous nucleic acid is incorporated in a genome of the modified bacteria. Further provided herein are modified bacteria, wherein the at least one guide nucleic acid comprises at least 90% sequence identity to the sequence in the genome of one or more species of plant-associated bacteria. Further provided herein are modified bacteria, wherein the at least one guide nucleic acid comprises less than 90% sequence identity to a sequence in a bacterial species other than that of the one or more species of plant-associated bacteria. Further provided herein are modified bacteria, wherein the at least one guide nucleic acid comprises 100% sequence identity to the sequence in the genome of one or more species of plant-associated bacteria. Further provided herein are modified bacteria, wherein the sequence in the genome of one or more species of plant-associated bacteria is in a region of an essential gene. Further provided herein are modified bacteria, wherein the essential gene comprises dnaA, gyrA, polA, or ftsZ. Further provided herein are modified bacteria, wherein the sequence in the genome of one or more species of plant-associated bacteria is in a region of a non-essential gene. Further provided herein are modified bacteria, wherein the sequence in the genome of one or more species of plant-associated bacteria is in a non-coding region of the genome. Further provided herein are modified bacteria, wherein the plant-associated bacteria is a soil bacteria. Further provided herein are modified bacteria, wherein the plant-associated bacteria is a plant bacteria. Further provided herein are modified bacteria, wherein the plant-associated bacteria is a plant pathogenic bacteria. Further provided herein are modified bacteria, wherein the expression vector further comprises a low copy origin of replication. Further provided herein are modified bacteria, wherein the second exogenous nucleic acid further comprises a low copy origin of replication. Further provided herein are modified bacteria, wherein the first expression vector does not replicate autonomously. Further provided herein are modified bacteria, wherein the first expression vector comprises an oriV origin of replication. Further provided herein are modified bacteria, wherein the first expression vector further comprises deletion or mutation of a sequence encoding for plasmid replication initiator protein (TrfA). Further provided herein are modified bacteria, wherein the second exogenous nucleic acid comprises a sequence encoding for plasmid replication initiator protein (TrfA). Further provided herein are modified bacteria, wherein the TrfA comprises a sequence comprising at least 80%, 85%, 90%, 95%, 97%, 98%, or 99% sequence identity to SEQ ID NO: 46. Further provided herein are modified bacteria, wherein the TrfA comprises a sequence of SEQ ID NO: 46. Further provided herein are modified bacteria, further comprising a genome modification comprising a domain essential to the replication of at least one of the first or second expression vectors. Further provided herein are modified bacteria, wherein the domain comprises a pirA gene. Further provided herein are modified bacteria, wherein the pirA gene comprises a sequence comprising at least 80%, 85%, 90%, 95%, 97%, 98%, or 99% sequence identity to SEQ ID NO: 49. Further provided herein are modified bacteria, wherein the pirA gene comprises a sequence of SEQ ID NO: 49. Further provided herein are modified bacteria, wherein each low copy origin of replication independently comprises RK2, RSAOri, pRO1600, pBR322, pACYC, pSC101, or any functional variant thereof. Further provided herein are modified bacteria, wherein the bacteria comprises less than 50 copies of each expression vector. Further provided herein are modified bacteria, wherein the low copy origin of replication is a broad host low copy origin of replication. Further provided herein are modified bacteria, wherein the broad host low copy origin of replication comprises an RSAOri. Further provided herein are modified bacteria, wherein the RSAOri comprises a sequence comprising at least 80%, 85%, 90%, 95%, 97%, 98%, or 99% sequence identity to SEQ ID NO: 43. Further provided herein are modified bacteria, wherein the RSAOri comprises the sequence of SEQ ID NO: 43.

Provided herein are modified bacteria as described herein, wherein the second nucleic acid further comprises an antibiotic resistance gene. Further provided herein are modified bacteria, wherein the antibiotic resistance gene comprises a tetracycline resistance gene, a kanamycin resistance gene, or a gentamicin resistance gene. Further provided herein are modified bacteria, wherein the second exogenous nucleic acid further comprises flippase recognition target (FRT) sites flanking the antibiotic resistance gene. Further provided herein are modified bacteria, wherein the sequence encoding for at least one guide nucleic acid encodes for 2, 3, 4, 5, 6, 7, 8, 9, 10, or more guide nucleic acids. Further provided herein are modified bacteria, wherein the at least one guide nucleic acid targets a sequence in a genome of 2, 3, 4, 5, 6, 7, 8, 9, 10 or more species of plant-associated bacteria. Further provided herein are modified bacteria, further comprising a genomic modification comprising a nucleic acid encoding a toxin, wherein the toxin is lethal to the modified bacteria, wherein the first expression vector further comprises a nucleic acid encoding for an antitoxin, and wherein the antitoxin inhibits the activity of the toxin. Further provided herein are modified bacteria, wherein the toxin is ccdB and the antitoxin is ccdA. Further provided herein are modified bacteria, wherein the first exogenous nucleic acid and the second exogenous nucleic acid are DNA. Further provided herein are modified bacteria, wherein the first exogenous nucleic acid and the second exogenous nucleic acid are RNA. Further provided herein are modified bacteria, wherein the endonuclease is a Cas enzyme. Further provided herein are modified bacteria, wherein the Cas enzyme is a Type I, Type II, or Type III Cas enzyme. Further provided herein are modified bacteria, wherein the Cas enzyme is a Type II enzyme. Further provided herein are modified bacteria, wherein the Type II enzyme is a Cas9 endonuclease. Further provided herein are modified bacteria, wherein the Cas enzyme is a TevSpCas9 endonuclease.

Provided herein are modified bacteria as described herein, further comprising a DNA barcode. Further provided herein are modified bacteria, wherein the DNA barcode is inserted in the modified bacterial genome. Further provided herein are modified bacteria, further comprising a genomic modification comprising a nucleic acid encoding a repressor protein, wherein the repressor protein represses expression of a promoter on the first exogenous nucleic acid or the second exogenous nucleic acid. Further provided herein are modified bacteria, wherein the repressor protein is Wphi. Further provided herein are modified bacteria, wherein the Wphi is encoded by a sequence comprising at least 80%, 85%, 90%, 95%, 97%, 98%, or 99% sequence identity to SEQ ID NO: 31. Further provided herein are modified bacteria, wherein the Wphi is encoded by a sequence comprising SEQ ID NO: 31 Further provided herein are modified bacteria, wherein the repressor protein is CymR. Further provided herein are modified bacteria, wherein the CymR is encoded by a sequence comprising at least 80%, 85%, 90%, 95%, 97%, 98%, or 99% sequence identity to SEQ ID NO: 32. Further provided herein are modified bacteria, wherein the CymR is encoded by a sequence comprising SEQ ID NO: 32. Further provided herein are modified bacteria, wherein the promoter is WphiPro. Further provided herein are modified bacteria, wherein the WphiPro is encoded by a sequence comprising at least 80%, 85%, 90%, 95%, 97%, 98%, or 99% sequence identity to SEQ ID NO: 39. Further provided herein are modified bacteria, wherein the WphiPro is encoded by a sequence comprising SEQ ID NO: 39. Further provided herein are modified bacteria, wherein the repressor protein is cI. Further provided herein are modified bacteria, wherein the cI is encoded by a sequence comprising at least 80%, 85%, 90%, 95%, 97%, 98%, or 99% sequence identity to SEQ ID NO: 26. Further provided herein are modified bacteria, wherein the cI is encoded by a sequence comprising SEQ ID NO: 26. Further provided herein are modified bacteria, wherein the promoter is cIPro. Further provided herein are modified bacteria, wherein the cIPro is encoded by a sequence comprising at least 80%, 85%, 90%, 95%, 97%, 98%, or 99% sequence identity to SEQ ID NO: 34. Further provided herein are modified bacteria, wherein the cIPro is encoded by a sequence comprising SEQ ID NO: 34.

Provided herein are modified bacteria as described herein, wherein the modified bacteria comprises Citrobacter spp., Bacillus spp., Enterobacter spp., Escherichia spp., Serratia spp., Stenotrophomonas spp., Paraburkholderia spp., Lysinibacillus spp., Acinetobacter spp., Erwinia spp., Pectobacterium spp., Dickeya spp., Pantoea spp., Agrobacterium spp., Pseudomonas spp., Ralstonia spp., Burkholderia spp., Acidovorax spp., Xanthomonas spp., Methylobacterium spp., Clavibacter spp., Streptomyces spp., Xylella spp., Spiroplasma spp., Chryseobacterium spp., Phytoplasma spp., or Rathayibacter spp. Further provided herein are modified bacteria, wherein the modified bacteria is Pseudomonas putida, Pseudomonas alloputida, E. coli, Paraburkholderia phytofirmans, Pantoea agglomerans, Methylobacterium oryzae, Methylobacterium radiotolerans, or Pantoea vagans. Further provided herein are modified bacteria, wherein the one or more species of plant-associated bacteria comprises Xanthomonas campestris, Xanthomonas euvesicatoria, Xanthomonas perforans, Xanthomonas citri, Xanthomonas oryzae, Xanthomonas axonopodis, Xanthomonas albileneans, Pseudomonas syringae, Pseudomonas savastanoi, Pseudomonas rubrilineans, Candidatus Liberibacter americanus, Candidatus Liberibacter africanus, Candidatus Liberibacter asiaticus, Xylella fastidiosa, Ralstonia solanacearum, Erwinia amylovora, Erwinia caratovora, Agrobacterium tumefaciens, Agrobacterium vitis, Pectobacterium carotovorum, Clavibacter michiganensis, Dickeya dadantii, Dickeya solani, Microdochium oryzae, or Guignardia bidwellii. Further provided herein are modified bacteria, wherein the one or more species of plant-associated bacteria comprises Xanthomonas campestris. Further provided herein are modified bacteria, wherein the plant-associated bacteria comprise Erwinia amylovora. Further provided herein are modified bacteria, wherein the plant-associated bacteria comprise Xanthomonas. perforans. Further provided herein are modified bacteria, wherein the plant-associated bacteria comprise Candidatus Liberibacter spp. Further provided herein are modified bacteria, wherein the plant-associated bacteria comprise Xanthomonas euvesicatoria.

Provided herein are compositions, wherein the composition comprises: at least one expression vector; a first exogenous nucleic acid, wherein the first exogenous nucleic acid comprises: a sequence encoding for an endonuclease; a sequence encoding for at least one guide nucleic acid, wherein the at least one guide nucleic acid targets a sequence in a genome of one or more species of plant bacteria; and a second exogenous nucleic acid encoding for a bacterial conjugation machinery, wherein the first exogenous nucleic acid and the second exogenous nucleic acid are incorporated into one or more of the at least one expression vector. Further provided herein are compositions, wherein the plant bacteria is a plant pathogenic bacteria. Further provided herein are compositions, wherein the first exogenous nucleic acid and the second exogenous nucleic acid are incorporated into a single expression vector of the at least one expression vector. Further provided herein are compositions, wherein the first exogenous nucleic acid and the second exogenous nucleic acid are incorporated into separate expression vectors of the at least one expression vector. Further provided herein are compositions, wherein the at least one expression vector comprises at least one plasmid. Further provided herein are compositions, wherein the at least one expression vector comprises at least two plasmids. Further provided herein are compositions, wherein the plasmid is a TAmob plasmid, a GFP reporter plasmid, an RP4 plasmid, an Rsa plasmid, an R702 plasmid, an IP113 plasmid, or any combination or variation thereof. Further provided herein are compositions, wherein the sequence encoding for at least one guide nucleic acid encodes for 2, 3, 4, 5, 6, 7, 8, 9, 10, or more guide nucleic acids. Further provided herein are compositions, wherein the at least one guide nucleic acid targets 2, 3, 4, 5, 6, 7, 8, 9, 10, or more species of plant bacteria. Further provided herein are compositions, wherein the at least one guide nucleic acid is selected based on target specificity, melting temperature, and preference for guanine in the first position. Further provided herein are compositions, wherein the at least one guide nucleic acid comprises at least 80%, 85%, 90%, 95%, 97%, 98%, or 99% sequence identity to one of SEQ ID NOs: 50 to 135. Further provided herein are compositions, wherein the at least one guide nucleic acid comprises any of SEQ ID NOs: 50 to 135. Further provided herein are compositions, wherein the first exogenous nucleic acid and the second exogenous nucleic acid are DNA. Further provided herein are compositions, wherein the endonuclease is a Cas enzyme. Further provided herein are compositions, wherein the Cas enzyme is a Type I, Type II, or Type III Cas enzyme. Further provided herein are compositions, wherein the Type II Cas enzyme is a Cas9 endonuclease. Further provided herein are compositions, wherein the endonuclease is a TevSpCas9 dual nuclease. Further provided herein are compositions, wherein the one or more species of plant pathogenic bacteria are selected from Xanthomonas perforans, Xanthomonas campestris, Xanthomonas euvesicatoria, Xanthomonas citri, Xanthomonas oryzae, Xanthomonas axonopodis, Xanthomonas albileneans, Pseudomonas syringae, Pseudomonas savastanoi, Candidatus Liberibacter americanus, Candidatus Liberibacter africanus, Candidatus Liberibacter asiaticus, Xylella fastidiosa, Ralstonia solanacearum, Erwinia amylovora, Agrobacterium tumefaciens, Agrobacterium vitis, Pectobacterium carotovorum, Clavibacter michiganensis, Dickeya dadantii, or Dickeya solani. Further provided herein are compositions, wherein the one or more species of plant pathogenic bacteria comprises Xanthomonas perforans. Further provided herein are compositions, wherein the at least one guide nucleic acid comprises at least 80%, 85%, 90%, 95%, 97%, 98%, or 99% sequence identity to SEQ ID NOs: 84-92, 94-98, or 116-118. Further provided herein are compositions, wherein the at least one guide nucleic acid is one of SEQ ID NOs: 84-92, 94-98, or 116-118. Further provided herein are compositions, wherein the one or more species of plant pathogenic bacteria comprises Xanthomonas campestris. Further provided herein are compositions, wherein the at least one guide nucleic acid comprises at least 80%, 85%, 90%, 95%, 97%, 98%, or 99% sequence identity to SEQ ID NOs: 71-77 or 92-98. Further provided herein are compositions, wherein the at least one guide nucleic acid is one of SEQ ID NOs: 71-77 or 92-98. Further provided herein are compositions, wherein the one or more species of plant pathogenic bacteria comprise Erwinia amylovora. Further provided herein are compositions, wherein the at least one guide nucleic acid comprises at least 80%, 85%, 90%, 95%, 97%, 98%, or 99% sequence identity to SEQ ID NOs: 53-57, 92-94, or 98. Further provided herein are compositions, wherein the at least one guide nucleic acid is one of SEQ ID NOs: 53-57, 92-94, or 98. Further provided herein are compositions, wherein the one or more species of plant pathogenic bacteria comprise Candidatus Liberibacter species. Further provided herein are compositions, wherein the Candidatus Liberibacter species comprises at least one of Candidatus Liberibacter americanus, Candidatus Liberibacter africanus, and Candidatus Liberibacter asiaticus. Further provided herein are compositions, wherein the at least one guide nucleic acid comprises at least 80%, 85%, 90%, 95%, 97%, 98%, or 99% sequence identity to SEQ ID NOs: 50-52, 99-115, or 119-133. Further provided herein are compositions, wherein the at least one guide nucleic acid is SEQ ID NOs: 50-52, 99-115, or 119-133. Further provided herein are compositions, wherein the one or more species of plant pathogenic bacteria comprise Xanthomonas euvesicatoria. Further provided herein are compositions, wherein the first exogenous nucleic acid comprises at least 80%, 85%, 90%, 95%, 97%, 98%, or 99% sequence identity to SEQ ID NOs: 78-85, 92, or 94-98. Further provided herein are compositions, wherein the at least one guide nucleic acid is one of SEQ ID NOs: 78-85, 92, or 94-98. Provided herein are compositions as described herein, further comprising a DNA barcode.

Provided herein are compositions, wherein the composition comprises: a first expression vector comprising a first exogenous nucleic acid, wherein the first exogenous nucleic acid comprises: a sequence encoding for an endonuclease; a sequence encoding for at least one guide nucleic acid, wherein the at least one guide nucleic acid targets a sequence in a genome of one or more species of plant-associated bacteria; a second expression vector comprising a second exogenous nucleic acid, wherein the second exogenous nucleic acid encodes for a bacterial conjugation machinery, wherein the first expression vector and the second expression vector each independently comprises a low copy origin of replication. Further provided herein are compositions, wherein the first expression vector does not replicate autonomously. Further provided herein are compositions, wherein the first expression vector comprises an oriV origin of replication. Further provided herein are compositions, wherein the first expression vector further comprises deletion or mutation of a sequence encoding for plasmid replication initiator protein (TrfA). Further provided herein are compositions, wherein the second expression vector comprises a sequence encoding for plasmid replication initiator protein (TrfA). Further provided herein are compositions, wherein the TrfA comprises a sequence comprising at least 80%, 85%, 90%, 95%, 97%, 98%, or 99% sequence identity to SEQ ID NO: 46. Further provided herein are compositions, wherein the TrfA comprises a sequence of SEQ ID NO: 46. Further provided herein are compositions, wherein the plant-associated bacteria is a soil bacteria. Further provided herein are compositions, wherein the plant-associated bacteria is a plant bacteria. Further provided herein are compositions, wherein the plant bacteria is a pathogenic bacteria. Further provided herein are compositions, wherein the plant-associated bacteria is a non-pathogenic bacteria. Further provided herein are compositions, wherein the second expression vector is a conjugative plasmid. Further provided herein are compositions, wherein the conjugative plasmid is a TAmob plasmid, a GFP reporter plasmid, an RP4 plasmid, an Rsa plasmid, an R702 plasmid, an IP113 plasmid, or any combination or variation thereof. Further provided herein are compositions, wherein the conjugative plasmid is the TAmob plasmid. Further provided herein are compositions, wherein each low copy origin of replication independently comprises RK2, RSAOri, pRO1600, pBR322, pACYC, pSC101, or any functional variant thereof. Further provided herein are compositions, wherein the composition comprises less than 50 copies of each expression vector. Further provided herein are compositions, wherein the low copy origin of replication is a broad host low copy origin of replication. Further provided herein are compositions, wherein the broad host low copy origin of replication comprises an RSAOri. Further provided herein are compositions, wherein the RSAOri comprises a sequence comprising at least 80%, 85%, 90%, 95%, 97%, 98%, or 99% sequence identity to SEQ ID NO: 43. Further provided herein are compositions, wherein the RSAOri comprises the sequence described in SEQ ID NO: 43. Further provided herein are compositions, wherein the second expression vector further comprises an antibiotic resistance gene. Further provided herein are compositions, wherein the antibiotic resistance gene comprises a tetracycline resistance gene, kanamycin resistance gene, or a gentamicin resistance gene. Further provided herein are compositions, wherein the second expression vector further comprises FRT sites flanking the antibiotic resistance gene. Further provided herein are compositions, wherein the sequence encoding for at least one guide nucleic acid encodes for 2, 3, 4, 5, 6, 7, 8, 9, 10, or more guide nucleic acids. Further provided herein are compositions, wherein the at least one guide nucleic acid targets a sequence in a genome of 2, 3, 4, 5, 6, 7, 8, 9, 10, or more species of plant-associated bacteria. Further provided herein are compositions, wherein the at least one guide nucleic acid is selected based on target specificity, melting temperature, preference for guanine in the first position, or any combination thereof. Further provided herein are compositions, wherein the at least one guide nucleic acid comprises at least 80%, 85%, 90%, 95%, 97%, 98%, or 99% sequence identity to one of SEQ ID NOs: 50 to 135. Further provided herein are compositions, wherein the at least one guide nucleic acid comprises the sequence of any of SEQ ID NOs: 50 to 135. Further provided herein are compositions, wherein the first exogenous nucleic acid and the second exogenous nucleic acid are DNA. Further provided herein are compositions, wherein the endonuclease is a Cas enzyme. Further provided herein are compositions, wherein the Cas enzyme is a Type I, Type II, or Type III Cas enzyme. Further provided herein are compositions, wherein the Type II Cas enzyme is a Cas9 endonuclease. Further provided herein are compositions, wherein the Cas enzyme is a TevSpCas9 dual nuclease. Further provided herein are compositions, wherein the one or more species of plant-associated bacteria are selected from Xanthomonas perforans, Xanthomonas campestris, Xanthomonas euvesicatoria, Xanthomonas citri, Xanthomonas oryzae, Xanthomonas axonopodis, Xanthomonas albileneans, Pseudomonas syringae, Pseudomonas savastanoi, Candidatus Liberibacter americanus, Candidatus Liberibacter africanus, Candidatus Liberibacter asiaticus, Xylella fastidiosa, Ralstonia solanacearum, Erwinia amylovora, Agrobacterium tumefaciens, Agrobacterium vitis, Pectobacterium carotovorum, Clavibacter michiganensis, Dickeya dadantii, or Dickeya solani. Further provided herein are compositions, wherein the one or more species of plant-associated bacteria comprises Xanthomonas perforans. Further provided herein are compositions, wherein the at least one guide nucleic acid comprises at least 80%, 85%, 90%, 95%, 97%, 98%, or 99% sequence identity to SEQ ID NOs: 84-92, 94-98, or 116-118. Further provided herein are compositions, wherein the at least one guide nucleic acid is one of SEQ ID NOs: 84-92, 94-98, or 116-118. Further provided herein are compositions, wherein the one or more species of plant-associated bacteria comprises Xanthomonas campestris. Further provided herein are compositions, wherein the at least one guide nucleic acid comprises at least 80%, 85%, 90%, 95%, 97%, 98%, or 99% sequence identity to SEQ ID NOs: 71-77 or 92-98. Further provided herein are compositions, wherein the at least one guide nucleic acid is one of SEQ ID NOs: 71-77 or 92-98. Further provided herein are compositions, wherein the one or more species of plant-associated bacteria comprise Erwinia amylovora. Further provided herein are compositions, wherein the at least one guide nucleic acid comprises at least 80%, 85%, 90%, 95%, 97%, 98%, or 99% sequence identity to SEQ ID NOs: 53-57, 92-94, or 98. Further provided herein are compositions, wherein the at least one guide nucleic acid is one of SEQ ID NOs: 53-57, 92-94, or 98. Further provided herein are compositions, wherein the one or more species of plant-associated bacteria comprise Candidatus Liberibacter species. Further provided herein are compositions, wherein the Candidatus Liberibacter species comprises at least one of Candidatus Liberibacter americanus, Candidatus Liberibacter africanus, and Candidatus Liberibacter asiaticus. Further provided herein are compositions, wherein the at least one guide nucleic acid comprises at least 80%, 85%, 90%, 95%, 97%, 98%, or 99% sequence identity to SEQ ID NOs: 50-52, 99-115, or 119-133. Further provided herein are compositions, wherein the at least one guide nucleic acid is SEQ ID NOs: 50-52, 99-115, or 119-133. Further provided herein are compositions, wherein the one or more species of plant-associated bacteria comprise Xanthomonas euvesicatoria. Further provided herein are compositions, wherein the at least one guide nucleic acid comprises at least 80%, 85%, 90%, 95%, 97%, 98%, or 99% sequence identity to SEQ ID NOs: 78-85, 92, or 94-98. Further provided herein are compositions, wherein the at least one guide nucleic acid is one of SEQ ID NOs: 78-85, 92, or 94-98. Further provided herein are compositions, further comprising a DNA barcode. Further provided herein are compositions, further comprising one or more gene transfer agents.

Provided herein are modified bacteria, wherein the modified bacteria comprises: at least one expression vector; a first exogenous nucleic acid, wherein the first exogenous nucleic acid comprises: a sequence encoding for an endonuclease; and a sequence encoding for at least one guide nucleic acid, wherein the at least one guide nucleic acid targets a sequence in a genome of one or more species of plant bacteria, and a second exogenous nucleic acid encoding for a bacterial conjugation machinery, wherein the first exogenous nucleotide and optionally, the second exogenous nucleic acid, are incorporated into one or more of the at least one an expression vector. Further provided herein are modified bacteria, wherein the plant bacteria is a plant pathogenic bacteria. Further provided herein are modified bacteria, wherein the first exogenous nucleic acid and the second exogenous nucleic acid are incorporated into a single expression vector of the at least one expression vector. Further provided herein are modified bacteria, wherein the first exogenous nucleic acid and the second exogenous nucleic acid are incorporated into separate expression vectors of the at least one expression vector. Further provided herein are modified bacteria, wherein the second exogenous nucleic acid encoding for the bacterial conjugation machinery is incorporated in the bacterial genome. Further provided herein are modified bacteria, wherein the at least one expression vector comprises at least one plasmid. Further provided herein are modified bacteria, wherein the at least one expression vector comprises at least two plasmids. Further provided herein are modified bacteria, wherein the plasmid is a TAmob plasmid, a GFP reporter plasmid, an RP4 plasmid, an Rsa plasmid, an R702 plasmid, an IP113 plasmid, or any combination or variation thereof. Further provided herein are modified bacteria, wherein the plasmid is a TAmob plasmid. Further provided herein are modified bacteria, wherein the modified bacteria transfer the expression vector to the plant pathogenic bacteria. Further provided herein are modified bacteria, wherein the transfer is a conjugative transfer. Further provided herein are modified bacteria, wherein the transfer is a viral transduction. Further provided herein are modified bacteria, wherein the transfer is by transformation. Further provided herein are modified bacteria, wherein the transfer is facilitated with gene transfer agents. Further provided herein are modified bacteria, wherein the transferred expression vector expresses the encoded endonuclease and the at least one guide nucleic acid, and wherein the endonuclease and the at least one guide nucleic acid complex to make a lethal double-stranded cut in the plant pathogenic bacteria genome. Further provided herein are modified bacteria, wherein the sequence encoding for at least one guide nucleic acid encodes for 2, 3, 4, 5, 6, 7, 8, 9, 10, or more guide nucleic acids. Further provided herein are modified bacteria, wherein the at least one guide nucleic acid targets 2, 3, 4, 5, 6, 7, 8, 9, 10 or more species of plant pathogenic bacteria. Further provided herein are modified bacteria, further comprising a genomic modification comprising a nucleic acid encoding a toxin, wherein the toxin is lethal to the modified bacteria, wherein the expression vector further comprises a nucleic acid encoding for an antitoxin, and wherein the antitoxin inhibits the activity of the toxin. Further provided herein are modified bacteria, wherein the toxin is ccdB and the antitoxin is ccdA. Further provided herein are modified bacteria, wherein the exogenous nucleic acid is DNA. Further provided herein are modified bacteria, wherein the exogenous nucleic acid is RNA. Further provided herein are modified bacteria, wherein the endonuclease is a Cas enzyme. Further provided herein are modified bacteria, wherein the Cas enzyme is a Type I, Type II, or Type III Cas enzyme. Further provided herein are modified bacteria, wherein the Cas enzyme is a Type II enzyme. Further provided herein are modified bacteria, wherein the Type II enzyme is a Cas9 endonuclease. Further provided herein are modified bacteria, wherein the Cas enzyme is a TevSpCas9 endonuclease. Further provided herein are modified bacteria, further comprising a DNA barcode. Further provided herein are modified bacteria, wherein the DNA barcode is inserted in the modified bacterial genome. Further provided herein are modified bacteria, further comprising a genomic modification comprising a nucleic acid encoding a repressor protein, wherein the repressor protein represses expression of a promoter on the expression vector. Further provided herein are modified bacteria, further comprising a genomic modification comprising a nucleic acid encoding a repressor protein, wherein the repressor protein represses expression of a promoter on the first exogenous nucleic acid or the second exogenous nucleic acid. Further provided herein are modified bacteria, wherein the repressor protein is Wphi. Further provided herein are modified bacteria, wherein the Wphi is encoded by a sequence comprising at least 80%, 85%, 90%, 95%, 97%, 98%, or 99% sequence identity to SEQ ID NO: 31. Further provided herein are modified bacteria, wherein the Wphi is encoded by a sequence comprising SEQ ID NO: 31. Further provided herein are modified bacteria, wherein the repressor protein is CymR. Further provided herein are modified bacteria, wherein the CymR is encoded by a sequence comprising at least 80%, 85%, 90%, 95%, 97%, 98%, or 99% sequence identity to SEQ ID NO: 32. Further provided herein are modified bacteria, wherein the CymR is encoded by a sequence comprising SEQ ID NO: 32. Further provided herein are modified bacteria, wherein the promoter is WphiPro. Further provided herein are modified bacteria, wherein the WphiPro is encoded by a sequence comprising at least 80%, 85%, 90%, 95%, 97%, 98%, or 99% sequence identity to SEQ ID NO: 39. Further provided herein are modified bacteria, wherein the WphiPro is encoded by a sequence comprising SEQ ID NO: 39. Further provided herein are modified bacteria, wherein the repressor protein is cI. Further provided herein are modified bacteria, wherein the cI is encoded by a sequence comprising at least 80%, 85%, 90%, 95%, 97%, 98%, or 99% sequence identity to SEQ ID NO: 26. Further provided herein are modified bacteria, wherein the cI is encoded by a sequence comprising SEQ ID NO: 26. Further provided herein are modified bacteria, wherein the promoter is cIPro. Further provided herein are modified bacteria, wherein the cIPro is encoded by a sequence comprising at least 80%, 85%, 90%, 95%, 97%, 98%, or 99% sequence identity to SEQ ID NO: 34. Further provided herein are modified bacteria, wherein the cIPro is encoded by a sequence comprising SEQ ID NO: 34. Further provided herein are modified bacteria, further comprising a genome modification comprising a domain essential to the replication of the at least one expression vector. Further provided herein are modified bacteria, wherein the domain comprises a pirA gene. Further provided herein are modified bacteria, wherein the pirA gene comprises a sequence comprising at least 80%, 85%, 90%, 95%, 97%, 98%, or 99% sequence identity to SEQ ID NO: 49. Further provided herein are modified bacteria, wherein the pirA gene comprises a sequence of SEQ ID NO: 49. Further provided herein are modified bacteria, wherein the modified bacteria is a modified non-pathogenic bacteria. Further provided herein are modified bacteria, wherein the modified donor bacteria comprises Citrobacter spp., Bacillus spp., Enterobacter spp., Serratia spp., Stenotrophomonas spp., Lysinibacillus spp., Acinetobacter spp., Erwinia spp., Pectobacterium spp., Dickeya spp., Pantoea spp., Agrobacterium spp., Pseudomonas spp., Ralstonia spp., Burkholderia spp., Acidovorax spp., Xanthomonas spp., Clavibacter spp., Streptomyces spp., Xylella spp., Spiroplasma spp., Phytoplasma spp., or Rathayibacter spp. Further provided herein are modified bacteria, wherein the modified donor bacteria is Pseudomonas putida. Further provided herein are modified bacteria, wherein the modified donor bacteria is E. coli. Further provided herein are modified bacteria, wherein the modified donor bacteria is Paraburkholderia phytofirmans. Further provided herein are modified bacteria, wherein the modified donor bacteria is Pantoea agglomerans. Further provided herein are modified bacteria, wherein the one or more species of plant pathogenic bacteria comprises Xanthomonas campestris, Xanthomonas euvesicatoria, Xanthomonas perforans, Xanthomonas citri, Xanthomonas oryzae, Xanthomonas axonopodis, Xanthomonas albileneans, Pseudomonas syringae, Pseudomonas savastanoi, Pseudomonas rubrilineans, Candidatus Liberibacter americanus, Candidatus Liberibacter africanus, Candidatus Liberibacter asiaticus, Xylella fastidiosa, Ralstonia solanacearum, Erwinia amylovora, Erwinia caratovora, Agrobacterium tumefaciens, Agrobacterium vitis, Pectobacterium carotovorum, Clavibacter michiganensis, Dickeya dadantii, Dickeya solani, Microdochium oryzae, or Guignardia bidwellii. Further provided herein are modified bacteria, wherein the one or more species of plant pathogenic bacteria comprises Xanthomonas. campestris. Further provided herein are modified bacteria, wherein the plant pathogenic bacteria comprise Erwinia amylovora. Further provided herein are modified bacteria, wherein the plant pathogenic bacteria comprise Xanthomonas. perforans. Further provided herein are modified bacteria, wherein the plant pathogenic bacteria comprise Candidatus. Liberibacter spp. Further provided herein are modified bacteria, wherein the plant pathogenic bacteria comprise Xanthomonas. euvesicatoria.

Provided herein are compositions, wherein the composition comprises: a modified bacteria as described herein; and a plant. Further provided herein are compositions, wherein the plant is of the genus Brassica, Solanum, Malus, Citrus, Vitis, Saccharum, Zea, Oryza, Triticum, or any combination, strain, or variant thereof.

Provided herein are methods of horizontal gene transfer (HGT), wherein the method comprises: introducing a modified bacteria to an ecosystem of a plant, wherein the modified bacteria comprises: at least one expression vector; a first exogenous nucleic acid, wherein the first exogenous nucleic acid comprises: a sequence encoding for an endonuclease; and a sequence encoding for at least one guide nucleic acid, wherein the at least one guide nucleic acid targets a sequence in a genome of one or more species of plant pathogenic bacteria; and a second exogenous nucleic acid encoding for a bacterial conjugation machinery, wherein the first exogenous nucleic acid and the second exogenous nucleic acid are incorporated into one or more of the at least one expression vector. Further provided herein are methods of HGT, wherein the first exogenous nucleic acid and the second exogenous nucleic acid are incorporated into a single expression vector of the at least one expression vector. Further provided herein are methods of HGT, wherein the first exogenous nucleic acid and the second exogenous nucleic acid are incorporated into separate expression vectors of the at least one expression vector. Further provided herein are methods of HGT, wherein the at least one expression vector comprises at least one plasmid. Further provided herein are methods of HGT, wherein the at least one expression vector comprises at least two plasmids. Further provided herein are methods of HGT, wherein the plasmid is a TAmob plasmid, a GFP reporter plasmid, an RP4 plasmid, an Rsa plasmid, an R702 plasmid, an IP113 plasmid, or any combination or variation thereof. Further provided herein are methods of HGT, wherein the plasmid a TAmob plasmid. Further provided herein are methods of HGT, wherein the modified bacteria transfer the expression vector to the plant pathogenic bacteria. Further provided herein are methods of HGT, wherein the transfer is a conjugative transfer. Further provided herein are methods of HGT, wherein the transfer is a viral transduction. Further provided herein are methods of HGT, wherein the transfer is by transformation. Further provided herein are methods of HGT, wherein the transfer is facilitated with gene transfer agents. Further provided herein are methods of HGT, wherein the transferred expression vector expresses the encoded endonuclease and the at least one guide nucleic acid, and wherein the endonuclease and the at least one guide nucleic acid complex to make a lethal double-stranded cut in the plant pathogenic bacteria genome. Further provided herein are methods of HGT, wherein the first exogenous nucleic acid comprises sequences encoding 2, 3, 4, 5, 6, 7, 8, 9, 10 or more guide nucleic acids. Further provided herein are methods of HGT, wherein the sequence encoding one or more guide nucleic acid targets 2, 3, 4, 5, 6, 7, 8, 9, 10 or more plant pathogenic bacteria. Further provided herein are methods of HGT, wherein the exogenous nucleic acid is DNA. Further provided herein are methods of HGT, wherein the endonuclease is a Cas enzyme. Further provided herein are methods of HGT, wherein the Cas enzyme is a Type I, Type II, or Type III Cas enzyme. Further provided herein are methods of HGT, wherein the Cas enzyme is a Type II enzyme. Further provided herein are methods of HGT, wherein the Type II enzyme is a Cas9 endonuclease. Further provided herein are methods of HGT, wherein the endonuclease is a TevSpCas9 dual nuclease. Further provided herein are methods of HGT, wherein the modified bacteria further comprises a genomic modification comprising a nucleic acid encoding a toxin, wherein the toxin is lethal to the modified bacteria, wherein the expression vector further comprises a nucleic acid sequence encoding for an antitoxin, and wherein the antitoxin inhibits the activity of the toxin. Further provided herein are methods of HGT, wherein the toxin is ccdB and the antitoxin is ccdA. Further provided herein are methods of HGT, wherein the modified bacteria further comprises a genomic modification comprising a nucleic acid encoding a repressor protein, wherein the repressor protein represses expression of a promoter on the expression vector. Further provided herein are methods of HGT, wherein the repressor protein is Wphi C. Further provided herein are methods of HGT, wherein the repressor protein is P2hd. Further provided herein are methods of HGT, wherein the repressor protein is P2 C. Further provided herein are methods of HGT, wherein the modified bacteria comprises a genomic domain essential to the replication of the expression vector. Further provided herein are methods of HGT, wherein the domain comprises a pirA gene. Further provided herein are methods of HGT, wherein the pirA gene comprises a sequence comprising at least 80%, 85%, 90%, 95%, 97%, 98%, or 99% sequence identity to SEQ ID NO: 49. Further provided herein are methods of HGT, wherein the pirA gene comprises a sequence of SEQ ID NO: 49. Further provided herein are methods of HGT, wherein the transfer of the expression vector from the modified bacteria to the plant pathogenic bacteria has a transfer efficiency greater than from about 10\hat{ }-3 to about 1. Further provided herein are methods of HGT, wherein the transfer of the expression vector from the modified bacteria to the plant pathogenic bacteria has a transfer efficiency greater than about 10\hat{ }-1.

Provided herein are methods of horizontal gene transfer (HGT), wherein the method of HGT comprises: introducing a modified bacteria to an ecosystem of a plant, wherein the modified bacteria comprises: a first expression vector comprising a first exogenous nucleic acid, wherein the first exogenous nucleic acid comprises: a sequence encoding for an endonuclease; and a sequence encoding for at least one guide nucleic acid, wherein the at least one guide nucleic acid targets a sequence in a genome of one or more species of plant-associated bacteria; and a second expression vector comprising a second exogenous nucleic acid, wherein the second exogenous nucleic acid encodes for a bacterial conjugation machinery, wherein the first expression vector and the second expression vector each independently comprises a low copy origin of replication. Further provided herein are methods of HGT, wherein the first expression vector does not replicate autonomously. Further provided herein are methods of HGT, wherein the first expression vector comprises an oriV origin of replication. Further provided herein are methods of HGT, wherein the first expression vector further comprises deletion or mutation of a sequence encoding for plasmid replication initiator protein (TrfA). Further provided herein are methods of HGT, wherein the second expression vector comprises a sequence encoding for plasmid replication initiator protein (TrfA). Further provided herein are methods of HGT, wherein the TrfA comprises a sequence comprising at least 80%, 85%, 90%, 95%, 97%, 98%, or 99% sequence identity to SEQ ID NO: 46. Further provided herein are methods of HGT, wherein the TrfA comprises a sequence of SEQ ID NO: 46. Further provided herein are methods of HGT, wherein the plant-associated bacteria is a soil bacteria. Further provided herein are methods of HGT, wherein the plant-associated bacteria is a plant bacteria. Further provided herein are methods of HGT, wherein the plant bacteria is a pathogenic bacteria. Further provided herein are methods of HGT, wherein the second expression vector is a plasmid. Further provided herein are methods of HGT, wherein the plasmid is a conjugative plasmid. Further provided herein are methods of HGT, wherein the conjugative plasmid is a TAmob plasmid, an RP4 plasmid, an Rsa plasmid, an R702 plasmid, an IP113 plasmid, or any combination or variation thereof. Further provided herein are methods of HGT, wherein the conjugative plasmid is the TAmob plasmid. Further provided herein are methods of HGT, further comprising transferring the first expression vector to the one or more species of plant-associated bacteria. Further provided herein are methods of HGT, wherein the transferring is a conjugative transfer. Further provided herein are methods of HGT, wherein the transferring is facilitated with one or more gene transfer agents. Further provided herein are methods of HGT, further comprising expressing the endonuclease and the at least one guide nucleic acid in the one or more species of plant-associated bacteria, and wherein the endonuclease and the at least one guide nucleic acid complex to make a lethal double-stranded cut in the plant-associated bacteria genome. Further provided herein are methods of HGT, wherein the sequence encoding for at least one guide nucleic acid encodes for 2, 3, 4, 5, 6, 7, 8, 9, 10 or more guide nucleic acids. Further provided herein are methods of HGT, wherein the at least one guide nucleic acid targets a sequence in a genome of 2, 3, 4, 5, 6, 7, 8, 9, 10 or more species of plant-associated bacteria. Further provided herein are methods of HGT, wherein the first exogenous nucleic acid and the second exogenous nucleic acid are DNA. Further provided herein are methods of HGT, wherein the first exogenous nucleic acid and the second exogenous nucleic acid are RNA. Further provided herein are methods of HGT, wherein the endonuclease is a Cas enzyme. Further provided herein are methods of HGT, wherein the Cas enzyme is a Type I, Type II, or Type III Cas enzyme. Further provided herein are methods of HGT, wherein the Cas enzyme is a Type II enzyme. Further provided herein are methods of HGT, wherein the Type II enzyme is a Cas9 endonuclease. Further provided herein are methods of HGT, wherein the Cas enzyme is a TevSpCas9 endonuclease. Further provided herein are methods of HGT, wherein the modified bacteria further comprises a genomic modification comprising a nucleic acid encoding a toxin, wherein the toxin is lethal to the modified bacteria, wherein the first expression vector further comprises a nucleic acid sequence encoding for an antitoxin, and wherein the antitoxin inhibits the activity of the toxin. Further provided herein are methods of HGT, wherein the toxin is ccdB and the antitoxin is ccdA. Further provided herein are methods of HGT, wherein the modified bacteria further comprises a genomic modification comprising a nucleic acid encoding a repressor protein, wherein the repressor protein represses expression of a promoter on the expression vector. Further provided herein are methods of HGT, wherein the repressor protein is Wphi. Further provided herein are methods of HGT, wherein the repressor protein is CymR. Further provided herein are methods of HGT, wherein the repressor protein is cI. Further provided herein are methods of HGT, wherein the modified bacteria further comprises a genome modification comprising a domain essential to the replication of at least one of the first or second expression vectors. Further provided herein are methods of HGT, wherein the domain comprises a pirA gene. Further provided herein are methods of HGT, wherein the pirA gene comprises a sequence comprising at least 80%, 85%, 90%, 95%, 97%, 98%, or 99% sequence identity to SEQ ID NO: 49. Further provided herein are methods of HGT, wherein the pirA gene comprises a sequence of SEQ ID NO: 49. Further provided herein are methods of HGT, wherein the method of HGT has a transfer efficiency from about 10\hat{ }-3 to about 1. Further provided herein are methods of HGT, wherein the transferring has a transfer efficiency greater than about 10\hat{ }-1.

Provided herein are bacterial formulations, wherein the bacterial formulation comprises: a modified bacteria as described herein, and an adjuvant. Further provided herein are bacterial formulations, wherein the adjuvant comprises, alone or in combination, a surfactant, an oil, a compatibility agent, a buffering agent, a conditioning agent, a defoaming agent, a deposition agent, a drift control agent, or a thickener. Further provided herein are bacterial formulations, wherein the adjuvant comprises Tween 20, Triton X-100, Silwet L77, or magnesium chloride. Further provided herein are bacterial formulations, wherein the adjuvant comprises from about 0.01% to about 10% Tween 20. Further provided herein are bacterial formulations, wherein adjuvant comprises about 0.1% Tween 20. Further provided herein are bacterial formulations, wherein the adjuvant comprises from about 0.01% to about 10% Triton X-100. Further provided herein are bacterial formulations, wherein the adjuvant comprises about 0.1% Triton X-100. Further provided herein are bacterial formulations, wherein the adjuvant comprises from about 0.002% to about 2% Silwet L77. Further provided herein are bacterial formulations, wherein the adjuvant comprises about 0.02% Silwet L77. Further provided herein are bacterial formulations, wherein the adjuvant comprises from about 1 mM to about 1M magnesium chloride. Further provided herein are bacterial formulations, wherein the adjuvant comprises about 10 mM magnesium chloride. Further provided herein are bacterial formulations, wherein the formulation is an emulsion, a colloid, a dust, a granule, a pellet, a powder, a spray, a mist, a gel, a paste, a fog or a solution.

Provided herein are methods of improving a condition on a plant, the method comprising applying to a plant a bacterial formulation as described herein, wherein application of the bacterial formulation improves the condition on the plant. Further provided herein are methods of improving a condition on a plant, further comprising reducing biomass of a plant-associated bacteria in a plant. Further provided herein are methods of improving a condition on a plant, wherein the condition on a plant is measured by a production of the plant. Further provided herein are methods of improving a condition on a plant, further comprising reducing a plant pathogenic bacteria in a plant. Further provided herein are methods of improving a condition on a plant, wherein a production of the plant is improved. Further provided herein are methods of improving a condition on a plant, wherein an availability of a nutrient or water is improved. Further provided herein are methods of improving a condition on a plant, wherein a level of a toxin is reduced. Further provided herein are methods of improving a condition on a plant, wherein the bacterial formulation is applied in an amount of CFU modified bacteria/mL liquid media of about 10\hat{ }2 CFU/mL, about 10\hat{ }3 CFU/mL, about 10\hat{ }4 CFU/mL, about 10\hat{ }5 CFU/mL, about 10\hat{ }6 CFU/mL, about 10\hat{ }7 CFU/mL, about 10\hat{ }8 CFU/mL, about 10\hat{ }9 CFU/mL, about 10\hat{ }10 CFU/mL. Further provided herein are methods of improving a condition on a plant, wherein the applying comprises a foliar spray. Further provided herein are methods of improving a condition on a plant, wherein the applying comprises an injection. Further provided herein are methods of improving a condition on a plant, wherein the applying comprises syringe infiltration. Further provided herein are methods of improving a condition on a plant, wherein the applying comprises a root drench. Further provided herein are methods of improving a condition on a plant, wherein the applying comprises a seed coating. Further provided herein are methods of improving a condition on a plant, wherein the application is a pre-emergent application. Further provided herein are methods of improving a condition on a plant, wherein the application is a post-emergent application. Further provided herein are methods of improving a condition on a plant, wherein the application is prophylactic of a pathogenic bacteria infection. Further provided herein are methods of improving a condition on a plant, wherein the application is for treatment of a pathogenic bacteria infection.

Provided herein are methods of improving the growth of a plant, the method comprising applying to a plant the modified bacteria as described herein, wherein application of the modified bacteria improves the growth of the plant. Further provided herein are methods of improving the growth of a plant, further comprising reducing biomass of a plant-associated bacteria in a plant. Further provided herein are methods of improving the growth of a plant, wherein the growth of a plant is measured by a production of the plant. Further provided herein are methods of improving the growth of a plant, wherein an availability of a nutrient or water is improved. Further provided herein are methods of improving the growth of a plant, wherein a level of a toxin is reduced. Further provided herein are methods of improving the growth of a plant, wherein the modified bacteria is applied in a liquid media, and the modified bacteria is applied in an amount of CFU modified bacteria/mL liquid media of about 10\hat{ }2 CFU/mL, about 10\hat{ }3 CFU/mL, about 10\hat{ }4 CFU/mL, about 10\hat{ }5 CFU/mL, about 10\hat{ }6 CFU/mL, about 10\hat{ }7 CFU/mL, about 10\hat{ }8 CFU/mL, about 10\hat{ }9 CFU/mL, about 10\hat{ }10 CFU/mL. Further provided herein are methods of improving the growth of a plant, wherein the applying comprises a foliar spray. Further provided herein are methods of improving the growth of a plant, wherein the applying comprises an injection. Further provided herein are methods of improving the growth of a plant, wherein the applying comprises syringe infiltration. Further provided herein are methods of improving the growth of a plant, wherein the applying comprises a root drench. Further provided herein are methods of improving the growth of a plant, wherein the applying comprises a seed coating. Further provided herein are methods of improving the growth of a plant, wherein the application is a pre-emergent application. Further provided herein are methods of improving the growth of a plant, wherein the application is a post-emergent application. Further provided herein are methods of improving the growth of a plant, wherein the application is prophylactic of a pathogenic bacteria infection. Further provided herein are methods of improving the growth of a plant, wherein the application is for treatment of a pathogenic bacteria infection.

Provided herein are methods of manufacture, wherein the method comprises: generating a modified bacteria as described herein; growing the modified bacteria; formulating the modified bacteria with an adjuvant. Further provided herein are methods of manufacture, wherein the adjuvant comprises, alone or in combination, a surfactant, an oil, a compatibility agent, a buffering agent, a conditioning agent, a defoaming agent, a deposition agent, a drift control agent, or a thickener. Further provided herein are methods of manufacture, wherein the adjuvant comprises phosphate buffered saline. Further provided herein are methods of manufacture, wherein the adjuvant comprises Tween 20, Triton X-100, Silwet L77, or magnesium chloride. Further provided herein are methods of manufacture, wherein the adjuvant comprises from about 0.01% to about 10% Tween 20. Further provided herein are methods of manufacture, wherein adjuvant comprises about 0.1% Tween 20. Further provided herein are methods of manufacture, wherein the adjuvant comprises from about 0.01% to about 10% Triton X-100. Further provided herein are methods of manufacture, wherein the adjuvant comprises about 0.1% Triton X-100. Further provided herein are methods of manufacture, wherein the adjuvant comprises from about 0.002% to about 2% Silwet L77. Further provided herein are methods of manufacture, wherein the adjuvant comprises about 0.02% Silwet L77. Further provided herein are methods of manufacture, wherein the adjuvant comprises from about 1 mM to about 1M magnesium chloride. Further provided herein are methods of manufacture, wherein the adjuvant comprises about 10 mM magnesium chloride. Further provided herein are methods of manufacture, wherein the formulation is an emulsion, a colloid, a dust, a granule, a pellet, a powder, a spray, a mist, a gel, a paste, a fog or a solution. Further provided herein are methods of manufacture, wherein the generating step comprises selection of the at least one guide nucleic acid. Further provided herein are methods of manufacture, wherein the selection of the at least one guide nucleic acid comprises analysis of target specificity, melting temperature, and guanine presence in first position. Further provided herein are methods of manufacture, wherein the generating step comprises performing one or more rounds of directed evolution on the modified bacteria to select for an improved conjugation plasmid, wherein the directed evolution is performed using a non-targeted mutagenesis system. Further provided herein are methods of manufacture, wherein the non-targeted mutagenesis system is the MP6 mutagenesis system. Further provided herein are methods of manufacture, wherein the directed evolution is by selective pressure.

Provided herein are modified bacteria, wherein the modified bacteria comprises: a modified Pseudomonas putida comprising: a modified pTAmob plasmid; a domain on the plasmid comprising at least 80%, 85%, 90%, 95%, 97%, 98%, or 99% sequence identity to SEQ ID NO: 85, wherein the domain is within a sequence comprising at least 80%, 85%, 90%, 95%, 97%, 98%, or 99% sequence identity to SEQ ID NO: 21; and a domain on the plasmid encoding for a cas9 endonuclease.

Provided herein are modified bacteria, wherein the modified bacteria comprises: a modified Pseudomonas putida comprising: a modified pTAmob plasmid, wherein the modified pTAmob plasmid is modified to comprise a low copy origin of replication; an RK2 plasmid comprising a cassette, wherein the cassette comprises: a promoter, a gRNA sequence, a gRNA scaffold, and a terminator, and a domain encoding for a Cas9 endonuclease. Further provided herein are modified bacteria, wherein the cassette comprises at least 80% sequence identity to SEQ ID NO: 21 or SEQ ID NO: 22. Further provided herein are modified bacteria, wherein the cassette comprises the sequence of SEQ ID NO: 21 or SEQ ID NO: 22. Further provided herein are modified bacteria, wherein the gRNA sequence comprises at least 90% sequence identity to any one of SEQ ID NOS: 50-135. Further provided herein are modified bacteria, wherein the modified pTAmob plasmid comprises an RSAOri origin of replication. Further provided herein are modified bacteria, wherein the RSAOri origin of replication comprises at least 80% sequence identity with SEQ ID NO: 43. Further provided herein are modified bacteria, wherein the RSAOri comprises the sequence of SEQ ID NO: 43.

Provided herein are modified bacteria comprising a modified pTAmob plasmid, wherein the modified pTAmob plasmid comprises a broad host low copy origin of replication. Further provided herein are modified bacteria, wherein the modified pTAmob plasmid comprises an RSAOri origin of replication. Further provided herein are modified bacteria, wherein the RSAOri origin of replication comprises at least 80% sequence identity with SEQ ID NO: 43. Further provided herein are modified bacteria, wherein the RSAOri origin of replication comprises a sequence of SEQ ID NO: 43. Further provided herein are modified bacteria, wherein the modified bacteria is Pseudomonas putida, Pseudomonas alloputida, E. coli, Paraburkholderia phytofirmans, Pantoea agglomerans, Methylobacterium oryzae, Methylobacterium radiotolerans, or Pantoea vagans. Further provided herein are methods of improving a condition on a plant, the methods comprise applying to a plant the modified bacteria described herein. Further provided herein are methods, wherein the method further comprises reducing biomass of a plant-associated bacteria in a plant. Further provided herein are methods, wherein the growth of a plant is measured by a production of the plant. Further provided herein are methods, wherein an availability of a nutrient or water is improved. Further provided herein are methods, wherein a level of a toxin is reduced. Further provided herein are methods, wherein the modified bacteria is applied in a liquid media, and the modified bacteria is applied in an amount of CFU modified bacteria/mL liquid of about 10\hat{ }2 CFU/mL, about 10\hat{ }3 CFU/mL, about 10\hat{ }4 CFU/mL, about 10\hat{ }5 CFU/mL, about 10\hat{ }6 CFU/mL, about 10\hat{ }7 CFU/mL, about 10\hat{ }8 CFU/mL, about 10\hat{ }9 CFU/mL, about 10\hat{ }10 CFU/mL.

### EXAMPLES

### Example 1: gRNA Selection

Guide RNA sequences were selected by screening of a publicly available database disclosing genomic data for 3,837 microbial genomes isolated from plant and soil microbiomes (available at http://labs.bio.unc.edu/Dangl/Resources/gfobap_website/index.html). gRNA sequences targeting E. coli, Pseudomonas protegens, Erwinia amylovora, Xanthomonas campestris, Xanthomonas euvesicatoria, Xanthomonas perforans, and Candidatus Liberibacter spp. Individually, as well as all possible combination subsets of these 5 microbes were generated. Screening first identified instances of Protospacer adjacent motif (PAM) sequences in each genome. This list of gRNA sequences was filtered for GC content of 40-60%. The Bowtie alignment software tool eliminated gRNA sequences that aligned with non-target DNA with up to 2 mismatches. In sequences targeting multiple bacteria, the Bowtie tool filtered out gRNA sequences that aligned with multiple sites across all targeted bacteria. Final selection of gRNA sequences included consideration of number of non-target hits, melting temperature, and incidence of guanine in the first nucleotide position. Table 9 shows gRNA sequences as the results of this in silico selection criteria.

### Example 2: Bacterial Strain Construction

Modified donor bacteria were generated by insertion of exogenous nucleic acids. Bacterial cultures were grown at 30 degrees Celsius with LB medium. As needed, antibiotics were added to the growth medium to the following concentrations: 100 μg/mL carbenicillin (Cb), 50 μg/mL kanamycin (Km), 40 μg/mL gentamicin (Gm), 10 μg/mL tetracycline (Tet).

Tet^{R} strains of E. coli EC100D, E. coli MG1655, Xanthomonas campestris (Xcc), Xanthomonas perforans (GEV), Xanthomonas euvesicatoria (E3), Erwinia amylovora (Earn), Pseudomonas protegens (Pf5), Paraburkholderia phytofirmans (PsJN), and Candidatus Liberibacter crescens BT-1 (BT-1) were generated by integrating a tetracycline resistance marker downstream of the glmS gene onto the genome of electrocompetent cells using the Tn7 transposase (pR.007) and plating on LB+Tet media.

P. putida KT2440 LP2 was generated by integrating two genomic landing pads via allelic exchange onto the genome of electrocompetent P. putida KT2440. The first landing pad was integrated in a neutral site between tadA (location: 1,182,833) and mltF (location: 1,182,963) and insulated with DT3 and DT54. The second landing pad att7B was integrated in a neutral site between Fe—S-oxidoreductase (location: 5,689,378) and the PhoPQ-activated pathogenicity-like protein (location: 5,689,477) and insulated with DT60 and DT104.

### Example 3: Plasmid Construction with Repressor/Activator System

A repressor expressed within the modified donor bacteria prevents expression of a plasmid gene within the donor bacteria. Following transfer into a recipient, the plasmid gene is allowed to turn on. A Cas9 gRNA plasmid is generated comprising a genetic switch that turns off Cas9 expression when the Cas9-gRNA plasmid is in the modified donor bacterial cell and activates Cas9 expression when the plasmid moves into the recipient cell. The repressor-activator system is advantageous because 1) small molecule inducers are eliminated, and 2) the burden on the modified bacterial cell is reduced, improving competitiveness of the engineered bacteria in a complex microbiome. P. putida KT2440 LP2, as described in Example 2, is modified to include Wphi in the first landing pad. The expressed protein repressed expression of a pWphiPro promoter inserted upstream of the Cas9 gene in the Cas9-gRNA plasmid.

### Example 4: Plasmid Construction with Toxin/Antitoxin System

A CcdA/CcdB Type II Toxin-Antitoxin system is incorporated to replace the use of an antibiotic selection marker for plasmid maintenance in the modified bacterial strain. The ccdB toxin gene is integrated into the modified bacterial genome at the first landing pad. The ccdA antitoxin gene is incorporated onto the Cas9-gRNA plasmid, thereby forcing the modified bacterium to maintain the plasmid, and thereby production of the antitoxin, in order to avoid toxin-induced cell death. The antibiotic selection marker is removed from the Cas9-gRNA plasmid.

### Example 5: Bacterial Modification to Incorporate Repressor, Plasmid Replication, and Toxin Genes

Triple modifications to a donor bacteria generate a secured plasmid with limited replication. By adding a toxin-antitoxin system to a modified bacteria, the bacteria is required to maintain the plasmid carrying the antitoxin gene. Incorporating a gene essential to plasmid replication into the bacterial genome limits replication of the plasmid to internal to the modified donor bacteria. P. putida KT2440 LP2RT is a modification of P. putida KT2440 LP2, as described in Example 2. The bacterial genome incorporates a repressor gene selected from P2hd, Wphi C, and P2 C, and the ccsB toxin gene into the first landing pad and the pirA gene and a DNA barcode into the second landing pad.

### Example 6: Cas9 Plasmid Construction

A Cas9-gRNA plasmid was a template for additional modifications. The Cas9-gRNA template plasmid (pR.029) was constructed by synthesizing the following fragment to include the gRNA template cassette (J23119 promoter, gRNA Spacer1 (random nucleotide sequence flanked by BsaI sites), gRNA scaffold, and ilv GEDA terminator) as in SEQ ID NO: 21, the Cas9 cassette (pCymRC promoter, RBS, Cas9, and DT36 terminator) as in SEQ ID NO: 1, the CymR cassette (LacIQ promoter, CymR, and M13 terminator), as in SEQ ID NO: 5, the oriT, as in SEQ ID NO: 2, the partitioning locus (parABCDE and trfA), and the RK2 oriV origin of replication, as in SEQ ID NO: 12.

The Cas9-gRNA template plasmid was used to construct plasmids targeting microbes (pR.036, pR.037, pR.093, pR.094, pR.116-pR.119, pR.150, pR.151). gRNA sequences were inserted into pR.029 in place of the gRNA Spacer1 using the BsaI sites. Two complementary oligos containing the 20 nucleotide target sequence flanked by BsaI restriction sites were synthesized (Integrated DNA Technologies, Coralville, IA, USA) and annealed at 98 degrees Celsius for 5 minutes at a concentration of 1 μM then cooled to room temperature to create dsDNA. Sequences for the oligos are listed in Table 10. The dsDNA and Cas9-gRNA template plasmid were assembled using standard Golden Gate reagents (NEB T4 DNA ligase, BsaI-HFv2, 10×NEB Ligase Buffer) and one of three assembly protocols:

TABLE 10
Oligonucleotides.

| SEQ ID | |
|---|---|
| NO: | Sequence in 5′ to 3′ order |
| 136 | CAAAAAAAAAGGCTCCAAAAGGAGCCTCAACGTTTGAATTT |
| | TGCATAACGTTCACGTGCA |
| 137 | AGCCCTCTTTTTGATAAATTTTCTCGAGGTGTACCTGCAAT |
| | TCGCGCGCGAAGGC |
| 138 | GAGTCAGCTAGGAGGTGACTGACAAATAAAACGAAAGGCTC |
| | AGTCGAAAGACTGGGC |
| 139 | AGCTAGCTGTCAAAGATCTTTAGAATTCCAGAATTCAAAAG |
| | GCCATCCGTCAGGATGGC |
| 140 | TGGAATTCTAAAGATCTTTGACAGCTAGCTCAGTCCTAGGT |
| | ATAATACTAGTGGAGACC |
| 141 | TGGAATTCTAAAGATCTTTGACAGCTAGCTCAGTCCTAGGT |
| | ATAATACTAGTGGAGACC |
| 142 | TGTCTGCTCCTCGGTTATGTTTTTAAGGTCAAAAAAAACCC |
| | CCGGACCTTTCGGTGCGGGGGTCTTAGTTCGTTAAGGCTTG |
| | ATCTCTAG |
| 143 | AGAGCTTCAATTTAATTATATCAGTTATTCACGTGGAGCTT |
| | ATCGGC |
| 144 | ACCTCGAGAAAATTTATCAAAAAGAGGGCTAAACAAACAGA |
| | C |
| 145 | ATCTCCGCCTATCTCATAATACAAACAGACCAGATTGTCTG |
| | TTTGTTGAATCTATTATAC |
| 146 | CTAGAGATCTGTTTAGCTTGCCTCGTCCCCG |
| 147 | CTAGAGATCTGTTTAGCTTGCCTCGTCCCCG |
| 148 | TGTCTGCTCCTCGGTTATGTTTTTAAGGTCAAAAAAAACCC |
| 149 | TGTCTGCTCCTCGGTTATGTTTTTAAGGTCAAAAAAAACCC |
| 150 | GACCTTAAAAACATAACCGAGGAGCAGACAGCGCAACGCAA |
| | TTAATGTGAGTTAGCTCAC |
| 151 | GGGGACGAGGCAAGCTAAACAGATCTCTAGCAGAAACGCCG |
| | TCGAAGCCGTGTG |
| 152 | TGCGAGGCTGGCCGATAAGCTCCACGTGAATAACTGATATA |
| | ATTAAATTGAAGCTCTGGG |

In the first protocol, samples were incubated at 37 degrees Celsius for 1 hour, followed by 55 degrees Celsius for 10 min, 65 degrees Celsius for 10 min, and finally 37 degrees Celsius for 10 min. In the second “shuffle” protocol, samples were incubated at 37 degrees Celsius for 5 min followed by 16 degrees Celsius for 5 min. The cycle was repeated for 20 cycles. Samples were then incubated at 55 degrees Celsius for 15 min, 65 degrees Celsius for 10 min, and finally 37 degrees Celsius for 15 min. In the third, “Rapid Shuffle (NEB)” protocol, samples were incubated at 37 degrees Celsius for 1 min followed by 16 degrees Celsius for 1 min. The cycle was repeated for 30 cycles. Samples were then incubated at 55 degrees Celsius for 15 min, 65 degrees Celsius for 10 min, and finally 37 degrees Celsius for 15 min.

### Example 7: Construction of Additional Cas9 Plasmids

The Cas9-gRNA template plasmid (pR.029) was used to construct additional killer plasmids. An exemplary killer plasmid (pR.240) was made by deleting two regions that were not necessary for the function of the plasmid, including the p15A origin of replication and a spacer area containing a fragment of the klaC gene. These changes allow better prediction and control of the replication of the plasmid. The changes also made the plasmid smaller, which allows for better conjugation efficiency.

The multimer resolution system ParABC and the toxin-antitoxin system ParDE, which served as the pR.029 plasmid maintenance systems, were removed. This approximately 3 kb region has 100% sequence identity with TA-Mob and therefore poses a risk of recombination. Additionally, ParDE is a toxin-antitoxin system which could lead to killer plasmid persistence in the environment. Those elements were replaced with the plasmid R1 derived ParCMR segregation system (SEQ ID NO: 47) and the gamma-delta multimer resolvase cassette from the E. coli F plasmid Tn1000 transposon (SEQ ID NO: 48). Those modifications allow for efficient maintenance of our killer plasmid in the chassis strain.

Another exemplary killer plasmid v3 (pR.241) was derived from killer plasmid v2 (pR.240). pR.240 has an oriV origin of replication derived from RP4/RK2. oriV requires the initiator protein TrfA for replication. TrfA (SEQ ID NO: 46) is encoded both on pR.240 and TA-Mob. trfA was deleted from the killer plasmid v2 (pR.240) to make pR.241. By doing this, the killer plasmid can no longer replicate autonomously and relies on the copy of TrfA encoded on TA-Mob. Consequently, the killer plasmid would not persist in the environment outside of the modified donor bacteria. The killer plasmid does not need to replicate inside a target cell in order to kill it. Removing homology between the killer plasmid and TA-Mob also decreases chances of recombination.

### Example 8: Dual-Plasmid System Construction and Validation

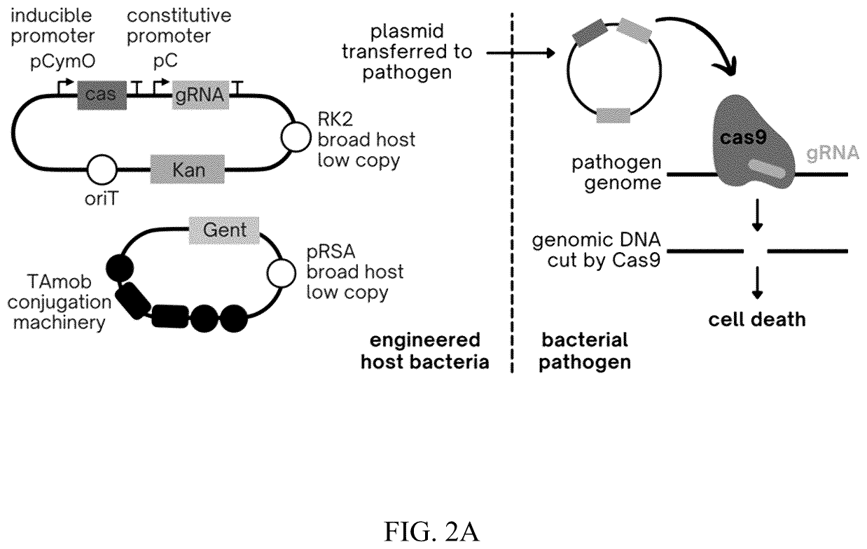

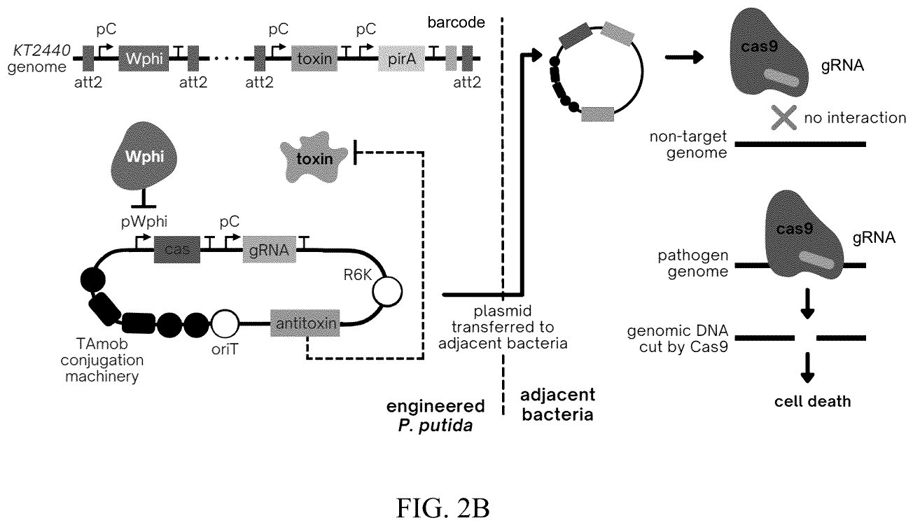

A dual-plasmid system separates conjugation and CRISPR functions, allowing for transfer of genome modification machinery into the recipient bacteria, without transfer of the propagation capability. The CRISPR and conjugation components were divided into separate plasmids with an inducible system with Cas9 under inducible expression (FIG. 2A). Multiple CRISPR plasmids were assembled with gRNAs targeting essential genes (dnaA, gyrA, polA, ftsZ) in Xanthomonas campestris.

### Example 9: Single-Plasmid System Construction with Combined Vector System

A single cis-acting plasmid, comprising conjugative machinery and genetic payload on a single plasmid was generated using a combined vector system. The combined vector system was constructed by splitting the 50 kb TAmob plasmid (pTAmob) into 7 smaller plasmids containing sub-fragments of pTAmob (pR.057—pR.061, pR.126, pR.127) and a “payload” cloning plasmid (pR.056) that contains a Type II molecular cloning site to rapidly insert any DNA sequence. The sub-fragment plasmids were constructed using two fragments PCR amplified from pTAmob using the primers as specified: pR.057 (sf-1Af/sf-1Ar; sf-1Bf/sf-1Br); pR.058 (sf-2Af/sf-2Ar; sf-2Bf/sf-2Br); pR.059 (sf-3Af/sf-3Ar; sf-3Bf/sf-3Br); pR.060 (sf-4Af/sf-4Ar; sf-4Bf/sf-4Br); pR.061 (sf-5Af/sf-5Ar; sf-5Bf/sf-5Br); pR.126 (sf-6Af/sf-6Ar; sf-6Bf/sf-6Br); and pR.127 (sf-7Af/sf-7Ar; sf-7Bf/sf-7Br). Primer sequences are shown in Table 11. All native BsaI sites were removed during the PCR amplification step. Each PCR product was purified and ligated by Gibson assembly. pR.056 was modified using Gibson cloning to construct plasmids with a genetic payload. Subsequently, Golden Gate assembly was used to assemble the full conjugative plasmid with the desired payload.

TABLE 11
Primer sequences.

| SEQ | | |
|---|---|---|
| ID | | |
| NO: | Identifier | Sequence |
| 153 | sf-1 Af | AAGGTCTCGAAGGGCTCGGCCCTTCCGGCTGGCTGGTTTATTGCTGATA |
| | | AATCTGG |
| 154 | sf-1Ar | GGTCTCGTGATTTCCTGGGTGTCGTCGTCAAGC |
| 155 | sf-1Bf | AGCCGGAAGGGCCGAGCCCTTCGAGACCTTGTGTCTCAAAATCTCTGAT |
| | | GTACATTGCAC |
| 156 | sf-1Br | TGACGACGACACCCAGGAAATCACGAGACCTAATTGGTAACGAATCAGA |
| | | CAATTGACGGC |
| 157 | sf-2Af | GGCTACGGTCTCGATCACGCGCGGCACGGTCAAGCTGCTGCGCGAGTTC |
| | | CTGGACGAGAAGGGCCGCGATCCCAACACCGTCGATGCCTT |
| 158 | sf-2Ar | GGCTACGGTCTCAGAGCGTCCCGTCGGTCGCCGTCGCCGCCTGTGGCGT |
| | | TGAGGGTGGTTCTGGCTGCG |
| 159 | sf-2Bf | ACCGTGCCGCGCGTGATCGAGACCGTAGCCTTGTGTCTCAAAATCTCTG |
| | | ATGTACATTGC |
| 160 | sf-2Br | CGACCGACGGGACGCTCTGAGACCGTAGCCTAATTGGTAACGAATCAGA |
| | | CAATTGACGGC |
| 161 | sf-3 Af | GGCTACGGTCTCAGCTCGCGCCGTCAGCGCCGGCGGAACCGTCGCACTC |
| | | C |
| 162 | sf-3 Ar | GGCTACGGTCTCTGGTGCTTCTTGCCGTCCATGATGGGGTGC |
| 163 | sf-3Bf | GCGCTGACGGCGCGAGCTGAGACCGTAGCCTTGTGTCTCAAAATCTCTG |
| | | ATGTACATTGC |
| 164 | sf-3Br | GGACGGCAAGAAGCACCAGAGACCGTAGCCTAATTGGTAACGAATCAGA |
| | | CAATTGACGGC |
| 165 | sf-4Af | GGCTACGGTCTCTCACCGCATCGAGACCGAG |
| 166 | sf-4Ar | GGCTACGGTCTCAACTGCTGAACATCCCCATGCCG |
| 167 | sf-4Bf | TCCGTCTCGATGCGGTGAGAGACCGTAGCCTTGTGTCTCAAAATCTCTG |
| | | ATGTACATTGC |
| 168 | sf-4Br | TGGGGATGTTCAGCAGTTGAGACCGTAGCCTAATTGGTAACGAATCAGA |
| | | CAATTGACGGC |
| 169 | sf-5 Af | GGCTACGGTCTCACAGTGCGCAGCCGGACAG |
| 170 | sf-5 Ar | GGCTACGGTCTCGTCTACGAGGTAGACAAGCTCGGGCAGACG |
| 171 | sf-5Bf | CTTGTCTACCTCGTAGACGAGACCGTAGCCTAATTGGTAACGAATCAGA |
| | | CAATTGACGGC |
| 172 | sf-5Br | CTTGTCTACCTCGTAGACGAGACCGTAGCCTAATTGGTAACGAATCAGA |
| | | CAATTGACGGC |
| 173 | sf-6Af | GGCTACGGTCTCGTAGACATAGGGCACGAACTTCG |
| 174 | sf-6Ar | GGTCTCGTGTAGTTGCGTCGCGGTGCATGGAGCCGGGCCACCTCGACCT |
| | | GAATGG |
| 175 | sf-6Bf | TTCGTGCCCTATGTCTACGAGACCGTAGCCTTGTGTCTCAAAATCTCTG |
| | | ATGTACATTGC |
| 176 | sf-6Br | CCATGCACCGCGACGCAACTACACGAGACCTAATTGGTAACGAATCAGA |
| | | CAATTGACGGC |
| 177 | sf-7Af | CGCGGCCGCTTCTAGAGGGTCTCGTAGACATAGGGCACGAACTTCGACT |
| | | GG |
| 178 | sf-7Ar | GCTACTAGTAGGTCTCGTTTGTTGATCTGTTGATGGCCTGGAACACCGG |
| | | G |
| 179 | sf-7Bf | GCTTCTAGAGGGTCTCGCAAAGGGCTTCGGGGCCACGTACCTC |
| 180 | sf-7Br | CGCTACTAGTAGGTCTCGTGTAGTTGCGTCGCGGTGCATGGAGC |
| 181 | 660 | TCATGGCTCTGCCCTCGG |
| 182 | 661 | TGGGTTGAAGGTGAAGCCGG |
| 183 | 705 | CCTGGCGGCGTTGTGACAATTTACCGAACAACTCCGCGGCCGGGAAGCC |
| | | GTAAACTTGGTCTGACAGTTACCTTAGAAAAAC |
| 184 | 706 | GAAGCCATCACAAACGGCATGATGAACCTGAATCGCCAGCGGCATCAGC |
| | | ACAATTCCACACAACATACGAGC |
| 185 | 687 | TCGGCGTCCTGCTCGTGATCGGGAGTATCTGGCTGGGCCAACGTTCCAA |
| | | CCGCACTCCTATTAATTAAACACGCGTTTGTCCTTTTCGG |
| 186 | 707 | GCACCCCAGGCTTTACACTTTATGCTTCCGGCTCGTATGTTGTGTGGAA |
| | | TTGTTAATTAACGGCCGGCCCTTTTCATC |
| 187 | 688 | GATGTTGTCTACATGGCTCTGCTGTAGTGAGTGGGTTGCGCTCCGGCAG |
| | | CGGTCCTGATAAACTTGGTCTGACAGTTACCTTAGAAAAAC |
| 188 | 708 | TTAAAAATTTTAAATTATAATTATTTTTATAGCACGTGATGAAAAGGGC |
| | | CGGCCGTTAATTAACAATTCCACACAACATACGAGC |

### Example 10: Single-Plasmid Construction Using Homologous Recombination

### Lambda Red Recombineering

To construct a single cis-acting plasmid, comprising conjugative machinery and genetic payload on a single plasmid, the pTAmob plasmid is engineered using the pKD46 plasmid and a modified version of the lambda red recombineering protocol from Datsenko and Wanner 2000. pTAmob (pR.003) is transformed into DH5alpha E. coli cells containing the pKD46 lambda-red recombineering plasmid. The lambda red recombination machinery is induced by the addition of arabinose. Induced, mid-log cells are then electroporated with a PCR amplicon containing the origin of transfer (oriT) amplified from the RP4 conjugative plasmid (pR.120) using primers 660 (SEQ ID NO. 181) and 661 (SEQ ID NO: 182). The oriT amplicon has ˜500 bp homology arms targeting the pTAmob plasmid. The electroporated cells are recovered in SOC media for 1 hr, then mixed with E.027 (tetracycline-resistant E. co/i) and spotted and dried onto an LB agar plate without antibiotics. Successful oriT-containing recombinant plasmids (Tamob+oriT) are able to transfer themselves into the E.027 cells while unmodified pTAmob plasmids are not able to do so. After overnight growth, the lawn of bacteria contains a mixture of unmodified pTAmob plasmids in tetracycline-sensitive cells only and Tamob+oriT recombinant plasmids in both tetracycline-sensitive cells and tetracycline-resistant cells. The lawn is spread onto a plate containing tetracycline and gentamicin to select for tetracycline-resistant E.027 cells that contain gentamicin-resistant TAmob+oriT.

Verified TAmob+oriT plasmids are transformed into DH5alpha E. coli cells containing the pKD46 lambda-red recombineering plasmid. The lambda red recombination machinery is induced by the addition of arabinose. Induced, mid-log cells are then electroporated with a PCR amplicon that replaces the gentamicin resistance marker with a recombination cassette containing the kanamycin resistance marker, the CymR repressor, Cas9 expressed from a pCym promoter, and a constitutively expressed gRNA_21 (SEQ ID NO: 88) targeting the dnaA gene of X. perforans. The cassette is amplified from pR.093 using primers 705 (SEQ ID NO: 183) and 706 (SEQ ID NO: 184). The electroporated cells are recovered in SOC media for 1 hr and plated on LB agar with gentamicin to select for recombinants with gentamicin resistance replaced with the recombination cassette (TAmob+oriT+delGent-Cas9-gRNA21). Gentamicin resistant colonies usually contain a mixture of unmodified TAmob+oriT and TAmob+oriT+delGent-Cas9-gRNA21. These colonies are grown, and the plasmids are extracted and then retransformed at low concentration into DH5alpha E. coli cells to isolate pure Tamob+oriT+delGent-Cas9-gRNA21 plasmids, which are then sequence verified.

### Yeast Assembly

To construct a single cis-acting plasmid, comprising conjugative machinery and genetic payload on a single plasmid, a linearized conjugative plasmid is assembled with the genetic payload and a yeast origin and auxotrophic marker (CEN6-ura3) using a yeast assembly protocol modified from Aparicio et al 2021. RP4-delTet-PacI-ChlorLacZa-PacI is generated using lambda red recombineering similarly to the protocol above to introduce a unique pair of PacI sites. RP4-delTet-PacI-ChlorLacZa-PacI is linearized with PacI restriction enzyme, then transformed into InvScl yeast cells along with a CEN6-ura3 amplicon (amplified from pTrans using primers 687 (SEQ ID NO: 185) and 707 (SEQ ID NO: 186)) and a recombination cassette containing the kanamycin resistance marker, the CymR repressor, Cas9 expressed from a pCym promoter, and a constitutively expressed gRNA_5 (SEQ ID NO: 71) targeting the polA gene of Xcc (amplified from pR.036 using primers 688 (SEQ ID NO: 187) and 708 (SEQ ID NO: 188)). Each fragment has ˜60 bp of overlap with the next. After recovery, the yeast are plated on CM-U plates, which lack uracil and select for assembled plasmids with the ura3 marker. Plasmid DNA is extracted from yeast colonies that grow on the CM-U plates. The desired plasmid is mixed with the yeast 2 μm and at a low concentration, so the plasmid DNA is transformed into DH5a E. coli to isolate the desired plasmid and improve yield for sequencing.

### Example 11: Single-Plasmid Construction with Integrated Conjugation Machinery

To construct a modified bacteria with TAmob integrated on the genome, the TAmob plasmid was edited to replace the pBBR1 origin of replication and gentamicin resistance marker with a tetracycline resistance marker flanked by FRT sites, Tn5 gene (transposon), the origin of transfer (OriT), and a p15A origin of replication. This TAmob-Tn5-p15A plasmid was conjugated into P. putida and the lambda red recombination machinery was induced by the addition of arabinose. Tn5 was used to insert the TAmob-TetR-oriT cassette into random loci in the P. putida genome. P. putida's natural chloramphenicol resistance was used as a counterselection to select for P. putida cells with an integrated TAmob cassette (intTAmob). Upon integration into the chromosome of the recipient cell, intTAmob is no longer capable of mobilizing itself. Genome integrated clones were sequenced to identify location of integration. Verified P. alloputida strains with integrated TAmob were transformed with a Cas9-gRNA plasmid.

### Example 12: Dual-Plasmid Construction Including Suicide Vector

The pirA gene is essential for R6K plasmid replication. A bacterium is modified to comprise pirA in the bacterial genome. A system with vectors as described in Examples 7, with an R6K origin of replication on the CRISPR plasmid, is introduced into the modified bacterium. The CRISPR plasmid acts as a “suicide” vector, relying on the pirA for replication and using the R6K origin of replication. The vector will only replicate when in proximity to the pirA gene, restricting replication outside of the cell or in a plant-associated bacteria.

### Example 13: Single-Plasmid Construction Including Suicide Vector

The pirA gene is essential for R6K plasmid replication. A bacterium is modified to comprise pirA in the bacterial genome. A vector as described in Examples 8 or 9 with an R6K origin of replication is introduced into the modified bacterium. The vector acts as a “suicide” vector, relying on the pirA for replication and using the R6K origin of replication. The vector will only replicate when in proximity to the pirA gene, restricting replication outside of the cell or in a plant-associated bacteria.

### Example 14: Modified Bacteria with DNA Barcode

Bacteria modified with plasmids as described in Examples 8, 10, or 11, are further modified in their genome to comprise a unique DNA barcode sequence for tracking and identification.

### Example 15: Plasmid Construction with gRNA Array

A plasmid system with multiple gRNA sites can bind to multiple sites within a single genome. Alternatively, a plasmid carrying multiple gRNAs can target multiple organisms. The plasmid as described in Examples 8, 10, or 11 is further modified to include multiple gRNA sites. Briefly, to the Cas9 template, gRNA template 1 (SEQ ID NO: 21) and gRNA template 2 (SEQ ID NO: 22) are added, followed by the RK2 vector with partitioning locus (parABCDE) (SEQ ID NO: 12).

### Example 16: Transformation Assays

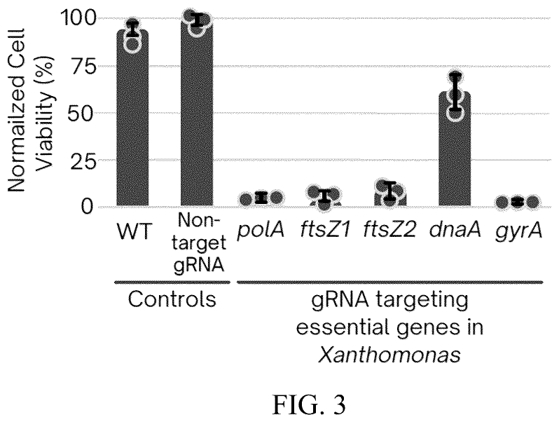

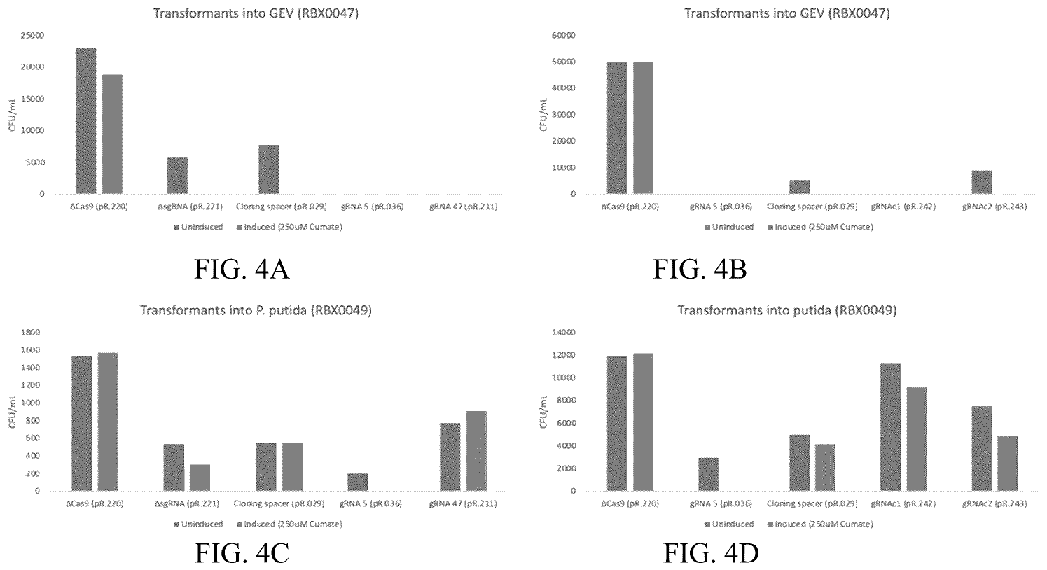

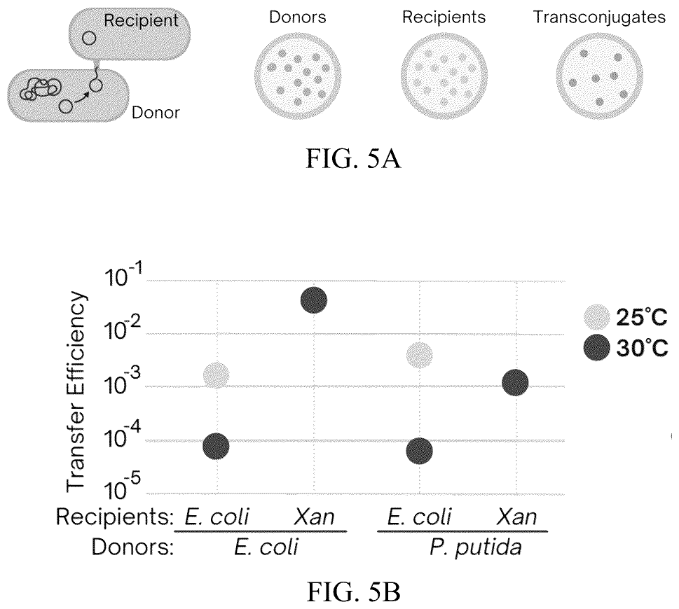

Overnight cultures were diluted 1:100 in fresh LB and grown to an optical density (OD600) of approximately 0.3-0.5. 2 mL cultured cells were centrifuged at 7,500 rpm for 10 minutes and pellets were washed in 1 mL 10% glycerol two times. After the final wash, cells were centrifuged for 10 minutes at 7,500 rpm and resuspended in 40 μL 10% glycerol ( 1/50th original volume) and stored on ice. Following 5 minutes incubation on ice, cells were electroporated and resuspended in 500 μL LB media. For the chromosomal target assay (FIG. 3 and FIG. 4), serial dilutions of cells were plated on LB+Kan to select for transformants. Plates were incubated at 30 degrees Celsius, and the number of colony forming units (CFU) were enumerated on the following day. Killing efficacy was used to assess whether the given gRNA plasmids were toxic to cells and was calculated as the CFU/mL of plasmid transformants divided by the CFU/mL of cells that received a non-target plasmid.

FIGS. 4A-4D show high levels of cell viability for pR.220 without Cas9 (ΔCas9) and no viability of X. perforans for Cas9-gRNA plasmids with gRNA designed to target X. perforans (pR.036 and pR.211) (FIG. 4A and FIG. 4B), whereas P. putida transformed with these plasmids shows moderate viability (FIG. 4C and FIG. 4D), where the reduction in viability is primarily attributed to toxicity as a result of overexpression of Cas9.

### Example 17: Conjugation Assays

The transfer efficiency of the horizontal gene transfer mechanism depicted in FIG. 5A was assayed. Plasmids pTAmob and a reporter plasmid containing GFP and OriT (pR.002) were co-transformed into competent NEB10 E. coli and P. putida as donor strains. E. coli MG1655 Tet^{R} or X. campestris (Xan) Tet^{R} were used as recipient strains. Donor and recipient strains grown overnight in LB with appropriate antibiotics were pelleted and resuspended in sterile PBS to a concentration of 1×10⁸ CFU/mL. Mating pairs were mixed at a donor to recipient ratio of 10:1, unless otherwise specified. 10 μL of mating mixtures were spotted onto LB agar plates and matings were performed for 24 hours at 25 degrees Celsius and 30 degrees Celsius. At the end of the mating, cells were recovered by scraping the spot from the LB plate and resuspending the cells in 400 μL sterile PBS. After vigorous vortexing, 200 μL was plated on selective media to select for recipients (Tet) and transconjugates (Tet+Kan). Colonies were enumerated after 24 hours of incubation at 30 degrees Celsius to determine viable cell counts and were averaged over three replicates. Transfer efficiency was calculated by dividing the number of transconjugates by the number of recipient cells (FIG. 5B). Conjugation at 30 degrees Celsius between the E. coli donor strain and X. campestris recipient strain showed transfer efficiencies approaching 10¹.

### Example 18: Killing Assay Using Two-Plasmid System

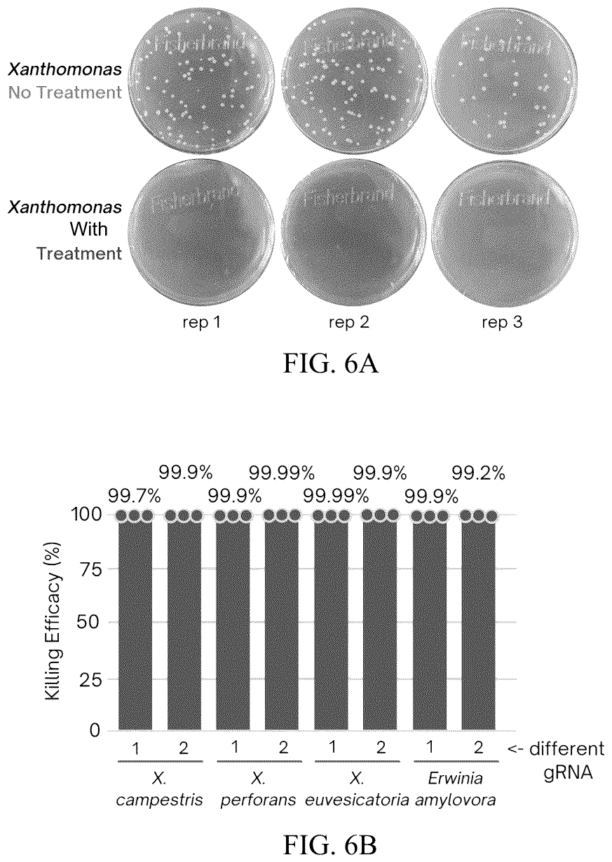

The killing efficiency of pathogens was evaluated using the donor cells engineered with the two-plasmid system. P. putida co-transformed with pTAmob and pR.036 (X. campestris 1) or pTAmob and pR.037 (X. campestris 2) were used to perform conjugation matings with X. campestris (Xcc Tet^{R}) at a ratio of 10:1 (P. putida:X. campestris cells) on solid agar media for 48 hours at 30 degrees Celsius. Results are visualized in FIG. 6A. Similarly, P. putida co-transformed with pTAmob and pR.093 (X. perforans 1) or pTAmob and pR.094 (X. perforans 2) were used to perform conjugation matings with X. perforans (GEV Tet^{R}). P. putida co-transformed with pTAmob and pR.116 (X. euvesicatoria 1) or pTAmob and pR.117 (X. euvesicatoria 2) were used to perform conjugation matings with X. euvesicatoria. P. putida co-transformed with pTAmob and pR.118 (Erwinia amylovora 1) or pTAmob and pR.119 (Erwinia amylovora 2) were used to perform conjugation matings with Erwinia amylovora. Surviving target cells were enumerated on LB media to determine killing efficiency and compared to cultures processed through the same protocol without treatment with modified P. putida (n=3). As shown in FIG. 6B, >99% of bacteria treated with modified P. putida using either of two gRNA specific for their respective strains were killed after 48 hours.

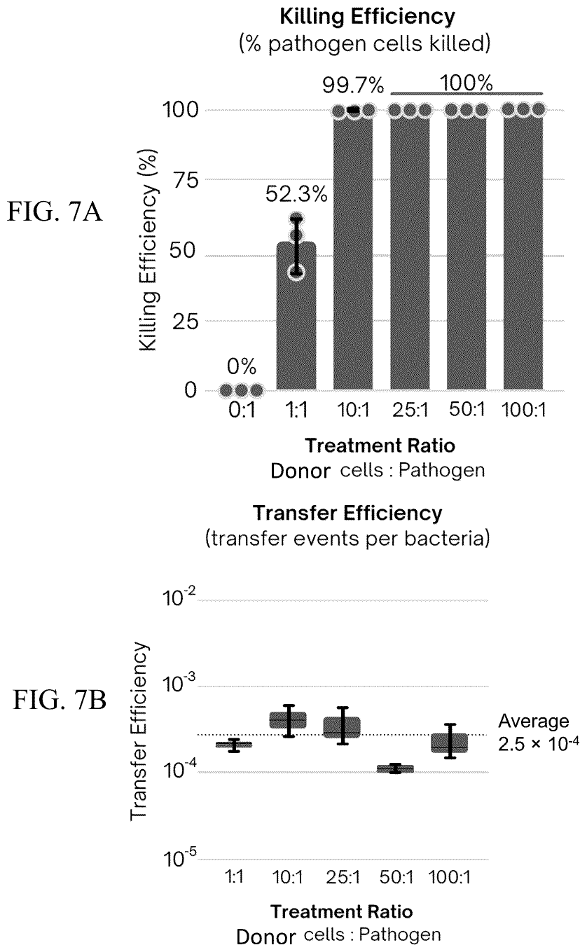

Modified strains were then assessed for killing efficiency at varying ratios of P. putida to X. campestris. P. putida co-transformed with pTAmob and pR.036 was used to perform conjugation matings with X. campestris as described above. Donor and recipient strains were combined at ratios of 0:1, 1:1, 10:1, 25:1, 50:1, and 100:1 (P. putida:X. campestris cells), diluted 1:100 in fresh PBS, plated at 10p per sample on to LB media, and allowed to mate for 24 hours. Surviving X. campestris cells were enumerated on LB media to determine killing efficacy and compared to X. campestris processed through the same protocol without P. putida (n=3). P. putida co-transformed with pTAmob and pR.029 (random gRNA template) was used as a control. Conjugation assays were carried out as described above. Killing efficacy was quantified by dividing the number of recipient cells that survived the mating by the number of surviving recipient cells in the control. The P. putida control (pTAmob+pR.029) was also used to measure transfer efficiency, which was calculated by dividing the number of transconjugates by the number of recipient cells. FIG. 7A shows increasing percent killing with increased ratio of modified donor cells to pathogens, with 99.7% killing at a 10:1 treatment ratio, and 100% killing at ratios at or above 25:1. FIG. 7B illustrates the transfer efficiency of different ratios of modified bacteria:pathogens, averaging 2.5×10⁴ across tested ratios.

### Example 19: Genome-Integrated TAmob Demonstrates Killing Comparable to Two Plasmid System

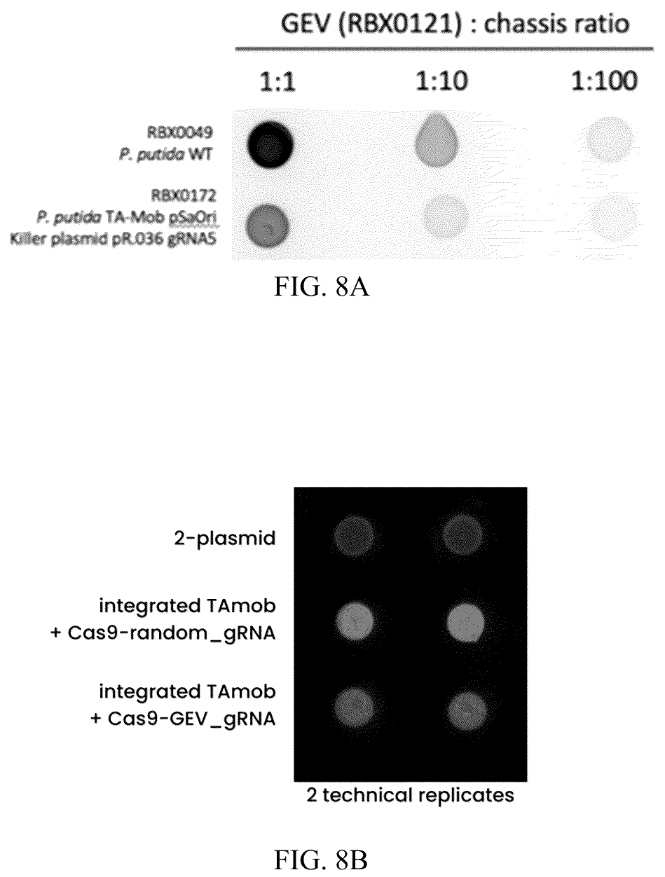

Target cell killing was compared between donor bacteria modified to comprise conjugation machinery incorporated within the bacterial genome and on a plasmid independent of the CRISPR mechanism. A conjugation “killing” assay was performed between modified P. putida and X. perforans carrying a GFP fluorescent reporter. P. putida was engineered with the (1) the two-plasmid system as described in Example 8, (2) a genome-integrated TAmob according to Example 10 and a CRISPR plasmid carrying a random gRNA (pR.029) according to Example 11, and (3) a genome-integrated TAmob according to Example 11 and a CRISPR plasmid carrying gRNA21 according to Example 6. Two technical replicates are shown in FIG. 8B.

A fluorescent X. perforans strain demonstrates a reduction in fluorescent signal as a measure of pathogen knockdown. The random gRNA showed no knockdown in fluorescent signal compared to untreated X. perforans, whereas the 2-plasmid and integrated TAmob system showed distinct knockdown in X. perforans viability.

### Example 20: Non-Target Killing Assay

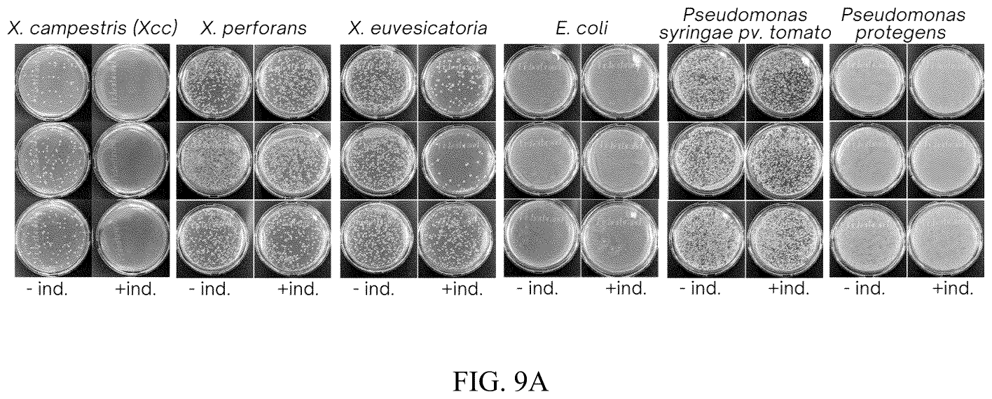

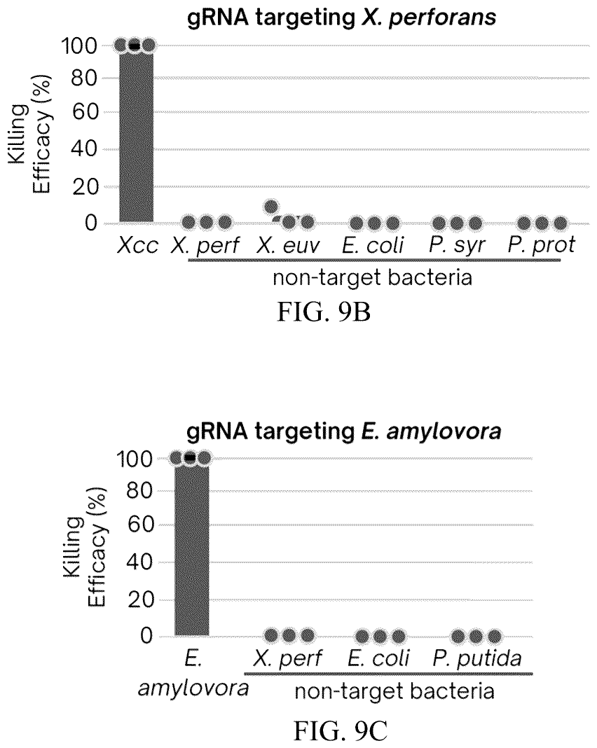

Killing efficacy in non-target strains was evaluated by performing the conjugation method described above with P. putida co-transformed with pTAmob and the Cas9-gRNA plasmid pR.036 designed to target X. campestris. Modified P. putida was used to perform conjugation matings with X. campestris and a panel of five non-target bacteria (X. perforans, Xanthomonas euvesicatoria, E. coli, Pseudomonas syringae, and Pseudomonas protegens) at a ratio of 10:1 (P. putida:recipient cell) on solid agar media for 48 hours at 30 degrees Celsius. Surviving recipient cells were enumerated on LB media to determine killing efficacy (+ind) and compared to recipient cells processed through the same protocol without P. putida (−ind, n=3). FIG. 9A shows the replicate plated cultures of treated and untreated target and non-target bacteria. Colony counts were compared between treated and untreated cultures to calculate the percent killing efficacy, as shown in FIG. 9B. Following co-transformation of CRISPR-gRNA and conjugative plasmids into P. putida, all strains showed an ability to conjugate into X. campestris with two gRNA showing average killing efficacy above 99.7% when the system was induced. Results showed no evidence of off-target killing in all strains tested except one closely related strain of Xanthomonas, Xanthomonas euvesicatoria (below 5% as shown in FIG. 9B).

Similarly, a donor P. putida strain was cotransformed with pTAmob and pR.118, containing gRNA designed to target Erwinia amylovora. Donor bacteria was applied to target bacteria and non-target strains X. perforans, E. coli, and P. putida. As shown in FIG. 9C, modified donor bacteria showed a killing efficacy of 100% again target strain E. amylovora and minimal to no killing against non-target bacteria.

### Example 21: TAmob Toxicity Against Xanthomonas spp.

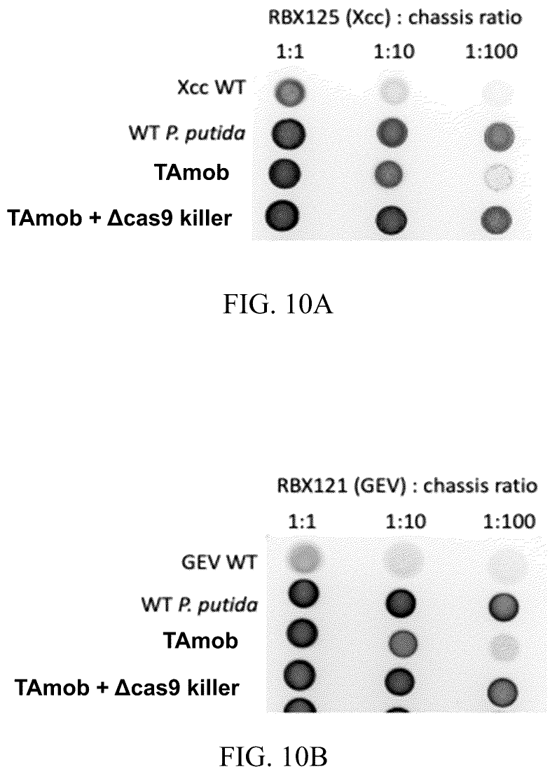

To further assess the mechanism of action, pTAmob was transformed into P. putida alone and with the Δcas9 CRISPR plasmid. Conjugation matings were performed as described above using X. campestris (Xcc) and X. perforans (GEV) as the recipients and wildtype P. putida (WT P. putida), P. putida alone, and (P. putida+Δcas9 CRISPR plasmid) as the donors. Results show no evidence of toxicity when wildtype P. putida or (P. putida+Δcas9 CRISPR plasmid) are used as the donors, however there is a significant decrease in fluorescence (measure of Xcc or GEV cell viability) when P. putida transformed with TAmob alone is used as the donor (FIG. 10A and FIG. 10B). The result suggests TAmob alone, without an origin of transfer (oriT, SEQ ID NO: 2) present in the modified donor, is non-specifically toxic to recipient bacterium in close proximity to the donor.

### Example 22: Needle Inoculation in Tomatoes

To assess initial mechanisms for plant colonization, a needle inoculation method based on previously published methods was implemented. Wounding through a needle puncture ensures access to the endosphere of the plant for the chassis organism (P. putida), ensuring P. putida will enter the endosphere and allow us to evaluate P. putida persistence within the plant. Initial testing was conducted on 3-week old MicroTom tomato plants.

An overnight culture of wild-type P. putida was grown in liquid LB medium. The optical density (OD₆₀₀) of the culture was adjusted to 1.0 and used as the inoculum for this assay. A 10 μL droplet of inoculum was added to a tomato stem. A 20 gauge sterile needle was used to puncture the stem forming a wound through which the droplet was absorbed into the tomato xylem.

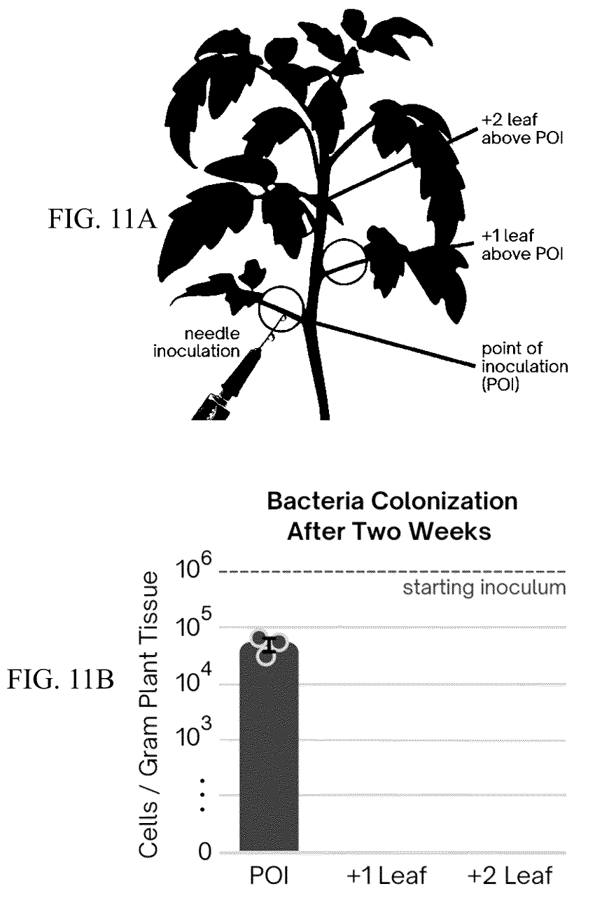

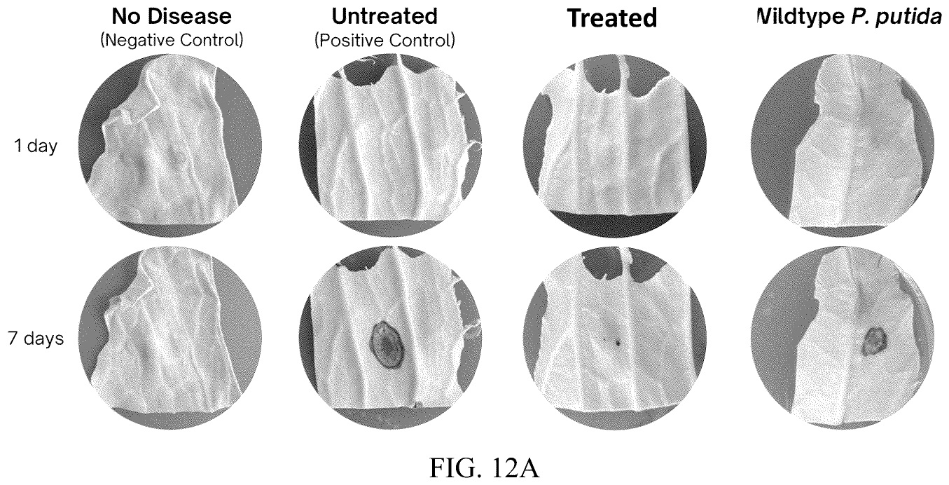

After 7 and 14 days, the plant segments at the point of inoculation and two subsequent nodes were harvested through destructive sampling (FIG. 11A). The samples were surface sterilized for 5 minutes in 70% ethanol and rinsed three times in sterile deionized water. The samples were homogenized in 2 mL of sterile 1×PBS in mesh bags (Agdia, Inc.) with a hammer. 200 μL of the homogenized tissue solution was plated onto LB media to enumerate P. putida persistence. Initial results suggested that cells persisted inside the tomato tissue at 10⁴-10⁵ CFU/g plant tissue (relatively high concentration) but did not move within the xylem beyond the point of inoculation (FIG. 11B).

### Example 23: Detached Leaf Assay in Cabbage

X. campestris (Xcc) infects members of the Brassica plant genus and is the causal agent of black rot of cabbage. In order to assess the efficacy of P. putida with the two-plasmid system inside plant tissue, a detached leaf assay was developed based on previously published work. Cabbage leaves from mature cabbage plants were cut into 1 inch by 1 inch slices and surface sterilized in 1% bleach for 5 minutes and rinsed three times in sterile deionized water. Detached leaves were maintained on sterile 1.5% water agar plates.

Overnight cultures of Xcc Tet^{R} (Xcc, recipient), wild-type P. putida (donor 1), and P. putida co-transformed with pTAmob and pR.036 (P.044, donor 2) were grown in liquid LB medium. Cell densities were adjusted to 1×10⁸ CFU/mL in fresh LB media and inocula were prepared by combining donor to recipient ratios of 1000:1 in sterile PBS. 40 μL of each inoculum was syringe infiltrated into the cabbage leaves (n=5 per treatment). In addition, detached leaves were also treated with sterile PBS (negative control) and Xcc (positive control), wild-type P. putida, or P.044 24 hours or 48 hours before inoculation with Xcc.

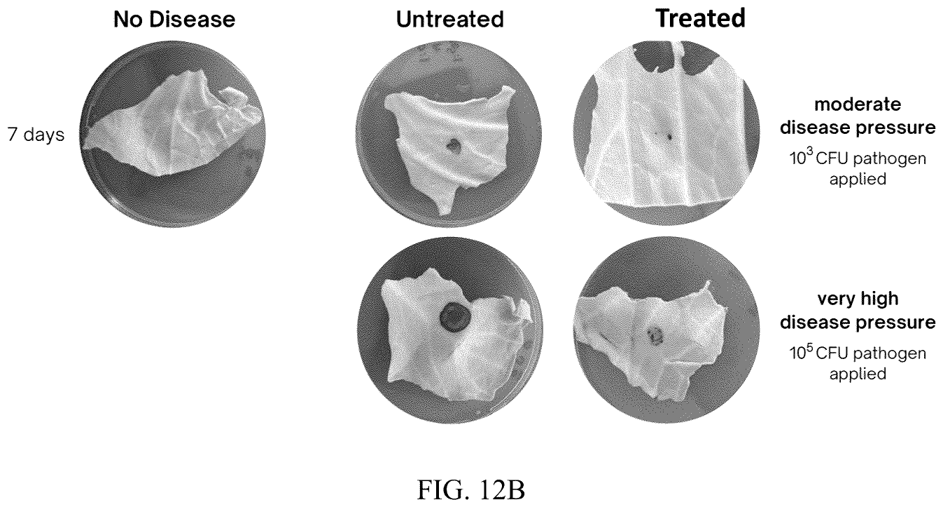

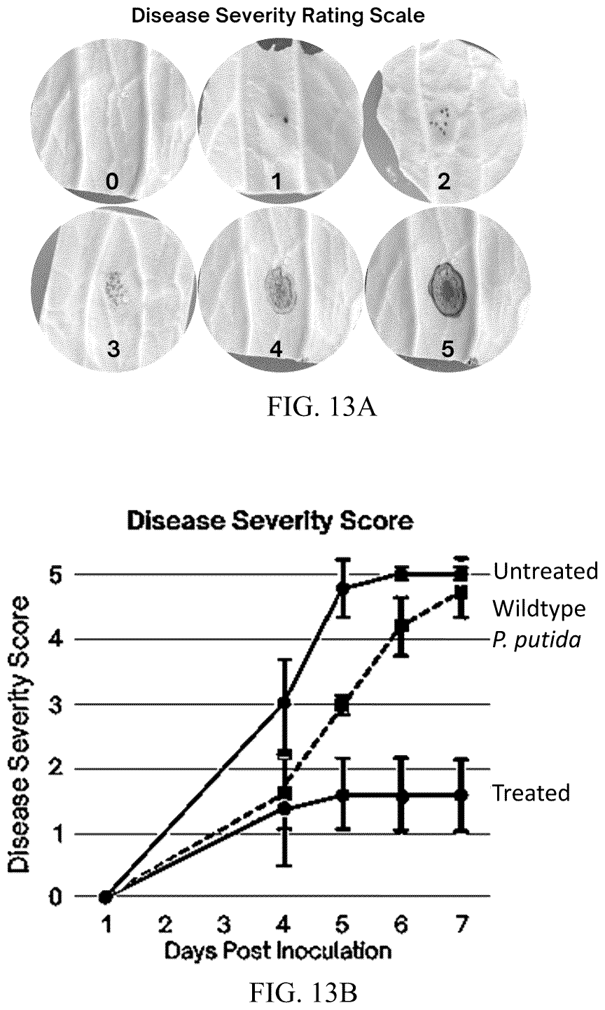

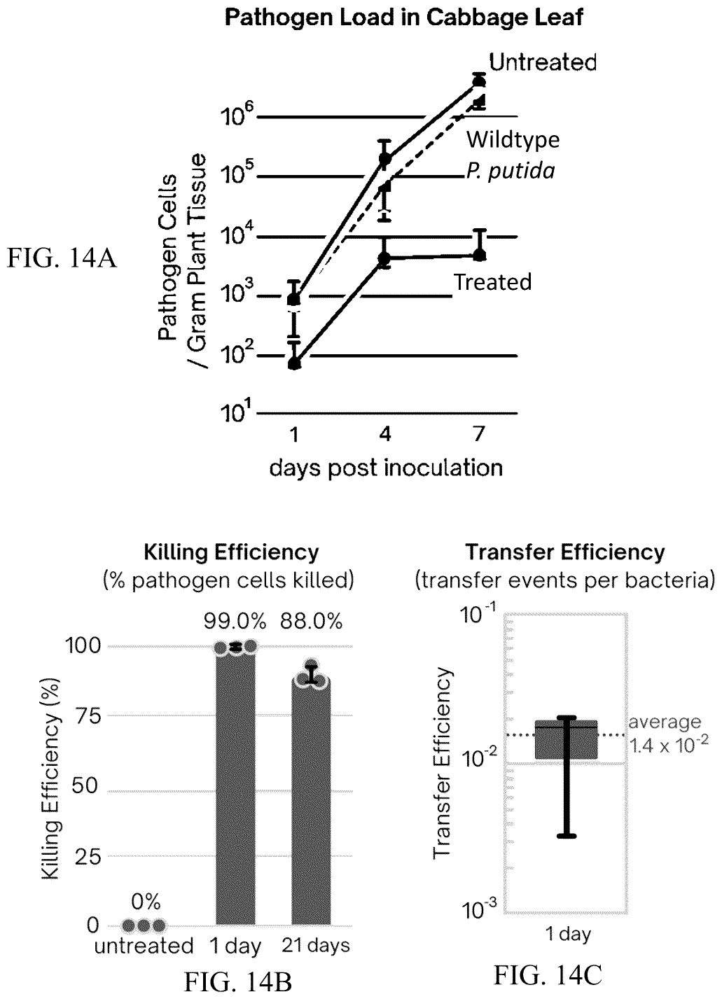

At 1, 4, and 7 days, cabbage leaf tissue around the sight of injection was excised, weighed, and macerated and X. campestris cells were enumerated on selective media to quantify the pathogen load in the cabbage leaves. FIG. 14A shows a suppression of growth of pathogenic bacteria on leaves treated with the P.044 strain. Detached leaves were monitored for 7 to 21 days for symptom development and sampled for pathogen titer. Disease severity was evaluated by rating the scale for black rot in cabbage leaves over time (FIG. 13A) On day 7, the treated detached cabbage leaves had smaller black rot (FIG. 12A) and therefore much lower disease severity score (FIG. 13B) as compared to the untreated detached cabbage. The samples were then homogenized in 5 mL of sterile 1×PBS in mesh bags (Agdia, Inc) with a hammer. 200 μL of the homogenized tissue solution was plated on nonselective and selective media to enumerate pathogen titer. Killing efficacy was quantified by normalizing the pathogen load under the engineered P. putida to the pathogen load without treatment at 7 and 21 days. Conjugation efficiency was quantified in the detached leaf environment using P. putida co-transformed with pTAmob and pR.029 as the donor and X. campestris as the recipient, and following the standard leaf inoculation protocol. The killing efficiency was 99.0% on day 1 and 88.0% after 21 days of treatment (FIG. 14B). Donor, recipient, and transconjugate cells were enumerated on selective media to determine conjugation efficiency (n=5). The average transfer efficiency was 1.4×10⁻² (FIG. 14C).

Detached leaves treated with P.044 exhibited fewer symptoms under both moderate disease pressure and very high disease pressure (FIG. 12B) and harbored fewer Xcc cells than both detached leaves infected with Xcc alone and leaves treated with wild-type P. putida (FIG. 14A). Fewer symptoms were also observed in detached leaves pre-treated with P.044 48 hours before Xcc inoculation compared with detached leaves pretreated with PBS or wild-type P. putida.

### Example 24: Detached Leaf Assay in Apple

E. amylovora infects members of the Rosaceae plant genus and is the causal agent of fire blight in apples and pears. Apple leaves from mature crab apple trees were surface sterilized in 1% bleach for 5 minutes and rinsed three times in sterile deionized water. Detached leaves were maintained on sterile 1.5% water agar plates.

Overnight cultures of E. amylovora Tet^{R} (Eam, recipient) were grown in liquid nutrient broth and wild-type P. putida (donor 1), P. putida co-transformed with pTAmob and pR.118 (P.063, donor 2) and pR.119 (P.064, donor 3), and Pantoea agglomerans were grown in liquid LB medium. Cell densities were adjusted to 1×10⁸ CFU/mL in fresh LB media and inocula were prepared by combining donor to recipient ratios of 1000:1 in sterile PBS. 40 μL of each inoculum was syringe infiltrated into the apple leaves (n=3 per treatment). In addition, detached leaves were also treated with sterile PBS (negative control), Eam (positive control), wild-type P. putida, and P. agglomerans (a known biocontrol for E. amylovora). Disease progression was visually monitored over 7 days. Fire Blight was controlled exceptionally in detached apple leaves treated with P.063.

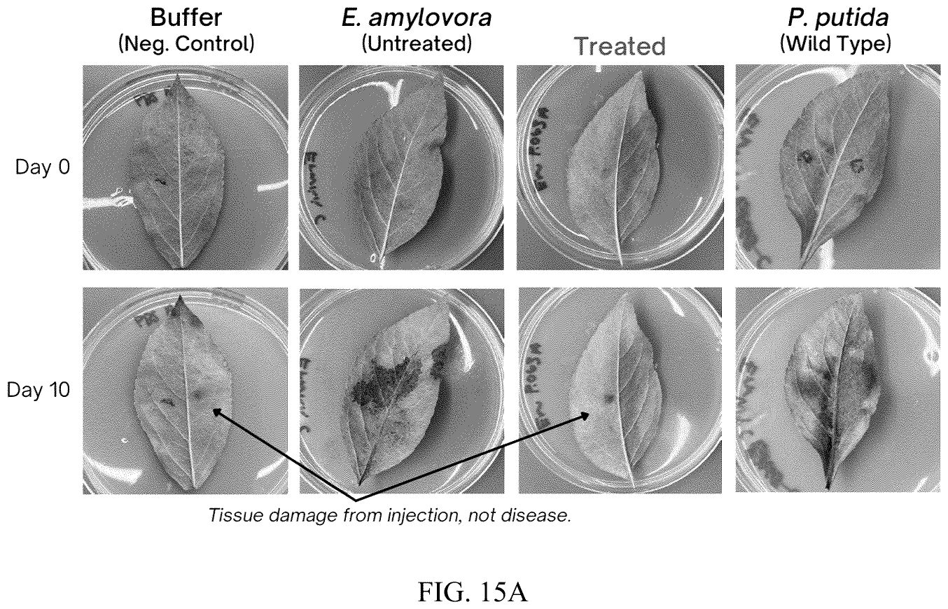

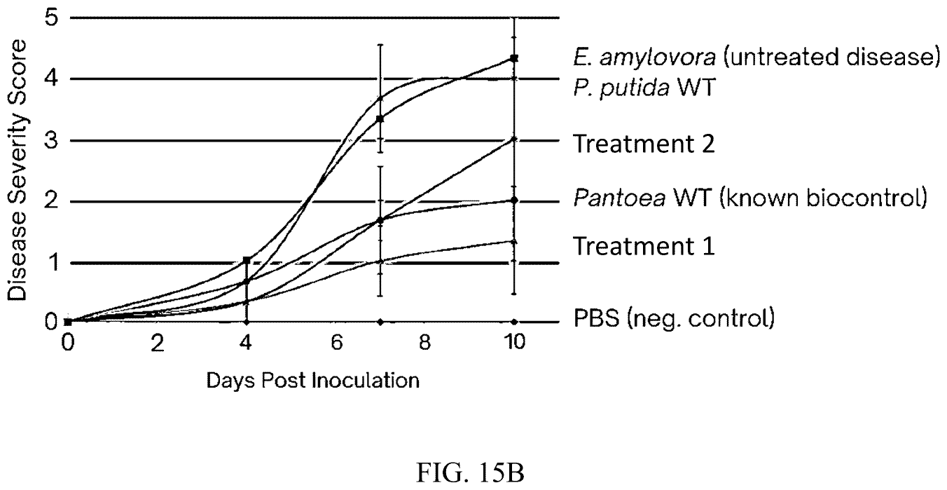

Detached leaves were monitored over 10 days for symptom development. Representative test leaves at Day 0 and Day 10 are shown in photos in FIG. 15A. As shown, leaves treated with a buffer (neg. control) showed no significant change. Leaves inoculated with E. amylovora alone (“Untreated”) or P. putida (“Wild Type”) exhibited significant blackening after 10 days. Leaves in the treatment group showed no significant disease. Disease severity was assessed visually using a previously established 0 to 5 rating scale. Disease ratings over 10 days is presented in the graph in FIG. 15B. Detached leaves treated with P.063 and P.064 exhibited fewer symptoms and harbored fewer Eam cells than both detached leaves infected with Eam alone and leaves treated with wild-type P. putida and P. agglomerans.

### Example 25: Comparison of Microbial Formulations

Six additives commonly used in microbial formulations were tested to evaluate their ability to improve bacterial colonization in tomato plants: 10 mM magnesium chloride (MgCl₂), 0.1% Tween 20 (non-ionic surfactant wetting agent), 0.1% Triton X-100 (non-ionic surfactant wetting agent), 0.02% Silwet L-77 (non-ionic surfactant wetting agent), 0.1% Span-85 (biological non-ionic surfactant wetting agent), and 0.1% molasses (humectant, stabilizer, and UV protectant). All additives have been previously identified and published as known components in microbial formulations. Cell viability was assessed by mixing overnight cultures of P. putida, P. phytofirmans (PsJN), and X. perforans (GEV) in each solution and incubating for 10 minutes, 24 hours, and 7 days at 4 degrees Celsius and 25 degrees Celsius (data not shown). MgCl₂, Tween, Triton, and Silwet showed no/low toxicity and were moved forward to in planta testing.

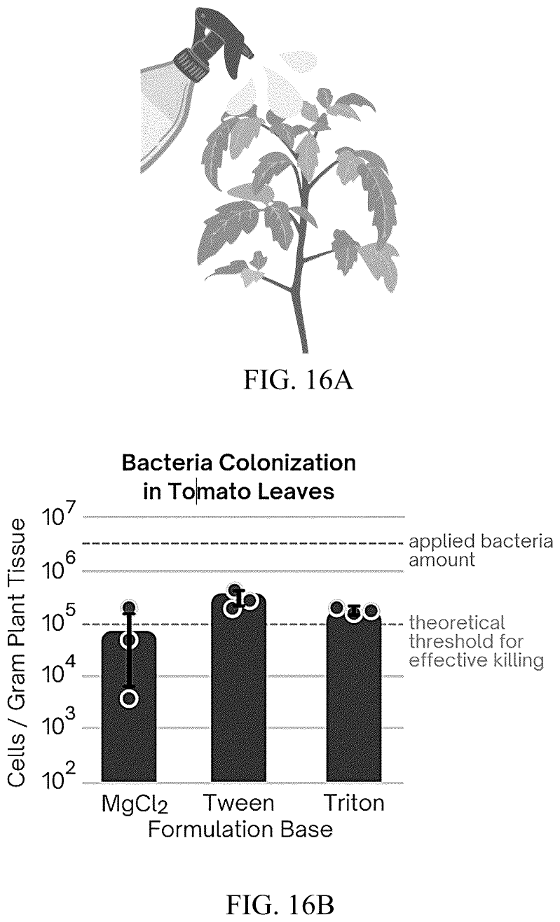

Additional testing was performed on 3-week old MicroTom tomato plants. Overnight cultures of wild-type P. putida, PsJN, and GEV were grown in liquid LB medium. Cell densities were adjusted to 1×10⁸ CFU/mL and prepared with each of the four formulations. Two sprays (equivalent to 250 μL) were applied to each leaf causing a slight amount of runoff (FIG. 16A).

After 24 hours and 7 days, individual leaves were harvested through destructive sampling (n=3 per condition). At 7 days, new growth leaves that had not been spray treated were also sampled. The samples were surface sterilized for 5 minutes in 70% ethanol and rinsed three times in sterile deionized water. The samples were homogenized in 2 mL of sterile 1×PBS in mesh bags (Agdia, Inc.) with a hammer. 200 μL of the homogenized tissue solution was plated onto LB media to enumerate bacterial colonization. Table 12 shows subjective assessment of effect of each additive on colonization.

TABLE 12
Effect of formulation additives

| Additives Tested | Outcome |
|---|---|
| 10 mM Magnesium Chloride | Variable |
| 0.1% Tween 20 | Effective |
| 0.1% Triton X-100 | Effective |
| 0.02% Silwet L77 | Variable |
| 0.1% Span 85 | Toxic |
| 0.1% Molasses | Toxic |

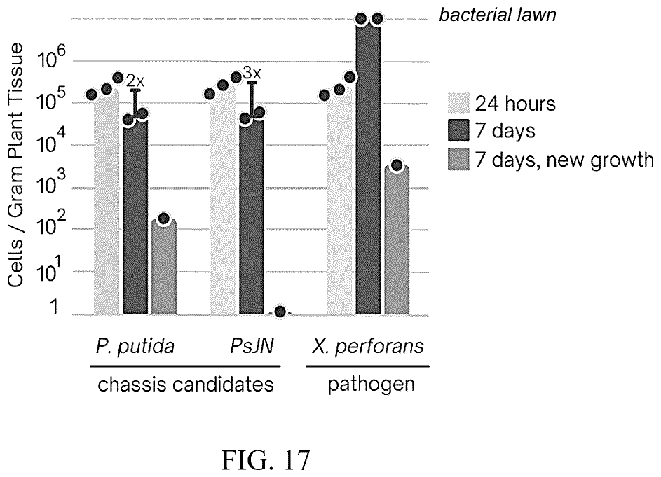

FIG. 16B is a bargraph indicating bacterial cells/gram plant tissue in plants treated with MgCl2, Tween 20, or Triton X-100. Initial results suggested that 0.1% Tween 20 provides the highest level of consistent colonization of all three bacteria. In addition, P. putida and PsJN persisted inside the tomato tissue at 10⁴-10⁵ CFU/g plant tissue (relatively high concentration) over 7 days while GEV increased colonized cell density over time (FIG. 17).

### Example 26: Challenge Assay in Solanaceae Plants (Tomato, Growth Chamber)

GE P. alloputida P.048 (pTAmob+pR.093) biocontrol of X. perforans (GEV, Bacterial Spot) in Tomato Early Cherry (Solanum lycopersicum) was evaluated. Seeds were sown in 2×2 inch pots under growth chamber conditions (16 hours of light at 25° C. and 8 hours of darkness at 22° C. and 50% humidity). Three weeks post emergence, the plants were spray inoculated with pathogen (when applicable), allowed to dry at ambient room conditions for 8 hours and then spray inoculated with the respective treatment and allowed to air dry overnight before being placed back into the growth chambers.

Overnight cultures of GEV, wild-type P. putida, and P. 048 were grown in liquid LB medium. Cell densities were adjusted to 1×108 CFU/mL in fresh LB media and inoculum was formulated to a final concentration of 0.1% Tween 20. Control plants were sprayed with PBS (negative control) and inoculated with GEV (positive control). For the challenge assay, plants were treated with a 1000:1 ratio of donor to recipient and arranged in a randomized block design. GEV was treated with the following: P.044, wild-type P. putida, and two industry standard, commercially available chemical pesticides Actigard® (Syngenta, a plant immune activator) and Kocide® (DuPont, a copper-based antimicrobial).

Plants were monitored over 14 days for symptom development and disease severity was assessed visually using a previously established 0 to 5 rating scale. After 14 days, bioassay measurements were collected (plant height, above ground biomass, and below ground biomass) and then the plants were destructively sampled to evaluate X. perforans and P.048 cell titers and plasmid maintenance. Plant samples were surface sterilized for 5 minutes in 70% ethanol and rinsed three times in sterile deionized water and then homogenized in 5 mL of sterile 1×PBS in mesh bags (Agdia, Inc.) with a hammer. 200 μL of the homogenized tissue solution was plated on nonselective and selective media to enumerate pathogen titer. Plasmid maintenance was evaluated by enumerating CFU on Gm+Kan (P.048 with both plasmids maintained), Gm (P.048 with pTAmob), Kan (P.048 with pR.093), and LB (all P.048 cells). GEV titer and surviving GEV transconjugates were enumerated on Tet and Tet+Kan, respectively. Plasmids from re-isolated cells were sequenced to evaluate any mutation that may have occurred in the plasmids while P. putida persisted in the plant over 14 days without antibiotic selection.

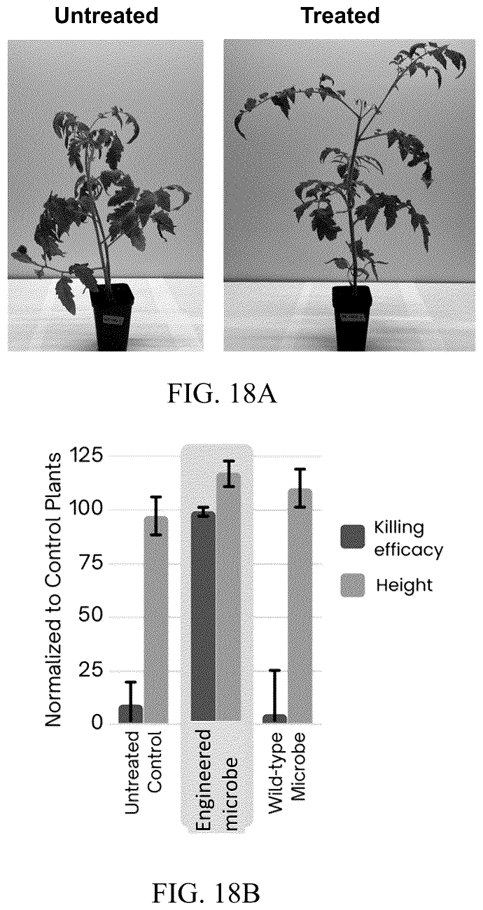

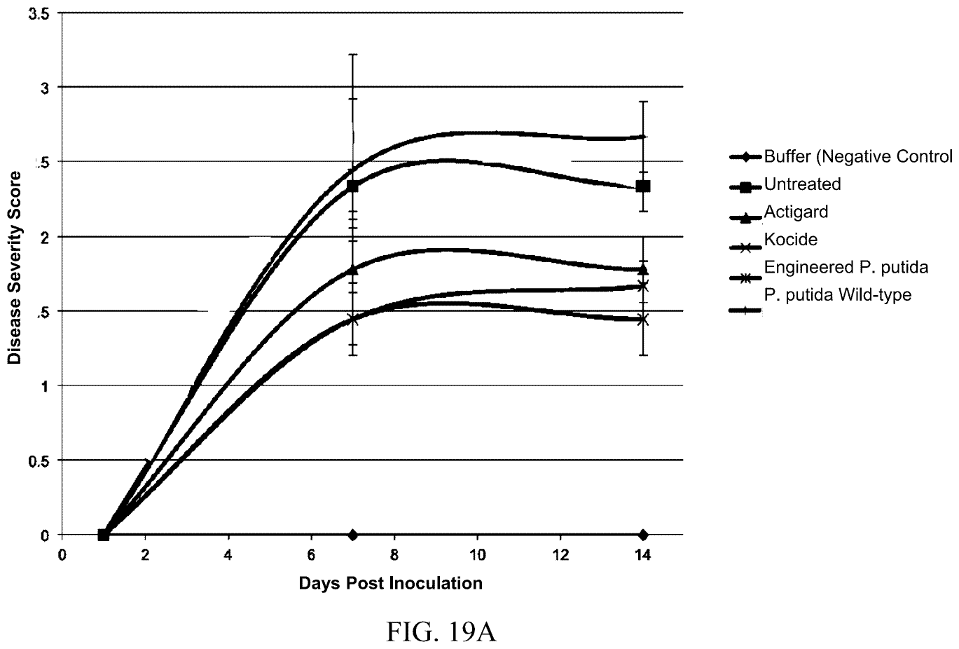

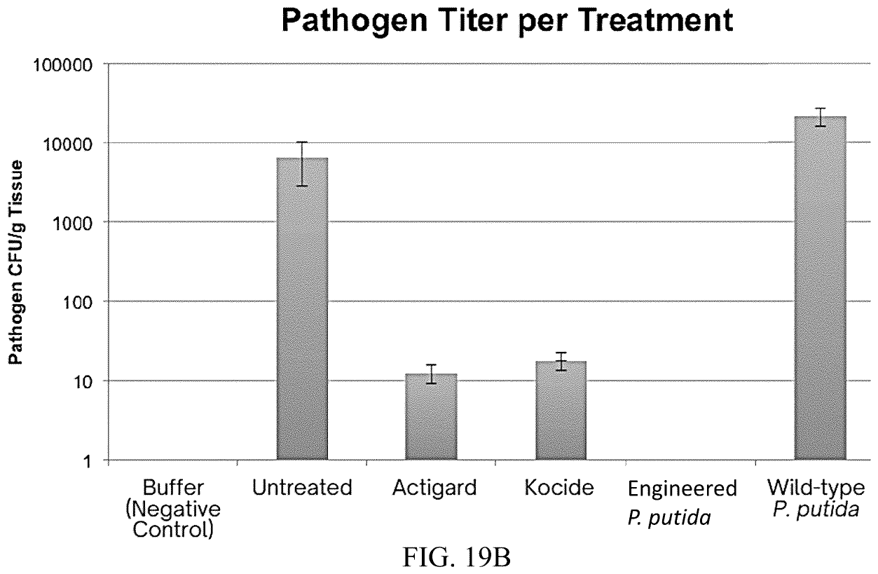

As shown in FIG. 18A, the size of the plant treated with engineered P. putida grew larger than the untreated. Plants treated with engineered P. putida exhibits better killing efficacy of pathogens (about 10 fold better) and increased plant height compared to the untreated plant (FIG. 18B). Moreover, plants treated with engineered P. putida showed the most reduction in disease symptom formation (FIG. 19A) and a reduction in pathogen titer compared to untreated plants (FIG. 19B). Thus, the results show GE P. putida P.048 has shown effective biocontrol of X. perforans in Solanaceae plants.

### Example 27: Challenge Assay in Solanaceae Plants (Tomato, Greenhouse)

The testing of the system was expanded to evaluate control of X. perforans (GEV, Bacterial Spot) in Jolene tomato plants (Solanum lycopersicum) in a greenhouse setting. Seeds were sown in 2×2 inch pots under greenhouse conditions. Two weeks post emergence (6 true leaves), the plants were treated by spray inoculation (10 plants per treatment).

Overnight cultures of GEV, P. putida co-transformed with pTAmob and pR.093 (“the modified P. putida”) were grown in liquid LB medium and LB with selective antibiotics. Cell densities were adjusted to 1×10⁷ and 1×10⁸ CFU/mL in fresh LB media and inoculum was formulated to a final concentration of 0.1% Tween 20. GEV was prepared in a similar fashion to 1×10⁶ CFU/mL. Buffer control plants were sprayed with PBS. “Untreated” plants were inoculated with GEV (positive control). For the challenge assay, plants were treated with two concentrations of the modified P. putida, 1×10⁷ and 1×10⁸ CFU/mL, and 0.05 g/L Actigard® and arranged in a randomized block design. Plants were challenged with GEV via spray inoculation two days later.

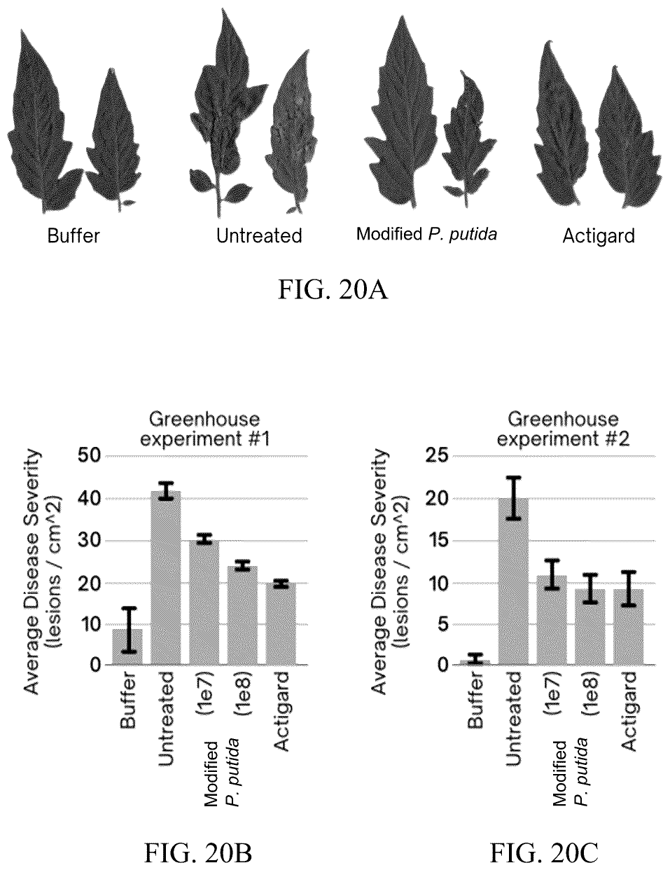

Plants were rated for disease severity at 13 days post inoculation. FIG. 20A shows images of the leaves from each treatment group. Untreated leaves showed significant spotting, yellowing, and texture. the modified P. putida-treated leaves appeared smooth with minimal to no spotting or yellowing. Actigard-treated leaves showed some spotting and yellowing. FIGS. 20B and 20C are bar graphs showing average disease severity as measured in lesions/cm\hat{ }2 for each treatment group in two greenhouse experiments. Tomatoes treated with the modified P. putida exhibited a reduction in necrotic lesions compared to untreated plants and performed similarly to Actigard®.

### Example 28: Persistence of Engineered P. putida in Tomato (Growth Chamber)

Donor bacteria were assessed for persistence and colonization levels. Engineered P. putida (as generated in Example 4) was compared to wild-type P. putida was assessed for colonization levels as well as the maintenance of the two-plasmid system. Tomato Early Cherry seeds were sown in 2×2 inch pots under growth chamber conditions of 16 hours of light at 25 degrees C. and 8 hours of darkness at 22 degrees C. and 50% humidity.

Overnight cultures of wild-type P. putida and P. putida co-transformed with pTAmob and pR.093 (P.048) were grown in liquid LB medium. Cell densities were adjusted to 1×10⁸ CFU/mL in fresh LB media and inoculum was formulated to a final concentration of 0.1% Tween 20. Three weeks post emergence, plants were spray inoculated with two concentrations of cells equivalent to 1000:1 and 100:1 but without any pathogen.

Plants were monitored over 28 days. Destructive leaf samples were taken every 24 hours for one week, and then at 14 and 28 days to evaluate cell titers and plasmid maintenance. Plant samples were surface sterilized for 5 minutes in 70% ethanol and rinsed three times in sterile deionized water and then homogenized in 5 mL of sterile 1×PBS in mesh bags (Agdia, Inc.) with a hammer. 200 μL of the homogenized tissue solution was plated on nonselective and selective media to enumerate cell titer. Plasmid maintenance was evaluated by enumerating CFU on Gm+Kan (P.048 with both plasmids maintained), Gm (P.048 with pTAmob), Kan (P.048 with pR.093), and LB (P.048 and wild-type P. putida, separately). Plasmids from re-isolated cells were sequenced to evaluate any mutation that may have occurred in the plasmids.

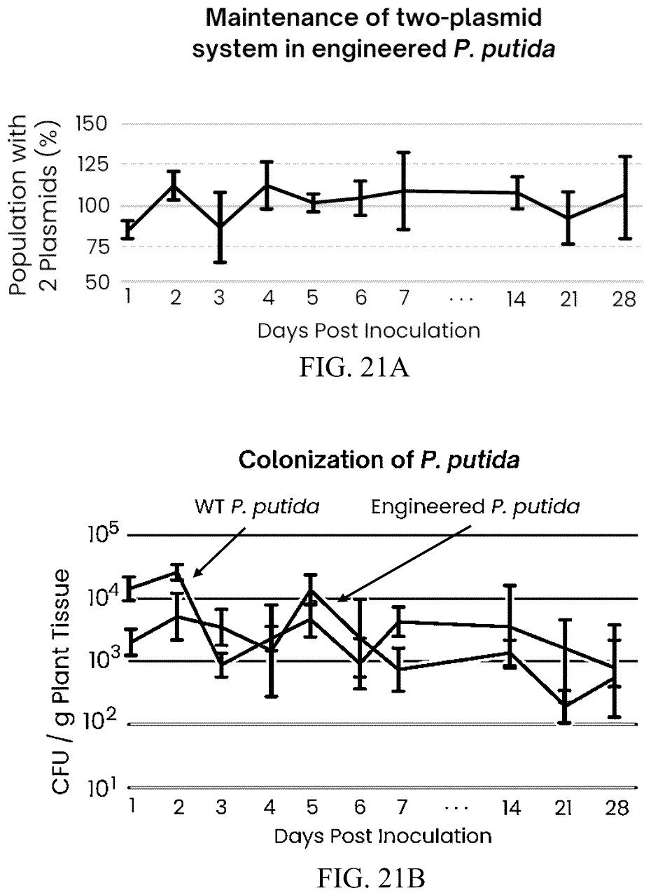

P.048 extracted from tomatoes show both plasmids being stable for at least 28 days (FIG. 21A). Tomatoes treated with P.048 or wild-type P. putida showed comparable levels of colonization over 28 days (FIG. 21B).

### Example 29: P. putida Colonization in Tomatoes and Cabbage in an Outdoor Environment

The persistence and colonization levels of wild-type P. putida were assessed in cabbage and tomato plants grown in an outdoor environment. Katarina F1 Cabbage (Brassica oleracea) seeds were sown in four rows of 7 plants with 4 inch spacing in container pots and grown outdoors in Somerville, MA. Six Big Beef tomato plants were purchased from Whole Foods and repotted in 12 inch terracotta containers and grown outdoors in Somerville, MA. Plants were watered every other day or as needed.

Overnight cultures of wild-type P. putida were grown in liquid LB medium. Cell densities were adjusted to 1×10⁸ CFU/mL in fresh LB media and inoculum was formulated to a final concentration of 0.1% Tween 20. Three weeks post cabbage emergence, plants were spray inoculated with 50 mL total volume.

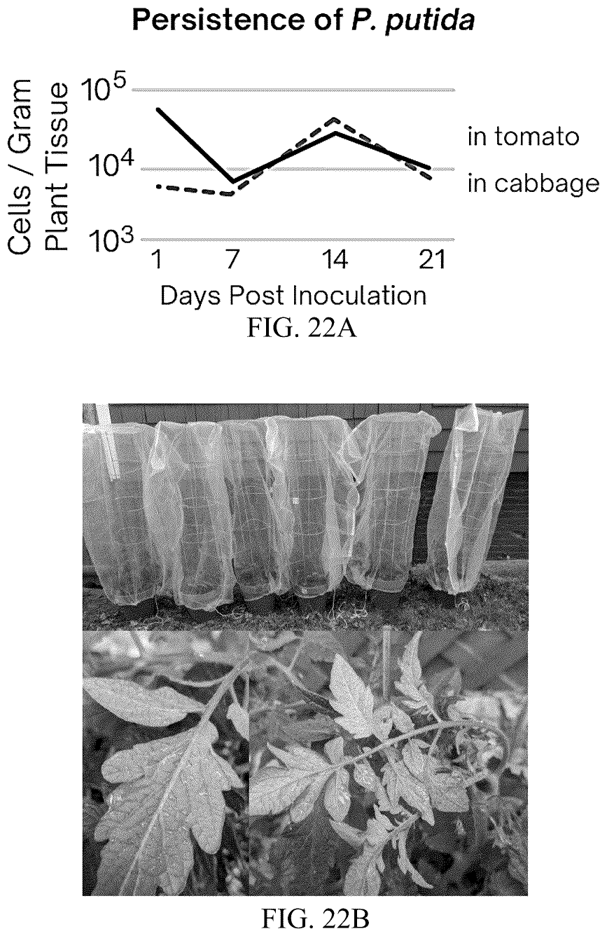

Plants were monitored over 21 days and destructive leaf samples were taken 24 hours post inoculation and then every 7 days. The experiment was terminated after all leaves initially treated were sampled. Plant samples were surface sterilized for 5 minutes in 70% ethanol and rinsed three times in sterile deionized water and then homogenized in 5 mL of sterile 1×PBS in mesh bags (Agdia, Inc.) with a hammer. 200 μL of the homogenized tissue solution was plated on nonselective media to enumerate the cell titer. Wild-type P. putida exhibited consistent colonization in both tomato and cabbage plants over the course of 21 days (FIGS. 22A-22B).

### Example 30: Persistence of Engineered P. putida in Tomato (Field Trial)

Donor bacteria were assessed for persistence and colonization levels. Engineered P. putida (as generated in Example 7) and assessed for colonization levels. The field trial was carried out in sandy soils in Central Florida, USA. Raised beds were prepared under a large insect netting-enclosed structure using standard commercial plasticulture practices, including fumigation and drip irrigation. Jolene tomato seeds were sown in 2×2 inch pots under greenhouse conditions. Jolene tomatoes were transplanted 3-weeks post germination.

Overnight cultures of P. putida co-transformed with pTAmob and pR.093 were grown in liquid LB medium with selective antibiotics and X. perforans in LB medium. Cell density was adjusted to 1×10⁸ CFU/mL (P. putida) and 1×10⁶ CFU/mL (X. perforans) in 1×PBS and inoculum was formulated to a final concentration of 0.1% Tween 20. Plants were first spray inoculated with P. putida and then challenged with X. perforans two days later. Each treatment included 10 plants per replicate and 4 replicates per treatment organized in a randomized block design.

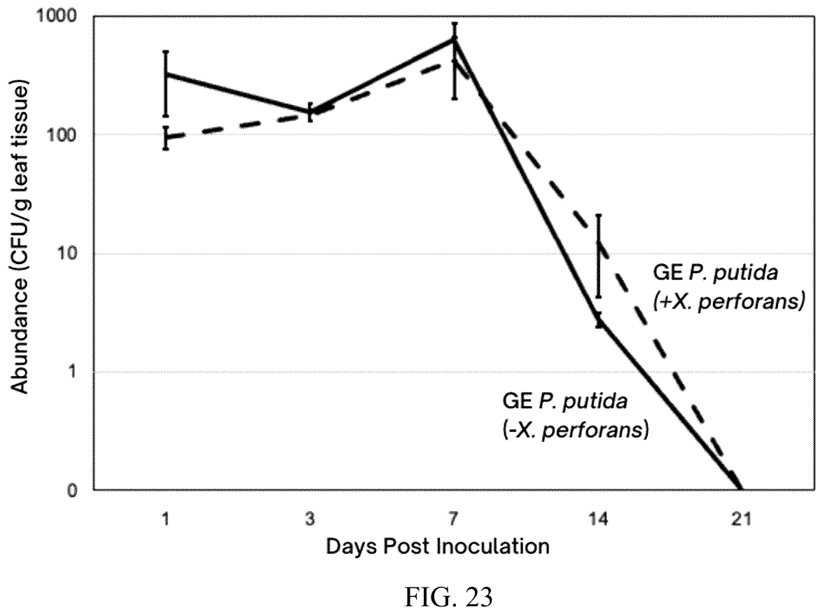

Plants were monitored over 21 days. Destructive leaf samples were taken 1, 3, 7, 14, and 21 days after P. putida application to evaluate cell titers. Plant samples were surface sterilized for 5 minutes in 70% ethanol and rinsed three times in sterile deionized water and then homogenized in 5 mL of sterile 1×PBS in mesh bags (Agdia, Inc.) with a hammer. 200 μL of the homogenized tissue solution was plated on nonselective and selective media to enumerate cell titer. Engineered P. putida showed persistence in tomato leaves for 14 days (FIG. 23).

### Example 31: Challenge Assay in Brassica Plants (Cabbage & Turnips, Growth Chamber)

To demonstrate that the system works in intact, whole plants, the engineered P. putida strain was tested for control of X. campestris (Black Rot) in Katarina F1 Cabbage (Brassica oleracea) and Turnip Just Right F1 (Brassica rapa).

Seeds were sown in 2×2 inch pots under growth chamber conditions of 16 hours of light at 25 degrees Celsius and 8 hours of darkness at 15 degrees Celsius. Three weeks post emergence, the plants were spray inoculated with pathogen (when applicable), allowed to dry at ambient room conditions for 8 hours and then spray inoculated with the respective treatment and allowed to air dry overnight before being placed back into the growth chambers.

Overnight cultures of X. campestris (Xcc), wild-type P. putida, and P. putida co-transformed with pTAmob and pR.036 (P.044) were grown in liquid LB medium. Cell densities were adjusted to 1×10⁸ CFU/mL in fresh LB media and inoculum was formulated to a final concentration of 0.1% Tween 20. Control plants were sprayed with PBS (negative control) and inoculated with Xcc (positive control). For the challenge assay, plants were treated with a 1000:1 ratio of donor to recipient and arranged in a randomized block design. Xcc was treated with the following: P.044, wild-type P. putida, and two industry standard, commercially available chemical pesticides Actigard® (Syngenta, a plant immune activator) and Kocide® (DuPont, a copper-based antimicrobial).

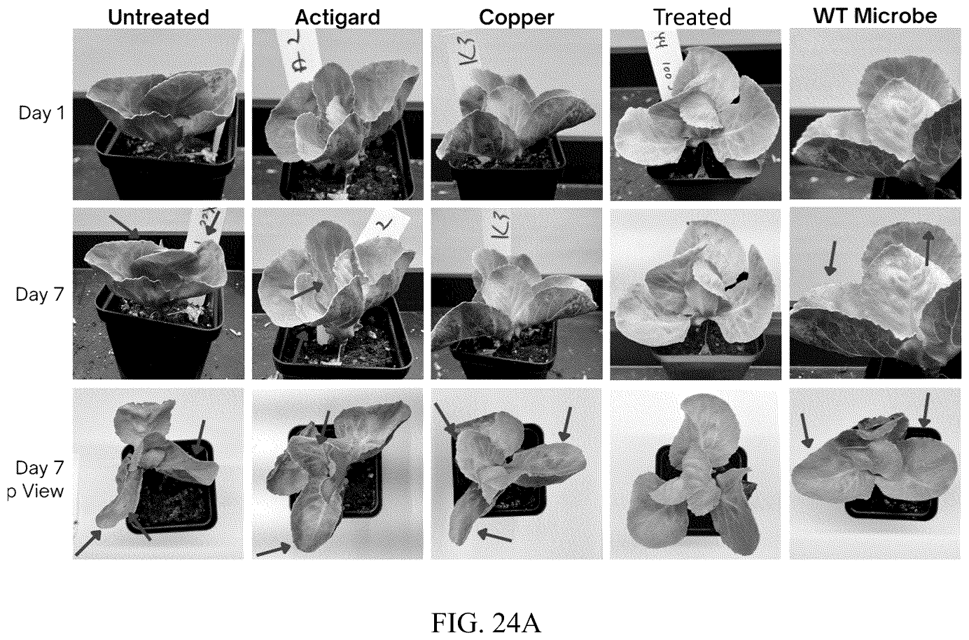

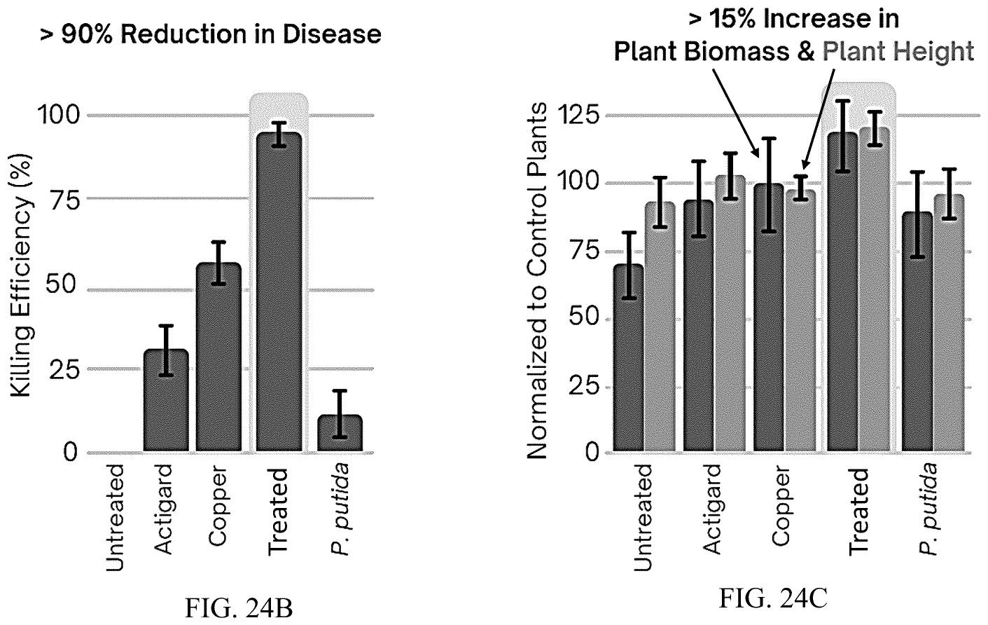

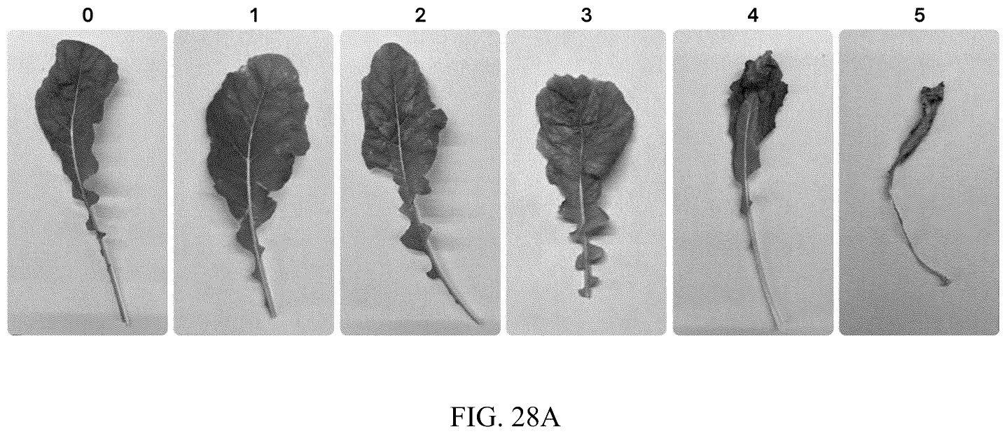

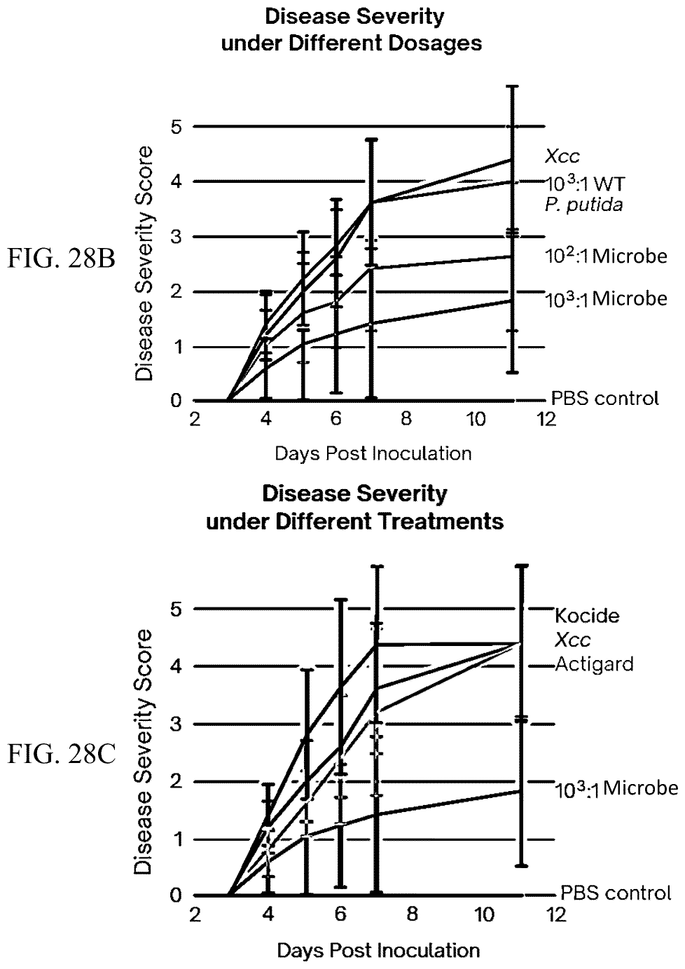

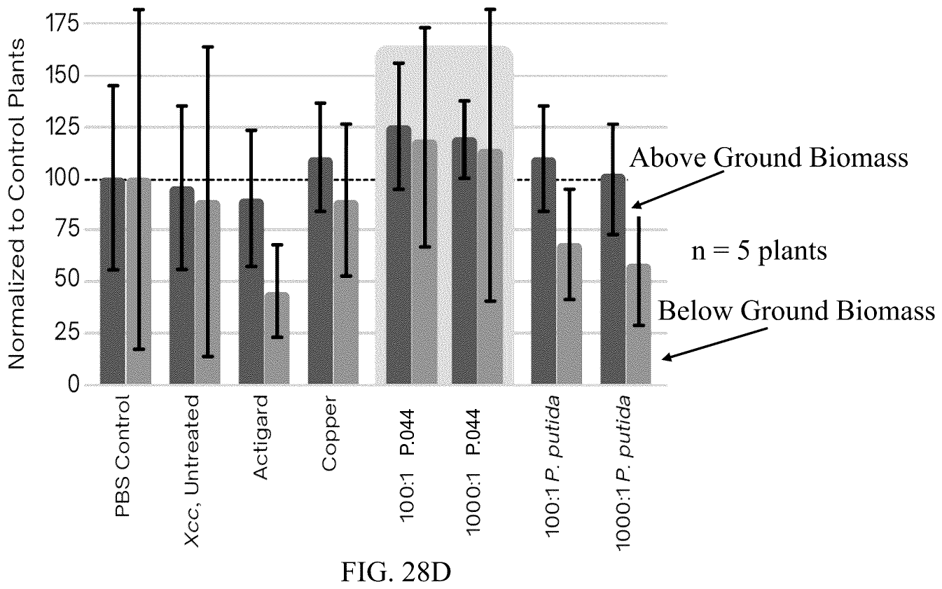

Plants were monitored over 14 days. FIG. 24A shows representative plants from each treatment group at Day 1 and Day 7 following treatment. Arrows indicate early formation of necrotic lesions indicative of black rot disease. Symptom development and disease severity were assessed visually using a previously established 0 to 5 rating scale (FIG. 28A). After 14 days, bioassay measurements were collected (plant height, above ground biomass, and below ground biomass) and then the plants were destructively sampled to evaluate Xcc and P.044 cell titers and plasmid maintenance. Plant samples were surface sterilized for 5 minutes in 70% ethanol and rinsed three times in sterile deionized water and then homogenized in 5 mL of sterile 1×PBS in mesh bags (Agdia, Inc.) with a hammer. 200 μL of the homogenized tissue solution was plated on nonselective and selective media to enumerate pathogen titer. Plasmid maintenance was evaluated by enumerating CFU on Gm+Kan (P.044 with both plasmids maintained), Gm (P.044 with pTAmob), Kan (P.044 with pR.036), and LB (all P.044 cells). Xcc titer and surviving Xcc transconjugates were enumerated on Tet and Tet+Kan, respectively. Plasmids from re-isolated cells were sequenced to evaluate any mutation that may have occurred in the plasmids while P. putida persisted in the plant over 14 days without antibiotic selection. Killing efficiency was quantified by normalizing the pathogen load under each treatment condition to the pathogen load without treatment at 14 days. 14 days post inoculation and spray treatment, cabbage plant height and the above ground biomass was measured and normalized to the control plants treated with phosphate buffered saline (PBS Control). Leaf samples were weighed and macerated and cells were enumerated on LB media (n=5).

Cabbage treated with P.044 exhibited over 90% reduction in pathogen load compared to untreated plants (FIG. 24B) and increased plant biomass and height by over 15% (FIG. 24C). Wild-type P. putida showed minimal disease control, while Actigard® and Kocide® exhibited approximately 30% and 55% reduction in disease, respectively.

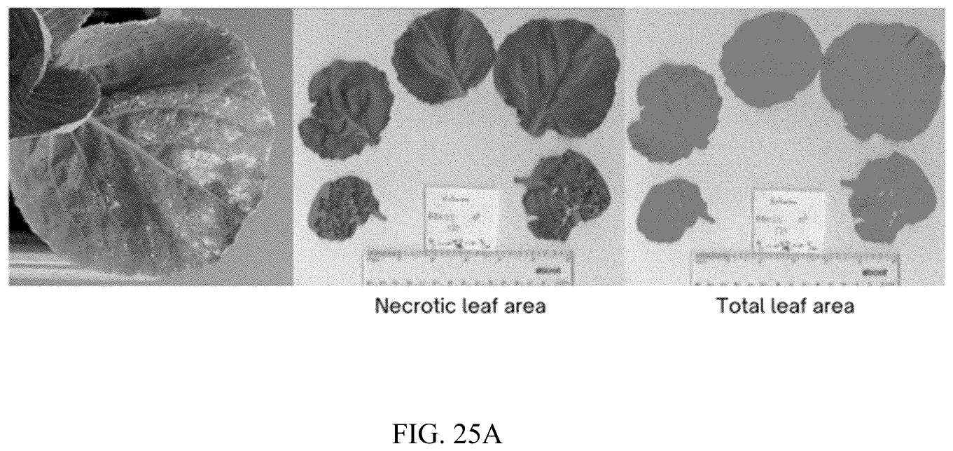

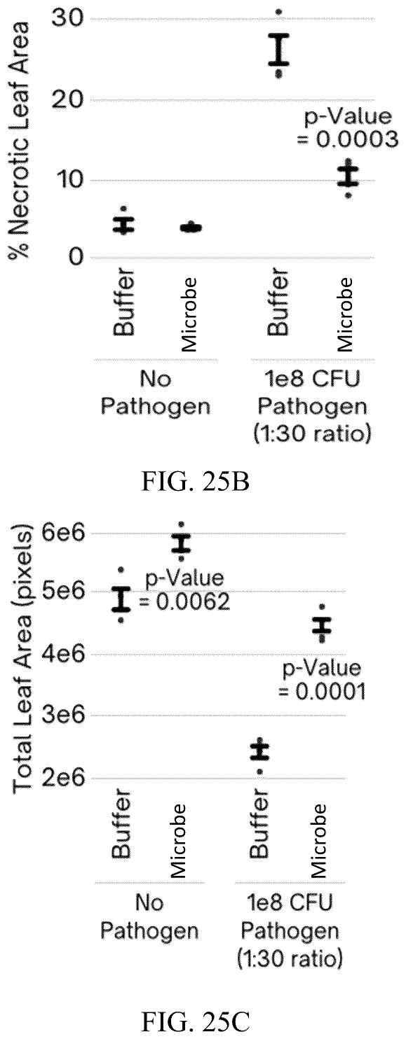

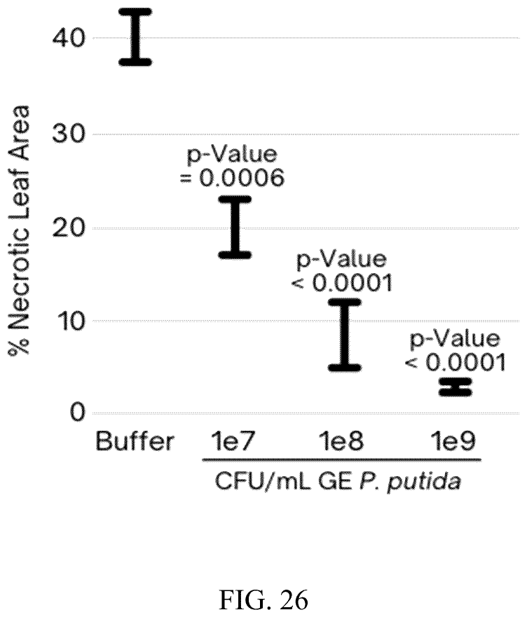

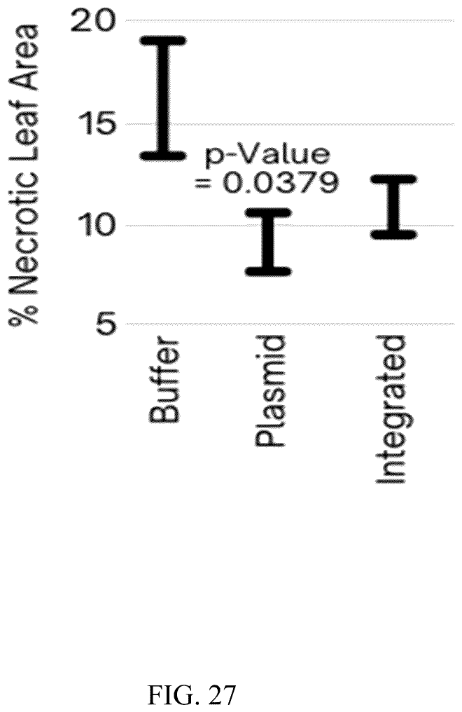

An image analysis process was applied to quantify disease symptoms on leaves removed from cabbage plants. 9 days after inoculation, leaves were removed from plants and scanned on a desktop scanner. ImageJ was used to quantify pixels associated with disease and total leaf area. The percentage of necrotic leaf area is calculated as the area of disease divided by the total leaf area (FIG. 25A-25C). When the concentration of donor bacteria is titrated from 1×107 CFU/mL to 1×109 CFU/mL and challenged with 1×108 CFU/mL X. campestris, a reduction in disease symptoms is observed (FIG. 26). When the two-plasmid system is compared to the integrated TAmob single plasmid system (pR.093), both engineered P. putida strains show biocontrol activity of X. campestris with the two-plasmid version showing stronger activity than the integrated version (FIG. 27).

In order to demonstrate the treatment effect in turnips, different donor to recipient ratios (1000:1 and 100:1) were also evaluated. Turnip plants were spray inoculated with X. campestris (Xcc) and spray treated 4 hours later with two concentrations of P. putida co-transformed with pTAmob and pR.036 (P.044) (10²:1 modified P. putida and 10³:1 modified P. putida), wild-type P. putida (10³:1 WT P. putida), Kocide®, or Actigard®. Disease progression was visually monitored over 11 days (n=5). Stronger disease control was observed at the higher ratio, but still statistically significant better control at 100:1 ratio for P.044 than Actigard® or Kocide® (FIGS. 28B and 28C). 14 days post inoculation and spray treatment, turnip plants were harvested and above ground and below ground masses were measured and normalized to the control plants treated with phosphate buffered saline (PBS Control). Conditions included: X. campestris (Xcc, Untreated), and X. campestris treated with two concentrations of P. putida co-transformed with pTAmob and pR.036 (100:1 Microbe and 1000:1 Microbe), two concentrations of wild-type P. putida (100:1 P. putida and 1000:1 WT P. putida), Kocide® (Copper), or Actigard® (n=5). Similar to the cabbage experiment, there was an observed increase in biomass in plants treated with P.044 compared to any of the other conditions (FIG. 28D).

### Example 32: GE P. putida P.105 Biocontrol of E. amylovora in Apple Trees

Treatment with genetically engineered GE P. putida P.105 (pTAmob+pR.119) and GE Pantoea agglomerans (pTAmob+pR.119) biocontrol of Erwinia amylovora (Fire Blight) in Malus domestica ‘Red Delicious’ was evaluated. Five 7- to 9-foot tall apple trees in 18-inch pots were purchased from a local nursery and placed indoors under ambient temperature and natural light. Each tree contained 40-165 flowers. Trees were inoculated by foliar spray at 40-70% bloom and 20 open blossoms were treated per treatment. Flowers were inoculated with bacterial suspension of 1×10\hat{ }8 CFU/mL until runoff A single application of bacterial treatment was inoculated at time=0. 24 hours later, E. amylovora was applied at a concentration of 1×10≡CFU/mL. Trees were monitored over 14 days for symptom development and disease severity was assessed visually using a previously established 0 to 4 rating scale. Five blossoms were destructively sampled 1, 3, 7, and 14 days post inoculation to evaluate E. amylovora, P. putida, and P. agglomerans cell titers. Plant samples were homogenized in 5 mL of sterile 1×PBS in mesh bags (Agdia, Inc.) with a hammer. 200 μL of the homogenized tissue solution was plated on selective media to enumerate cell titer.

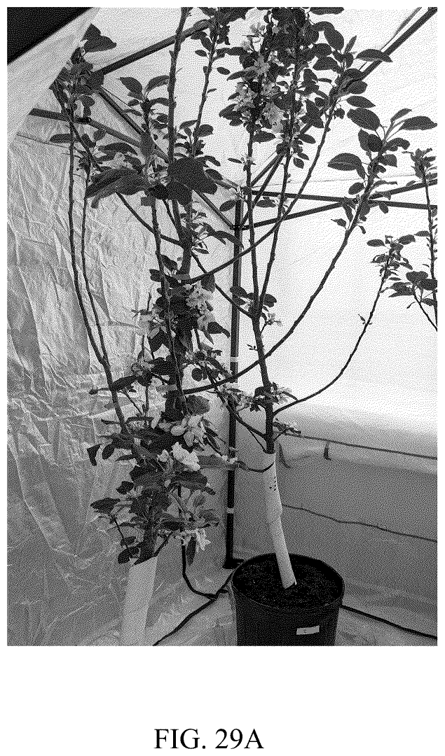

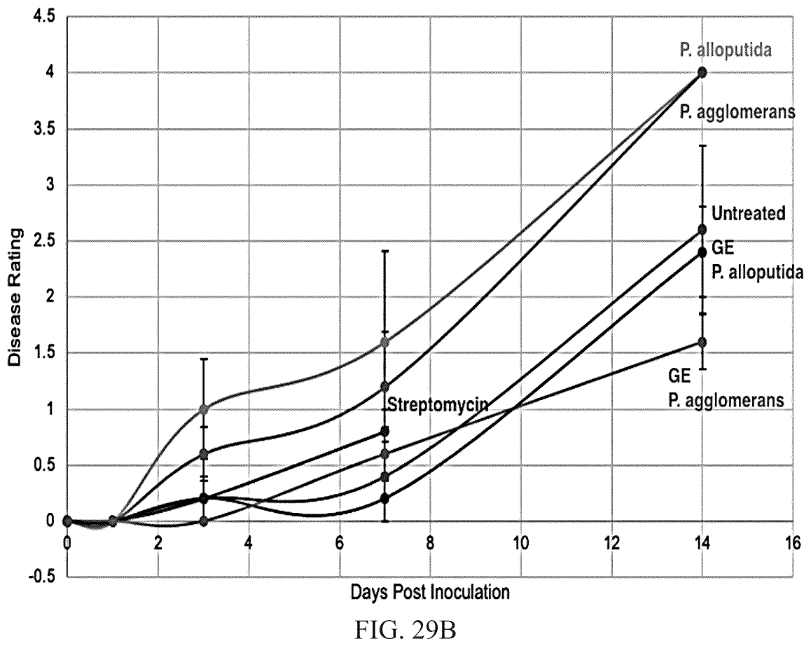

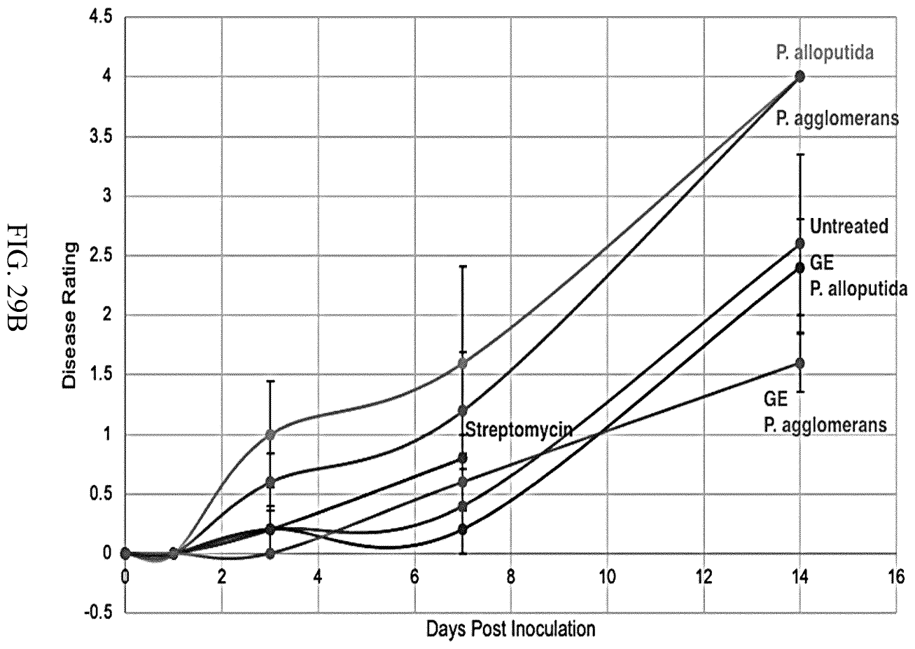

As shown in FIG. 29A, GE P. putida and GE Pantoea agglomerans treated apple trees looked healthy and did not show symptoms of fire blight such as brown to black leaves and dying blossoms. Additionally, as shown in FIG. 29B, disarmed P. putida and P. agglomerans showed the highest incidence of disease (potentially exacerbated by phytotoxicity response), reaching the maximum disease rating of 4 by 14 days post inoculation. The disease rating for untreated apple trees, GE P. alloputida treated apple trees, and GE P. agglomerans treated apple tree is 2.6, 2.4, and 1.6, respectively. Results show treatment with GE P. putida or GE P. agglomerans improved disease control compared to the untreated E. amylovora.

### Example 33: Directed Evolution to Improve Bacteria Motility

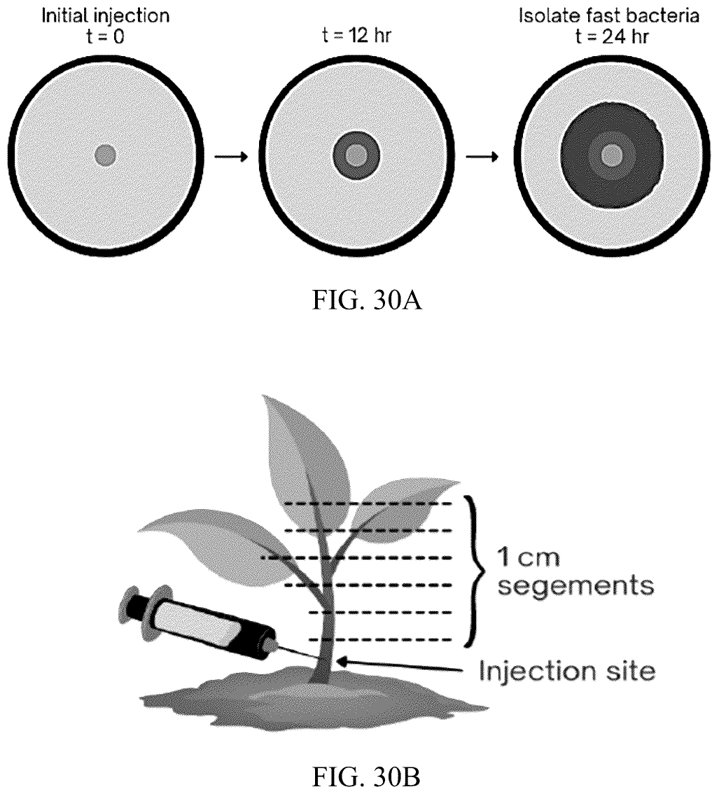

To improve bacteria motility, 3-5 rounds of directed evolution is performed on strains that show natural motility using non-targeted mutagenesis. The MP6 mutagenesis system is used to introduce chromosomal mutations into each strain, and soft agar motility experiments are performed to isolate faster moving bacteria. As illustrated in FIG. 30A, Bacteria in the outermost ring of the assay (dashed line) suggests they move faster than others (higher swimming speeds). Mutagenesis on the faster bacteria is continued to yield further motility improvements. The genome of the isolated population is sequenced after each iteration to identify common point mutations. The minimum goal is to obtain bacteria with swimming speeds greater than 100 μm/s, with optimal rates above 150 μm/s (approximately 1 cm/min, which would allow the bacteria to travel tree-sized distances (meters) on the order of hours). Growth rates of the evolved bacteria are measured to confirm mutagenesis did not negative impact essential cellular functions.

To validate that soft agar assay motility translates to motility in plants, the motility in juvenile plants was measured. The top three evolved bacteria (50 μL of 1×10\hat{ }8 CFU/mL) were applied into the xylem by needle inoculation at the base of the trunks of five 6-month-old potted plants grown in growth chambers, with controlled temperature (28-35 degrees Celsius) and a relative humidity of 80%. Samples are taken at the site of injection, as well as 1 cm increments along the stem 1 h after inoculation (FIG. 30B). The strain that exhibits strong motility, while being easily culturable and engineerable is selected.

### Example 34: Identification of Mobile Chassis Strains for Citrus Greening Product

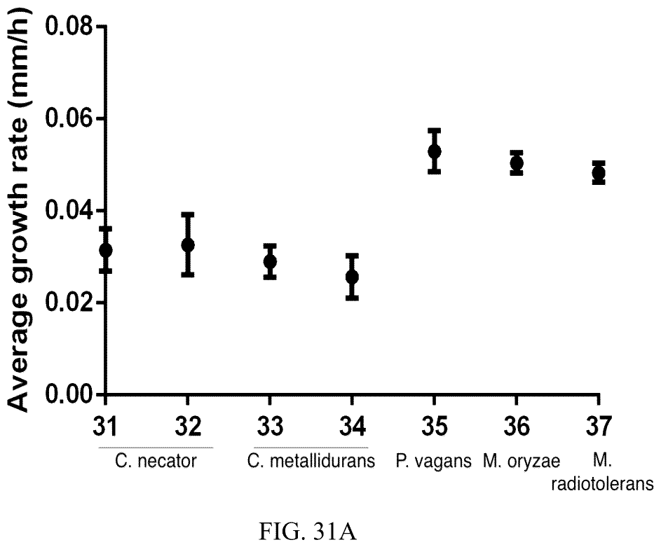

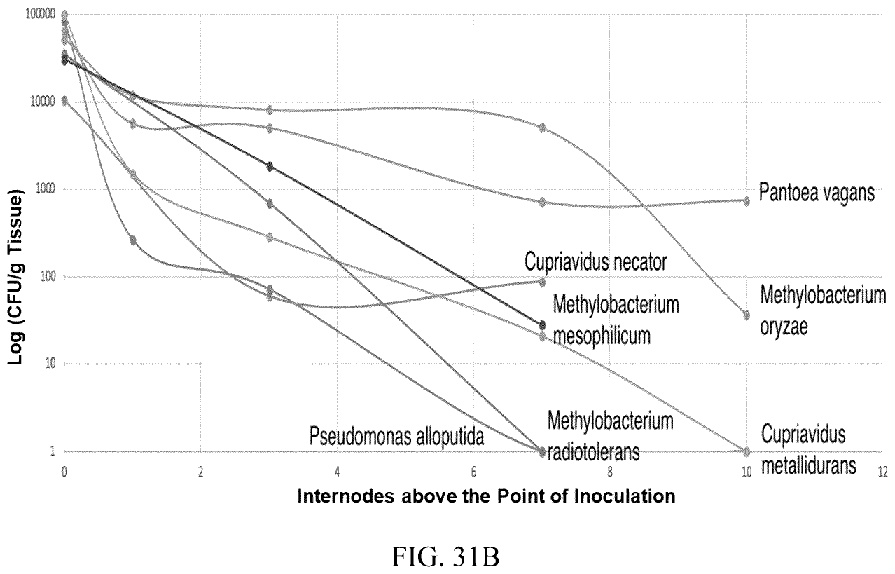

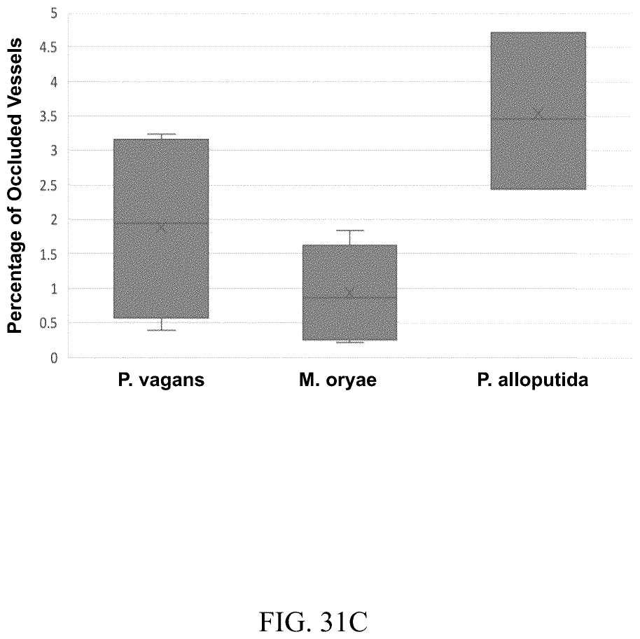

Eight bacterial strains were screened for motility and persistence in citrus (Citrus sinensis, sweet Valencia orange) and two strains, Pantoea vagans and Methylobacterium oryzae were identified as showing a strong ability to move within the citrus vessels and persist at moderate levels after 7 days. First, a soft agar motility assay was performed in vitro using a published protocol. Bacterial cultures were prepared in liquid media and 10 μL of culture was pipetted into the center of a soft agar plate. Bacterial plugs were incubated and the size of the colony was measured daily for 7 days. Average growth (motility) rate was reported in mm per hour (FIG. 31A). Pantoea vagans, Methylobacterium oryzae, and Methylobacterium radiotolerans showed the fastest motility of the strains evaluated.

Strains were needle inoculated into juvenile Sweet Valencia orange trees (1-2 ft tall) grown at ambient room temperature (25° C.) with 16 h light:9 h dark. 20 μL of overnight culture was needle inoculated at the base of each three (3 trees/strain). After 7 days, destructive samples were taken and trees were segmented into 0, 1, 3, 7, and 10 internodes above the point of inoculation. Samples were homogenized and bacterial cells were enumerated on LB agar plates. Colonies were visually characterized as well as verified by 16s sequencing. The movement of bacterial strains in citrus vasculature after needle injection at the base of the trunk is illustrated in FIG. 31B. P. vagans and M. oryzae moved 10 internodes above the point of inoculation, demonstrating strong promise as motile chassis strains.

Sections of citrus stems were sampled to evaluate the vasculature structure for tylose (occlusions that can block the vasculature and can be triggered by microbial infections). Sections were imaged by optical microscope. The percentage of occluded vessels was calculated from microscope images. FIG. 31C shows the percentage range of tyloses in sections treated with P. vagans, M. oryzae, and P. putida. Citrus trees inoculated with M. oryzae had significantly fewer tyloses (p<0.05) compared to P. vagans and P. putida. As shown, sections treated with P. vagans developed about 0.6% to about 3.2% tyloses. M. oryzae developed about 0.3% to about 1.6% occluded vessels. P. alloputida developed about 2.4% to about 4.7% occluded vessels.

### Example 37 Treatment of HLB in Citrus

A robust database of citrus-specific microbial genomes is developed to design gRNAs that effectively target the HLB pathogens while not negatively impact the beneficial citrus microbiome. The full genomic sequence of four target Liberibacter strains, Liberibacter americanus, Liberibacter africanus, Liberibacter asiaticus and Liberibacter crescens, is taken to generate all possible gRNAs within those genomes, cross-reference across the four gRNA sets to identify gRNA that are shared across the four strains, and then cross-reference those gRNA against the full database, allowing for up to two nucleotide mismatches in the gRNA sequence (SEQ ID NOs: 50-52, 98-115, 119-133). The output is a ranked list of gRNA that are scored based on binding affinity to the target genomes and weighted against sequences that have low target specificity (i.e. hit many non-target organisms). The top two gRNAs that hit the four Liberibacter strains but have no-to-low non-target hits are selected. If gRNA generated that hit all four strains also produce high non-target scores (i.e. also hit >20 non-target organisms), gRNA specific to each individual HLB Liberibacter strain and L. crescens is alternatively identified.

Two Cas9 plasmids each containing the single constitutively expressed gRNA sequences generated above is built so that Cas9 expression will be regulated by a repressor-activator system that suppresses Cas9 production when the plasmid resides in P. putida (FIG. 2B). Antibiotic resistance markers are engineered simultaneously onto the genome of L. crescens to enable selection of the pathogens in subsequent experiment. Tn7 transposon is used to integrate a constitutively expressed tetracycline resistance gene and the mCherry gene from an R6K suicide plasmid into each pathogen genome. Successful clones are selected for on tetracycline agar and validated by flow cytometry and Sanger sequencing.

The engineered P. putida strain is validated in vitro for selective and robust killing of the target pathogen. Prior to performing conjugation experiments, culturing of P. putida is tested on BM7 media to confirm the growth. To perform conjugations, overnight liquid cultures of P. putida carrying the conjugation-Cas9-gRNA plasmid and L. crescens are prepared. For slow growing of L. crescens, cell biomass is grown on solid BM7 agar plates and isolated and resuspended into phosphate buffered saline (PBS) ahead of the conjugation experiment. Both cultures are washed in PBS and resuspended to quantify a baseline for how many cells were present at the start of the experiment. Each of the P. putida strains is mixed with L. crescens at ratios of 100:1, 20:1, 10:1, and 1:1, then 10 μL of cell mixture is spotted onto solid agar plates and incubated for 24 or 48 hours at 28 degree Celsius. After 24 or 48, the cells and plate serial dilutions are isolated on antibiotic agar to select for total L. crescens and L. crescens that have received the conjugative plasmid (transconjugates).

Conjugation efficiency is calculated by dividing the number of transconjugates by the number of recipients at the start of the experiment. To measure conjugation efficiency, the template version of the Cas9 Plasmid containing a random gRNA sequence is used, allowing the measurement of the transfer rate of the plasmid from P. putida into L. crescens without results being biased by Cas9 killing the recipient cells.

Killing efficacy is measured through the same experimental process but using the plasmids containing the designated gRNAs. Killing efficiency is quantified by dividing the number of recipient cells that survived the mating by the number of recipient cells calculated at the start of the assay. As a control, P. putida containing the template Cas9 plasmid (empty gRNA site) will be used to measure transfer efficiency.

The two P. putida strains are also screened for non-target effects using a panel of eight bacteria that are representative of the eight bacterial families. Ultimately, the best performing plasmid that contains the gRNA with the highest killing efficacy and no detectable non-target killing is selected.

### Example 36: Screening Conjugative Plasmids to Select for Increased Conjugation Efficiency

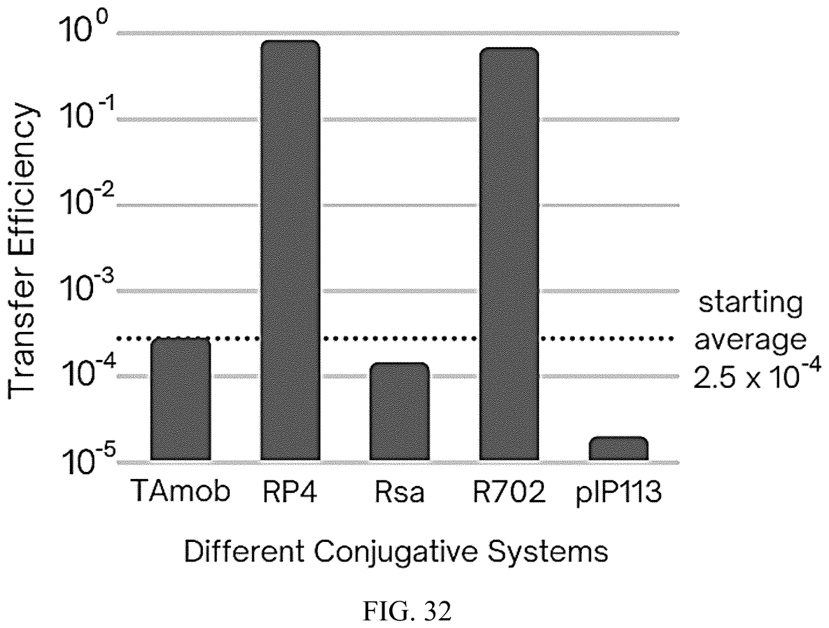

To select conjugative plasmids with higher conjugation efficiency, 21 plasmids were obtained from the Pasteur Institute, sequenced, and screened for conjugation efficiency using E. coli transformed with each plasmid as the donor strain and P. putida Gm^{R} (P.058) as the recipient and selected for transconjugates on Km+Gm selective media. RP4 (pR.120) and R702 (pR.122) showed over 1000× improvement in transfer efficiency compared to pTAmob (FIG. 32).

### Example 37: Directed Evolution to Improve Transfer Efficiency

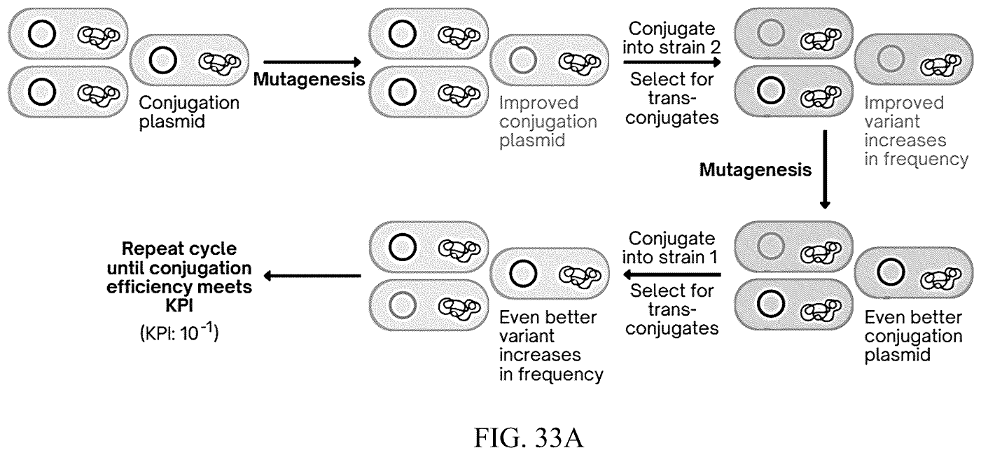

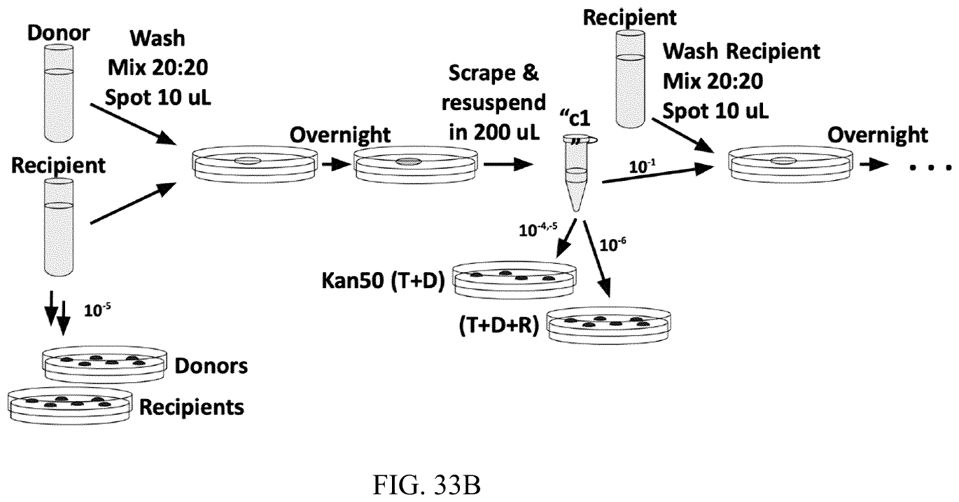

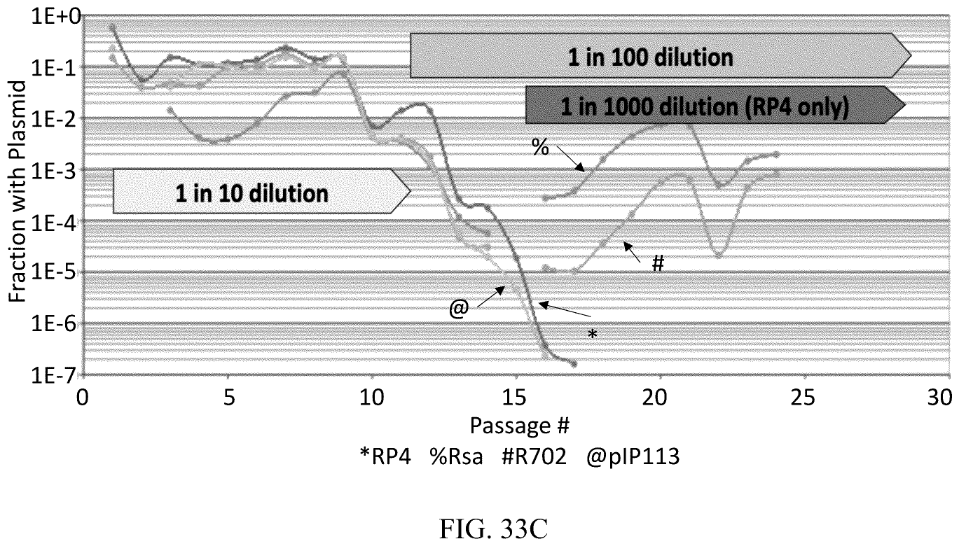

To improve transfer efficiency, directed evolution was performed on strains grown on medium plates (FIG. 33B). As illustrated in FIG. 33B, a culture of each plasmid containing strain in the appropriate antibiotic was started to theoretically select for 100% plasmid prevalence and 0% plasmid free cells. 0.5 μL of the stationary overnight was then passaged into 5 mL of LB media without antibiotics and grown overnight. Various dilutions of the overnight culture were sampled and plated onto LB agar plates with or without plasmid-selecting antibiotic to determine the number of plasmid containing cells versus the number of total cells, respectively. Sever passages were obtained by repeatedly passaging 0.5 μL of overnight into a new 5 mL culture (10,000× dilution). The fraction of plasmid containing cells at each passage was measured to calculate the rate of plasmid loss per passage.

Overnight cultures of a donor strain (containing the plasmid) and a plasmidless recipient strain were grown. The stationary phase overnight cultures were then pelleted and resuspended in LB media to remove the antibiotics. The washed cells were then mixed in a 1:10 ratio and spotted 10 μL of the mixture on an LB agar plate without antibiotics. The plate was incubated at 30 degree Celsius overnight until a lawn of bacteria grew from the dried spot. This lawn was scraped up and resuspended in 200 μL of LB media and plated at various dilutions onto LB agar plates with or without plasmid-selecting antibiotic in order to determine the number of plasmid containing cells versus the number of total cells, respectively. the resuspended lawn was then mixed with fresh, washed recipient cells in a 1:10 ratio and spotted 10 μL of the mixture on an LB agar plate. The plate was incubated overnight to form a new spot. The conjugation plasmid was continuously diluted and passaged in this way, and the prevalence of the plasmid after each passage was monitored. The dilution ratio was decreased from 1:10 (donor:recipient) to lower dilution ratios, such as 1:100 or 1:1000, until the prevalence of the plasmid was no longer stable and decreased steadily with each passage (FIG. 33C). The conjugation plasmids were then continued to be passaged until their prevalence dropped to 000 or until the trend in prevalence decrease changed; which suggested the appearance of a mutant with improved conjugation efficiency that allowed the conjugation plasmid to propagate faster than the rate ofdilution.

### Example 38: gRNA Sequence Analysis

Sequence analysis was performed to analyze the specificity of gRNA selected against Xanthomonas perforans. Nucleotide BLAST was performed on the sequence GGGAGTGCTATAATTCTGAG (SEQ ID NO: 90) while selecting for “Standard databases” and optimizing for “Highly similar sequences (megablast).” No additional parameters were selected for. All hits with up to two base pair mismatches are shown in Table 13, including 3 sequences from Xanthomonas perforans and 33 sequences from other non-bacterial organisms. The analysis demonstrates organisms at risk for non-target activity are non-bacterial and not compatible with conjugation.

TABLE 13
BLAST results for gRNA GGGAGTGCTATAATTCTGAG (SEQ ID NO: 90)

| | | | Query |
|---|---|---|---|
| Organism | Scientific Name | Description | Coverage |
| Bacteria, gram neg | Xanthomonas perforans | Xanthomonas perforans strain GEV872 | 100% |
| | | chromosome, complete genome | |
| Bacteria, gram neg | Xanthomonas perforans | Xanthomonas perforans 91-118, | 100% |
| | 91-118 | complete genome | |
| Bacteria, gram neg | Xanthomonas perforans | Xanthomonas perforans strain LH3 | 100% |
| | | chromosome, complete genome | |
| Worm, earthworm | Lumbricus terrestris | Lumbricus terrestris genome assembly, | 100% |
| | | chromosome: 3 | |
| Insect, hoverfly | Eristalinus sepulchralis | Eristalinus sepulchralis genome | 95% |
| | | assembly, chromosome: 1 | |
| Crustacea, sea louse | Caligus rogercresseyi | Caligus rogercresseyi isolate FCH | 90% |
| | | chromosome 7 | |
| Insect, mosquito | Anopheles stephensi | Anopheles stephensi strain Indian | 90% |
| | | chromosome 2R | |
| Insect, mosquito | Anopheles stephensi | Anopheles stephensi strain SDA-500 | 90% |
| | | chromosome 2R | |
| Mammal, cactus mouse | Peromyscus eremicus | Peromyscus eremicus genome | 90% |
| | | assembly, chromosome: 14 | |
| Nematode | Caenorhabditis briggsae | Caenorhabditis briggsae strain AF16 | 90% |
| | | chromosome I | |
| Fish, minnow | Phoxinus | Phoxinus genome assembly, | 90% |
| | | chromosome: 2 | |
| Fish, minnow | Phoxinus | Phoxinus genome assembly, | 90% |
| | | chromosome: 1 | |
| Fish, meagre | Argyrosomus regius | Argyrosomus regius genome assembly, | 90% |
| | | chromosome: 16 | |
| Insect, orange | Halyzia sedecimguttata | Halyzia sedecimguttata genome | 90% |
| ladybird | | assembly, chromosome: 8 | |
| Mollusc, clam | Hippopus | Hippopus genome assembly, | 90% |
| | | chromosome: 1 | |
| Insect, harlequin | Chironomus riparius | Chironomus riparius genome assembly, | 90% |
| fly | | chromosome: 1 | |
| Nematode | Caenorhabditis briggsae | Caenorhabditis briggsae isolate | 90% |
| | | QX1410_ONT chromosome I | |
| Insect, butterfly | Lysandra coridon | Lysandra coridon genome assembly, | 90% |
| | | chromosome: 47 | |
| Fish | Nibea albiflora | Nibea albiflora genome assembly, | 90% |
| | | chromosome: 14 | |
| Fish, striped bass | Morone saxatilis | PREDICTED: Morone saxatilis tet | 90% |
| | | methylcytosine dioxygenase 1 (tet1), | |
| | | mRNA | |
| Mammal, horse | Mus musculus | Mus musculus BAC clone RP24-90M17 | 90% |
| mouse | | from chromosome 15, complete | |
| | | sequence | |
| Plant | Arabis alpina | Arabis alpina genome assembly, | 90% |
| | | chromosome: 4 | |
| Mammal, dolphin | Lagenorhynchus | Lagenorhynchus albirostris genome | 90% |
| | albirostris | assembly, chromosome: 13 | |
| Mammal, whale | Hyperoodon ampullatus | Hyperoodon ampullatus genome | 90% |
| | | assembly, chromosome: 15 | |
| Fish, eel | Melanostigma | Melanostigma gelatinosum genome | 90% |
| | gelatinosum | assembly, chromosome: 19 | |
| Fish, eel | Melanostigma | Melanostigma gelatinosum genome | 90% |
| | gelatinosum | assembly, chromosome: 14 | |
| Mollusc, clam | Tridacna crocea | Tridacna crocea genome assembly, | 90% |
| | | chromosome: 7 | |
| Mammal, orca | Orcinus orca | Orcinus orca genome assembly, | 90% |
| | | chromosome: 13 | |
| Insect, moth | Plutella xylostella | Plutella xylostella genome assembly, | 90% |
| | | chromosome: 30 | |
| Mammal, lemur | Galeopterus varie gatus | PREDICTED: Galeopterus variegatus | 90% |
| | | alveolar macrophage chemotactic | |
| | | factor-like (LOC103591378), mRNA | |
| Insect, moth | Dryobotodes eremita | Dryobotodes eremita genome assembly, | 90% |
| | | chromosome: 5 | |
| Fish | Pholis gunnellus | Pholis gunnellus genome assembly, | 90% |
| | | chromosome: 19 | |
| Insect, moth | Chrysoteuchia culmella | Chrysoteuchia culmella genome | 90% |
| | | assembly, chromosome: 5 | |
| Insect, butterfly | Lysandra bellargus | Lysandra bellargus genome assembly, | 90% |
| | | chromosome: 31 | |
| Fish, mackarel | Trachurus | Trachurus genome assembly, | 90% |
| | | chromosome: 14 | |
| Mammal, horse mouse | Mus musculus | Mus musculus chromosome 15, clone | 90% |
| | | RP24-434C10, complete sequence | |

### Example 39: Modified Conjugation System Construction

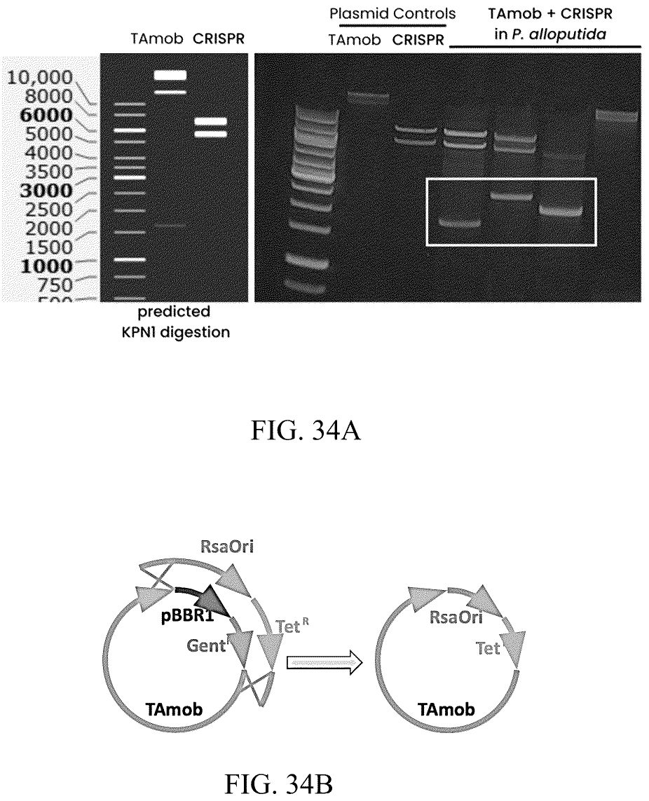

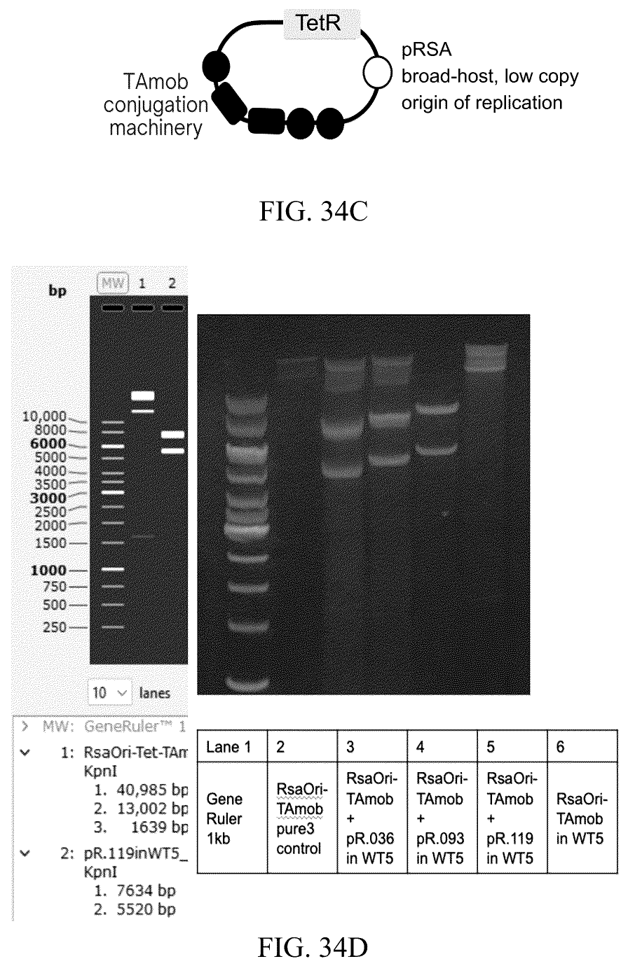

Plasmids for use in Horizontal Gene Transfer were modified to optimize stability of the vector, control expression of the gene targeting machinery, and allow for simple customization of the targeting nucleic acids. TAmob having a pBBR1 origin of replication demonstrate fragmentation after incorporation in to a bacterium. P. putida was transformed with a two-plasmid system as described in Example 7. Transformed bacteria and TAmob and CRISPR plasmid controls were digested with KPN1 and fragments were separated by electrophoresis. Results are shown in FIG. 34A. Transformed samples in lanes 3, 4, and 5 show expected bands between 4000 bp and 6000 bp representative of the CRISPR plasmid. Notably missing is a doublet at greater than 10,000 bp representing the TAmob plasmid. Samples in lanes 3, 4, and 5 (see white box) present a band between 1500 bp and 3000 bp, indicating truncation of the TAmob plasmid after transformation into a bacterium.

(1) Plasmid Construction without Excess Antibiotic Resistance Genes

TAmob conjugative plasmid previously derived from the E. coli RP4 conjugative plasmid was minimized to remove excess antibiotic resistance genes (ampicillin and tetracycline) and the origin of transfer (thus making the TAmob plasmid non-mobile). Further, the pBBR1 origin of replication was replaced with a pRSA origin of replication (FIG. 34B). The TAmob-RSA plasmid, comprising a low copy broad-host pRSA origin of replication and a tetracycline resistance marker were used to transform conjugation machinery (FIG. 34C). Approximately two to three copies of the pTAmob-RSA plasmid was expected to be maintained in the chassis.

The modified pTAmob-RSA plasmid showed notable stability through bacterial transformation. The size of the pTAmob-RSA plasmid was validated by performing a KPN1 digestion of samples comprising transformed pTAmob-RSA. FIG. 34D provides an image of the resulting fragment sizes. The left gel image shows the predicted KPN1 digested fragments for pTAmob-RSA and pR.119 (Cas9 plasmid). The right gel image shows the actual KPN1 digestions for different P. putida strains transformed with pTAmob-RSA and Cas9 plasmids (pR.036, pR.093, pR.119). Lanes 3, 4, and 6 show expected bands for pTAmob-RSA digest at approximately 13 and 41 kbp. Lane 5 shows an example of a failed pTAmob-RSA transformation (no pTAmob-RSA plasmid detected).

### (2) Cumate-Inducible Cas9-gRNA Plasmid

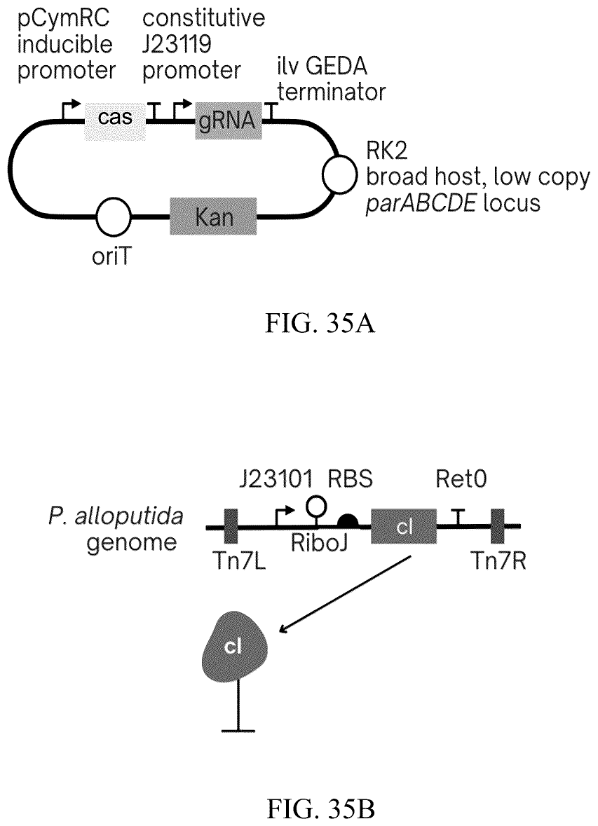

The cumate-inducible Cas9-gRNA template plasmid (pR.029) was constructed to include the gRNA template cassette (J23119 promoter, gRNA Spacer1 (random 20 nucleotide sequence flanked by BsaI sites), gRNA scaffold, and ilv GEDA terminator), the Cas9 cassette (pCymRC promoter, RBS, Cas9, and DT36 terminator), the CymR cassette (LacIQ promoter, CymR, and M13 terminator), the origin of transfer (oriT), the partitioning locus (parABCDE and trfA), and the low copy broad-host RK2 oriV origin of replication (FIG. 35A). The CymR transcriptional repressor protein is constitutively expressed and blocks transcription of Cas9. Cas9 expression can be titrated by supplementing cells with cuminic acid, however low levels of transcriptional “leakiness” are sufficient for functional Cas9 expression. Approximately two to five copies of the Cas9-gRNA plasmid is expected to be maintained in the microbial host.

(3) cI-Repressible Cas9-gRNA Plasmid and Genomic Integration

The cI-repressed template plasmid (pR.141) was constructed to include gRNA template cassette (J23119 promoter, gRNA Spacer1 (random 20 nucleotide sequence flanked by BsaI sites), gRNA scaffold, and ilv GEDA terminator), the Cas9 cassette (pCI promoter, RBS, Cas9, and DT36 terminator), the origin of transfer (oriT), the partitioning locus (parABCDE and trfA), and the RK2 oriV origin of replication (FIG. 35B). In addition to this plasmid, the cI transcriptional repressor cassette (Tn7 homology L arm-promoter-cI-terminator-Tn7 homology R arm) was genomically integrated onto the chassis genome using the miniaturized Tn7 transposon system, which inserts genes downstream of the glmS gene (FIG. 35B). The cI protein was constitutively expressed and blocked transcription of Cas9 when the Cas9-gRNA plasmid was in the chassis strain. After transfer of the Cas9-gRNA plasmid to a recipient gram negative bacteria in which cI protein is not expressed, Cas9 was expressed.

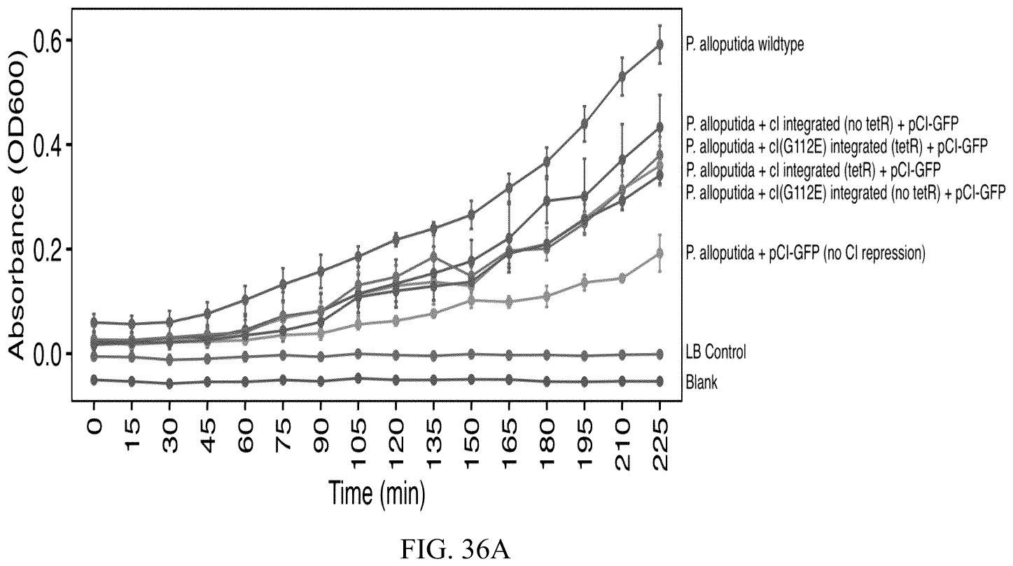

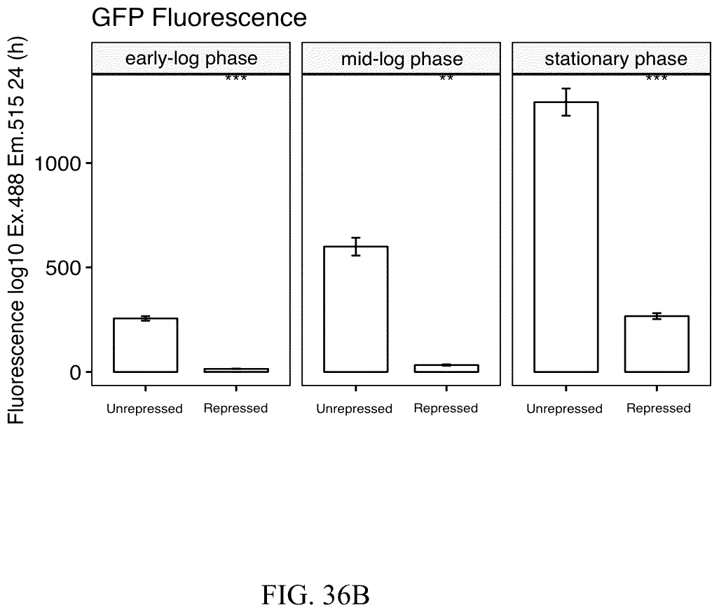

The validation of cI-repressor/activator system is shown in FIGS. 36A-36B. The cI repressor/activator system was built driving GFP as a control system. Top) Growth curves for P. putida wildtype (gray) compared to P. putida transformed with the pCI-GFP plasmid (purple) and four P. putida strains engineered with cI repressor integrated onto the genome and transformed with the pCI-GFP plasmid. Repressing GFP expression using the cI-repressor/activator system reduces metabolic burden of expressing GFP and increases P. putida growth rate. Bottom) When pCI-GFP plasmid is transformed into P. putida with the cI repressor integrated onto the genome, GFP is low/off (Repressed). When the same plasmid is transformed into P. putida with no cI repressor, GFP expression is high (Unrepressed), illustrating the off-to-on switch that GFP expression will undergo when the plasmid is transferred from the repressed donor cell to any non-repressed recipient cell.

### (4) Facile Plasmid Customization to Target Specific Pathogens

To construct the final Cas9-gRNA plasmids that target specific pathogens (pR.036 and pR.093), gRNA sequences were inserted into pR.029 in place of the gRNA Spacer1 using the BsaI sites and Golden Gate (type IIs restriction enzyme) assembly as described in Example 6. The gRNA sequences targeting specific pathogens were selected based on factors comprising GC content, percent match, off-target homology, and melting temperature.

### Example 40 Detection of GE P. putida with qPCR Assay

Primer Design and qPCR Parameters. Primers and probes were designed according to Thermo Fisher guidelines and target 100-150 bp fragments of DNA in pTAmob (at the engineered junction of the pBBR1 and gentR), Cas9, and the P. alloputida genome. Using a microbial database, a P. putida-specific primer was computationally designed to target a 20 nucleotide region that is unique to P. putida (rev primer) and a compatible fwd primer and probe were manually designed.

TABLE 14
Primer and probe sequences for detecting GE P. putida.

| | SEQ | | SEQ | | SEQ | |
|---|---|---|---|---|---|---|
| | ID | Fwd Primer | ID | Rev Primer | ID | TaqMan Probe |
| Target | NO | Sequence | NO | Sequence | NO | Sequence |
| pTAmob | 189 | GCGGCTACAGCCG | 192 | GATGGCGAGCCGT | 195 | ABY- |
| | | ATAGTCT | | TGGAG | | ACTTACGGGTTGCT |
| | | | | | | GCGCAACCCAAGTG |
| | | | | | | CTACCG-QSY |
| Cas9 | 190 | GGGAAACTGGAGA | 193 | CGCCTGTCTGTAC | 196 | 6FAM- |
| | | AATTGTCTGGGAT | | TTCTGTTTTCTTG | | GCGAGATTTTGCCA |
| | | AAA | | AC | | CAGTGCGCAAAGTA |
| | | | | | | TTGTCCATGCCCC- |
| | | | | | | MGB |
| P. putida | 191 | GGCAAGTCCAGTC | 194 | GTTTGCTGGGCAA | 197 | 6FAM- |
| genome | | ACAATGTCAGG | | CCAGCTG | | AAATGGCTCGACCC |
| | | | | | | GGTACTGCGCTGGC |
| | | | | | | CATA-MGB |

For qPCR reactions containing TaqMan probes, 10 μL reactions contained final concentrations of 1× TaqPath ProAmp Master Mix, 900 nM forward and reverse primers, 250 nM probe, and 0.1× template DNA. Thermocycling was performed on an Applied Biosystems QuantStudio 7 Pro in 96 well plates according to the following parameters: 50° C. for 2 min→95° C. for 5 min→40× cycles of 95° C. for 15 sec, 55° C. for 15 sec, 72° C. for 30 sec→95° C. for 15 sec, 60° C. for 1 min, continuous ramp at 0.15° C./s to 95° C.

Detecting GE P. putida P.048 in Complex Soil. Overnight cultures of P.048 were grown in LB with antibiotics and then diluted to a concentration of 1×10⁸ CFU/mL in 1×PBS. A serial dilution of P.048 was prepared in 1×PBS and then 1 mL of each dilution was mixed with approximately 1 g of complex soil from GCREC (exact weight recorded). As a negative control, a complex soil slurry was prepared with sterile PBS. Slurries were mixed with gentle shaking on a tube rocker for 1 hour at room temperature and then 250 μl of the resultant slurry was sampled and used as the starting material for a DNA extraction using the QIAGEN DNeasy PowerSoil kit according to the manufacturer's instructions. 200 μL of each slurry was also plated on LB agar with antibiotics to enumerate P.048 with the pTAmob or Cas9 plasmids. Effluent from the DNA extraction was then used as the DNA template in the qPCR reaction.

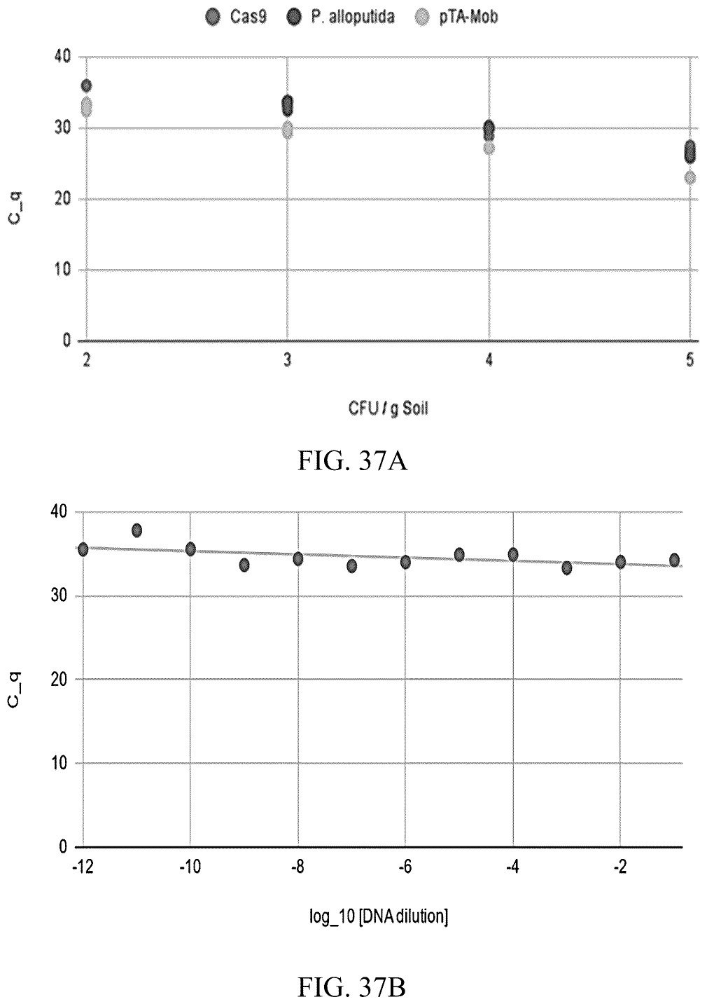

The qPCR LOD for all three primers+TaqMan probes is approximately 10² CFU/g complex soil. No non-specific amplification was detected from complex soil with sterile PBS using any of the three primers+TaqMan probes (FIG. 37A). An example of a no non-specific amplification readout is provided using pTAmob primers+TaqMan probe without GE P. putida (FIG. 37B).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A modified bacteria, wherein the modified bacteria comprises:
a first expression vector comprising a first exogenous nucleic acid, wherein the first exogenous nucleic acid comprises:
a sequence encoding for an endonuclease; and
a sequence encoding for at least one guide nucleic acid, wherein the at least one guide nucleic acid targets a sequence in a genome of one or more species of plant-associated bacteria; and
a second expression vector comprising a second exogenous nucleic acid, wherein
the second exogenous nucleic acid encodes for a bacterial conjugation machinery,
wherein the first expression vector and the second expression vector each independently comprises a low copy origin of replication.

2. The modified bacteria of claim 1, wherein the at least one guide nucleic acid comprises at least 90% sequence identity to the sequence in the genome of one or more species of plant-associated bacteria.

3. The modified bacteria of claim 2, wherein the at least one guide nucleic acid comprises less than 90% sequence identity to a sequence in a bacterial species other than that of the one or more species of plant-associated bacteria.

4. The modified bacteria of claim 2, wherein the at least one guide nucleic acid comprises 100% sequence identity to the sequence in the genome of one or more species of plant-associated bacteria.

5. The modified bacteria of claim 1, wherein the sequence in the genome of one or more species of plant-associated bacteria is in a region of an essential gene.

6. The modified bacteria of claim 5, wherein the essential gene comprises dnaA, gyrA, polA, or ftsZ.

7. The modified bacteria of claim 1, wherein the first expression vector does not replicate autonomously.

8. The modified bacteria of claim 7, wherein the first expression vector comprises an oriV origin of replication.

9. The modified bacteria of claim 7, wherein the first expression vector further comprises deletion or mutation of a sequence encoding for plasmid replication initiator protein (TrfA).

10. The modified bacteria of claim 7, wherein the second expression vector comprises a sequence encoding for plasmid replication initiator protein (TrfA).

11. The modified bacteria of claim 10, wherein the TrfA comprises a sequence comprising at least 80%, 85%, 90%, 95%, 97%, 98%, 99%, or 100% sequence identity to SEQ ID NO: 46.

12. The modified bacteria of claim 1, wherein the sequence in the genome of the one or more species of plant-associated bacteria is in a region of a non-essential gene.

13. The modified bacteria of claim 1, wherein the sequence in the genome of the one or more species of plant-associated bacteria is in a non-coding region of the genome.

14. The modified bacteria of claim 1, wherein the plant-associated bacteria is a soil bacteria.

15. The modified bacteria of claim 1, wherein the plant-associated bacteria is a plant bacteria.

16. The modified bacteria of claim 15, wherein the plant bacteria is a plant pathogenic bacteria.

17. The modified bacteria of claim 1, wherein the second expression vector is a conjugative plasmid, and wherein the conjugative plasmid is a TAmob plasmid, an RP4 plasmid, an Rsa plasmid, an R702 plasmid, or an IP113 plasmid.

18. The modified bacteria of claim 17, wherein the conjugative plasmid is the TAmob plasmid.

19. The modified bacteria of claim 1, further comprising a genome modification comprising a domain essential to the replication of at least one of the first or second expression vectors.

20. The modified bacteria of claim 19, wherein the domain essential to the replication of at least one of the first or second expression vectors comprises a pirA gene, wherein the pirA gene comprises a sequence having at least 80%, 85%, 90%, 95%, 97%, 98%, 99%, or 100% sequence identity to SEQ ID NO: 49.

21. A method of horizontal gene transfer (HGT), wherein the method of HGT comprises introducing the modified bacteria of claim 1 to an ecosystem of a plant.
